SEQUENCE LISTING (415 sequences)

SEQ ID NO: 1 - protein, 22 aa, synthetic construct
CRRTSTGQISTLRVNITAPLSQ

SEQ ID NO: 2 - protein, 15 aa, synthetic construct
RTSTGDITSLRVITA

SEQ ID NO: 3 - protein, 19 aa, synthetic construct
TSTGQISTLRVNITAPLSQ

SEQ ID NO: 4 - protein, 20 aa, synthetic construct
STLRVNITAPLSQRYRVRIR

SEQ ID NO: 5 - protein, 18 aa, synthetic construct
STGQISTLRVNITAPLSQ

SEQ ID NO: 6 - protein, 18 aa, synthetic construct
STGQISTLAVNITAPLSQ

SEQ ID NO: 7 - protein, 18 aa, synthetic construct
STGQISTLRVNITAALSQ

SEQ ID NO: 8 - protein, 22 aa, synthetic construct
CHHTSTGQISTLRVNITAPLSQ

SEQ ID NO: 9 - protein, 18 aa, synthetic construct
STGQISTARVNITAPLSQ

SEQ ID NO: 10 - protein, 11 aa, synthetic construct
QISTLRVNITA

SEQ ID NO: 11 - protein, 20 aa, synthetic construct
QTRTVPMPKIHHPPWQNVVP

SEQ ID NO: 12 - protein, 20 aa, synthetic construct
HHHQVYQVRSHWTGMHSGHD

SEQ ID NO: 13 - protein, 21 aa, synthetic construct
WNLPASFHNHHIRPHEHEWIQ

SEQ ID NO: 14 - protein, 20 aa, synthetic construct
SSYHHFKMPELHFGKNTFHQ

SEQ ID NO: 15 - protein, 22 aa, synthetic construct
CRRTSTGQISTARVNITAPLSQ

SEQ ID NO: 16 - protein, 15 aa, synthetic construct
YSAYQCWCWRQQGTS

SEQ ID NO: 17 - protein, 15 aa, synthetic construct
YHQYSCWCWRPPGPY

SEQ ID NO: 18 - protein, 15 aa, synthetic construct
YASYHCWCWRDPGRS

SEQ ID NO: 19 - protein, 15 aa, synthetic construct
TAYWNCWCWREKAGQ

SEQ ID NO: 20 - protein, 15 aa, synthetic construct
TYGYSCWCWRQHPWT

SEQ ID NO: 21 - protein, 15 aa, synthetic construct
STAYHCWCWREPYGN

SEQ ID NO: 22 - protein, 15 aa, synthetic construct
TTRYSCWCWRDDAYA

SEQ ID NO: 23 - protein, 12 aa, synthetic construct
HYPFRPHANQAS

SEQ ID NO: 24 - protein, 12 aa, synthetic construct
WHRSYYTWNLNT

SEQ ID NO: 25 - protein, 12 aa, synthetic construct
WHFSYNWRWLPP

SEQ ID NO: 26 - protein, 12 aa, synthetic construct
DYHDPSLPTLRK

SEQ ID NO: 27 - protein, 18 aa, synthetic construct
FNWDYSWKSERLKEAYDL

SEQ ID NO: 28 - protein, 17 aa, synthetic construct
FNWDYSLEELREKAKYK

SEQ ID NO: 29 - protein, 18 aa, synthetic construct
TEKDYRHGNIRMKLAYDL

SEQ ID NO: 30 - protein, 15 aa, synthetic construct
GNWDYNSQRAQLYNQ

SEQ ID NO: 31 - protein, 11 aa, synthetic construct
LDYVNRRKMYQ

SEQ ID NO: 32 - protein, 12 aa, synthetic construct
NYSKPTDRQYHF

SEQ ID NO: 33 - protein, 12 aa, synthetic construct
KHAHHTHNLRLP

SEQ ID NO: 34 - protein, 12 aa, synthetic construct
AAKMDGHLHGGQ

SEQ ID NO: 35 - protein, 12 aa, synthetic construct
MRNRERYPKPYY

SEQ ID NO: 36 - protein, 12 aa, synthetic construct
TYLQRPSTNLER

SEQ ID NO: 37 - protein, 14 aa, synthetic construct
SCFPNWSLRPMNQM

SEQ ID NO: 38 - protein, 20 aa, synthetic construct
MDEKAQKGPAKLVFFACEKG

SEQ ID NO: 39 - protein, 14 aa, synthetic construct
SCFPNWSLRPMNQM

SEQ ID NO: 40 - protein, 20 aa, synthetic construct
MDEKAQKGPAKLVFFACEKG

SEQ ID NO: 41 - protein, 15 aa, synthetic construct
MLNWNRLSPSNQTEK

SEQ ID NO: 42 - protein, 12 aa, synthetic construct
GADYKRITVKVN

SEQ ID NO: 43 - protein, 9 aa, synthetic construct
CNMHTPMVC

SEQ ID NO: 44 - protein, 9 aa, synthetic construct
CNWMINKEC

SEQ ID NO: 45 - protein, 9 aa, synthetic construct
CVPMTYRAC

SEQ ID NO: 46 - protein, 12 aa, synthetic construct
ADFRVMADMSMY

SEQ ID NO: 47 - protein, 12 aa, synthetic construct
SHTTTEHAKDKI

SEQ ID NO: 48 - protein, 13 aa, synthetic construct
GLIPLTTHMHIGK

SEQ ID NO: 49 - protein, 12 aa, synthetic construct
TFLNSVPTYSYW

SEQ ID NO: 50 - protein, 12 aa, synthetic construct
SHDTRSPFTWGR

SEQ ID NO: 51 - protein, 12 aa, synthetic construct
GSWNTFRAQPTI

SEQ ID NO: 52 - protein, 12 aa, synthetic construct
SFAGVHQFRPAP

SEQ ID NO: 53 - protein, 12 aa, synthetic construct
ALWPSSSMSSTI

SEQ ID NO: 54 - protein, 12 aa, synthetic construct
HDAFPRPHLFRN

SEQ ID NO: 55 - protein, 12 aa, synthetic construct
LTPHKHHKHLHA

SEQ ID NO: 56 - protein, 13 aa, synthetic construct
CKWYSHSKEKIQN

SEQ ID NO: 57 - protein, 24 aa, Homo sapiens
GVLLTQRTLLSLVLALLFPSMASM

SEQ ID NO: 58 - protein, 15 aa, synthetic construct
KCLLYLAFLFIGVNC

SEQ ID NO: 59 - protein, 20 aa, Mus musculus
ETDTLLLWVLLLWVPGSTGD

SEQ ID NO: 60 - protein, 18 aa, Homo sapiens
GWSCIILFLVATATGVHS

SEQ ID NO: 61 - protein, 16 aa, synthetic construct
RAWIFFLLCLAGRALA

SEQ ID NO: 62 - protein, 20 aa, synthetic construct
WWRLWWLLLLLLLLWPMVWA

SEQ ID NO: 63 - protein, 21 aa, Homo sapiens
DMRVPAQLLGLLLLWLRGARC

SEQ ID NO: 64 - protein, 15 aa, synthetic construct
PLLLLLPLLWAGALA

SEQ ID NO: 65 - protein, 22 aa, synthetic construct
DAMKRGLCCVLLLCGAVFVSPS

SEQ ID NO: 66 - protein, 34 aa, synthetic construct
DAMKRGLCCVLLLCGAVFVSPSQEIHARFRRGAR

SEQ ID NO: 67 - protein, 17 aa, Homo sapiens
AFLWLLSCWALLGTTFG

SEQ ID NO: 68 - protein, 14 aa, Homo sapiens
NLLLILTFVAAAVA

SEQ ID NO: 69 - protein, 19 aa, Homo sapiens
YRMQLLSCIALSLALVTNS

SEQ ID NO: 70 - protein, 16 aa, synthetic construct
GVKVLFALICIAVAEA

SEQ ID NO: 71 - protein, 15 aa, synthetic construct
KWVTFISLLFSSAYS

SEQ ID NO: 72 - protein, 15 aa, synthetic construct
KTIIALSYIFCLVLG

SEQ ID NO: 73 - protein, 23 aa, Homo sapiens
ALWMRLLPLLALLALWGPDPAAA

SEQ ID NO: 74 - protein, 8 aa, synthetic construct
KPIFLVLL

SEQ ID NO: 75 - protein, 21 aa, synthetic construct
LGPCMLLLLLLLGLRLQLSLG

SEQ ID NO: 76 - protein, 22 aa, synthetic construct
RPTWAWWLFLVLLLALWAPARG

SEQ ID NO: 77 - protein, 27 aa, Homo sapiens
AGPLRAPLLLLAILAVALAVSPAAGSS

SEQ ID NO: 78 - protein, 19 aa, synthetic construct
HKLVFLVLLFLGALGLCLA

SEQ ID NO: 79 - protein, 26 aa, synthetic construct
GVHECPAWLWLLLSLLSLPLGLPVLG

SEQ ID NO: 80 - protein, 22 aa, Homo sapiens
ERMLPLLALGLLAAGFCPAVLC

SEQ ID NO: 81 - protein, 16 aa, synthetic construct
HLGIWTLLPLVLTSVA

SEQ ID NO: 82 - protein, 27 aa, Homo sapiens
NIKGSPWKGSLLLLLVSNLLLCQSVAP

SEQ ID NO: 83 - protein, 15 aa, synthetic construct
RLAVVCLCLFGLASC

SEQ ID NO: 84 - protein, 22 aa, synthetic construct
GRMLPLLALLLLAAGFCPAVLA

SEQ ID NO: 85 - protein, 20 aa, synthetic construct
RSLSVLALLLLLLLAPASAA

SEQ ID NO: 86 - protein, 21 aa, synthetic construct
KSLSALVLLLLLLLLPGALAA

SEQ ID NO: 87 - protein, 24 aa, synthetic construct
RGAALVLLLLLLLLLALALAAPVP

SEQ ID NO: 88 - protein, 22 aa, synthetic construct
RGAALVLLLLLLLLLAGVLAAP

SEQ ID NO: 89 - protein, 20 aa, synthetic construct
RGAALVLLLLLLLLLSPALA

SEQ ID NO: 90 - protein, 20 aa, synthetic construct
RSLSVLALLLLLLLAPASAA

SEQ ID NO: 91-92 - skipped (2 entries)

SEQ ID NO: 93 - protein, 4 aa, synthetic construct
RAKR

SEQ ID NO: 94 - protein, 4 aa, synthetic construct
RRRR

SEQ ID NO: 95 - protein, 4 aa, synthetic construct
RKRR

SEQ ID NO: 96 - protein, 4 aa, synthetic construct
RRKR

SEQ ID NO: 97 - protein, 4 aa, synthetic construct
RKKR

SEQ ID NO: 98 - protein, 8 aa, synthetic construct
  SITE 2: note = misc_feature - X can be any naturally occurring amino acid
  SITE 4: note = misc_feature - X can be any naturally occurring amino acid
DXEXNPGP

SEQ ID NO: 99 - protein, 8 aa, synthetic construct
DVEENPGP

SEQ ID NO: 100 - protein, 35 aa, synthetic construct
PSDARHKQRIVAPAKQLLNFDLLKLAGDVESNPGP

SEQ ID NO: 101 - protein, 35 aa, Avisivirus A
ARRTLEWARREVGAIDETDHKDILLGGDIEENPGP

SEQ ID NO: 102 - protein, 35 aa, Avihepatovirus A
  REGION 1..35: note = Avihepatovirus: Duck hepatitis A virus
RLKTLAFELNLEIESDQIRNKKDLTTEGVEPNPGP

SEQ ID NO: 103 - protein, 35 aa, synthetic construct
VREENVFGLYRIFNAHYAGYFADLLIHDIETNPGP

SEQ ID NO: 104 - protein, 35 aa, Cosavirus A
IMADSVLPRPLTRAERDVARDLLLIAGDIESNPGP

SEQ ID NO: 105 - protein, 35 aa, synthetic construct
SEPIPEATLSTILSEGATNFSLLKLAGDVELNPGP

SEQ ID NO: 106 - protein, 35 aa, synthetic construct
RYKNARAWCPSMLPFRSYKQKMLMQSGDIETNPGP

SEQ ID NO: 107 - protein, 35 aa, Hunnivirus A
CPRPGMAIDPPAQSSGATNFSLLRLAGDVELNPGP

SEQ ID NO: 108 - protein, 35 aa, Kunsagivirus A
SPRSLLHFLIGRPRPRVPPSPSLLLSGDVEPNPGP

SEQ ID NO: 109 - protein, 35 aa, Mischivirus A
DSYPASGEEEEDDFHDMEDHSDILLGGDVEENPGP

SEQ ID NO: 110 - protein, 35 aa, Mosavirus A
TNSRAKLMVDEDYVIQRSAHRSVLLDGDVESNPGP

SEQ ID NO: 111 - protein, 35 aa, Pasivirus A
DIPSFQRDFINWLGSKEELQNMILQCGDVEQNPGP

SEQ ID NO: 112 - protein, 35 aa, synthetic construct
EGLSSAMTVMAFQGPGATNFSLLKQAGDVEENPGP

SEQ ID NO: 113 - protein, 35 aa, synthetic construct
NYPLVPSIGNVARTLTRAEIEDELIRAGIESNPGP

SEQ ID NO: 114 - protein, 35 aa, synthetic construct
RSRRLRGPRPQNLGVRAEGRGSLLTCGDVEENPGP

SEQ ID NO: 115 - protein, 35 aa, synthetic construct
FQQWKLVSSNDECRAFLRKRTQLLMSGDVESNPGP

SEQ ID NO: 116 - protein, 35 aa, Rotavirus C
LKKHNGAGYPLIVANSKFQIDKILISGDIELNPGP

SEQ ID NO: 117 - protein, 35 aa, synthetic construct
TDFLSMTAFDFQQAVFRSNYDLLKLCGDVESNPGP

SEQ ID NO: 118 - protein, 22 aa, synthetic construct
VKQTLNFDLLKLAGDVESNPGP

SEQ ID NO: 119 - protein, 21 aa, synthetic construct
GSGEGRGSLLTCGDVEENPGP

SEQ ID NO: 120 - protein, 22 aa, synthetic construct
GSGATNFSLLKQAGDVEENPGP

SEQ ID NO: 121 - protein, 23 aa, synthetic construct
GSGQCTNYALLKLAGDVESNPGP

SEQ ID NO: 122 - protein, 25 aa, synthetic construct
GSGVKQTLNFDLLKLAGDVESNPGP

SEQ ID NO: 123 - protein, 26 aa, synthetic construct
RAKRGSGATNFSLLKQAGDVEENPGP

SEQ ID NO: 124 - protein, 26 aa, synthetic construct
RRRRGSGATNFSLLKQAGDVEENPGP

SEQ ID NO: 125 - protein, 26 aa, synthetic construct
RKRRGSGATNFSLLKQAGDVEENPGP

SEQ ID NO: 126 - protein, 26 aa, synthetic construct
RRKRGSGATNFSLLKQAGDVEENPGP

SEQ ID NO: 127 - protein, 26 aa, synthetic construct
RKKRGSGATNFSLLKQAGDVEENPGP

SEQ ID NO: 128 - DNA, 67 bp, synthetic construct
  source 1..67: mol_type = other DNA
aaaaaatgaaaataaatacaaaggttcttgagggttgtgttaaattgaaagcgagaaataatcataa

SEQ ID NO: 129 - DNA, 70 bp, synthetic construct
  source 1..70: mol_type = other DNA
aaaaaatgaaaataaatacaaaggttcttgagggttgtgttaaattgaaagcgagaaataatcataaatt

SEQ ID NO: 130 - DNA, 67 bp, synthetic construct
  source 1..67: mol_type = other DNA
aaaaattgaaaataaatacaaaggttcttgagggttgtgttaaattgaaagcgagaaataatcataa

SEQ ID NO: 131 - DNA, 70 bp, synthetic construct
  source 1..70: mol_type = other DNA
aaaaattgaaaataaatacaaaggttcttgagggttgtgttaaattgaaagcgagaaataatcataaata

SEQ ID NO: 132 - DNA, 70 bp, synthetic construct
  source 1..70: mol_type = other DNA
aaaaaatgaaaataaatacaaaggttcttgagggttgtgttaaattgaaagcgagaaataatcataaata

SEQ ID NO: 133 - DNA, 15 bp, synthetic construct
  source 1..15: mol_type = other DNA
aaagtagaaaatata

SEQ ID NO: 134 - DNA, 15 bp, synthetic construct
  source 1..15: mol_type = other DNA
tatccggagacgtca

SEQ ID NO: 135 - DNA, 15 bp, synthetic construct
  source 1..15: mol_type = other DNA
attactgaattaata

SEQ ID NO: 136 - DNA, 15 bp, synthetic construct
  source 1..15: mol_type = other DNA
gcaacgtaaaacaca

SEQ ID NO: 137 - DNA, 15 bp, synthetic construct
  source 1..15: mol_type = other DNA
aaaaaataaaaaaaa

SEQ ID NO: 138 - DNA, 15 bp, synthetic construct
  source 1..15: mol_type = other DNA
agtaaagaaaaagaa

SEQ ID NO: 139 - DNA, 15 bp, synthetic construct
  source 1..15: mol_type = other DNA
aaaattgataaataa

SEQ ID NO: 140 - DNA, 15 bp, synthetic construct
  source 1..15: mol_type = other DNA
aaattagacatttga

SEQ ID NO: 141 - DNA, 15 bp, synthetic construct
  source 1..15: mol_type = other DNA
ataactgaaatgaaa

SEQ ID NO: 142 - DNA, 15 bp, synthetic construct
  source 1..15: mol_type = other DNA
aaagatgaaaaagta

SEQ ID NO: 143 - DNA, 15 bp, synthetic construct
  source 1..15: mol_type = other DNA
attaatgaaataata

SEQ ID NO: 144 - DNA, 15 bp, synthetic construct
  source 1..15: mol_type = other DNA
aaaaatgaaaatgga

SEQ ID NO: 145 - DNA, 15 bp, synthetic construct
  source 1..15: mol_type = other DNA
aaaacataaaaatta

SEQ ID NO: 146 - DNA, 15 bp, synthetic construct
  source 1..15: mol_type = other DNA
ataacataaaaataa

SEQ ID NO: 147 - DNA, 15 bp, synthetic construct
  source 1..15: mol_type = other DNA
aagatagatttccta

SEQ ID NO: 148 - DNA, 15 bp, synthetic construct
  source 1..15: mol_type = other DNA
aaaaatgaaaaaata

SEQ ID NO: 149 - DNA, 15 bp, synthetic construct
  source 1..15: mol_type = other DNA
gaaaaagaaattcct

SEQ ID NO: 150 - DNA, 15 bp, synthetic construct
  source 1..15: mol_type = other DNA
aatggtgaaaaaatg

SEQ ID NO: 151 - DNA, 15 bp, synthetic construct
  source 1..15: mol_type = other DNA
aaaacataaaaataa

SEQ ID NO: 152 - DNA, 15 bp, synthetic construct
  source 1..15: mol_type = other DNA
ataattgtaaaaaca

SEQ ID NO: 153 - DNA, 15 bp, synthetic construct
  source 1..15: mol_type = other DNA
ataattgaaaatgga

SEQ ID NO: 154 - DNA, 15 bp, synthetic construct
  source 1..15: mol_type = other DNA
aaaaatttaattaca

SEQ ID NO: 155 - DNA, 15 bp, synthetic construct
  source 1..15: mol_type = other DNA
aaaagtgaaaaacaa

SEQ ID NO: 156 - DNA, 15 bp, synthetic construct
  source 1..15: mol_type = other DNA
aaaaaagaaaataga

SEQ ID NO: 157 - DNA, 15 bp, synthetic construct
  source 1..15: mol_type = other DNA
gtagaagaaaataat

SEQ ID NO: 158 - DNA, 15 bp, synthetic construct
  source 1..15: mol_type = other DNA
aaaaatgaaacgtaa

SEQ ID NO: 159 - DNA, 15 bp, synthetic construct
  source 1..15: mol_type = other DNA
aaaaaacaaaatgaa

SEQ ID NO: 160 - DNA, 15 bp, synthetic construct
  source 1..15: mol_type = other DNA
gagacagtagtttta

SEQ ID NO: 161 - DNA, 15 bp, synthetic construct
  source 1..15: mol_type = other DNA
aaaaatgataaaata

SEQ ID NO: 162 - DNA, 15 bp, synthetic construct
  source 1..15: mol_type = other DNA
aataatgaaaaaata

SEQ ID NO: 163 - DNA, 15 bp, synthetic construct
  source 1..15: mol_type = other DNA
acaaaagtgaatata

SEQ ID NO: 164 - DNA, 15 bp, synthetic construct
  source 1..15: mol_type = other DNA
ttaaatgaaaatata

SEQ ID NO: 165 - DNA, 15 bp, synthetic construct
  source 1..15: mol_type = other DNA
aataatgaaaaaaca

SEQ ID NO: 166 - DNA, 15 bp, synthetic construct
  source 1..15: mol_type = other DNA
taaagtgaaaatata

SEQ ID NO: 167 - DNA, 15 bp, synthetic construct
  source 1..15: mol_type = other DNA
attaatgaaaagtta

SEQ ID NO: 168 - DNA, 15 bp, synthetic construct
  source 1..15: mol_type = other DNA
ataacaaaaataaaa

SEQ ID NO: 169 - DNA, 15 bp, synthetic construct
  source 1..15: mol_type = other DNA
aaaaatgataagata

SEQ ID NO: 170 - DNA, 15 bp, synthetic construct
  source 1..15: mol_type = other DNA
aaaactgtaacacga

SEQ ID NO: 171 - DNA, 15 bp, synthetic construct
  source 1..15: mol_type = other DNA
aaaactgaaaatata

SEQ ID NO: 172 - DNA, 15 bp, synthetic construct
  source 1..15: mol_type = other DNA
taaagtgaacaataa

SEQ ID NO: 173 - DNA, 15 bp, synthetic construct
  source 1..15: mol_type = other DNA
aaaagggaaatttga

SEQ ID NO: 174 - DNA, 15 bp, synthetic construct
  source 1..15: mol_type = other DNA
agaattgaaaacgaa

SEQ ID NO: 175 - DNA, 15 bp, synthetic construct
  source 1..15: mol_type = other DNA
aaaaatgaaaataaa

SEQ ID NO: 176 - DNA, 15 bp, synthetic construct
  source 1..15: mol_type = other DNA
gtaaatgaaaaaaaa

SEQ ID NO: 177 - DNA, 15 bp, synthetic construct
  source 1..15: mol_type = other DNA
gaaaatgaaaaggta

SEQ ID NO: 178 - DNA, 15 bp, synthetic construct
  source 1..15: mol_type = other DNA
aaaactgatgaaata

SEQ ID NO: 179 - DNA, 15 bp, synthetic construct
  source 1..15: mol_type = other DNA
aaaaatgaaatgata

SEQ ID NO: 180 - DNA, 15 bp, synthetic construct
  source 1..15: mol_type = other DNA
aataatgaaaacaaa

SEQ ID NO: 181 - DNA, 15 bp, synthetic construct
  source 1..15: mol_type = other DNA
aattctgaaactaga

SEQ ID NO: 182 - DNA, 15 bp, synthetic construct
  source 1..15: mol_type = other DNA
aaaattgaattgcga

SEQ ID NO: 183 - DNA, 15 bp, synthetic construct
  source 1..15: mol_type = other DNA
taaagtgaaaatcta

SEQ ID NO: 184 - DNA, 15 bp, synthetic construct
  source 1..15: mol_type = other DNA
gcaatagaaaagatg

SEQ ID NO: 185 - DNA, 15 bp, synthetic construct
  source 1..15: mol_type = other DNA
aagaatgaaataaca

SEQ ID NO: 186 - DNA, 15 bp, synthetic construct
  source 1..15: mol_type = other DNA
aaaaatgtaataacg

SEQ ID NO: 187 - DNA, 15 bp, synthetic construct
  source 1..15: mol_type = other DNA
aaagtcgaaaaagaa

SEQ ID NO: 188 - DNA, 15 bp, synthetic construct
  source 1..15: mol_type = other DNA
aaaacataaatataa

SEQ ID NO: 189 - DNA, 15 bp, synthetic construct
  source 1..15: mol_type = other DNA
aatatggaaaactaa

SEQ ID NO: 190 - DNA, 15 bp, synthetic construct
  source 1..15: mol_type = other DNA
aaaaatgaattaata

SEQ ID NO: 191 - DNA, 15 bp, synthetic construct
  source 1..15: mol_type = other DNA
aaaattgaagtaata

SEQ ID NO: 192 - DNA, 15 bp, synthetic construct
  source 1..15: mol_type = other DNA
aatacttaaaatgta

SEQ ID NO: 193 - DNA, 15 bp, synthetic construct
  source 1..15: mol_type = other DNA
aaaatataaaataaa

SEQ ID NO: 194 - DNA, 15 bp, synthetic construct
  source 1..15: mol_type = other DNA
aaaaatgaactctta

SEQ ID NO: 195 - DNA, 15 bp, synthetic construct
  source 1..15: mol_type = other DNA
aaaatagaataagta

SEQ ID NO: 196 - DNA, 15 bp, synthetic construct
  source 1..15: mol_type = other DNA
ataaatgaaaagata

SEQ ID NO: 197 - DNA, 15 bp, synthetic construct
  source 1..15: mol_type = other DNA
aaaactgaaaataaa

SEQ ID NO: 198 - DNA, 15 bp, synthetic construct
  source 1..15: mol_type = other DNA
aaattgtaaaaaata

SEQ ID NO: 199 - DNA, 15 bp, synthetic construct
  source 1..15: mol_type = other DNA
aaatattaaaaaaaa

SEQ ID NO: 200 - DNA, 15 bp, synthetic construct
  source 1..15: mol_type = other DNA
gaaataaaaaacata

SEQ ID NO: 201 - DNA, 15 bp, synthetic construct
  source 1..15: mol_type = other DNA
aaaaataaaaatata

SEQ ID NO: 202 - DNA, 15 bp, synthetic construct
  source 1..15: mol_type = other DNA
aattttgtaaaaata

SEQ ID NO: 203 - DNA, 15 bp, synthetic construct
  source 1..15: mol_type = other DNA
attacatattatata

SEQ ID NO: 204 - DNA, 15 bp, synthetic construct
  source 1..15: mol_type = other DNA
aaaacttaaaattta

SEQ ID NO: 205 - DNA, 15 bp, synthetic construct
  source 1..15: mol_type = other DNA
ataaaaattaaaaaa

SEQ ID NO: 206 - DNA, 15 bp, synthetic construct
  source 1..15: mol_type = other DNA
atatctaaaaatctt

SEQ ID NO: 207 - DNA, 15 bp, synthetic construct
  source 1..15: mol_type = other DNA
aaaaataatgaccaa

SEQ ID NO: 208 - DNA, 15 bp, synthetic construct
  source 1..15: mol_type = other DNA
attattcaaaatatg

SEQ ID NO: 209 - DNA, 15 bp, synthetic construct
  source 1..15: mol_type = other DNA
gaaaatgaaaatata

SEQ ID NO: 210 - DNA, 15 bp, synthetic construct
  source 1..15: mol_type = other DNA
aaaacataaaaaaca

SEQ ID NO: 211 - DNA, 15 bp, synthetic construct
  source 1..15: mol_type = other DNA
aagattgaaattata

SEQ ID NO: 212 - DNA, 15 bp, synthetic construct
  source 1..15: mol_type = other DNA
aaatatgtaaatatg

SEQ ID NO: 213 - DNA, 15 bp, synthetic construct
  source 1..15: mol_type = other DNA
aaaactgatattata

SEQ ID NO: 214 - DNA, 15 bp, synthetic construct
  source 1..15: mol_type = other DNA
ataaatgtagactct

SEQ ID NO: 215 - DNA, 15 bp, synthetic construct
  source 1..15: mol_type = other DNA
taaactgaagtttaa

SEQ ID NO: 216 - DNA, 50 bp, synthetic construct
  source 1..50: mol_type = other DNA
atttttataccgaacataaaaataaggttaattattaataccataaaatc

SEQ ID NO: 217 - DNA, 50 bp, synthetic construct
  source 1..50: mol_type = other DNA
ggatttttaatagagtgaagtgatataggattattcttttaacaaataaa

SEQ ID NO: 218 - DNA, 50 bp, synthetic construct
  source 1..50: mol_type = other DNA
attctagaatcgttgatagaacaggatgtataagtttttatgttaactaa

SEQ ID NO: 219 - DNA, 50 bp, synthetic construct
  source 1..50: mol_type = other DNA
tttgtatcatttgtccatcaacgtcatttcaataatattggatgatataa

SEQ ID NO: 220 - DNA, 50 bp, synthetic construct
  source 1..50: mol_type = other DNA
actaaagagttaaataagtcgagatagttttatatcacttaaatattaaa

SEQ ID NO: 221 - DNA, 50 bp, synthetic construct
  source 1..50: mol_type = other DNA
gtgcctaatattactatatcaagtaatgctgaataaaaatatttataaat

SEQ ID NO: 222 - DNA, 50 bp, synthetic construct
  source 1..50: mol_type = other DNA
ttctactactattgatatatttgtatttaaaagttgtttggtgaacttaa

SEQ ID NO: 223 - DNA, 50 bp, synthetic construct
  source 1..50: mol_type = other DNA
atacaactaggactttgtcacatattctttgatctaatttttagatataa

SEQ ID NO: 224 - DNA, 50 bp, synthetic construct
  source 1..50: mol_type = other DNA
tgtgatatgtgataaattaactacaaaattaaatagaatagtaaacgacg

SEQ ID NO: 225 - DNA, 50 bp, synthetic construct
  source 1..50: mol_type = other DNA
cagtgatttattttccagcagtaacgattttaagtttttgatacccataa

SEQ ID NO: 226 - DNA, 50 bp, synthetic construct
  source 1..50: mol_type = other DNA
aattacacgcgtttaccgataaagtagttttatccatttgtacgttataa

SEQ ID NO: 227 - DNA, 50 bp, synthetic construct
  source 1..50: mol_type = other DNA
aaaatataactcgtattaaagagttgtatatgattaatttcaataactaa

SEQ ID NO: 228 - DNA, 50 bp, synthetic construct
  source 1..50: mol_type = other DNA
aattcccatactaagagctatttttaaacagttatcatttcatttttact

SEQ ID NO: 229 - DNA, 50 bp, synthetic construct
  source 1..50: mol_type = other DNA
taaactactgctgtgatttttaaaacatagttattacttatcactcataa

SEQ ID NO: 230 - DNA, 50 bp, synthetic construct
  source 1..50: mol_type = other DNA
gatatttctctacggagtttattgtaagctttttccattttaaatagaaa

SEQ ID NO: 231 - DNA, 50 bp, synthetic construct
  source 1..50: mol_type = other DNA
aggttttctacttgctcattagaagtataaaaaaatagttccgtaattaa

SEQ ID NO: 232 - DNA, 50 bp, synthetic construct
  source 1..50: mol_type = other DNA
aaaatgtttttatataaaatattggacgacgagatacgtagagtgttaac

SEQ ID NO: 233 - DNA, 50 bp, synthetic construct
  source 1..50: mol_type = other DNA
agattggatattaaaatcacgctttcgagtaaaaactacgaatataaata

SEQ ID NO: 234 - DNA, 50 bp, synthetic construct
  source 1..50: mol_type = other DNA
aactctggaagagcacaaataaattaaacaactaaatctgtaaataaata

SEQ ID NO: 235 - DNA, 50 bp, synthetic construct
  source 1..50: mol_type = other DNA
tataatctagttaaatcttctgtataaataaaaatatttttagcttctaa

SEQ ID NO: 236 - DNA, 50 bp, synthetic construct
  source 1..50: mol_type = other DNA
ctattttatatctatttattcgcgtcctaaaattaaaacaaatgatataa

SEQ ID NO: 237 - DNA, 50 bp, synthetic construct
  source 1..50: mol_type = other DNA
gatgttgatataccaacatttaacagtttaaatactgacgattattaaga

SEQ ID NO: 238 - DNA, 50 bp, synthetic construct
  source 1..50: mol_type = other DNA
ttgcacgatcgtgttatagggcatattctgacttattttttactacctaa

SEQ ID NO: 239 - DNA, 50 bp, synthetic construct
  source 1..50: mol_type = other DNA
aattcgaaagaaaaagaatcacagtcctaaaagctgaacttcggaaatct

SEQ ID NO: 240 - DNA, 50 bp, synthetic construct
  source 1..50: mol_type = other DNA
atctagaatatcagatcttgaaagacagttgaacgactgtagacgtaata

SEQ ID NO: 241 - DNA, 50 bp, synthetic construct
  source 1..50: mol_type = other DNA
ttataattacccgattgtagttaagttttgaataaaattttttataataa

SEQ ID NO: 242 - DNA, 50 bp, synthetic construct
  source 1..50: mol_type = other DNA
taccaaatataaataacgcagagtgtcagtttctaaaatctgtactttaa

SEQ ID NO: 243 - DNA, 50 bp, synthetic construct
  source 1..50: mol_type = other DNA
tccataaaagacgaataagatacaaacacaaatgtttatataatatttaa

SEQ ID NO: 244 - DNA, 50 bp, synthetic construct
  source 1..50: mol_type = other DNA
atgttttttccaaaaacctaagtgtatttaaaatagatgccatgttaaaa

SEQ ID NO: 245 - DNA, 50 bp, synthetic construct
  source 1..50: mol_type = other DNA
tccatattttgatttattatcaaattaatttagtaactgtaaatataatt

SEQ ID NO: 246 - DNA, 50 bp, synthetic construct
  source 1..50: mol_type = other DNA
caaaatagaataaaataaataacaaaggtatcattttaaataaataaaaa

SEQ ID NO: 247 - DNA, 51 bp, synthetic construct
  source 1..51: mol_type = other DNA
gatatccatggtatagaccaaacaataacgatatatatcataaataaataa

SEQ ID NO: 248 - DNA, 50 bp, synthetic construct
  source 1..50: mol_type = other DNA
taattattagaataagagtgtagtatcatagataactctcttctataaaa

SEQ ID NO: 249 - DNA, 50 bp, synthetic construct
  source 1..50: mol_type = other DNA
tatacatagatataattatcacatattaaaaattcacacatttttgataa

SEQ ID NO: 250 - DNA, 50 bp, synthetic construct
  source 1..50: mol_type = other DNA
acataaaaactcattacatagttgataaaaagcggtaggatataaatatt

SEQ ID NO: 251 - DNA, 50 bp, synthetic construct
  source 1..50: mol_type = other DNA
tagttctggtattttactaattactaaatctgtatatctttccatttatc

SEQ ID NO: 252 - DNA, 50 bp, synthetic construct
  source 1..50: mol_type = other DNA
cgacgctgttctgcagccatttaactttaaataatttacaaaaatttaaa

SEQ ID NO: 253 - DNA, 50 bp, synthetic construct
  source 1..50: mol_type = other DNA
tttgtaacatcggtacgggtattcatttatcacaaaaaaaacttctctaa

SEQ ID NO: 254 - DNA, 50 bp, synthetic construct
  source 1..50: mol_type = other DNA
tagtaaaccgatagtgtataaagattgtgcaaagcttttgcgatcaataa

SEQ ID NO: 255 - DNA, 50 bp, synthetic construct
  source 1..50: mol_type = other DNA
ctacggatggatgatatagatctttacacaaataattacaaaaccgataa

SEQ ID NO: 256 - DNA, 50 bp, synthetic construct
  source 1..50: mol_type = other DNA
atctccgtaaatatatgctcatatatttatagaagatatcacatatctaa

SEQ ID NO: 257 - DNA, 50 bp, synthetic construct
  source 1..50: mol_type = other DNA
gataaatacgaatatctgtcttatatttataatatgctagttaatagtaa

SEQ ID NO: 258 - DNA, 50 bp, synthetic construct
  source 1..50: mol_type = other DNA
caatattgaaaatactaattgtttaaataacccgagtattgaaactatat

SEQ ID NO: 259 - DNA, 50 bp, synthetic construct
  source 1..50: mol_type = other DNA
tatttttgtgttaaaacaatgaactaatatttatttttgtacattaataa

SEQ ID NO: 260 - DNA, 50 bp, synthetic construct
  source 1..50: mol_type = other DNA
gatacgatactatatgtattcttcgatagtccgcattatgtacctattct

SEQ ID NO: 261 - DNA, 50 bp, synthetic construct
  source 1..50: mol_type = other DNA
caagtttattccaatagatgtcttattaaaaacatatataataaataaca

SEQ ID NO: 262 - DNA, 50 bp, synthetic construct
  source 1..50: mol_type = other DNA
aactggtaattaaaataaaaagtaatattcatatgtagtgtcaattttaa

SEQ ID NO: 263 - DNA, 50 bp, synthetic construct
  source 1..50: mol_type = other DNA
tttttgatggtggtttaacgttttaaaaaaagattttgttattgtagtat

SEQ ID NO: 264 - DNA, 50 bp, synthetic construct
  source 1..50: mol_type = other DNA
taacattgttaattgaaaagggataacatgttacagaatataaattatat

SEQ ID NO: 265 - DNA, 50 bp, synthetic construct
  source 1..50: mol_type = other DNA
tgcatattatacactggttaacgcccttataggctctaaccattttcaag

SEQ ID NO: 266 - DNA, 50 bp, synthetic construct
  source 1..50: mol_type = other DNA
ttgcagtgttcatctcccaactgcaagtgaaggattgataactgaaggca

SEQ ID NO: 267 - DNA, 50 bp, synthetic construct
  source 1..50: mol_type = other DNA
ctcttctccctttcccagaaacaaactttttttacccactataaaataaa

SEQ ID NO: 268 - DNA, 50 bp, synthetic construct
  source 1..50: mol_type = other DNA
aatagtataaactaaaaattaaacaaatcgttattataagtaatatcaaa

SEQ ID NO: 269 - DNA, 50 bp, synthetic construct
  source 1..50: mol_type = other DNA
ttctgtttttctttcacatctttaattatgaaaaagtaaatcattatgag

SEQ ID NO: 270 - DNA, 50 bp, synthetic construct
  source 1..50: mol_type = other DNA
cacttactaaatagccaaggtgattattcgtatttttttaaggagtaacc

SEQ ID NO: 271 - DNA, 50 bp, synthetic construct
  source 1..50: mol_type = other DNA
ttttattatttgtacgatgtccaggataacatttttacggataaataaat

SEQ ID NO: 272 - DNA, 50 bp, synthetic construct
  source 1..50: mol_type = other DNA
tagtttcttggaaaaatttattatgagagacattttctcagactggataa

SEQ ID NO: 273 - DNA, 50 bp, synthetic construct
  source 1..50: mol_type = other DNA
tctatcaaacctggactttcgtttgtaaattggggctttttgtacaataa

SEQ ID NO: 274 - DNA, 50 bp, synthetic construct
  source 1..50: mol_type = other DNA
atgaatatgatgaagatagcgataaagaaaagccaatattcaatgtataa

SEQ ID NO: 275 - DNA, 51 bp, synthetic construct
  source 1..51: mol_type = other DNA
saacgcagtttggaaaaaagaagatatctggtaaattcttttccatgataa

SEQ ID NO: 276 - DNA, 50 bp, synthetic construct
  source 1..50: mol_type = other DNA
tacgatgataacgacatacgaacattacttcctattttactccttagtaa

SEQ ID NO: 277 - DNA, 50 bp, synthetic construct
  source 1..50: mol_type = other DNA
atcttctgtaagtaggaatttggacaagttgaacaaaattagatctctaa

SEQ ID NO: 278 - DNA, 50 bp, synthetic construct
  source 1..50: mol_type = other DNA
atttttatacggatgctcattttaaatttttgtaaattatttaaagttaa

SEQ ID NO: 279 - DNA, 50 bp, synthetic construct
  source 1..50: mol_type = other DNA
atgaggttttctagcagtagactcatttagagaagttttttttgtgataa

SEQ ID NO: 280 - DNA, 50 bp, synthetic construct
  source 1..50: mol_type = other DNA
ttattacaactataaaaataatagttatatttacactttaaatttttatc

SEQ ID NO: 281 - DNA, 50 bp, synthetic construct
  source 1..50: mol_type = other DNA
taaaaaaattatacatcataaaccaatttcctagttgtttgtaactttaa

SEQ ID NO: 282 - DNA, 50 bp, synthetic construct
  source 1..50: mol_type = other DNA
cgttatcgtcgttatctactttgggatacttattatccttaactataaaa

SEQ ID NO: 283 - DNA, 50 bp, synthetic construct
  source 1..50: mol_type = other DNA
tatattagcgctagacatattacagaactattttagattatgatatttaa

SEQ ID NO: 284 - DNA, 50 bp, synthetic construct
  source 1..50: mol_type = other DNA
aagacttacatcatcggtagtagattttcactttaccccacgatataaat

SEQ ID NO: 285 - DNA, 50 bp, synthetic construct
  source 1..50: mol_type = other DNA
aaaatctaaatatgacagatggtgactctgtctcttttgatgatgaataa

SEQ ID NO: 286 - DNA, 50 bp, synthetic construct
  source 1..50: mol_type = other DNA
atcgttttgtatatccgtcactggtacggtcgtcatttaatactaaataa

SEQ ID NO: 287 - DNA, 50 bp, synthetic construct
  source 1..50: mol_type = other DNA
aaaagatgatatattgcatacttgatcaatagtgaagttattgtcaataa

SEQ ID NO: 288 - DNA, 50 bp, synthetic construct
  source 1..50: mol_type = other DNA
gtttatattccactttgttcattcggcgatttaaaatttttattagttaa

SEQ ID NO: 289 - DNA, 50 bp, synthetic construct
  source 1..50: mol_type = other DNA
attcgtattatttgagcaagaaaatatcccaccaccttttcgtctagtaa

SEQ ID NO: 290 - DNA, 50 bp, synthetic construct
  source 1..50: mol_type = other DNA
ggcataaagattatactccatctttaatagtgacattttttaatatataa

SEQ ID NO: 291 - DNA, 50 bp, synthetic construct
  source 1..50: mol_type = other DNA
tgtacagactaagtaattcttttaagttagttaaatcagcgctagaagtc

SEQ ID NO: 292 - DNA, 50 bp, synthetic construct
  source 1..50: mol_type = other DNA
acttaactcttttgttaattaaaagtatattcaaaaaatgagttatataa

SEQ ID NO: 293 - DNA, 50 bp, synthetic construct
  source 1..50: mol_type = other DNA
cattgtctgatgcgtgtaaaaaaattttgtcagcttctaatagattataa

SEQ ID NO: 294 - DNA, 50 bp, synthetic construct
  source 1..50: mol_type = other DNA
tgtatgtaaaaatatagtagaatttcattttgtttttttctatgctataa

SEQ ID NO: 295 - DNA, 50 bp, synthetic construct
  source 1..50: mol_type = other DNA
taatgcaccgaacatccatttatagaatttagaaatatattttcatttaa

SEQ ID NO: 296 - DNA, 50 bp, synthetic construct
  source 1..50: mol_type = other DNA
agaacctcaacgtaacttaacagtgcaacctctattggatataaactaat

SEQ ID NO: 297 - DNA, 50 bp, synthetic construct
  source 1..50: mol_type = other DNA
gatcaacatctttatggcgtttttagattaatactttcaatgagataaat

SEQ ID NO: 298 - DNA, 50 bp, synthetic construct
  source 1..50: mol_type = other DNA
tcagtttattatctctcttggtaatatggatactaattgtagctatttaa

SEQ ID NO: 299 - DNA, 50 bp, synthetic construct
  source 1..50: mol_type = other DNA
aaaagaatattcctctaacagatattccgacaaaggattgattactataa

SEQ ID NO: 300 - DNA, 50 bp, synthetic construct
  source 1..50: mol_type = other DNA
gtagtagtaagtatttatacaaacttttcttatccatttataacgtacaa

SEQ ID NO: 301 - DNA, 50 bp, synthetic construct
  source 1..50: mol_type = other DNA
agggaaaatctaaagttgttcgtaaaaaagttaaaacttgtaagaagtaa

SEQ ID NO: 302 - DNA, 50 bp, synthetic construct
  source 1..50: mol_type = other DNA
ataaaatactactgttgagtaaatcagttattttttttatatcgatattg

SEQ ID NO: 303 - DNA, 50 bp, synthetic construct
  source 1..50: mol_type = other DNA
ttgatcaagagtaactattgacttaataggcatcatttatttagtattaa

SEQ ID NO: 304 - DNA, 50 bp, synthetic construct
  source 1..50: mol_type = other DNA
ccaatttccatctaatatactttgtcggattatctatagtacacggaata

SEQ ID NO: 305 - DNA, 50 bp, synthetic construct
  source 1..50: mol_type = other DNA
ccattgctgccactcataatatcagactacttattctattttactaaata

SEQ ID NO: 306 - DNA, 50 bp, synthetic construct
  source 1..50: mol_type = other DNA
tttgtataaataattatttcaatatactagttaaaattttaagattttaa

SEQ ID NO: 307 - DNA, 50 bp, synthetic construct
  source 1..50: mol_type = other DNA
tccatccacagacgttaccgaaccgattagtgatgtgacaccatcggtgg

SEQ ID NO: 308 - DNA, 50 bp, synthetic construct
  source 1..50: mol_type = other DNA
atacgaggacgtgtatagagtaagtaaagaaaaagaatgtggaatttgct

SEQ ID NO: 309 - protein, 83 aa, synthetic construct
  REPEAT 2..83: note = repeated 2, 3, 4, 5, 6, 7, 8, 9, 10 times, or more
MDAMKRGLCCVLLLCGAVFVSPSQEIHARFRRGARCRRTSTGQISTLRVNITAPLSQRAKRGSGATNFSLLKQAGDVEENPGP

SEQ ID NO: 310 - protein, 83 aa, synthetic construct
  REPEAT 2..83: note = repeated 2, 3, 4, 5, 6, 7, 8, 9, 10 times, or more
MDAMKRGLCCVLLLCGAVFVSPSQEIHARFRRGARCRRTSTGQISTLRVNITAPLSQRRRRGSGATNFSLLKQAGDVEENPGP

SEQ ID NO: 311 - protein, 83 aa, synthetic construct
  REPEAT 2..83: note = repeated 2, 3, 4, 5, 6, 7, 8, 9, 10 times, or more
MDAMKRGLCCVLLLCGAVFVSPSQEIHARFRRGARCRRTSTGQISTLRVNITAPLSQRKRRGSGATNFSLLKQAGDVEENPGP

SEQ ID NO: 312 - protein, 83 aa, synthetic construct
  REPEAT 2..83: note = repeated 2, 3, 4, 5, 6, 7, 8, 9, 10 times, or more
MDAMKRGLCCVLLLCGAVFVSPSQEIHARFRRGARCRRTSTGQISTLRVNITAPLSQRRKRGSGATNFSLLKQAGDVEENPGP

SEQ ID NO: 313 - protein, 79 aa, synthetic construct
  REPEAT 2..79: note = repeated 2, 3, 4, 5, 6, 7, 8, 9, 10 times, or more
MDAMKRGLCCVLLLCGAVFVSPSQEIHARFRRGARSTGQISTLRVNITAPLSQRAKRGSGATNFSLLKQAGDVEENPGP

SEQ ID NO: 314 - protein, 79 aa, synthetic construct
  REPEAT 2..79: note = repeated 2, 3, 4, 5, 6, 7, 8, 9, 10 times, or more
MDAMKRGLCCVLLLCGAVFVSPSQEIHARFRRGARSTGQISTLRVNITAPLSQRRRRGSGATNFSLLKQAGDVEENPGP

SEQ ID NO: 315 - protein, 79 aa, synthetic construct
  REPEAT 2..79: note = repeated 2, 3, 4, 5, 6, 7, 8, 9, 10 times, or more
MDAMKRGLCCVLLLCGAVFVSPSQEIHARFRRGARSTGQISTLRVNITAPLSQRKRRGSGATNFSLLKQAGDVEENPGP

SEQ ID NO: 316 - protein, 79 aa, synthetic construct
  REPEAT 2..79: note = repeated 2, 3, 4, 5, 6, 7, 8, 9, 10 times, or more
MDAMKRGLCCVLLLCGAVFVSPSQEIHARFRRGARSTGQISTLRVNITAPLSQRRKRGSGATNFSLLKQAGDVEENPGP

SEQ ID NO: 317 - protein, 139 aa, synthetic construct
  REPEAT 2..83: note = repeated 2, 3, 4, 5, 6, 7, 8, 9, 10 times, or more
MDAMKRGLCCVLLLCGAVFVSPSQEIHARFRRGARCRRTSTGQISTLRVNITAPLSQRAKRGSGATNFSLLKQAGDVEENPGPDAMKRGLCCVLLLCGAVFVSPSQEIHARFRRGARCRRTSTGQISTLRVNITAPLSQ

SEQ ID NO: 318 - protein, 139 aa, synthetic construct
  REPEAT 2..83: note = repeated 2, 3, 4, 5, 6, 7, 8, 9, 10 times, or more
MDAMKRGLCCVLLLCGAVFVSPSQEIHARFRRGARCRRTSTGQISTLRVNITAPLSQRRRRGSGATNFSLLKQAGDVEENPGPDAMKRGLCCVLLLCGAVFVSPSQEIHARFRRGARCRRTSTGQISTLRVNITAPLSQ

SEQ ID NO: 319 - protein, 139 aa, synthetic construct
  REPEAT 2..83: note = repeated 2, 3, 4, 5, 6, 7, 8, 9, 10 times, or more
MDAMKRGLCCVLLLCGAVFVSPSQEIHARFRRGARCRRTSTGQISTLRVNITAPLSQRKRRGSGATNFSLLKQAGDVEENPGPDAMKRGLCCVLLLCGAVFVSPSQEIHARFRRGARCRRTSTGQISTLRVNITAPLSQ

SEQ ID NO: 320 - protein, 139 aa, synthetic construct
  REPEAT 2..83: note = repeated 2, 3, 4, 5, 6, 7, 8, 9, 10 times, or more
MDAMKRGLCCVLLLCGAVFVSPSQEIHARFRRGARCRRTSTGQISTLRVNITAPLSQRRKRGSGATNFSLLKQAGDVEENPGPDAMKRGLCCVLLLCGAVFVSPSQEIHARFRRGARCRRTSTGQISTLRVNITAPLSQ

SEQ ID NO: 321 - protein, 131 aa, synthetic construct
  REPEAT 2..79: note = repeated 2, 3, 4, 5, 6, 7, 8, 9, 10 times, or more
MDAMKRGLCCVLLLCGAVFVSPSQEIHARFRRGARSTGQISTLRVNITAPLSQRAKRGSGATNFSLLKQAGDVEENPGPDAMKRGLCCVLLLCGAVFVSPSQEIHARFRRGARSTGQISTLRVNITAPLSQ

SEQ ID NO: 322 - protein, 131 aa, synthetic construct
  REPEAT 2..79: note = repeated 2, 3, 4, 5, 6, 7, 8, 9, 10 times, or more
MDAMKRGLCCVLLLCGAVFVSPSQEIHARFRRGARSTGQISTLRVNITAPLSQRRRRGSGATNFSLLKQAGDVEENPGPDAMKRGLCCVLLLCGAVFVSPSQEIHARFRRGARSTGQISTLRVNITAPLSQ

SEQ ID NO: 323 - protein, 131 aa, synthetic construct
  REPEAT 2..79: note = repeated 2, 3, 4, 5, 6, 7, 8, 9, 10 times, or more
MDAMKRGLCCVLLLCGAVFVSPSQEIHARFRRGARSTGQISTLRVNITAPLSQRKRRGSGATNFSLLKQAGDVEENPGPDAMKRGLCCVLLLCGAVFVSPSQEIHARFRRGARSTGQISTLRVNITAPLSQ

SEQ ID NO: 324 - protein, 131 aa, synthetic construct
  REPEAT 2..79: note = repeated 2, 3, 4, 5, 6, 7, 8, 9, 10 times, or more
MDAMKRGLCCVLLLCGAVFVSPSQEIHARFRRGARSTGQISTLRVNITAPLSQRRKRGSGATNFSLLKQAGDVEENPGPDAMKRGLCCVLLLCGAVFVSPSQEIHARFRRGARSTGQISTLRVNITAPLSQ

SEQ ID NO: 325 - protein, 411 aa, synthetic construct
MDAMKRGLCCVLLLCGAVFVSPSQEIHARFRRGARCRRTSTGQISTLRVNITAPLSQRAKRGSGATNFSLLKQAGDVEENPGPDAMKRGLCCVLLLCGAVFVSPSQEIHARFRRGARCRRTSTGQISTLRVNITAPLSQRAKRGSGATNFSLLKQAGDVEENPGPDAMKRGLCCVLLLCGAVFVSPSQEIHARFRRGARCRRTSTGQISTLRVNITAPLSQRAKRGSGATNFSLLKQAGDVEENPGPDAMKRGLCCVLLLCGAVFVSPSQEIHARFRRGARCRRTSTGQISTLRVNITAPLSQRAKRGSGATNFSLLKQAGDVEENPGPDAMKRGLCCVLLLCGAVFVSPSQEIHARFRRGARCRRTSTGQISTLRVNITAPLSQRAKRGSGATNFSLLKQAGDVEENPGP

SEQ ID NO: 326 - protein, 411 aa, synthetic construct
MDAMKRGLCCVLLLCGAVFVSPSQEIHARFRRGARCRRTSTGQISTLRVNITAPLSQRRRRGSGATNFSLLKQAGDVEENPGPDAMKRGLCCVLLLCGAVFVSPSQEIHARFRRGARCRRTSTGQISTLRVNITAPLSQRRRRGSGATNFSLLKQAGDVEENPGPDAMKRGLCCVLLLCGAVFVSPSQEIHARFRRGARCRRTSTGQISTLRVNITAPLSQRRRRGSGATNFSLLKQAGDVEENPGPDAMKRGLCCVLLLCGAVFVSPSQEIHARFRRGARCRRTSTGQISTLRVNITAPLSQRRRRGSGATNFSLLKQAGDVEENPGPDAMKRGLCCVLLLCGAVFVSPSQEIHARFRRGARCRRTSTGQISTLRVNITAPLSQRRRRGSGATNFSLLKQAGDVEENPGP

SEQ ID NO: 327 - protein, 411 aa, synthetic construct
MDAMKRGLCCVLLLCGAVFVSPSQEIHARFRRGARCRRTSTGQISTLRVNITAPLSQRKRRGSGATNFSLLKQAGDVEENPGPDAMKRGLCCVLLLCGAVFVSPSQEIHARFRRGARCRRTSTGQISTLRVNITAPLSQRKRRGSGATNFSLLKQAGDVEENPGPDAMKRGLCCVLLLCGAVFVSPSQEIHARFRRGARCRRTSTGQISTLRVNITAPLSQRKRRGSGATNFSLLKQAGDVEENPGPDAMKRGLCCVLLLCGAVFVSPSQEIHARFRRGARCRRTSTGQISTLRVNITAPLSQRKRRGSGATNFSLLKQAGDVEENPGPDAMKRGLCCVLLLCGAVFVSPSQEIHARFRRGARCRRTSTGQISTLRVNITAPLSQRKRRGSGATNFSLLKQAGDVEENPGP

SEQ ID NO: 328 - protein, 411 aa, synthetic construct
MDAMKRGLCCVLLLCGAVFVSPSQEIHARFRRGARCRRTSTGQISTLRVNITAPLSQRRKRGSGATNFSLLKQAGDVEENPGPDAMKRGLCCVLLLCGAVFVSPSQEIHARFRRGARCRRTSTGQISTLRVNITAPLSQRRKRGSGATNFSLLKQAGDVEENPGPDAMKRGLCCVLLLCGAVFVSPSQEIHARFRRGARCRRTSTGQISTLRVNITAPLSQRRKRGSGATNFSLLKQAGDVEENPGPDAMKRGLCCVLLLCGAVFVSPSQEIHARFRRGARCRRTSTGQISTLRVNITAPLSQRRKRGSGATNFSLLKQAGDVEENPGPDAMKRGLCCVLLLCGAVFVSPSQEIHARFRRGARCRRTSTGQISTLRVNITAPLSQRRKRGSGATNFSLLKQAGDVEENPGP

SEQ ID NO: 329 - protein, 385 aa, synthetic construct
MDAMKRGLCCVLLLCGAVFVSPSQEIHARFRRGARCRRTSTGQISTLRVNITAPLSQRAKRGSGATNFSLLKQAGDVEENPGPDAMKRGLCCVLLLCGAVFVSPSQEIHARFRRGARCRRTSTGQISTLRVNITAPLSQRAKRGSGATNFSLLKQAGDVEENPGPDAMKRGLCCVLLLCGAVFVSPSQEIHARFRRGARCRRTSTGQISTLRVNITAPLSQRAKRGSGATNFSLLKQAGDVEENPGPDAMKRGLCCVLLLCGAVFVSPSQEIHARFRRGARCRRTSTGQISTLRVNITAPLSQRAKRGSGATNFSLLKQAGDVEENPGPDAMKRGLCCVLLLCGAVFVSPSQEIHARFRRGARCRRTSTGQISTLRVNITAPLSQ

SEQ ID NO: 330 - protein, 385 aa, synthetic construct
MDAMKRGLCCVLLLCGAVFVSPSQEIHARFRRGARCRRTSTGQISTLRVNITAPLSQRRRRGSGATNFSLLKQAGDVEENPGPDAMKRGLCCVLLLCGAVFVSPSQEIHARFRRGARCRRTSTGQISTLRVNITAPLSQRRRRGSGATNFSLLKQAGDVEENPGPDAMKRGLCCVLLLCGAVFVSPSQEIHARFRRGARCRRTSTGQISTLRVNITAPLSQRRRRGSGATNFSLLKQAGDVEENPGPDAMKRGLCCVLLLCGAVFVSPSQEIHARFRRGARCRRTSTGQISTLRVNITAPLSQRRRRGSGATNFSLLKQAGDVEENPGPDAMKRGLCCVLLLCGAVFVSPSQEIHARFRRGARCRRTSTGQISTLRVNITAPLSQ

SEQ ID NO: 331 - protein, 385 aa, synthetic construct
MDAMKRGLCCVLLLCGAVFVSPSQEIHARFRRGARCRRTSTGQISTLRVNITAPLSQRKRRGSGATNFSLLKQAGDVEENPGPDAMKRGLCCVLLLCGAVFVSPSQEIHARFRRGARCRRTSTGQISTLRVNITAPLSQRKRRGSGATNFSLLKQAGDVEENPGPDAMKRGLCCVLLLCGAVFVSPSQEIHARFRRGARCRRTSTGQISTLRVNITAPLSQRKRRGSGATNFSLLKQAGDVEENPGPDAMKRGLCCVLLLCGAVFVSPSQEIHARFRRGARCRRTSTGQISTLRVNITAPLSQRKRRGSGATNFSLLKQAGDVEENPGPDAMKRGLCCVLLLCGAVFVSPSQEIHARFRRGARCRRTSTGQISTLRVNITAPLSQ

SEQ ID NO: 332 - protein, 385 aa, synthetic construct
MDAMKRGLCCVLLLCGAVFVSPSQEIHARFRRGARCRRTSTGQISTLRVNITAPLSQRRKRGSGATNFSLLKQAGDVEENPGPDAMKRGLCCVLLLCGAVFVSPSQEIHARFRRGARCRRTSTGQISTLRVNITAPLSQRRKRGSGATNFSLLKQAGDVEENPGPDAMKRGLCCVLLLCGAVFVSPSQEIHARFRRGARCRRTSTGQISTLRVNITAPLSQRRKRGSGATNFSLLKQAGDVEENPGPDAMKRGLCCVLLLCGAVFVSPSQEIHARFRRGARCRRTSTGQISTLRVNITAPLSQRRKRGSGATNFSLLKQAGDVEENPGPDAMKRGLCCVLLLCGAVFVSPSQEIHARFRRGARCRRTSTGQISTLRVNITAPLSQ

SEQ ID NO: 333 - protein, 391 aa, synthetic construct
MDAMKRGLCCVLLLCGAVFVSPSQEIHARFRRGARSTGQISTLRVNITAPLSQRAKRGSGATNFSLLKQAGDVEENPGPDAMKRGLCCVLLLCGAVFVSPSQEIHARFRRGARSTGQISTLRVNITAPLSQRAKRGSGATNFSLLKQAGDVEENPGPDAMKRGLCCVLLLCGAVFVSPSQEIHARFRRGARSTGQISTLRVNITAPLSQRAKRGSGATNFSLLKQAGDVEENPGPDAMKRGLCCVLLLCGAVFVSPSQEIHARFRRGARSTGQISTLRVNITAPLSQRAKRGSGATNFSLLKQAGDVEENPGPDAMKRGLCCVLLLCGAVFVSPSQEIHARFRRGARSTGQISTLRVNITAPLSQRAKRGSGATNFSLLKQAGDVEENPGP

SEQ ID NO: 334 - protein, 391 aa, synthetic construct
MDAMKRGLCCVLLLCGAVFVSPSQEIHARFRRGARSTGQISTLRVNITAPLSQRRRRGSGATNFSLLKQAGDVEENPGPDAMKRGLCCVLLLCGAVFVSPSQEIHARFRRGARSTGQISTLRVNITAPLSQRRRRGSGATNFSLLKQAGDVEENPGPDAMKRGLCCVLLLCGAVFVSPSQEIHARFRRGARSTGQISTLRVNITAPLSQRRRRGSGATNFSLLKQAGDVEENPGPDAMKRGLCCVLLLCGAVFVSPSQEIHARFRRGARSTGQISTLRVNITAPLSQRRRRGSGATNFSLLKQAGDVEENPGPDAMKRGLCCVLLLCGAVFVSPSQEIHARFRRGARSTGQISTLRVNITAPLSQRRRRGSGATNFSLLKQAGDVEENPGP

SEQ ID NO: 335 - protein, 391 aa, synthetic construct
MDAMKRGLCCVLLLCGAVFVSPSQEIHARFRRGARSTGQISTLRVNITAPLSQRKRRGSGATNFSLLKQAGDVEENPGPDAMKRGLCCVLLLCGAVFVSPSQEIHARFRRGARSTGQISTLRVNITAPLSQRKRRGSGATNFSLLKQAGDVEENPGPDAMKRGLCCVLLLCGAVFVSPSQEIHARFRRGARSTGQISTLRVNITAPLSQRKRRGSGATNFSLLKQAGDVEENPGPDAMKRGLCCVLLLCGAVFVSPSQEIHARFRRGARSTGQISTLRVNITAPLSQRKRRGSGATNFSLLKQAGDVEENPGPDAMKRGLCCVLLLCGAVFVSPSQEIHARFRRGARSTGQISTLRVNITAPLSQRKRRGSGATNFSLLKQAGDVEENPGP

SEQ ID NO: 336 - protein, 391 aa, synthetic construct
MDAMKRGLCCVLLLCGAVFVSPSQEIHARFRRGARSTGQISTLRVNITAPLSQRRKRGSGATNFSLLKQAGDVEENPGPDAMKRGLCCVLLLCGAVFVSPSQEIHARFRRGARSTGQISTLRVNITAPLSQRRKRGSGATNFSLLKQAGDVEENPGPDAMKRGLCCVLLLCGAVFVSPSQEIHARFRRGARSTGQISTLRVNITAPLSQRRKRGSGATNFSLLKQAGDVEENPGPDAMKRGLCCVLLLCGAVFVSPSQEIHARFRRGARSTGQISTLRVNITAPLSQRRKRGSGATNFSLLKQAGDVEENPGPDAMKRGLCCVLLLCGAVFVSPSQEIHARFRRGARSTGQISTLRVNITAPLSQRRKRGSGATNFSLLKQAGDVEENPGP

SEQ ID NO: 337 - protein, 365 aa, synthetic construct
MDAMKRGLCCVLLLCGAVFVSPSQEIHARFRRGARSTGQISTLRVNITAPLSQRAKRGSGATNFSLLKQAGDVEENPGPDAMKRGLCCVLLLCGAVFVSPSQEIHARFRRGARSTGQISTLRVNITAPLSQRAKRGSGATNFSLLKQAGDVEENPGPDAMKRGLCCVLLLCGAVFVSPSQEIHARFRRGARSTGQISTLRVNITAPLSQRAKRGSGATNFSLLKQAGDVEENPGPDAMKRGLCCVLLLCGAVFVSPSQEIHARFRRGARSTGQISTLRVNITAPLSQRAKRGSGATNFSLLKQAGDVEENPGPDAMKRGLCCVLLLCGAVFVSPSQEIHARFRRGARSTGQISTLRVNITAPLSQ

SEQ ID NO: 338 - protein, 365 aa, synthetic construct
MDAMKRGLCCVLLLCGAVFVSPSQEIHARFRRGARSTGQISTLRVNITAPLSQRRRRGSGATNFSLLKQAGDVEENPGPDAMKRGLCCVLLLCGAVFVSPSQEIHARFRRGARSTGQISTLRVNITAPLSQRRRRGSGATNFSLLKQAGDVEENPGPDAMKRGLCCVLLLCGAVFVSPSQEIHARFRRGARSTGQISTLRVNITAPLSQRRRRGSGATNFSLLKQAGDVEENPGPDAMKRGLCCVLLLCGAVFVSPSQEIHARFRRGARSTGQISTLRVNITAPLSQRRRRGSGATNFSLLKQAGDVEENPGPDAMKRGLCCVLLLCGAVFVSPSQEIHARFRRGARSTGQISTLRVNITAPLSQ

SEQ ID NO: 339 - protein, 365 aa, synthetic construct
MDAMKRGLCCVLLLCGAVFVSPSQEIHARFRRGARSTGQISTLRVNITAPLSQRKRRGSGATNFSLLKQAGDVEENPGPDAMKRGLCCVLLLCGAVFVSPSQEIHARFRRGARSTGQISTLRVNITAPLSQRKRRGSGATNFSLLKQAGDVEENPGPDAMKRGLCCVLLLCGAVFVSPSQEIHARFRRGARSTGQISTLRVNITAPLSQRKRRGSGATNFSLLKQAGDVEENPGPDAMKRGLCCVLLLCGAVFVSPSQEIHARFRRGARSTGQISTLRVNITAPLSQRKRRGSGATNFSLLKQAGDVEENPGPDAMKRGLCCVLLLCGAVFVSPSQEIHARFRRGARSTGQISTLRVNITAPLSQ

SEQ ID NO: 340 - protein, 365 aa, synthetic construct
MDAMKRGLCCVLLLCGAVFVSPSQEIHARFRRGARSTGQISTLRVNITAPLSQRRKRGSGATNFSLLKQAGDVEENPGPDAMKRGLCCVLLLCGAVFVSPSQEIHARFRRGARSTGQISTLRVNITAPLSQRRKRGSGATNFSLLKQAGDVEENPGPDAMKRGLCCVLLLCGAVFVSPSQEIHARFRRGARSTGQISTLRVNITAPLSQRRKRGSGATNFSLLKQAGDVEENPGPDAMKRGLCCVLLLCGAVFVSPSQEIHARFRRGARSTGQISTLRVNITAPLSQRRKRGSGATNFSLLKQAGDVEENPGPDAMKRGLCCVLLLCGAVFVSPSQEIHARFRRGARSTGQISTLRVNITAPLSQ

SEQ ID NO: 341 - protein, 83 aa, synthetic construct
  REPEAT 2..83: note = repeated 2, 3, 4, 5, 6, 7, 8, 9, 10 times, or more
MDAMKRGLCCVLLLCGAVFVSPSQEIHARFRRGARCRRTSTGQISTLRVNITAPLSQRKKRGSGATNFSLLKQAGDVEENPGP

SEQ ID NO: 342 - protein, 79 aa, synthetic construct
  REPEAT 2..79: note = repeated 2, 3, 4, 5, 6, 7, 8, 9, 10 times, or more
MDAMKRGLCCVLLLCGAVFVSPSQEIHARFRRGARSTGQISTLRVNITAPLSQRKKRGSGATNFSLLKQAGDVEENPGP

SEQ ID NO: 343 - protein, 139 aa, synthetic construct
  REPEAT 2..83: note = repeated 2, 3, 4, 5, 6, 7, 8, 9, 10 times, or more
MDAMKRGLCCVLLLCGAVFVSPSQEIHARFRRGARCRRTSTGQISTLRVNITAPLSQRKKRGSGATNFSLLKQAGDVEENPGPDAMKRGLCCVLLLCGAVFVSPSQEIHARFRRGARCRRTSTGQISTLRVNITAPLSQ

SEQ ID NO: 344 - protein, 131 aa, synthetic construct
  REPEAT 2..79: note = repeated 2, 3, 4, 5, 6, 7, 8, 9, 10 times, or more
MDAMKRGLCCVLLLCGAVFVSPSQEIHARFRRGARSTGQISTLRVNITAPLSQRKKRGSGATNFSLLKQAGDVEENPGPDAMKRGLCCVLLLCGAVFVSPSQEIHARFRRGARSTGQISTLRVNITAPLSQ

SEQ ID NO: 345 - protein, 411 aa, synthetic construct
MDAMKRGLCCVLLLCGAVFVSPSQEIHARFRRGARCRRTSTGQISTLRVNITAPLSQRKKRGSGATNFSLLKQAGDVEENPGPDAMKRGLCCVLLLCGAVFVSPSQEIHARFRRGARCRRTSTGQISTLRVNITAPLSQRKKRGSGATNFSLLKQAGDVEENPGPDAMKRGLCCVLLLCGAVFVSPSQEIHARFRRGARCRRTSTGQISTLRVNITAPLSQRKKRGSGATNFSLLKQAGDVEENPGPDAMKRGLCCVLLLCGAVFVSPSQEIHARFRRGARCRRTSTGQISTLRVNITAPLSQRKKRGSGATNFSLLKQAGDVEENPGPDAMKRGLCCVLLLCGAVFVSPSQEIHARFRRGARCRRTSTGQISTLRVNITAPLSQRKKRGSGATNFSLLKQAGDVEENPGP

SEQ ID NO: 346 - protein, 391 aa, synthetic construct
MDAMKRGLCCVLLLCGAVFVSPSQEIHARFRRGARSTGQISTLRVNITAPLSQRKKRGSGATNFSLLKQAGDVEENPGPDAMKRGLCCVLLLCGAVFVSPSQEIHARFRRGARSTGQISTLRVNITAPLSQRKKRGSGATNFSLLKQAGDVEENPGPDAMKRGLCCVLLLCGAVFVSPSQEIHARFRRGARSTGQISTLRVNITAPLSQRKKRGSGATNFSLLKQAGDVEENPGPDAMKRGLCCVLLLCGAVFVSPSQEIHARFRRGARSTGQISTLRVNITAPLSQRKKRGSGATNFSLLKQAGDVEENPGPDAMKRGLCCVLLLCGAVFVSPSQEIHARFRRGARSTGQISTLRVNITAPLSQRKKRGSGATNFSLLKQAGDVEENPGP

SEQ ID NO: 347 - protein, 385 aa, synthetic construct
MDAMKRGLCCVLLLCGAVFVSPSQEIHARFRRGARCRRTSTGQISTLRVNITAPLSQRKKRGSGATNFSLLKQAGDVEENPGPDAMKRGLCCVLLLCGAVFVSPSQEIHARFRRGARCRRTSTGQISTLRVNITAPLSQRKKRGSGATNFSLLKQAGDVEENPGPDAMKRGLCCVLLLCGAVFVSPSQEIHARFRRGARCRRTSTGQISTLRVNITAPLSQRKKRGSGATNFSLLKQAGDVEENPGPDAMKRGLCCVLLLCGAVFVSPSQEIHARFRRGARCRRTSTGQISTLRVNITAPLSQRKKRGSGATNFSLLKQAGDVEENPGPDAMKRGLCCVLLLCGAVFVSPSQEIHARFRRGARCRRTSTGQISTLRVNITAPLSQ

SEQ ID NO: 348 - protein, 365 aa, synthetic construct
MDAMKRGLCCVLLLCGAVFVSPSQEIHARFRRGARSTGQISTLRVNITAPLSQRKKRGSGATNFSLLKQAGDVEENPGPDAMKRGLCCVLLLCGAVFVSPSQEIHARFRRGARSTGQISTLRVNITAPLSQRKKRGSGATNFSLLKQAGDVEENPGPDAMKRGLCCVLLLCGAVFVSPSQEIHARFRRGARSTGQISTLRVNITAPLSQRKKRGSGATNFSLLKQAGDVEENPGPDAMKRGLCCVLLLCGAVFVSPSQEIHARFRRGARSTGQISTLRVNITAPLSQRKKRGSGATNFSLLKQAGDVEENPGPDAMKRGLCCVLLLCGAVFVSPSQEIHARFRRGARSTGQISTLRVNITAPLSQ

SEQ ID NO: 349 - protein, 435 aa, Homo sapiens
TPGTQSPFFLLLLLTVLTVVTGSGHASSTPGGEKETSATQRSSVPSSTEKNAVSMTSSVLSSHSPGSGSSTTQGQDVTLAPATEPASGSAATWGQDVTSVPVTRPALGSTTPPAHDVTSAPDNKPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDNRPALGSTAPPVHNVTSASGSASGSASTLVHNGTSARATTTPASKSTPFSIPSHHSDTPTTLASHSTKTDASSTHHSTVPPLTSSNHSTSPQLSTGVSFFFLSFHISNLQFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAGV

SEQ ID NO: 350 - protein, 436 aa, synthetic construct
LPGMALRVTRNSKINAENKAKINMAGAKRVPTAPAATSKPGLRPRTALGDIGNKVSEQLQAKMPMKKEAKPSATGKVIDKKLPKPLEKVPMLVPVPVSEPVPEPEPEPEPEPVKEEKLSPEPILVDTASPSPMETSGCAPAEEDLCQAFSDVILAVNDVDAEDGADPNLCSEYVKDIYAYLRQLEEEQAVRPKYLLGREVTGNMRAILIDWLVQVQMKFRLLQETMYMTVSIIDRFMQNNCVPKKMLQLVGVTAMFIASKYEEMYPPEIGDFAFVTDNTYTKHQIRQMEMKILRALNFGLGRPLPLHFLRRASKIGEVDVEQHTLAKYLMELTMLDYDMVHFPPSQIAAGAFCLALKILDNGEWTPTLQHYLSYTEESLLPVMQHLAKNVVMVNQGLTKHMTVKNKYATSKHAKISTLPQLNSALVQDLAKAVAKV

SEQ ID NO: 351 - protein, 280 aa, Hepatitis B virus
QWNSTTFHQTLQDPRVRGLYFPAGGSSSGAVNPVPTTASPLSSIFSRIGDPALNMENITSGFLGPLLVLQAGFFLLTRILTIPQSLDSWWTSLNFLGGTTVCLGQNSQSPTSNHSPTSCPPTCPGYRWMCLRRFIIFLFILLLCLIFLLVLLDYQGMLPVCPLIPGSSTTSTGPCRTCMTTAQGTSMYPSCCCTKPSDGNCTCIPIPSSWAFGKFLWEWASARFSWLSLLVPFVQWFVGLSPTVWLSVIWMMWYWGPSLYSILSPFLPLLPIFFCLWVYI

SEQ ID NO: 352 - protein, 211 aa, Hepatitis B virus
HLFHLCLIISCSCPTVQASKLCLGWLWGMDIDPYKEFGATVELLSFLPSDFFPSVRDLLDTASALYREALESPEHCSPHHTALRQAILCWGELMTLATWVGVNLEDPASRDLVVSYVNTNMGLKFRQLLWFHISCLTFGRETVIEYLVSFGVWIRTPPAYRPPNAPILSTLPETTVVRRRGRSPRRRTPSPRRRRSQSPRRRRSQSREPQC

SEQ ID NO: 353 - protein, 111 aa, Hepatitis B virus
AARLCCQLDPARDVLCLRPVGAESCGRPFSGSLETLSSPSPSAVPTDHGAHLSLRGLPAMSTTDLEAYFKDCLFKDWEELGEEIRLKVFVLGGCRHKLVCAPAPCTFFTSA

SEQ ID NO: 354 - protein, 225 aa, synthetic construct
EAKLFVLFCAFTALKAMGTNLSVPNPLGFFPDHQLDPAFGANSNNPDWDFNPIKDHWPAANQVGVGAFGPGLTPPHGGILGWSPQAQGILTTVSTIPPPASTNRQSGRQPTPISPPLRDSHPQAMQWNSTAFHQALQDPRVRGLYLPAGGSSSGTVNPAPNIASHISSISARTGDPVTNKLESVGVHQILAIYSTVASSLVLLVSLGAISFWMCSNGSLQCRICI

SEQ ID NO: 355 - protein, 223 aa, synthetic construct
ARPGMMRKLAILSVSSFLFVEALFQEYQCYGSSSNTRVLNELNYDNAGTNLYNELEMNYYGKQENWYSLKKNSRSLGENDDGNNEDNEKLRKPKHKKLKQPADGNPDPGGGSNKNNQGNGQGHNMPNDPNRNVDENANANSAVKNNNNEEPSDKHIKEYLNKIQNSLSTEWSPCSVTCGNGIQVRIKPGSANKPKDELDYANDIEKKICKMEKCSSVFNVVNS

SEQ ID NO: 356 - protein, 319 aa, synthetic construct
  REGION 1..319: note = Plasmodium sp. CSP CSP21R (21 Repeats)
PIPGMMRKLAILSVSSFLFVEALFQEYQCYGSSSNTRVLNELNYDNAGTNLYNELEMNYYGKQENWYSLKKNSRSLGENDDGNNEDNEKLRKPKHKKLKQPADGNPDPNANPNVDPNANPNVDPNANPNVDPNANPNANPNANPNANPNANPNANPNANPNANPNANPNANPNANPNANPNANPNANPNANPNANPNANPNVDPNANPNKNNQGNGQGHNMPNDPNRNVDENANANSAVKNNNNEEPSDKHIKEYLNKIQNSLSTEWSPCSVTCGNGIQVRIKPGSANKPKDELDYANDIEKKICKMEKCSSVFNVVNS

SEQ ID NO: 357 - protein, 200 aa, synthetic construct
PGATMSVLQSGALPSVGVDELDKIDLSYETTESGDTAVSEDSYDKYASNNTNKEYVCDFTDQLKPTESGPKVKKCEVKVNEPLIKVKIICPLKGSVEKLYDNIEYVPKKSPYVVLTKEETKLKEKLLSKLIYGLLISPTVNEKENNFKEGVIEFTLPPVVHKATVFYFICDNSKTEDDNKKGNRGIVEVYVEPYGNKING

SEQ ID NO: 358 - protein, 20 aa, Homo sapiens
AHGVTSAPDTRPAPGSTAPP

SEQ ID NO: 359 - protein, 20 aa, Homo sapiens
AHGVTSAPDNRPALGSTAPP

SEQ ID NO: 360 - protein, 40 aa, synthetic construct
AHGVTSAPDTRPAPGSTAPPAHGVTSAPDNRPALGSTAPP

SEQ ID NO: 361 - protein, 100 aa, Homo sapiens
AHGVTSAPDTRPAPGSTAPPAHGVTSAPDTRPAPGSTAPPAHGVTSAPDTRPAPGSTAPPAHGVTSAPDTRPAPGSTAPPAHGVTSAPDNRPALGSTAPP

SEQ ID NO: 362 - protein, 69 aa, Homo sapiens
RRKNYGQLDIFPARDTYHPMSEYPTYHTHGRYVPPSSTDRSPYEKVSAGNGGSSLSYTNPAVAATSANL

SEQ ID NO: 363 - protein, 476 aa, synthetic construct
TPGTQSPFFLLLLLTVLTVVTGSGHASSTPGGEKETSATQRSSVPSSTEKNAVSMTSSVLSSHSPGSGSSTTQGQDVTLAPATEPASGSAATWGQDVTSVPVTRPALGSTTPPAHDVTSAPDNKPAPGSTAPPIAHGVTSAPDTRPAPGSTAPPAAHGVTSAPDNRPALGSTAPPVHNVTSASGSASGSASTLVHNGTSARATTTPASKSTPFSIPSHHSDTPTTLASHSTKTDASSTHHSTVPPLTSSNHSTSPQLSTGVSFFFLSFHISNLQFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAATSANL

SEQ ID NO: 364 - protein, 532 aa, synthetic construct
TPGTQSPFFLLLLLTVLTVVTGSGHASSTPGGEKETSATQRSSVPSSTEKNAVSMTSSVLSSHSPGSGSSTTQGQDVTLAPATEPASGSAATWGQDVTSVPVTRPALGSTTPPAHDVTSAPDNKPAPGSTAPPAHGVTSAPDTRPAPGSTPPAHGVTSAPDTRPAPGSTAPPAHGVTSAPDTRPAPGSTAPPAHGVTSAPDTRPAPGSTAPPHGVTSAPDNRPALGSTAPPVHNVTSASGSASGSASTLVHNGTSARATTTPASKSTPFSIPSHHSDTPTTLASHSTKTDASSTHHSTVPPLTSSNHSTSPQLSTGVSFFFLSFHISNLQFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAATSANL

SEQ ID NO: 365 - protein, 490 aa, synthetic construct
GQIVTFFQEVPHVIEEVMNIVLIALSVLAVLKGLYNFATCGLVGLVTFLLLCGRSCTTSLYKGVYELQTLELNMETLNMTMPLSCTKNNSHHYIMVGNETGLELTLTNTSIINHKFCNLSDAHKKNLYDHALMSIISTFHLSIPNFNQYEAMSCDFNGGKISVQYNLSHSYAGDAANHCGTVANGVLQTFMRMAWGGSYIALDSGRGNWDCIMTSYQYLIIQNTTWEDHCQFSRPSPIGYLGLLSQRTRDIYISRRLLGTFTWTLSDSEGKDTPGGYCLTRWMLIEAELKCFGNTAVAKCNEKHDEEFCDMLRLFDFNKQAIQRLKAEAQMSIQLINKAVNALINDQLIMKNHLRDIMGIPYCNYSKYWYLNHTTTGRTSLPKCWLVSNGSYLNETHFSDDIEQQADNMITEMLQKEYMERQGKTPLGLVDLFVFSTSFYLISIFLHLVKIPTHRHIVGKSCPKPHRLNHMGICSCGLYKQPGVPVKWKR

SEQ ID NO: 366 - protein, 98 aa, synthetic construct
GNKQAKAPESKDSPRASLIPDATHLGPQFCKSCWFENKGLVECNNHYLCLNCLTLLLSVSNRCPICKMPLPTKLRPSAAPTAPPTGAADSIRPPPYSP

SEQ ID NO: 367 - protein, 675 aa, synthetic construct
GVTGILQLPRDRFKRTSFFLWVIILFQRTFSIPLGVIHNSTLQVSDVDKLVCRDKLSSTNQLRSVGLNLEGNGVATDVPSVTKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYASGKRSNTTGKLIWKVNPEIDTTIGEWAFWETKKNLTRKIRSEELSFTAVSNGPKNISGQSPARTSSDPETNTTNEDHKIMASENSSAMVQVHSQGRKAAVSHLTTLATISTSPQPPTTKTGPDNSTHNTPVYKLDISEATQVGQHHRRADNDSTASDTPPATTAAGPLKAENTNTSKSADSLDLATTTSPQNYSETAGNNNTHHQDTGEESASSGKLGLITNTIAGVAGLITGGRRTRREVIVNAQPKCNPNLHYWTTQDEGAAIGLAWIPYFGPAAEGIYTEGLMHNQDGLICGLRQLANETTQALQLFLRATTELRTFSILNRKAIDFLLQRWGGTCHILGPDCCIEPHDWTKNITDKIDQIIHDFVDKTLPDQGDNDNWWTGWRQWIPAGIGVTGVIIAVIALFCICKFVF

SEQ ID NO: 368 - protein, 325 aa, synthetic construct
RRVILPTAPPEYMEAIYPARSNSTIARGGNSNTGFLTPESVNGDTPSNPLRPIADDTIDHASHTPGSVSSAFILEAMVNVISGPKVLMKQIPIWLPLGVADQKTYSFDSTTAAIMLASYTITHFGKATNPLVRVNRLGPGIPDHPLRLLRIGNQAFLQEFVLPPVQLPQYFTFDLTALKLITQPLPAATWTDDTPTGSNGALRPGISFHPKLRPILLPNKSGKKGNSADLTSPEKIQAIMTSLQDFKIVPIDPTKNIMGIEVPETLVHKLTGKKVTSKNGQPIIPVLLPKYIGLDPVAPGDLTMVITQDCDTCHSPASLPAVVEK

SEQ ID NO: 369 - protein, 3422 aa, Zika virus
KNPKKKSGGFRIVNMLKRGVARVSPFGGLKRLPAGLLLGHGPIRMVLAILAFLRFTAIKPSLGLINRWGSVGKKEAMEIIKKFKKDLAAMLRIINARKEKKRRGADTSVGIVGLLLTTAMAAEVTRRGSAYYMYLDRNDAGEAISFPTTLGMNKCYIQIMDLGHTCDATMSYECPMLDEGVEPDDVDCWCNTTSTWVVYGTCHHKKGEARRSRRAVTLPSHSTRKLQTRSQTWLESREYTKHLIRVENWIFRNPGFALAAAAIAWLLGSSTSQKVIYLVMILLIAPAYSIRCIGVSNRDFVEGMSGGTWVDVVLEHGGCVTVMAQDKPTVDIELVTTTVSNMAEVRSYCYEASISDMASDSRCPTQGEAYLDKQSDTQYVCKRTLVDRGWGNGCGLFGKGSLVTCAKFACSKKMTGKSIQPENLEYRIMLSVHGSQHSGMIVNDTGHETDENRAKVEITPNSPRAEATLGGFGSLGLDCEPRTGLDFSDLYYLTMNNKHWLVHKEWFHDIPLPWHAGADTGTPHWNNKEALVEFKDAHAKRQTVVVLGSQEGAVHTALAGALEAEMDGAKGRLSSGHLKCRLKMDKLRLKGVSYSLCTAAFTFTKIPAETLHGTVTVEVQYAGTDGPCKVPAQMAVDMQTLTPVGRLITANPVITESTENSKMMLELDPPFGDSYIVIGVGEKKITHHWHRSGSTIGKAFEATVRGAKRMAVLGDTAWDFGSVGGALNSLGKGIHQIFGAAFKSLFGGMSWFSQILIGTLLMWLGLNAKNGSISLMCLALGGVLIFLSTAVSADVGCSVDFSKKETRCGTGVFVYNDVEAWRDRYKYHPDSPRRLAAAVKQAWEDGICGISSVSRMENIMWRSVEGELNAILEENGVQLTVVVGSVKNPMWRGPQRLPVPVNELPHGWKAWGKSYFVRAAKTNNSFVVDGDTLKECPLKHRAWNSFLVEDHGFGVFHTSVWLKVREDYSLECDPAVIGTAVKGKEAVHSDLGYWIESEKNDTWRLKRAHLIEMKTCEWPKSHTLWTDGIEESDLIIPKSLAGPLSHHNTREGYRTQMKGPWHSEELEIRFEECPGTKVHVEETCGTRGPSLRSTTASGRVIEEWCCRECTMPPLSFRAKDGCWYGMEIRPRKEPESNLVRSMVTAGSTDHMDHFSLGVLVILLMVQEGLKKRMTTKIIISTSMAVLVAMILGGFSMSDLAKLAILMGATFAEMNTGGDVAHLALIAAFKVRPALLVSFIFRANWTPRESMLLALASCLLQTAISALEGDLMVLINGFALAWLAIRAMVVPRTDNITLAILAALTPLARGTLLVAWRAGLATCGGFMLLSLKGKGSVKKNLPFVMALGLTAVRLVDPINVVGLLLLTRSGKRSWPPSEVLTAVGLICALAGGFAKADIEMAGPMAAVGLLIVSYVVSGKSVDMYIERAGDITWEKDAEVTGNSPRLDVALDESGDFSLVEDDGPPMREIILKVVLMTICGMNPIAIPFAAGAWYVYVKTGKRSGALWDVPAPKEVKKGETTDGVYRVMTRRLLGSTQVGVGVMQEGVFHTMWHVTKGSALRSGEGRLDPYWGDVKQDLVSYCGPWKLDAAWDGHSEVQLLAVPPGERARNIQTLPGIFKTKDGDIGAVALDYPAGTSGSPILDKCGRVIGLYGNGVVIKNGSYVSAITQGRREEETPVECFEPSMLKKKQLTVLDLHPGAGKTRRVLPEIVREAIKTRLRTVILAPTRVVAAEMEEALRGLPVRYMTTAVNVTHSGTEIVDLMCHATFTSRLLQPIRVPNYNLYIMDEAHFTDPSSIAARGYISTRVEMGEAAAIFMTATPPGTRDAFPDSNSPIMDTEVEVPERAWSSGFDWVTDHSGKTVWFVPSVRNGNEIAACLTKAGKRVIQLSRKTFETEFQKTKHQEWDFVVTTDISEMGANFKADRVIDSRRCLKPVILDGERVILAGPMPVTHASAAQRRGRIGRNPNKPGDEYLYGGGCAETDEDHAHWLEARMLLDNIYLQDGLIASLYRPEADKVAAIEGEFKLRTEQRKTFVELMKRGDLPVWLAYQVASAGITYTDRRWCFDGTTNNTIMEDSVPAEVWTRHGEKRVLKPRWMDARVCSDHAALKSFKEFAAGKRGAAFGVMEALGTLPGHMTERFQEAIDNLAVLMRAETGSRPYKAAAAQLPETLETIMLLGLLGTVSLGIFFVLMRNKGIGKMGFGMVTLGASAWLMWLSEIEPARIACVLIVVFLLLVVLIPEPEKQRSPQDNQMAIIIMVAVGLLGLITANELGWLERTKSDLSHLMGRREEGATIGFSMDIDLRPASAWAIYAALTTFITPAVQHAVTTSYNNYSLMAMATQAGVLFGMGKGMPFYAWDFGVPLLMIGCYSQLTPLTLIVAIILLVAHYMYLIPGLQAAAARAAQKRTAAGIMKNPVVDGIVVTDIDTMTIDPQVEKKMGQVLLIAVAVSSAILSRTAWGWGEAGALITAATSTLWEGSPNKYWNSSTATSLCNIFRGSYLAGASLIYTVTRNAGLVKRRGGGTGETLGEKWKARLNQMSALEFYSYKKSGITEVCREEARRALKDGVATGGHAVSRGSAKLRWLVERGYLQPYGKVIDLGCGRGGWSYYAATIRKVQEVKGYTKGGPGHEEPVLVQSYGWNIVRLKSGVDVFHMAAEPCDTLLCDIGESSSSPEVEEARTLRVLSMVGDWLEKRPGAFCIKVLCPYTSTMMETLERLQRRYGGGLVRVPLSRNSTHEMYWVSGAKSNTIKSVSTTSQLLLGRMDGPRRPVKYEEDVNLGSGTRAVVSCAEAPNMKIIGNRIERIRSEHAETWFFDENHPYRTWAYHGSYEAPTQGSASSLINGVVRLLSKPWDVVTGVTGIAMTDTTPYGQQRVFKEKVDTRVPDPQEGTRQVMSMVSSWLWKELGKHKRPRVCTKEEFINKVRSNAALGAIFEEEKEWKTAVEAVNDPRFWALVDKEREHHLRGECQSCVYNMMGKREKKQGEFGKAKGSRAIWYMWLGARFLEFEALGFLNEDHWMGRENSGGGVEGLGLQRLGYVLEEMSRIPGGRMYADDTAGWDTRISRFDLENEALITNQMEKGHRALALAIIKYTYQNKVVKVLRPAEKGKTVMDIISRQDQRGSGQVVTYALNTFTNLVVQLIRNMEAEEVLEMQDLWLLRRSEKVTNWLQSNGWDRLKRMAVSGDDCVVKPIDDRFAHALRFLNDMGKVRKDTQEWKPSTGWDNWEEVPFCSHHFNKLHLKDGRSIVVPCRHQDELIGRARVSPGAGWSIRETACLAKSYAQMWQLLYFHRRDLRLMANAICSSVPVDWVPTGRTTWSIHGKGEWMTTEDMLVVWNRVWIEENDHMEDKTPVTKWTDIPYLGKREDLWCGSLIGHRPRTTWAENIKNTVNMVRRIIGDEEKYMDYLSTQVRYLGEEGSTPGVL

SEQ ID NO: 370 - protein, 669 aa, synthetic construct
TRRGSAYYMYLDRNDAGEAISFPTTLGMNKCYIQIMDLGHTCDATMSYECPMLDEGVEPDDVDCWCNTTSTWVVYGTCHHKKGEARRSRRAVTLPSHSTRKLQTRSQTWLESREYTKHLIRVENWIFRNPGFALAAAAIAWLLGSSTSQKVIYLVMILLIAPAYSIRCIGVSNRDFVEGMSGGTWVDVVLEHGGCVTVMAQDKPTVDIELVTTTVSNMAEVRSYCYEASISDMASDSRCPTQGEAYLDKQSDTQYVCKRTLVDRGWGNGCGLFGKGSLVTCAKFACSKKMTGKSIQPENLEYRIMLSVHGSQHSGMIVNDTGHETDENRAKVEITPNSPRAEATLGGFGSLGLDCEPRTGLDFSDLYYLTMNNKHWLVHKEWFHDIPLPWHAGADTGTPHWNNKEALVEFKDAHAKRQTVVVLGSQEGAVHTALAGALEAEMDGAKGRLSSGHLKCRLKMDKLRLKGVSYSLCTAAFTFTKIPAETLHGTVTVEVQYAGTDGPCKVPAQMAVDMQTLTPVGRLITANPVITESTENSKMMLELDPPFGDSYIVIGVGEKKITHHWHRSGSTIGKAFEATVRGAKRMAVLGDTAWDFGSVGGALNSLGKGIHQIFGAAFKSLFGGMSWFSQILIGTLLMWLGLNAKNGSISLMCLALGGVLIFLSTAVSA

SEQ ID NO: 371 - protein, 23 aa, synthetic construct
GKRSAGSIMWLASLAVVIACAGA

SEQ ID NO: 372 - protein, 692 aa, synthetic construct
GKRSAGSIMWLASLAVVIACAGATRRGSAYYMYLDRNDAGEAISFPTTLGMNKCYIQIMDLGHTCDATMSYECPMLDEGVEPDDVDCWCNTTSTWVVYGTCHHKKGEARRSRRAVTLPSHSTRKLQTRSQTWLESREYTKHLIRVENWIFRNPGFALAAAAIAWLLGSSTSQKVIYLVMILLIAPAYSIRCIGVSNRDFVEGMSGGTWVDVVLEHGGCVTVMAQDKPTVDIELVTTTVSNMAEVRSYCYEASISDMASDSRCPTQGEAYLDKQSDTQYVCKRTLVDRGWGNGCGLFGKGSLVTCAKFACSKKMTGKSIQPENLEYRIMLSVHGSQHSGMIVNDTGHETDENRAKVEITPNSPRAEATLGGFGSLGLDCEPRTGLDFSDLYYLTMNNKHWLVHKEWFHDIPLPWHAGADTGTPHWNNKEALVEFKDAHAKRQTVVVLGSQEGAVHTALAGALEAEMDGAKGRLSSGHLKCRLKMDKLRLKGVSYSLCTAAFTFTKIPAETLHGTVTVEVQYAGTDGPCKVPAQMAVDMQTLTPVGRLITANPVITESTENSKMMLELDPPFGDSYIVIGVGEKKITHHWHRSGSTIGKAFEATVRGAKRMAVLGDTAWDFGSVGGALNSLGKGIHQIFGAAFKSLFGGMSWFSQILIGTLLMWLGLNAKNGSISLMCLALGGVLIFLSTAVSA

SEQ ID NO: 373 - protein, 352 aa, Zika virus
VGCSVDFSKKETRCGTGVFVYNDVEAWRDRYKYHPDSPRRLAAAVKQAWEDGICGISSVSRMENIMWRSVEGELNAILEENGVQLTVVVGSVKNPMWRGPQRLPVPVNELPHGWKAWGKSYFVRAAKTNNSFVVDGDTLKECPLKHRAWNSFLVEDHGFGVFHTSVWLKVREDYSLECDPAVIGTAVKGKEAVHSDLGYWIESEKNDTWRLKRAHLIEMKTCEWPKSHTLWTDGIEESDLIIPKSLAGPLSHHNTREGYRTQMKGPWHSEELEIRFEECPGTKVHVEETCGTRGPSLRSTTASGRVIEEWCCRECTMPPLSFRAKDGCWYGMEIRPRKEPESNLVRSMVTAG

SEQ ID NO: 374 - protein, 692 aa, synthetic construct; identical to SEQ ID NO: 372

SEQ ID NO: 375 - protein, 642 aa, synthetic construct
GKRSAGSIMWLASLAVVIACAGATRRGSAYYMYLDRNDAGEAISFPTTLGMNKCYIQIMDLGHTCDATMSYECPMLDEGVEPDDVDCWCNTTSTWVVYGTCHHKKGEARRSRRAVTLPSHSTRKLQTRSQTWLESREYTKHLIRVENWIFRNPGFALAAAAIAWLLGSSTSQKVIYLVMILLIAPAYSIRCIGVSNRDFVEGMSGGTWVDVVLEHGGCVTVMAQDKPTVDIELVTTTVSNMAEVRSYCYEASISDMASDSRCPTQGEAYLDKQSDTQYVCKRTLVDRGWGNGCGLFGKGSLVTCAKFACSKKMTGKSIQPENLEYRIMLSVHGSQHSGMIVNDTGHETDENRAKVEITPNSPRAEATLGGFGSLGLDCEPRTGLDFSDLYYLTMNNKHWLVHKEWFHDIPLPWHAGADTGTPHWNNKEALVEFKDAHAKRQTVVVLGSQEGAVHTALAGALEAEMDGAKGRLSSGHLKCRLKMDKLRLKGVSYSLCTAAFTFTKIPAETLHGTVTVEVQYAGTDGPCKVPAQMAVDMQTLTPVGRLITANPVITESTENSKMMLELDPPFGDSYIVIGVGEKKITHHWHRSGSTIGKAFEATVRGAKRMAVLGDTAWDFGSVGGALNSLGKGIHQIFGAAFK

SEQ ID NO: 376 - protein, 619 aa, synthetic construct
TRRGSAYYMYLDRNDAGEAISFPTTLGMNKCYIQIMDLGHTCDATMSYECPMLDEGVEPDDVDCWCNTTSTWVVYGTCHHKKGEARRSRRAVTLPSHSTRKLQTRSQTWLESREYTKHLIRVENWIFRNPGFALAAAAIAWLLGSSTSQKVIYLVMILLIAPAYSIRCIGVSNRDFVEGMSGGTWVDVVLEHGGCVTVMAQDKPTVDIELVTTTVSNMAEVRSYCYEASISDMASDSRCPTQGEAYLDKQSDTQYVCKRTLVDRGWGNGCGLFGKGSLVTCAKFACSKKMTGKSIQPENLEYRIMLSVHGSQHSGMIVNDTGHETDENRAKVEITPNSPRAEATLGGFGSLGLDCEPRTGLDFSDLYYLTMNNKHWLVHKEWFHDIPLPWHAGADTGTPHWNNKEALVEFKDAHAKRQTVVVLGSQEGAVHTALAGALEAEMDGAKGRLSSGHLKCRLKMDKLRLKGVSYSLCTAAFTFTKIPAETLHGTVTVEVQYAGTDGPCKVPAQMAVDMQTLTPVGRLITANPVITESTENSKMMLELDPPFGDSYIVIGVGEKKITHHWHRSGSTIGKAFEATVRGAKRMAVLGDTAWDFGSVGGALNSLGKGIHQIFGAAFK

SEQ ID NO: 377 - protein, 1272 aa, synthetic construct
FVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 378 - protein, 1272 aa, synthetic construct
FVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGTIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 379 - protein, 1270 aa, synthetic construct
FVFLVLLPLVSSQCVNLRTRTQLPPAYTNSFTRGVYYPDKVFRSSVLHSTQDLFLPFFSNVTWFHAIHVSGTNGTKRFDNPVLPFNDGVYFASIEKSNIIRGWIFGTTLDSKTQSLLIVNNATNVVIKVCEFQFCNDPFLDVYYHKNNKSWMESGVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSRRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQNVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 380 - protein, 1270 aa, synthetic construct
FVFLVLLPLVSSQCVNLRTRTQLPPAYTNSFTRGVYYPDKVFRSSVLHSTQDLFLPFFSNVTWFHAIHFSGTNGTKRFDNPVLPFNDGVYFASIEKSNIIRGWIFGTTLDSKTQSLLIVNNATNVVIKVCEFQFCNDPFLDVYYHKNNKSWMESGVYSSANNCTFEYVSQPFLMDLEGKQGNFKNLREFVFKNIDGYFKIYSKHTPINLVRDLPQGFSVLEPLVDLPIGINITRFQTLLALHRSYLTPGDSSSGLTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSRRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQNVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 381 - protein, 1272 aa, synthetic construct
FVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 382 - protein, 1272 aa, synthetic construct
FVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGTIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 383 - protein, 1270 aa, synthetic construct
FVFLVLLPLVSSQCVNLRTRTQLPPAYTNSFTRGVYYPDKVFRSSVLHSTQDLFLPFFSNVTWFHAIHVSGTNGTKRFDNPVLPFNDGVYFASIEKSNIIRGWIFGTTLDSKTQSLLIVNNATNVVIKVCEFQFCNDPFLDVYYHKNNKSWMESGVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSRRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQNVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 384 - protein, 1270 aa, synthetic construct
FVFLVLLPLVSSQCVNLRTRTQLPPAYTNSFTRGVYYPDKVFRSSVLHSTQDLFLPFFSNVTWFHAIHFSGTNGTKRFDNPVLPFNDGVYFASIEKSNIIRGWIFGTTLDSKTQSLLIVNNATNVVIKVCEFQFCNDPFLDVYYHKNNKSWMESGVYSSANNCTFEYVSQPFLMDLEGKQGNFKNLREFVFKNIDGYFKIYSKHTPINLVRDLPQGFSVLEPLVDLPIGINITRFQTLLALHRSYLTPGDSSSGLTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSRRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQNVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 385 - protein, 74 aa, synthetic construct
YSFVSEETGTLIVNSVLLFLAFVVFLLVTLAILTALRLCAYCCNIVNVSLVKPSFYVYSRVKNLNSSRVPDLLV

SEQ ID NO: 386 - protein, 221 aa, synthetic construct
ADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQ

SEQ ID NO: 387 - protein, 7095 aa, synthetic construct
ESLVPGFNEKTHVQLSLPVLQVRDVLVRGFGDSVEEVLSEARQHLKDGTCGLVEVEKGVLPQLEQPYVFIKRSDARTAPHGHVMVELVAELEGIQYGRSGETLGVLVPHVGEIPVAYRKVLLRKNGNKGAGGHSYGADLKSFDLGDELGTDPYEDFQENWNTKHSSGVTRELMRELNGGAYTRYVDNNFCGPDGYPLECIKDLLARAGKASCTLSEQLDFIDTKRGVYCCREHEHEIAWYTERSEKSYELQTPFEIKLAKKFDTFNGECPNFVFPLNSIIKTIQPRVEKKKLDGFMGRIRSVYPVASPNECNQMCLSTLMKCDHCGETSWQTGDFVKATCEFCGTENLTKEGATTCGYLPQNAVVKIYCPACHNSEVGPEHSLAEYHNESGLKTILRKGGRTIAFGGCVFSYVGCHNKCAYWVPRASANIGCNHTGVVGEGSEGLNDNLLEILQKEKVNINIVGDFKLNEEIAIILASFSASTSAFVETVKGLDYKAFKQIVESCGNFKVTKGKAKKGAWNIGEQKSILSPLYAFASEAARVVRSIFSRTLETAQNSVRVLQKAAITILDGISQYSLRLIDAMMFTSDLATNNLVVMAYITGGVVQLTSQWLTNIFGTVYEKLKPVLDWLEEKFKEGVEFLRDGWEIVKFISTCACEIVGGQIVTCAKEIKESVQTFFKLVNKFLALCADSIIIGGAKLKALNLGETFVTHSKGLYRKCVKSREETGLLMPLKAPKEIIFLEGETLPTEVLTEEVVLKTGDLQPLEQPTSEAVEAPLVGTPVCINGLMLLEIKDTEKYCALAPNMMVTNNTFTLKGGAPTKVTFGDDTVIEVQGYKSVNITFELDERIDKVLNEKCSAYTVELGTEVNEFACVVADAVIKTLQPVSELLTPLGIDLDEWSMATYYLFDESGEFKLASHMYCSFYPPDEDEEEGDCEEEEFEPSTQYEYGTEDDYQGKPLEFGATSAALQPEEEQEEDWLDDDSQQTVGQQDGSEDNQTTTIQTIVEVQPQLEMELTPVVQTIEVNSFSGYLKLTDNVYIKNADIVEEAKKVKPTVVVNAANVYLKHGGGVAGALNKATNNAMQVESDDYIATNGPLKVGGSCVLSGHNLAKHCLHVVGPNVNKGEDIQLLKSAYENFNQHEVLLAPLLSAGIFGADPIHSLRVCVDTVRTNVYLAVFDKNLYDKLVSSFLEMKSEKQVEQKIAEIPKEEVKPFITESKPSVEQRKQDDKKIKACVEEVTTTLEETKFLTENLLLYIDINGNLHPDSATLVSDIDITFLKKDAPYIVGDVVQEGVLTAVVIPTKKAGGTTEMLAKALRKVPTDNYITTYPGQGLNGYTVEEAKTVLKKCKSAFYILPSIISNEKQEILGTVSWNLREMLAHAEETRKLMPVCVETKAIVSTIQRKYKGIKIQEGVVDYGARFYFYTSKTTVASLINTLNDLNETLVTMPLGYVTHGLNLEEAARYMRSLKVPATVSVSSPDAVTAYNGYLTSSSKTPEEHFIETISLAGSYKDWSYSGQSTQLGIEFLKRGDKSVYYTSNPTTFHLDGEVITFDNLKTLLSLREVRTIKVFTTVDNINLHTQVVDMSMTYGQQFGPTYLDGADVTKIKPHNSHEGKTFYVLPNDDTLRVEAFEYYHTTDPSFLGRYMSALNHTKKWKYPQVNGLTSIKWADNNCYLATALLTLQQIELKFNPPALQDAYYRARAGEAANFCALILAYCNKTVGELGDVRETMSYLFQHANLDSCKRVLNVVCKTCGQQQTTLKGVEAVMYMGTLSYEQFKKGVQIPCTCGKQATKYLVQQESPFVMMSAPPAQYELKHGTFTCASEYTGNYQCGHYKHITSKETLYCIDGALLTKSSEYKGPITDVFYKENSYTTTIKPVTYKLDGVVCTEIDPKLDNYYKKDNSYFTEQPIDLVPNQPYPNASFDNFKFVCDNIKFADDLNQLTGYKKPASRELKVTFFPDLNGDVVAIDYKHYTPSFKKGAKLLHKPIVWHVNNATNKATYKPNTWCIRCLWSTKPVETSNSFDVLKSEDAQGMDNLACEDLKPVSEEVVENPTIQKDVLECNVKTTEVVGDIILKPANNSLKITEEVGHTDLMAAYVDNSSLTIKKPNELSRVLGLKTLATHGLAAVNSVPWDTIANYAKPFLNKVVSTTTNIVTRCLNRVCTNYMPYFFTLLLQLCTFTRSTNSRIKASMPTTIAKNTVKSVGKFCLEASFNYLKSPNFSKLINIIIWFLLLSVCLGSLIYSTAALGVLMSNLGMPSYCTGYREGYLNSTNVTIATYCTGSIPCSVCLSGLDSLDTYPSLETIQITISSFKWDLTAFGLVAEWFLAYILFTRFFYVLGLAAIMQLFFSYFAVHFISNSWLMWLIINLVQMAPISAMVRMYIFFASFYYVWKSYVHVVDGCNSSTCMMCYKRNRATRVECTTIVNGVRRSFYVYANGGKGFCKLHNWNCVNCDTFCAGSTFISDEVARDLSLQFKRPINPTDQSSYIVDSVTVKNGSIHLYFDKAGQKTYERHSLSHFVNLDNLRANNTKGSLPINVIVFDGKSKCEESSAKSASVYYSQLMCQPILLLDQALVSDVGDSAEVAVKMFDAYVNTFSSTFNVPMEKLKTLVATAEAELAKNVSLDNVLSTFISAARQGFVDSDVETKDVVECLKLSHQSDIEVTGDSCNNYMLTYNKVENMTPRDLGACIDCSARHINAQVAKSHNIALIWNVKDFMSLSEQLRKQIRSAAKKNNLPFKLTCATTRQVVNVVTTKIALKGGKIVNNWLKQLIKVTLVFLFVAAIFYLITPVHVMSKHTDFSSEIIGYKAIDGGVTRDIASTDTCFANKHADFDTWFSQRGGSYTNDKACPLIAAVITREVGFVVPGLPGTILRTTNGDFLHFLPRVFSAVGNICYTPSKLIEYTDFATSACVLAAECTIFKDASGKPVPYCYDTNVLEGSVAYESLRPDTRYVLMDGSIIQFPNTYLEGSVRVVTTFDSEYCRHGTCERSEAGVCVSTSGRWVLNNDYYRSLPGVFCGVDAVNLLTNMFTPLIQPIGALDISASIVAGGIVAIVVTCLAYYFMRFRRAFGEYSHVVAFNTLLFLMSFTVLCLTPVYSFLPGVYSVIYLYLTFYLTNDVSFLAHIQWMVMFTPLVPFWITIAYIICISTKHFYWFFSNYLKRRVVFNGVSFSTFEEAALCTFLLNKEMYLKLRSDVLLPLTQYNRYLALYNKYKYFSGAMDTTSYREAACCHLAKALNDFSNSGSDVLYQPPQTSITSAVLQSGFRKMAFPSGKVEGCMVQVTCGTTTLNGLWLDDVVYCPRHVICTSEDMLNPNYEDLLIRKSNHNFLVQAGNVQLRVIGHSMQNCVLKLKVDTANPKTPKYKFVRIQPGQTFSVLACYNGSPSGVYQCAMRPNFTIKGSFLNGSCGSVGFNIDYDCVSFCYMHHMELPTGVHAGTDLEGNFYGPFVDRQTAQAAGTDTTITVNVLAWLYAAVINGDRWFLNRFTTTLNDFNLVAMKYNYEPLTQDHVDILGPLSAQTGIAVLDMCASLKELLQNGMNGRTILGSALLEDEFTPFDVVRQCSGVTFQSAVKRTIKGTHHWLLLTILTSLLVLVQSTQWSLFFFLYENAFLPFAMGIIAMSAFAMMFVKHKHAFLCLFLLPSLATVAYFNMVYMPASWVMRIMTWLDMVDTSLSGFKLKDCVMYASAVVLLILMTARTVYDDGARRVWTLMNVLTLVYKVYYGNALDQAISMWALIISVTSNYSGVVTTVMFLARGIVFMCVEYCPIFFITGNTLQCIMLVYCFLGYFCTCYFGLFCLLNRYFRLTLGVYDYLVSTQEFRYMNSQGLLPPKNSIDAFKLNIKLLGVGGKPCIKVATVQSKMSDVKCTSVVLLSVLQQLRVESSSKLWAQCVQLHNDILLAKDTTEAFEKMVSLLSVLLSMQGAVDINKLCEEMLDNRATLQAIASEFSSLPSYAAFATAQEAYEQAVANGDSEVVLKKLKKSLNVAKSEFDRDAAMQRKLEKMADQAMTQMYKQARSEDKRAKVTSAMQTMLFTMLRKLDNDALNNIINNARDGCVPLNIIPLTTAAKLMVVIPDYNTYKNTCDGTTFTYASALWEIQQVVDADSKIVQLSEISMDNSPNLAWPLIVTALRANSAVKLQNNELSPVALRQMSCAAGTTQTACTDDNALAYYNTTKGGRFVLALLSDLQDLKWARFPKSDGTGTIYTELEPPCRFVTDTPKGPKVKYLYFIKGLNNLNRGMVLGSLAATVRLQAGNATEVPANSTVLSFCAFAVDAAKAYKDYLASGGQPITNCVKMLCTHTGTGQAITVTPEANMDQESFGGASCCLYCRCHIDHPNPKGFCDLKGKYVQIPTTCANDPVGFTLKNTVCTVCGMWKGYGCSCDQLREPMLQSADAQSFLNRVCGVSAARLTPCGTGTSTDVVYRAFDIYNDKVAGFAKFLKTNCCRFQEKDEDDNLIDSYFVVKRHTFSNYQHEETIYNLLKDCPAVAKHDFFKFRIDGDMVPHISRQRLTKYTMADLVYALRHFDEGNCDTLKEILVTYNCCDDDYFNKKDWYDFVENPDILRVYANLGERVRQALLKTVQFCDAMRNAGIVGVLTLDNQDLNGNWYDFGDFIQTTPGSGVPVVDSYYSLLMPILTLTRALTAESHVDTDLTKPYIKWDLLKYDFTEERLKLFDRYFKYWDQTYHPNCVNCLDDRCILHCANFNVLFSTVFPPTSFGPLVRKIFVDGVPFVVSTGYHFRELGVVHNQDVNLHSSRLSFKELLVYAADPAMHAASGNLLLDKRTTCFSVAALTNNVAFQTVKPGNFNKDFYDFAVSKGFFKEGSSVELKHFFFAQDGNAAISDYDYYRYNLPTMCDIRQLLFVVEVVDKYFDCYDGGCINANQVIVNNLDKSAGFPFNKWGKARLYYDSMSYEDQDALFAYTKRNVIPTITQMNLKYAISAKNRARTVAGVSICSTMTNRQFHQKLLKSIAATRGATVVIGTSKFYGGWHNMLKTVYSDVENPHLMGWDYPKCDRAMPNMLRIMASLVLARKHTTCCSLSHRFYRLANECAQVLSEMVMCGGSLYVKPGGTSSGDATTAYANSVFNICQAVTANVNALLSTDGNKIADKYVRNLQHRLYECLYRNRDVDTDFVNEFYAYLRKHFSMMILSDDAVVCFNSTYASQGLVASIKNFKSVLYYQNNVFMSEAKCWTETDLTKGPHEFCSQHTMLVKQGDDYVYLPYPDPSRILGAGCFVDDIVKTDGTLMIERFVSLAIDAYPLTKHPNQEYADVFHLYLQYIRKLHDELTGHMLDMYSVMLTNDNTSRYWEPEFYEAMYTPHTVLQAVGACVLCNSQTSLRCGACIRRPFLCCKCCYDHVISTSHKLVLSVNPYVCNAPGCDVTDVTQLYLGGMSYYCKSHKPPISFPLCANGQVFGLYKNTCVGSDNVTDFNAIATCDWTNAGDYILANTCTERLKLFAAETLKATEETFKLSYGIATVREVLSDRELHLSWEVGKPRPPLNRNYVFTGYRVTKNSKVQIGEYTFEKGDYGDAVVYRGTTTYKLNVGDYFVLTSHTVMPLSAPTLVPQEHYVRITGLYPTLNISDEFSSNVANYQKVGMQKYSTLQGPPGTGKSHFAIGLALYYPSARIVYTACSHAAVDALCEKALKYLPIDKCSRIIPARARVECFDKFKVNSTLEQYVFCTVNALPETTADIVVFDEISMATNYDLSVVNARLRAKHYVYIGDPAQLPAPRTLLTKGTLEPEYFNSVCRLMKTIGPDMFLGTCRRCPAEIVDTVSALVYDNKLKAHKDKSAQCFKMFYKGVITHDVSSAINRPQIGVVREFLTRNPAWRKAVFISPYNSQNAVASKILGLPTQTVDSSQGSEYDYVIFTQTTETAHSCNVNRFNVAITRAKVGILCIMSDRDLYDKLQFTSLEIPRRNVATLQAENVTGLFKDCSKVITGLHPTQAPTHLSVDTKFKTEGLCVDIPGIPKDMTYRRLISMMGFKMNYQVNGYPNMFITREEAIRHVRAWIGFDVEGCHATREAVGTNLPLQLGFSTGVNLVAVPTGYVDTPNNTDFSRVSAKPPPGDQFKHLIPLMYKGLPWNVVRIKIVQMLSDTLKNLSDRVVFVLWAHGFELTSMKYFVKIGPERTCCLCDRRATCFSTASDTYACWHHSIGFDYVYNPFMIDVQQWGFTGNLQSNHDLYCQVHGNAHVASCDAIMTRCLAVHECFVKRVDWTIEYPIIGDELKINAACRKVQHMVVKAALLADKFPVLHDIGNPKAIKCVPQADVEWKFYDAQPCSDKAYKIEELFYSYATHSDKFTDGVCLFWNCNVDRYPANSIVCRFDTRVLSNLNLPGCDGGSLYVNKHAFHTPAFDKSAFVNLKQLPFFYYSDSPCESHGKQVVSDIDYVPLKSATCITRCNLGGAVCRHHANEYRLYLDAYNMMISAGFSLWVYKQFDTYNLWNTFTRLQSLENVAFNVVNKGHFDGQQGEVPVSIINNTVYTKVDGVDVELFENKTTLPVNVAFELWAKRNIKPVPEVKILNNLGVDIAANTVIWDYKRDAPAHISTIGVCSMTDIAKKPTETICAPLTVFFDGRVDGQVDLFRNARNGVLITEGSVKGLQPSVGPKQASLNGVTLIGEAVKTQFNYYKKVDGVVQQLPETYFTQSRNLQEFKPRSQMEIDFLELAMDEFIERYKLEGYAFEHIVYGDFSHSQLGGLHLLIGLAKRFKESPFELEDFIPMDSTVKNYFITDAQTGSSKCVCSVIDLLLDDFVEIIKSQDLSVVSKVVKVTIDYTEISFMLWCKDGHVETFYPKLQSSQAWQPGVAMPNLYKMQRMLLEKCDLQNYGDSATLPKGIMMNVAKYTQLCQYLNTLTLAVPYNMRVIHFGAGSDKGVAPGTAVLRQWLPTGTLLVDSDLNDFVSDADSTLIGDCATVHTANKWDLIISDMYDPKTKNVTKENDSKEGFFTYICGFIQQKLALGGSVAIKITEHSWNADLYKLMGHFAWWTAFVTNVNASSSEAFLIGCNYLGKPREQIDGYVMHANYIFWRNTNPIQLSSYSLFDMSKFPLKLRGTAVMSLKEGQINDMILSLLSKGRLIIRENNRVVISSDVLVNN

SEQ ID NO: 388 - protein, 4403 aa, synthetic construct
ESLVPGFNEKTHVQLSLPVLQVRDVLVRGFGDSVEEVLSEARQHLKDGTCGLVEVEKGVLPQLEQPYVFIKRSDARTAPHGHVMVELVAELEGIQYGRSGETLGVLVPHVGEIPVAYRKVLLRKNGNKGAGGHSYGADLKSFDLGDELGTDPYEDFQENWNTKHSSGVTRELMRELNGGAYTRYVDNNFCGPDGYPLECIKDLLARAGKASCTLSEQLDFIDTKRGVYCCREHEHEIAWYTERSEKSYELQTPFEIKLAKKFDTFNGECPNFVFPLNSIIKTIQPRVEKKKLDGFMGRIRSVYPVASPNECNQMCLSTLMKCDHCGETSWQTGDFVKATCEFCGTENLTKEGATTCGYLPQNAVVKIYCPACHNSEVGPEHSLAEYHNESGLKTILRKGGRTIAFGGCVFSYVGCHNKCAYWVPRASANIGCNHTGVVGEGSEGLNDNLLEILQKEKVNINIVGDFKLNEEIAIILASFSASTSAFVETVKGLDYKAFKQIVESCGNFKVTKGKAKKGAWNIGEQKSILSPLYAFASEAARVVRSIFSRTLETAQNSVRVLQKAAITILDGISQYSLRLIDAMMFTSDLATNNLVVMAYITGGVVQLTSQWLTNIFGTVYEKLKPVLDWLEEKFKEGVEFLRDGWEIVKFISTCACEIVGGQIVTCAKEIKESVQTFFKLVNKFLALCADSIIIGGAKLKALNLGETFVTHSKGLYRKCVKSREETGLLMPLKAPKEIIFLEGETLPTEVLTEEVVLKTGDLQPLEQPTSEAVEAPLVGTPVCINGLMLLEIKDTEKYCALAPNMMVTNNTFTLKGGAPTKVTFGDDTVIEVQGYKSVNITFELDERIDKVLNEKCSAYTVELGTEVNEFACVVADAVIKTLQPVSELLTPLGIDLDEWSMATYYLFDESGEFKLASHMYCSFYPPDEDEEEGDCEEEEFEPSTQYEYGTEDDYQGKPLEFGATSAALQPEEEQEEDWLDDDSQQTVGQQDGSEDNQTTTIQTIVEVQPQLEMELTPVVQTIEVNSFSGYLKLTDNVYIKNADIVEEAKKVKPTVVVNAANVYLKHGGGVAGALNKATNNAMQVESDDYIATNGPLKVGGSCVLSGHNLAKHCLHVVGPNVNKGEDIQLLKSAYENFNQHEVLLAPLLSAGIFGADPIHSLRVCVDTVRTNVYLAVFDKNLYDKLVSSFLEMKSEKQVEQKIAEIPKEEVKPFITESKPSVEQRKQDDKKIKACVEEVTTTLEETKFLTENLLLYIDINGNLHPDSATLVSDIDITFLKKDAPYIVGDVVQEGVLTAVVIPTKKAGGTTEMLAKALRKVPTDNYITTYPGQGLNGYTVEEAKTVLKKCKSAFYILPSIISNEKQEILGTVSWNLREMLAHAEETRKLMPVCVETKAIVSTIQRKYKGIKIQEGVVDYGARFYFYTSKTTVASLINTLNDLNETLVTMPLGYVTHGLNLEEAARYMRSLKVPATVSVSSPDAVTAYNGYLTSSSKTPEEHFIETISLAGSYKDWSYSGQSTQLGIEFLKRGDKSVYYTSNPTTFHLDGEVITFDNLKTLLSLREVRTIKVFTTVDNINLHTQVVDMSMTYGQQFGPTYLDGADVTKIKPHNSHEGKTFYVLPNDDTLRVEAFEYYHTTDPSFLGRYMSALNHTKKWKYPQVNGLTSIKWADNNCYLATALLTLQQIELKFNPPALQDAYYRARAGEAANFCALILAYCNKTVGELGDVRETMSYLFQHANLDSCKRVLNVVCKTCGQQQTTLKGVEAVMYMGTLSYEQFKKGVQIPCTCGKQATKYLVQQESPFVMMSAPPAQYELKHGTFTCASEYTGNYQCGHYKHITSKETLYCIDGALLTKSSEYKGPITDVFYKENSYTTTIKPVTYKLDGVVCTEIDPKLDNYYKKDNSYFTEQPIDLVPNQPYPNASFDNFKFVCDNIKFADDLNQLTGYKKPASRELKVTFFPDLNGDVVAIDYKHYTPSFKKGAKLLHKPIVWHVNNATNKATYKPNTWCIRCLWSTKPVETSNSFDVLKSEDAQGMDNLACEDLKPVSEEVVENPTIQKDVLECNVKTTEVVGDIILKPANNSLKITEEVGHTDLMAAYVDNSSLTIKKPNELSRVLGLKTLATHGLAAVNSVPWDTIANYAKPFLNKVVSTTTNIVTRCLNRVCTNYMPYFFTLLLQLCTFTRSTNSRIKASMPTTIAKNTVKSVGKFCLEASFNYLKSPNFSKLINIIIWFLLLSVCLGSLIYSTAALGVLMSNLGMPSYCTGYREGYLNSTNVTIATYCTGSIPCSVCLSGLDSLDTYPSLETIQITISSFKWDLTAFGLVAEWFLAYILFTRFFYVLGLAAIMQLFFSYFAVHFISNSWLMWLIINLVQMAPISAMVRMYIFFASFYYVWKSYVHVVDGCNSSTCMMCYKRNRATRVECTTIVNGVRRSFYVYANGGKGFCKLHNWNCVNCDTFCAGSTFISDEVARDLSLQFKRPINPTDQSSYIVDSVTVKNGSIHLYFDKAGQKTYERHSLSHFVNLDNLRANNTKGSLPINVIVFDGKSKCEESSAKSASVYYSQLMCQPILLLDQALVSDVGDSAEVAVKMFDAYVNTFSSTFNVPMEKLKTLVATAEAELAKNVSLDNVLSTFISAARQGFVDSDVETKDVVECLKLSHQSDIEVTGDSCNNYMLTYNKVENMTPRDLGACIDCSARHINAQVAKSHNIALIWNVKDFMSLSEQLRKQIRSAAKKNNLPFKLTCATTRQVVNVVTTKIALKGGKIVNNWLKQLIKVTLVFLFVAAIFYLITPVHVMSKHTDFSSEIIGYKAIDGGVTRDIASTDTCFANKHADFDTWFSQRGGSYTNDKACPLIAAVITREVGFVVPGLPGTILRTTNGDFLHFLPRVFSAVGNICYTPSKLIEYTDFATSACVLAAECTIFKDASGKPVPYCYDTNVLEGSVAYESLRPDTRYVLMDGSIIQFPNTYLEGSVRVVTTFDSEYCRHGTCERSEAGVCVSTSGRWVLNNDYYRSLPGVFCGVDAVNLLTNMFTPLIQPIGALDISASIVAGGIVAIVVTCLAYYFMRFRRAFGEYSHVVAFNTLLFLMSFTVLCLTPVYSFLPGVYSVIYLYLTFYLTNDVSFLAHIQWMVMFTPLVPFWITIAYIICISTKHFYWFFSNYLKRRVVFNGVSFSTFEEAALCTFLLNKEMYLKLRSDVLLPLTQYNRYLALYNKYKYFSGAMDTTSYREAACCHLAKALNDFSNSGSDVLYQPPQTSITSAVLQSGFRKMAFPSGKVEGCMVQVTCGTTTLNGLWLDDVVYCPRHVICTSEDMLNPNYEDLLIRKSNHNFLVQAGNVQLRVIGHSMQNCVLKLKVDTANPKTPKYKFVRIQPGQTFSVLACYNGSPSGVYQCAMRPNFTIKGSFLNGSCGSVGFNIDYDCVSFCYMHHMELPTGVHAGTDLEGNFYGPFVDRQTAQAAGTDTTITVNVLAWLYAAVINGDRWFLNRFTTTLNDFNLVAMKYNYEPLTQDHVDILGPLSAQTGIAVLDMCASLKELLQNGMNGRTILGSALLEDEFTPFDVVRQCSGVTFQSAVKRTIKGTHHWLLLTILTSLLVLVQSTQWSLFFFLYENAFLPFAMGIIAMSAFAMMFVKHKHAFLCLFLLPSLATVAYFNMVYMPASWVMRIMTWLDMVDTSLSGFKLKDCVMYASAVVLLILMTARTVYDDGARRVWTLMNVLTLVYKVYYGNALDQAISMWALIISVTSNYSGVVTTVMFLARGIVFMCVEYCPIFFITGNTLQCIMLVYCFLGYFCTCYFGLFCLLNRYFRLTLGVYDYLVSTQEFRYMNSQGLLPPKNSIDAFKLNIKLLGVGGKPCIKVATVQSKMSDVKCTSVVLLSVLQQLRVESSSKLWAQCVQLHNDILLAKDTTEAFEKMVSLLSVLLSMQGAVDINKLCEEMLDNRATLQAIASEFSSLPSYAAFATAQEAYEQAVANGDSEVVLKKLKKSLNVAKSEFDRDAAMQRKLEKMADQAMTQMYKQARSEDKRAKVTSAMQTMLFTMLRKLDNDALNNIINNARDGCVPLNIIPLTTAAKLMVVIPDYNTYKNTCDGTTFTYASALWEIQQVVDADSKIVQLSEISMDNSPNLAWPLIVTALRANSAVKLQNNELSPVALRQMSCAAGTTQTACTDDNALAYYNTTKGGRFVLALLSDLQDLWARFPKSDGTGTIYTELEPPCRFVTDTPKGPKVKYLYFIKGLNNLNRGMVLGSLAATVRLQAGNATEVPANSTVLSFCAFAVDAAKAYKDYLASGGQPITNCVKMLCTHTGTGQAITVTPEANMDQESFGGASCCLYCRCHIDHPNPKGFCDLKGKYVQIPTTCANDPVGFTLKNTVCTVCGMWKGYGCSCDQLREPMLQSADAQSFLNGFAV

SEQ ID NO: 389 - protein, 2762 aa, synthetic construct
ESLVPGFNEKTHVQLSLPVLQVRDVLVRGFGDSVEEVLSEARQHLKDGTCGLVEVEKGVLPQLEQPYVFIKRSDARTAPHGHVMVELVAELEGIQYGRSGETLGVLVPHVGEIPVAYRKVLLRKNGNKGAGGHSYGADLKSFDLGDELGTDPYEDFQENWNTKHSSGVTRELMRELNGGAYTRYVDNNFCGPDGYPLECIKDLLARAGKASCTLSEQLDFIDTKRGVYCCREHEHEIAWYTERSEKSYELQTPFEIKLAKKFDTFNGECPNFVFPLNSIIKTIQPRVEKKKLDGFMGRIRSVYPVASPNECNQMCLSTLMKCDHCGETSWQTGDFVKATCEFCGTENLTKEGATTCGYLPQNAVVKIYCPACHNSEVGPEHSLAEYHNESGLKTILRKGGRTIAFGGCVFSYVGCHNKCAYWVPRASANIGCNHTGVVGEGSEGLNDNLLEILQKEKVNINIVGDFKLNEEIAIILASFSASTSAFVETVKGLDYKAFKQIVESCGNFKVTKGKAKKGAWNIGEQKSILSPLYAFASEAARVVRSIFSRTLETAQNSVRVLQKAAITILDGISQYSLRLIDAMMFTSDLATNNLVVMAYITGGVVQLTSQWLTNIFGTVYEKLKPVLDWLEEKFKEGVEFLRDGWEIVKFISTCACEIVGGQIVTCAKEIKESVQTFFKLVNKFLALCADSIIIGGAKLKALNLGETFVTHSKGLYRKCVKSREETGLLMPLKAPKEIIFLEGETLPTEVLTEEVVLKTGDLQPLEQPTSEAVEAPLVGTPVCINGLMLLEIKDTEKYCALAPNMMVTNNTFTLKGGAPTKVTFGDDTVIEVQGYKSVNITFELDERIDKVLNEKCSAYTVELGTEVNEFACVVADAVIKTLQPVSELLTPLGIDLDEWSMATYYLFDESGEFKLASHMYCSFYPPDEDEEEGDCEEEEFEPSTQYEYGTEDDYQGKPLEFGATSAALQPEEEQEEDWLDDDSQQTVGQQDGSEDNQTTTIQTIVEVQPQLEMELTPVVQTIEVNSFSGYLKLTDNVYIKNADIVEEAKKVKPTVVVNAANVYLKHGGGVAGALNKATNNAMQVESDDYIATNGPLKVGGSCVLSGHNLAKHCLHVVGPNVNKGEDIQLLKSAYENFNQHEVLLAPLLSAGIFGADPIHSLRVCVDTVRTNVYLAVFDKNLYDKLVSSFLEMKSEKQVEQKIAEIPKEEVKPFITESKPSVEQRKQDDKKIKACVEEVTTTLEETKFLTENLLLYIDINGNLHPDSATLVSDIDITFLKKDAPYIVGDVVQEGVLTAVVIPTKKAGGTTEMLAKALRKVPTDNYITTYPGQGLNGYTVEEAKTVLKKCKSAFYILPSIISNEKQEILGTVSWNLREMLAHAEETRKLMPVCVETKAIVSTIQRKYKGIKIQEGVVDYGARFYFYTSKTTVASLINTLNDLNETLVTMPLGYVTHGLNLEEAARYMRSLKVPATVSVSSPDAVTAYNGYLTSSSKTPEEHFIETISLAGSYKDWSYSGQSTQLGIEFLKRGDKSVYYTSNPTTFHLDGEVITFDNLKTLLSLREVRTIKVFTTVDNINLHTQVVDMSMTYGQQFGPTYLDGADVTKIKPHNSHEGKTFYVLPNDDTLRVEAFEYYHTTDPSFLGRYMSALNHTKKWKYPQVNGLTSIKWADNNCYLATALLTLQQIELKFNPPALQDAYYRARAGEAANFCALILAYCNKTVGELGDVRETMSYLFQHANLDSCKRVLNVVCKTCGQQQTTLKGVEAVMYMGTLSYEQFKKGVQIPCTCGKQATKYLVQQESPFVMMSAPPAQYELKHGTFTCASEYTGNYQCGHYKHITSKETLYCIDGALLTKSSEYKGPITDVFYKENSYTTTIKPVTYKLDGVVCTEIDPKLDNYYKKDNSYFTEQPIDLVPNQPYPNASFDNFKFVCDNIKFADDLNQLTGYKKPASRELKVTFFPDLNGDVVAIDYKHYTPSFKKGAKLLHKPIVWHVNNATNKATYKPNTWCIRCLWSTKPVETSNSFDVLKSEDAQGMDNLACEDLKPVSEEVVENPTIQKDVLECNVKTTEVVGDIILKPANNSLKITEEVGHTDLMAAYVDNSSLTIKKPNELSRVLGLKTLATHGLAAVNSVPWDTIANYAKPFLNKVVSTTTNIVTRCLNRVCTNYMPYFFTLLLQLCTFTRSTNSRIKASMPTTIAKNTVKSVGKFCLEASFNYLKSPNFSKLINIIIWFLLLSVCLGSLIYSTAALGVLMSNLGMPSYCTGYREGYLNSTNVTIATYCTGSIPCSVCLSGLDSLDTYPSLETIQITISSFKWDLTAFGLVAEWFLAYILFTRFFYVLGLAAIMQLFFSYFAVHFISNSWLMWLIINLVQMAPISAMVRMYIFFASFYYVWKSYVHVVDGCNSSTCMMCYKRNRATRVECTTIVNGVRRSFYVYANGGKGFCKLHNWNCVNCDTFCAGSTFISDEVARDLSLQFKRPINPTDQSSYIVDSVTVKNGSIHLYFDKAGQKTYERHSLSHFVNLDNLRANNTKGSLPINVIVFDGKSKCEESSAKSASVYYSQLMCQPILLLDQALVSDVGDSAEVAVKMFDAYVNTFSSTFNVPMEKLKTLVATAEAELAKNVSLDNVLSTFISAARQGFVDSDVETKDVVECLKLSHQSDIEVTGDSCNNYMLTYNKVENMTPRDLGACIDCSARHINAQVAKSHNIALIWNVKDFMSLSEQLRKQIRSAAKKNNLPFKLTCATTRQVVNVVTTKIALKGG

SEQ ID NO: 390 - protein, 1642 aa, synthetic construct
KIVNNWLKQLIKVTLVFLFVAAIFYLITPVHVMSKHTDFSSEIIGYKAIDGGVTRDIASTDTCFANKHADFDTWFSQRGGSYTNDKACPLIAAVITREVGFVVPGLPGTILRTTNGDFLHFLPRVFSAVGNICYTPSKLIEYTDFATSACVLAAECTIFKDASGKPVPYCYDTNVLEGSVAYESLRPDTRYVLMDGSIIQFPNTYLEGSVRVVTTFDSEYCRHGTCERSEAGVCVSTSGRWVLNNDYYRSLPGVFCGVDAVNLLTNMFTPLIQPIGALDISASIVAGGIVAIVVTCLAYYFMRFRRAFGEYSHVVAFNTLLFLMSFTVLCLTPVYSFLPGVYSVIYLYLTFYLTNDVSFLAHIQWMVMFTPLVPFWITIAYIICISTKHFYWFFSNYLKRRVVFNGVSFSTFEEAALCTFLLNKEMYLKLRSDVLLPLTQYNRYLALYNKYKYFSGAMDTTSYREAACCHLAKALNDFSNSGSDVLYQPPQTSITSAVLQSGFRKMAFPSGKVEGCMVQVTCGTTTLNGLWLDDVVYCPRHVICTSEDMLNPNYEDLLIRKSNHNFLVQAGNVQLRVIGHSMQNCVLKLKVDTANPKTPKYKFVRIQPGQTFSVLACYNGSPSGVYQCAMRPNFTIKGSFLNGSCGSVGFNIDYDCVSFCYMHHMELPTGVHAGTDLEGNFYGPFVDRQTAQAAGTDTTITVNVLAWLYAAVINGDRWFLNRFTTTLNDFNLVAMKYNYEPLTQDHVDILGPLSAQTGIAVLDMCASLKELLQNGMNGRTILGSALLEDEFTPFDVVRQCSGVTFQSAVKRTIKGTHHWLLLTILTSLLVLVQSTQWSLFFFLYENAFLPFAMGIIAMSAFAMMFVKHKHAFLCLFLLPSLATVAYFNMVYMPASWVMRIMTWLDMVDTSLSGFKLKDCVMYASAVVLLILMTARTVYDDGARRVWTLMNVLTLVYKVYYGNALDQAISMWALIISVTSNYSGVVTTVMFLARGIVFMCVEYCPIFFITGNTLQCIMLVYCFLGYFCTCYFGLFCLLNRYFRLTLGVYDYLVSTQEFRYMNSQGLLPPKNSIDAFKLNIKLLGVGGKPCIKVATVQSKMSDVKCTSVVLLSVLQQLRVESSSKLWAQCVQLHNDILLAKDTTEAFEKMVSLLSVLLSMQGAVDINKLCEEMLDNRATLQAIASEFSSLPSYAAFATAQEAYEQAVANGDSEVVLKKLKKSLNVAKSEFDRDAAMQRKLEKMADQAMTQMYKQARSEDKRAKVTSAMQTMLFTMLRKLDNDALNNIINNARDGCVPLNIIPLTTAAKLMVVIPDYNTYKNTCDGTTFTYASALWEIQQVVDADSKIVQLSEISMDNSPNLAWPLIVTALRANSAVKLQNNELSPVALRQMSCAAGTTQTACTDDNALAYYNTTKGGRFVLALLSDLQDLKWARFPKSDGTGTIYTELEPPCRFVTDTPKGPKVKYLYFIKGLNNLNRGMVLGSLAATVRLQAGNATEVPANSTVLSFCAFAVDAAKAYKDYLASGGQPITNCVKMLCTHTGTGQAITVTPEANMDQESFGGASCCLYCRCHIDHPNPKGFCDLKGKYVQIPTTCANDPVGFTLKNTVCTVCGMWKGYGCSCDQLREPMLQSADAQSFLNGFAV

SEQ ID NO: 391 - protein, 2704 aa, synthetic construct
SADAQSFLNRVCGVSAARLTPCGTGTSTDVVYRAFDIYNDKVAGFAKFLKTNCCRFQEKDEDDNLIDSYFVVKRHTFSNYQHEETIYNLLKDCPAVAKHDFFKFRIDGDMVPHISRQRLTKYTMADLVYALRHFDEGNCDTLKEILVTYNCCDDDYFNKKDWYDFVENPDILRVYANLGERVRQALLKTVQFCDAMRNAGIVGVLTLDNQDLNGNWYDFGDFIQTTPGSGVPVVDSYYSLLMPILTLTRALTAESHVDTDLTKPYIKWDLLKYDFTEERLKLFDRYFKYWDQTYHPNCVNCLDDRCILHCANFNVLFSTVFPPTSFGPLVRKIFVDGVPFVVSTGYHFRELGVVHNQDVNLHSSRLSFKELLVYAADPAMHAASGNLLLDKRTTCFSVAALTNNVAFQTVKPGNFNKDFYDFAVSKGFFKEGSSVELKHFFFAQDGNAAISDYDYYRYNLPTMCDIRQLLFVVEVVDKYFDCYDGGCINANQVIVNNLDKSAGFPFNKWGKARLYYDSMSYEDQDALFAYTKRNVIPTITQMNLKYAISAKNRARTVAGVSICSTMTNRQFHQKLLKSIAATRGATVVIGTSKFYGGWHNMLKTVYSDVENPHLMGWDYPKCDRAMPNMLRIMASLVLARKHTTCCSLSHRFYRLANECAQVLSEMVMCGGSLYVKPGGTSSGDATTAYANSVFNICQAVTANVNALLSTDGNKIADKYVRNLQHRLYECLYRNRDVDTDFVNEFYAYLRKHFSMMILSDDAVVCFNSTYASQGLVASIKNFKSVLYYQNNVFMSEAKCWTETDLTKGPHEFCSQHTMLVKQGDDYVYLPYPDPSRILGAGCFVDDIVKTDGTLMIERFVSLAIDAYPLTKHPNQEYADVFHLYLQYIRKLHDELTGHMLDMYSVMLTNDNTSRYWEPEFYEAMYTPHTVLQAVGACVLCNSQTSLRCGACIRRPFLCCKCCYDHVISTSHKLVLSVNPYVCNAPGCDVTDVTQLYLGGMSYYCKSHKPPISFPLCANGQVFGLYKNTCVGSDNVTDFNAIATCDWTNAGDYILANTCTERLKLFAAETLKATEETFKLSYGIATVREVLSDRELHLSWEVGKPRPPLNRNYVFTGYRVTKNSKVQIGEYTFEKGDYGDAVVYRGTTTYKLNVGDYFVLTSHTVMPLSAPTLVPQEHYVRITGLYPTLNISDEFSSNVANYQKVGMQKYSTLQGPPGTGKSHFAIGLALYYPSARIVYTACSHAAVDALCEKALKYLPIDKCSRIIPARARVECFDKFKVNSTLEQYVFCTVNALPETTADIVVFDEISMATNYDLSVVNARLRAKHYVYIGDPAQLPAPRTLLTKGTLEPEYFNSVCRLMKTIGPDMFLGTCRRCPAEIVDTVSALVYDNKLKAHKDKSAQCFKMFYKGVITHDVSSAINRPQIGVVREFLTRNPAWRKAVFISPYNSQNAVASKILGLPTQTVDSSQGSEYDYVIFTQTTETAHSCNVNRFNVAITRAKVGILCIMSDRDLYDKLQFTSLEIPRRNVATLQAENVTGLFKDCSKVITGLHPTQAPTHLSVDTKFKTEGLCVDIPGIPKDMTYRRLISMMGFKMNYQVNGYPNMFITREEAIRHVRAWIGFDVEGCHATREAVGTNLPLQLGFSTGVNLVAVPTGYVDTPNNTDFSRVSAKPPPGDQFKHLIPLMYKGLPWNVVRIKIVQMLSDTLKNLSDRVVFVLWAHGFELTSMKYFVKIGPERTCCLCDRRATCFSTASDTYACWHHSIGFDYVYNPFMIDVQQWGFTGNLQSNHDLYCQVHGNAHVASCDAIMTRCLAVHECFVKRVDWTIEYPIIGDELKINAACRKVQHMVVKAALLADKFPVLHDIGNPKAIKCVPQADVEWKFYDAQPCSDKAYKIEELFYSYATHSDKFTDGVCLFWNCNVDRYPANSIVCRFDTRVLSNLNLPGCDGGSLYVNKHAFHTPAFDKSAFVNLKQLPFFYYSDSPCESHGKQVVSDIDYVPLKSATCITRCNLGGAVCRHHANEYRLYLDAYNMMISAGFSLWVYKQFDTYNLWNTFTRLQSLENVAFNVVNKGHFDGQQGEVPVSIINNTVYTKVDGVDVELFENKTTLPVNVAFELWAKRNIKPVPEVKILNNLGVDIAANTVIWDYKRDAPAHISTIGVCSMTDIAKKPTETICAPLTVFFDGRVDGQVDLFRNARNGVLITEGSVKGLQPSVGPKQASLNGVTLIGEAVKTQFNYYKKVDGVVQQLPETYFTQSRNLQEFKPRSQMEIDFLELAMDEFIERYKLEGYAFEHIVYGDFSHSQLGGLHLLIGLAKRFKESPFELEDFIPMDSTVKNYFITDAQTGSSKCVCSVIDLLLDDFVEIIKSQDLSVVSKVVKVTIDYTEISFMLWCKDGHVETFYPKLQSSQAWQPGVAMPNLYKMQRMLLEKCDLQNYGDSATLPKGIMMNVAKYTQLCQYLNTLTLAVPYNMRVIHFGAGSDKGVAPGTAVLRQWLPTGTLLVDSDLNDFVSDADSTLIGDCATVHTANKWDLIISDMYDPKTKNVTKENDSKEGFFTYICGFIQQKLALGGSVAIKITEHSWNADLYKLMGHFAWWTAFVTNVNASSSEAFLIGCNYLGKPREQIDGYVMHANYIFWRNTNPIQLSSYSLFDMSKFPLKLRGTAVMSLKEGQINDMILSLLSKGRLIIRENNRVVISSDVLVNN

SEQ ID NO: 392 - protein, 932 aa, synthetic construct
SADAQSFLNRVCGVSAARLTPCGTGTSTDVVYRAFDIYNDKVAGFAKFLKTNCCRFQEKDEDDNLIDSYFVVKRHTFSNYQHEETIYNLLKDCPAVAKHDFFKFRIDGDMVPHISRQRLTKYTMADLVYALRHFDEGNCDTLKEILVTYNCCDDDYFNKKDWYDFVENPDILRVYANLGERVRQALLKTVQFCDAMRNAGIVGVLTLDNQDLNGNWYDFGDFIQTTPGSGVPVVDSYYSLLMPILTLTRALTAESHVDTDLTKPYIKWDLLKYDFTEERLKLFDRYFKYWDQTYHPNCVNCLDDRCILHCANFNVLFSTVFPPTSFGPLVRKIFVDGVPFVVSTGYHFRELGVVHNQDVNLHSSRLSFKELLVYAADPAMHAASGNLLLDKRTTCFSVAALTNNVAFQTVKPGNFNKDFYDFAVSKGFFKEGSSVELKHFFFAQDGNAAISDYDYYRYNLPTMCDIRQLLFVVEVVDKYFDCYDGGCINANQVIVNNLDKSAGFPFNKWGKARLYYDSMSYEDQDALFAYTKRNVIPTITQMNLKYAISAKNRARTVAGVSICSTMTNRQFHQKLLKSIAATRGATVVIGTSKFYGGWHNMLKTVYSDVENPHLMGWDYPKCDRAMPNMLRIMASLVLARKHTTCCSLSHRFYRLANECAQVLSEMVMCGGSLYVKPGGTSSGDATTAYANSVFNICQAVTANVNALLSTDGNKIADKYVRNLQHRLYECLYRNRDVDTDFVNEFYAYLRKHFSMMILSDDAVVCFNSTYASQGLVASIKNFKSVLYYQNNVFMSEAKCWTETDLTKGPHEFCSQHTMLVKQGDDYVYLPYPDPSRILGAGCFVDDIVKTDGTLMIERFVSLAIDAYPLTKHPNQEYADVFHLYLQYIRKLHDELTGHMLDMYSVMLTNDNTSRYWEPEFYEAMYTPHTVLQ

SEQ ID NO: 393 - protein, 1128 aa, synthetic construct
AVGACVLCNSQTSLRCGACIRRPFLCCKCCYDHVISTSHKLVLSVNPYVCNAPGCDVTDVTQLYLGGMSYYCKSHKPPISFPLCANGQVFGLYKNTCVGSDNVTDFNAIATCDWTNAGDYILANTCTERLKLFAAETLKATEETFKLSYGIATVREVLSDRELHLSWEVGKPRPPLNRNYVFTGYRVTKNSKVQIGEYTFEKGDYGDAVVYRGTTTYKLNVGDYFVLTSHTVMPLSAPTLVPQEHYVRITGLYPTLNISDEFSSNVANYQKVGMQKYSTLQGPPGTGKSHFAIGLALYYPSARIVYTACSHAAVDALCEKALKYLPIDKCSRIIPARARVECFDKFKVNSTLEQYVFCTVNALPETTADIVVFDEISMATNYDLSVVNARLRAKHYVYIGDPAQLPAPRTLLTKGTLEPEYFNSVCRLMKTIGPDMFLGTCRRCPAEIVDTVSALVYDNKLKAHKDKSAQCFKMFYKGVITHDVSSAINRPQIGVVREFLTRNPAWRKAVFISPYNSQNAVASKILGLPTQTVDSSQGSEYDYVIFTQTTETAHSCNVNRFNVAITRAKVGILCIMSDRDLYDKLQFTSLEIPRRNVATLQAENVTGLFKDCSKVITGLHPTQAPTHLSVDTKFKTEGLCVDIPGIPKDMTYRRLISMMGFKMNYQVNGYPNMFITREEAIRHVRAWIGFDVEGCHATREAVGTNLPLQLGFSTGVNLVAVPTGYVDTPNNTDFSRVSAKPPPGDQFKHLIPLMYKGLPWNVVRIKIVQMLSDTLKNLSDRVVFVLWAHGFELTSMKYFVKIGPERTCCLCDRRATCFSTASDTYACWHHSIGFDYVYNPFMIDVQQWGFTGNLQSNHDLYCQVHGNAHVASCDAIMTRCLAVHECFVKRVDWTIEYPIIGDELKINAACRKVQHMVVKAALLADKFPVLHDIGNPKAIKCVPQADVEWKFYDAQPCSDKAYKIEELFYSYATHSDKFTDGVCLFWNCNVDRYPANSIVCRFDTRVLSNLNLPGCDGGSLYVNKHAFHTPAFDKSAFVNLKQLPFFYYSDSPCESHGKQVVSDIDYVPLKSATCITRCNLGGAVCRHHANEYRLYLDAYNMMISAGFSLWVYKQFDTYNLWNTFTRLQ

SEQ ID NO: 394 - protein, 644 aa, synthetic construct
SLENVAFNVVNKGHFDGQQGEVPVSIINNTVYTKVDGVDVELFENKTTLPVNVAFELWAKRNIKPVPEVKILNNLGVDIAANTVIWDYKRDAPAHISTIGVCSMTDIAKKPTETICAPLTVFFDGRVDGQVDLFRNARNGVLITEGSVKGLQPSVGPKQASLNGVTLIGEAVKTQFNYYKKVDGVVQQLPETYFTQSRNLQEFKPRSQMEIDFLELAMDEFIERYKLEGYAFEHIVYGDFSHSQLGGLHLLIGLAKRFKESPFELEDFIPMDSTVKNYFITDAQTGSSKCVCSVIDLLLDDFVEIIKSQDLSVVSKVVKVTIDYTEISFMLWCKDGHVETFYPKLQSSQAWQPGVAMPNLYKMQRMLLEKCDLQNYGDSATLPKGIMMNVAKYTQLCQYLNTLTLAVPYNMRVIHFGAGSDKGVAPGTAVLRQWLPTGTLLVDSDLNDFVSDADSTLIGDCATVHTANKWDLIISDMYDPKTKNVTKENDSKEGFFTYICGFIQQKLALGGSVAIKITEHSWNADLYKLMGHFAWWTAFVTNVNASSSEAFLIGCNYLGKPREQIDGYVMHANYIFWRNTNPIQLSSYSLFDMSKFPLKLRGTAVMSLKEGQINDMILSLLSKGRLIIRENNRVVISSDVLVNN

SEQ ID NO: 395 - protein, 680 aa, Marburg marburgvirus
WTTCFFISLILIQGIKTLPILEIASNDQPQNVDSVCSGTLQKTEDVHLMGFTLSGQKVADSPLEASKRWAFRTGVPPKNVEYTEGEEAKTCYNISVTDPSGKSLLLDPPTNVRDYPKCKTIHHIQGQNPHAQGIALHLWGAFFLYDRIASTTMYRGKVFTEGNIAAMIVNKTVHKMIFSRQGQGYRHMNLTSTNKYWTSSNGTQTNDTGCFGTLQEYNSTKNQTCAPSKTPPPPPTAHPEIKPTSTPTDATRLNTTNPNSDDEDLTTSGSGSGEQEPYTTSDAVTKQGLSSTMPPTLSPQPGTPQQGGNNTNHSQDAATELDNTNTTAQPPMPSHNTTTISTNNTSKHNLSTLSEPPQNTTNPNTQSMATENEKTSAPPKTTLPPTESPTTEKSTNNTKSPTTMEPNTTNGHFTSPSSTPNSTTQHLIYFRRKRSILWREGDMFPFLDGLINAPIDFDPVPNTKTIFDESSSSGASAEEDQHASSNISLTLSYLPHTSENTAYSGENENDCDAELRIWSVQEDDLAAGLSWIPFFGPGIEGLYTAGLIKNQNNLVCRLRRLANQTAKSLELLLRVTTEERTFSLINRHAIDFLLTRWGGTCKVLGPDCCIGIEDLSRNISEQIDQIKKDEQKEGTGWGLGGKWWTSDWGVLTNLGILLLLSIAVLIALSCICRIFTKYIG

SEQ ID NO: 396 - protein, 18 aa, Marburg marburgvirus
WTTCFFISLILIQGIKTL

SEQ ID NO: 397 - DNA, 54 bp, synthetic construct
  source 1..54: mol_type = other DNA
tggacgacctgcttcttcatctccctaatcctaatccagggaatcaagacccta

SEQ ID NO: 398 - protein, 38 aa, Marburg marburgvirus
WWTSDWGVLTNLGILLLLSIAVLIALSCICRIFTKYIG

SEQ ID NO: 399 - DNA, 114 bp, synthetic construct
  source 1..114: mol_type = other DNA
tggtggacatctgactggggagtcctaacgaacctaggaatcctactactattgtcgatcgcggtcctaatcgcgctatcctgtatctgtagaatcttcaccaagtacatcgga

SEQ ID NO: 400 - protein, 302 aa, Marburg marburgvirus
ASSSNYNTYMQYLNPPPYADHGANQLIPADQLSNQHGITPNYVGDLNLDDQFKGNVCHAFTLEAIIDISAYNERTVKGVPAWLPLGIMSNFEYPLAHTVAALLTGSYTITQFTHNGQKFVRVNRLGTGIPAHPLRMLREGNQAFIQNMVIPRNFSTNQFTYNLTNLVLSVQKLPDDAWRPSKDKLIGNTMHPAISIHPNLPPIVLPTVKKQAYRQHKNPNNGPLLAISGILHQLRVEKVPEKTSLFRISLPADMFSVKEGMMKKRGESSPVVYFQAPENFPLNGFNNRQVVLAYANPTLSAI

SEQ ID NO: 401 - DNA, 912 bp, synthetic construct
  source 1..912: mol_type = other DNA
gcgtctagttctaattataatacttatatgcaatatctaaatccaccaccatatgcggatcatggtgctaatcaactaattccagcggatcaactatctaatcaacatggaattacaccaaattatgttggagatctaaatctagatgatcagtttaaaggaaatgtttgtcatgcgtttacactagaagcgattattgatatttctgcgtataatgaaagaacagtaaaaggtgtaccagcttggctaccactaggaattatgtctaattttgaatatccactagcgcatacagtagcggcgctattgacaggatcttatacaattacacagtttacacataatggacaaaagtttgttagagtaaatagactaggaactggaataccagcgcatccactaagaatgctaagagaaggaaatcaagcttttattcaaaatatggttattccaagaaatttctctacaaatcagtttacttataatctaactaatctagtactatctgtacaaaagctaccagatgatgcttggagaccatctaaagataaactaattggaaatacaatgcatccagcgatttctattcatccaaatctaccaccaatagtactaccaactgtaaagaaacaagcgtatagacaacataagaatccaaataatggaccactattggcgatttctggaattctacatcaactaagagtagaaaaggtaccagaaaagacatctttgtttagaatttctctaccagcggatatgttttctgtaaaagaaggaatgatgaagaaaagaggagaatcttctccagtagtatattttcaagcgccagaaaattttccattgaatggttttaataatagacaagtagtactagcgtatgcgaatccaacactatctgcgatataataa

SEQ ID NO: 402 - DNA, 1608 bp, synthetic construct
  source 1..1608: mol_type = other DNA
atgacacctggaacacaatctccattcttcctactactactattgacagtactaacagtagtaacaggatctggacatgcgtctagtacaccaggtggagagaaggaaacatctgcgactcaaagatcttctgtaccatcttctacagagaagaatgcggtatctatgacatctagtgtactatcttctcattctcctggatctggatcttctactacacaaggacaagatgtaacactagcgccagctacagaaccagcttctggatctgctgctacttggggtcaagatgttacttctgttccagtaacaagaccagcgctaggatctacaacaccaccagcgcatgatgtaacaagtgcgccagataataagccagcgcctggttctactgctccaccagctcatggtgttacttcagcgcctgatacaagacccgcacccggatctaccgctccgcctgcacacggcgtcacatctgctcccgacactcgtccagctcctggtagcacagcacctccagcgcatggagtaaccagtgcaccagatacccgacctgcgccgggcagtactgccccaccggcccacggggtgacgagcgccccggacacgcgcccagctccagggtcaacggcgccccctgctcatggtgttacaagtgcacctgataatagacctgcgttgggatctactgcgcctccagttcataatgtaacatcagcgtctggaagtgcgtctggttctgcgtctacattggttcataatggtacatctgcgagagcgacaacaactccagcgtctaagtctacaccattctctattccatctcatcattctgatacaccaacaacattggcgagtcattctacaaagacagatgcgagttctacacatcattctactgtaccaccactaacatcttctaatcatagtacatctccacaactatctactggtgtatctttcttcttcctatcctttcatatttctaatctacagttcaattctagtttggaagatccatctacagattattatcaagaactacaaagagatatttctgaaatgtttctacaaatatataaacaaggaggatttctaggactatctaatattaagtttagaccaggatctgtagtagttcaactaactctagcgtttagagaaggtactattaatgtacatgatgttgaaacacagtttaatcaatataagacagaagcggcgtctagatataatctaacaatttctgatgtatctgtatctgatgttccatttccattctctgcgcaatctggtgctggtgtatggtggacatctgattggggagtactaactaatctaggaattctactattgctatctattgcggtactaattgcgctatcttgtatatgtagaagaaagaattatggacaactagatattttcccagcgagagatacttatcatccaatgtctgaatatccaacatatcatacacatggaagatatgtaccaccttcttcaacagatagatctccatatgagaaggtatctgcgggaaatggtggttcttctctatcttatacaaatccagcggtagcggcgacttctgcgaatctataa

SEQ ID NO: 403 - protein, 535 aa, synthetic construct
MTPGTQSPFFLLLLLTVLTVVTGSGHASSTPGGEKETSATQRSSVPSSTEKNAVSMTSSVLSSHSPGSGSSTTQGQDVTLAPATEPASGSAATWGQDVTSVPVTRPALGSTTPPAHDVTSAPDNKPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDNRPALGSTAPPVHNVTSASGSASGSASTLVHNGTSARATTTPASKSTPFSIPSHHSDTPTTLASHSTKTDASSTHHSTVPPLTSSNHSTSPQLSTGVSFFFLSFHISNLQFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAGVWWTSDWGVLTNLGILLLLSIAVLIALSCICRRKNYGQLDIFPARDTYHPMSEYPTYHTHGRYVPPSSTDRSPYEKVSAGNGGSSLSYTNPAVAATSANL

SEQ ID NO: 404 - DNA, 912 bp, Marburg marburgvirus
  source 1..912: mol_type = other DNA
atggcgtctagttctaattataatacttatatgcaatatctaaatccaccaccatatgcggatcatggtgctaatcaactaattccagcggatcaactatctaatcaacatggaattacaccaaattatgttggagatctaaatctagatgatcagtttaaaggaaatgtttgtcatgcgtttacactagaagcgattattgatatttctgcgtataatgaaagaacagtaaaaggtgtaccagcttggctaccactaggaattatgtctaattttgaatatccactagcgcatacagtagcggcgctattgacaggatcttatacaattacacagtttacacataatggacaaaagtttgttagagtaaatagactaggaactggaataccagcgcatccactaagaatgctaagagaaggaaatcaagcttttattcaaaatatggttattccaagaaatttctctacaaatcagtttacttataatctaactaatctagtactatctgtacaaaagctaccagatgatgcttggagaccatctaaagataaactaattggaaatacaatgcatccagcgatttctattcatccaaatctaccaccaatagtactaccaactgtaaagaaacaagcgtatagacaacataagaatccaaataatggaccactattggcgatttctggaattctacatcaactaagagtagaaaaggtaccagaaaagacatctttgtttagaatttctctaccagcggatatgttttctgtaaaagaaggaatgatgaagaaaagaggagaatcttctccagtagtatattttcaagcgccagaaaattttccattgaatggttttaataatagacaagtagtactagcgtatgcgaatccaacactatctgcgatataa

SEQ ID NO: 405 - protein, 303 aa, Marburg marburgvirus
MASSSNYNTYMQYLNPPPYADHGANQLIPADQLSNQHGITPNYVGDLNLDDQFKGNVCHAFTLEAIIDISAYNERTVKGVPAWLPLGIMSNFEYPLAHTVAALLTGSYTITQFTHNGQKFVRVNRLGTGIPAHPLRMLREGNQAFIQNMVIPRNFSTNQFTYNLTNLVLSVQKLPDDAWRPSKDKLIGNTMHPAISIHPNLPPIVLPTVKKQAYRQHKNPNNGPLLAISGILHQLRVEKVPEKTSLFRISLPADMFSVKEGMMKKRGESSPVVYFQAPENFPLNGFNNRQVVLAYANPTLSAI

SEQ ID NO: 406 - protein, 9 aa, synthetic construct
SYVPSAEQI

SEQ ID NO: 407 - protein, 9 aa, synthetic construct
YLNKIQNSL

SEQ ID NO: 408 - DNA, 1158 bp, synthetic construct
  source 1..1158: mol_type = other DNA
atggacgccatgaagagaggactttgttgcgtcctactactatgcggagcggtattcgtatctccgtcgcaagaaattcacgcgagattcagaagaggtgccagatgcagaagaacatctaccggacagatctccaccttgagagtaaatatcacagcgccgctatctcagagagccaagagaggatcgggagcgacaaacttctcgctattgaaacaagcgggagatgtcgaagagaacccaggaccagatgctatgaagagaggactttgctgcgtattgctattgtgcggagccgtcttcgtctcaccatctcaagaaatccatgccagattcagaagaggtgctagatgtagaagaacctccacgggacaaatcagtaccctaagagttaacatcaccgcgccgttgagtcaaagagctaagagaggttccggagccaccaacttcagtttgctaaagcaggcgggagatgtggaagagaatcctggtcctgacgcaatgaagagaggactttgctgcgttctattgctatgcggtgccgtctttgtttctccgagtcaagagatacacgctagattcagaagaggtgcaagatgtagaagaacctcgaccggtcaaatctcgacgcttagagtcaatattaccgcgccattgtcgcagagagcgaagagaggatcgggagccactaatttcagtctacttaagcaagcgggagatgtagaggagaatcctggaccggatgccatgaagagaggactttgttgcgttctgttgctttgcggagctgtgttcgtcagtccttctcaagagattcatgcaagattcagaagaggtgcaagatgcagaagaaccagtacgggacagatttcgacattaagagtgaacattactgcgcctttgtctcaaagagcgaagagaggttccggagcgacgaatttctcgttgctcaagcaagcgggagatgtagaagagaacccaggacctgatgcaatgaagagaggactttgttgcgtattacttctttgcggtgcagtgtttgtctcgccgtcacaagagatccacgcaagattcagaagaggtgccagatgtagaagaactagtacaggacaaatctccacgctaagagtaaacataacggcaccactatctcaataa

SEQ ID NO: 409 - DNA, 1101 bp, synthetic construct
  source 1..1101: mol_type = other DNA
atggacgccatgaagagaggactttgttgcgtcctactactatgcggagcggtattcgtatctccgtcgcaagaaattcacgcgagattcagaagaggtgccagatctacaggacagatctctaccctaagagtcaatatcacagcgccgctatctcagagagcgaagagaggatcgggagcgacaaacttctcgctattgaaacaagcgggagatgtcgaggagaacccaggaccagatgctatgaagagaggactttgctgcgtattgctattgtgcggagccgtgttcgtctcgccatctcaagaaatccatgccagattcagaagaggtgctagaagtaccggacaaatctccacgttgagagtaaacattaccgcgccgttgtcgcaaagagctaagagaggttccggagccactaacttcagtttgctaaagcaggcgggagatgtggaagagaatcctggtcctgacgcaatgaagagaggactttgctgcgttctattgctatgcggtgccgtctttgtttctccgagtcaagagatacacgctagattcagaagaggtgctagatccacgggacaaatcagtacccttagagtgaacatcacggcgccactttctcaaagagccaagagaggttccggagcgaccaatttctcgttgctaaagcaagcgggagatgtagaagagaatcccggaccggatgccatgaagagaggactttgttgcgtgctgttgctttgcggagctgtgttcgtcagtccttctcaagagattcatgcaagattcagaagaggtgcaagatcgaccggtcaaatttcgacgctaagagttaacataacggcgcccttgagtcagagagccaagagaggatcgggagccactaacttctcgttgttgaagcaggcgggagatgtagaagagaatccgggtccagatgcaatgaagagaggactttgttgcgtattacttctttgcggtgcagtgtttgtctcgccgtcacaagagatccacgcaagattcagaagaggtgccagaagtacgggtcaaattagtaccttgagagtcaatattacggcgcctttgtcacagtaatga

SEQ ID NO: 410 - DNA, 21 bp, synthetic construct
  source 1..21: mol_type = other DNA
agatcggagatgactgcgatg

SEQ ID NO: 411 - DNA, 21 bp, synthetic construct
  source 1..21: mol_type = other DNA
cgatggatggtcagattgtcc

SEQ ID NO: 412 - DNA, 22 bp, synthetic construct
  source 1..22: mol_type = other DNA
gagaggacgggagaattaacta

SEQ ID NO: 413 - DNA, 22 bp, synthetic construct
  source 1..22: mol_type = other DNA
tggtaggaataccagatacgac

SEQ ID NO: 414 - DNA, 23 bp, synthetic construct
  source 1..23: mol_type = other DNA
ggagcagagtttacatcttccaa

SEQ ID NO: 415 - DNA, 22 bp, synthetic construct
  source 1..22: mol_type = other DNA
ctccgtgagaatatccttgctc

(19) United States
(12) Patent Application Publication
(10) Pub. No.: US 2024/0344082 A1
(43) Pub. Date: Oct. 17, 2024

(54) VIRAL CONSTRUCTS FOR USE IN ENHANCING T-CELL PRIMING DURING VACCINATION

(71) Applicants: GeoVax, Inc., Smyrna, GA (US); Leidos, Inc., Reston, VA (US)

(72) Inventors: Mary Jo Hauser, Marietta, GA (US); Arban Domi, Atlanata, GA (US); Gabriel Gutierrez, Reston, VA (US); James Pannucci, Reston, VA (US); Kotraiah Vinayaka, Reston, VA (US); Timothy Phares, Reston, VA (US); Peter Buontempo, Reston, VA (US); Cecille Browne, Reston, VA (US)

(73) Assignees: GeoVax, Inc., Smyrna, GA (US); Leidos, Inc., Reston, VA (US)

(21) Appl. No.: 18/229,070

(22) Filed: Aug. 1, 2023

Related U.S. Application Data
(63) Continuation of application No. PCT/US2022/014970, filed on Feb. 2, 2022.
(60) Provisional application No. 63/144,834, filed on Feb. 2, 2021.

(51) Int. Cl. C12N 15/86; A61K 39/00; C07K 14/47

(52) U.S. Cl. CPC C12N 15/86; C07K 14/4703; A61K 39/00; C07K 2319/036; C12N 2710/24143

(57) ABSTRACT

The invention provides virus-based expression vectors comprising immune-checkpoint inhibitor inserts for use as effective adjuvants in enhancing T-cell priming to an antigen in a host during a vaccination regimen. In particular, the compositions described herein are novel recombinant modified vaccinia Ankara (MVA) viral constructs encoding one or more peptides which, upon administration, are expressed in a multimer conformation and subsequently cleaved and secreted from the cell. Such peptides are capable of downregulating an immune checkpoint pathway, for example, by inhibiting the activation of programmed-cell death protein 1 (PD-1), programed cell death ligand 1 (PD-L1), cytotoxic T-lymphocyte-associated protein 4 (CTLA-4), or another immune checkpoint regulator, or a combination thereof. When used in concert with the administration of an antigen during a vaccination strategy, the immune checkpoint expressing MVA viral construct provides significantly improved antigen-specific CD8+ T cell expansion, increased antigenic responses, and improved vaccination efficacy.

### CROSS REFERENCE TO RELATED APPLICATIONS

This application is a continuation of International Patent Application No. PCT/US2022/014970, filed Feb. 2, 2022, which claims benefit of U.S. Provisional Application No. 63/144,834, filed Feb. 2, 2021. The entirety of each of these applications is hereby incorporated by reference herein for all purposes.

### FIELD OF THE INVENTION

The invention provides virus-based expression vectors comprising immune-checkpoint inhibitor encoding nucleic acid inserts for use as effective adjuvants in enhancing T-cell priming to an antigen in a host during a vaccination regimen. In particular, the compositions described herein are novel recombinant modified vaccinia Ankara (MVA) viral constructs encoding immune checkpoint inhibitor peptides which, upon administration, are expressed in a multimer conformation and subsequently cleaved and secreted from the cell.

### INCORPORATION BY REFERENCE

The contents of the XML file named “19101-014WO1US1_ST26_2023-07-17” which was created on Jul. 17, 2023, and is 426 KB in size, are hereby incorporated by reference in their entirety.

### BACKGROUND OF THE INVENTION

Vaccines are considered one of the most important advances in modern medicine and have greatly improved quality of life by reducing or eliminating many serious infectious diseases.

Vaccines have been developed against a wide assortment of human pathogens, including, for example, bacterial toxins (e.g., tetanus and diphtheria toxins), acute viral pathogens (e.g., measles, mumps, rubella), latent or chronic viral pathogens (e.g., varicella zoster virus [VZV] and human papilloma virus [HPV], respectively), respiratory pathogens (e.g., influenza, Bordetella pertussis), and enteric pathogens (e.g., poliovirus, Salmonella typhi). Most approved vaccines can be categorized as live, attenuated vaccines, non-replicating whole-particle vaccines (including virus-like particles, or VLPs), and subunit vaccines.

In order to develop a successful vaccine, however, a powerful and long-lasting protective immunity that consists of humoral and cellular immune responses is needed. Both elements of immunity are essential for effectively eliminating pathogens. While advances have been made in developing vaccines against a number of pathogens, the inability to elicit potent, durable, and protective T cell immunity, particularly CD8+ T cell responses, has been a major obstacle and is the primary reason that many vaccine development efforts fail, particularly for intracellular pathogens (see, e.g., Seder et al., Vaccines against intracellular infections requiring cellular immunity. Nature. 2000 Aug. 17; 406(6797):793-8).

One strategy to overcome these inherent obstacles has been the identification and use of adjuvants that augment immunogenicity, and considerable work has gone into evaluating the impact of putative adjuvants on innate immune activation and on adaptive immune responses to model antigens and potential vaccines (see, e.g., Halbroth et al., Development of a Molecular Adjuvant to Enhance Antigen-Specific CD8+ T Cell Responses. Sci Rep. 2018 Oct. 9; 8(1):15020; Counoupas et al., Delta inulin-based adjuvants promote the generation of polyfunctional CD4+ T cell responses and protection against Mycobacterium tuberculosis infection. Sci Rep. 2017 Aug. 17; 7(1):8582; Thakur et al., Intracellular Pathogens: Host Immunity and Microbial Persistence Strategies. Immunol Res. 2019 Apr. 14; 2019:1356540).

For example, alhydrogel is a well-characterized aluminum hydroxide adjuvant, which is currently contained in several FDA-approved vaccines. Alhydrogel provides a depot effect whereby antigen is released more slowly in vivo, resulting in prolonged antigen exposure, which may or may not contribute to adjuvantcy (Hutchison et al., Antigen depot is not required for alum adjuvanticity. FASEB J. 2012; 26:1272-1279). Additionally, alhydrogel has been shown to activate the inflammasome, which may contribute to the immunogenicity of alhydrogel-based vaccines (Guven et al., Aluminum hydroxide adjuvant differentially activates the three complement pathways with major involvement of the alternative pathway. PLoS One. 2013; 8:e74445).

PolyICLC is a double-strand RNA stabilized by poly-L-lysine in carboxymethylcellulose (Levy et al., A modified polyriboinosinic-polyribocytidylic acid complex that induces interferon in primates. J. Infect. Dis. 1975; 132:434-439). It signals through toll-like receptor-3 (TLR3) and potentially melanoma differentiation-associated protein 5 (MDA5) receptors, eliciting a strong type I IFN response, and it skews the immune response toward a Th1 profile response (Wang et al., Cutting edge: polyinosinic:polycytidylic acid boosts the generation of memory CD8 T cells through melanoma differentiation-associated protein 5 expressed in stromal cells. J. Immunol. 2010; 184:2751-2755). PolyICLC has been in multiple clinical trials for both therapeutic and vaccine purposes (Martins et al., Vaccine adjuvant uses of poly-ic and derivatives. Expert Rev. Vaccines. 2015; 14:447-459).

CpG oligodeoxynucleotides (or CpG ODN) are short single-stranded synthetic DNA molecules that contain a cytosine triphosphate deoxynucleotide (“C”) followed by a guanine triphosphate deoxynucleotide (“G”). The “p” refers to the phosphodiester link between consecutive nucleotides, although some ODN have a modified phosphorothioate (PS) backbone instead. When these CpG motifs are unmethylated, they act as immunostimulants, and have also been examined as adjuvants (Marshall et al., Identification of a novel cpg DNA class and motif that optimally stimulate B cell and plasmacytoid dendritic cell functions. J. Leukoc. Biol. 2003; 73:781-792).

MPL is a TLR4 agonist, and the active component of the GSK adjuvant AS04 (Einstein et al., Comparative humoral and cellular immunogenicity and safety of human papillomavirus (HPV)-16/18 AS04-adjuvanted vaccine and HPV-6/11/16/18 vaccine in healthy women aged 18-45 years: follow-up through month 48 in a Phase III randomized study. Hum. Vaccines Immunother. 2014; 10:3455-3465). MPL has been shown to be highly effective as an adjuvant, particularly in combination with an aluminum-based adjuvant like alhydrogel or a nanoparticle formulation (Bohannon et al., The immunobiology of Toll-Like receptor 4 agonists: from endotoxin tolerance to immunoadjuvants. Shock. 2013; 40:451-462).

Other well-known adjuvants include alum-based adjuvants, oil based adjuvants, Freund's adjuvant, specol, Ribi adjuvant, myobacterium vaccae, immune stimulating complexes (ISCOMS), MF-59, SBAS-2, SBAS-4, detox B SE (Enhanzyn®), lipid-A mimetic RC-529, amino-alkyl glucosaminide 4-phosphates (AGPs), CRX-527, monophosphoryl lipid A (e.g., MPL-SE), detoxified saponin derivatives (e.g., QS-21, QS7), escin, gigitonin, gypsophila, and Chenopodium quinoa saponins (see, e.g., Alving et al., Adjuvants for Human Vaccines. Curr Opin Immunol. 2012 June; 24(3): 310-315).

Despite significant advances in the formulation of and use of adjuvants, the majority of adjuvants are designed to generate innate inflammatory danger signals. While these danger signals are essential for innate immune activation, including antigen presentation and cytokine production, there is limited effect directly on T-cell priming (Powell et al. Polyionic vaccine adjuvants: another look at aluminum salts and polyelectrolytes. Clin Exp Vaccine Res. 2015 January; 4(1):23-45; Petrovsky N. Comparative Safety of Vaccine Adjuvants: A Summary of Current Evidence and Future Needs. Drug Saf. 2015 November; 38(11):1059-74), with most vaccination strategies using common adjuvants failing to elicit long-term memory CD8⁺ T cells (Kamphorst et al., Beyond Adjuvants: Immunomodulation strategies to enhance T cell immunity. Vaccine. 2015 Jun. 8; 33(0 2): B21-B28). This is especially true during vaccinations targeting chronic infections and cancer, which require immunomodulation strategies to enhance T-cell responses necessary to overcome the immunosuppressive microenvironment.

One such strategy has been to downregulate immune checkpoint inhibitory receptors such as programmed-cell death protein 1 (PD-1) or programed cell death ligand 1 (PD-L1). For example, PD-1 functions in regulating the threshold, strength, and duration of T-cell responses to antigen presentation (Okazaki et al., A rheostat for immune responses: the unique properties of PD-1 and their advantages for clinical application. Nat Immunol. 2013 December; 14(12):1212-8). PD1 is rapidly upregulated upon naïve T-cell activation, which is required to minimize damage to the host from uncontrolled inflammation during infection and after the infection (Ahn et al., Role of PD-1 during effector CD8 T cell differentiation. PNAS 2018 May 1; 115(18):4749-4754). In non-human primates, immunization with a SIVgag adenovirus-based vaccine in combination with an anti-PD1 mAb significantly elevated peak Gag-specific T-cell responses (Finnefrock et al., PD-1 blockade in rhesus macaques: impact on chronic infection and prophylactic vaccination. J Immunol. 2009 Jan. 15; 182(2):980-7).

While monoclonal antibody (mAb)-based checkpoint inhibitors developed to treat cancer can effectively restore immune function, they do not, however, readily lend themselves to the field of infectious disease vaccinology. Due to their long serum half-life, anti-PD1 mAbs can trigger severe immune-related adverse events (irAEs) and precipitate autoimmune disease (Brahmer et al., Phase I study of single-agent anti-programmed death-1 (MDX-1106) in refractory solid tumors: safety, clinical activity, pharmacodynamics, and immunologic correlates. J Clin Oncol. 2010 Jul. 1; 28(19):3167-75; Topalian et al., Safety, activity, and immune correlates of anti-PD-1 antibody in cancer. N Engl J Med. 2012 Jun. 28; 366(26):2443-54), making their use as prophylactic vaccine adjuvants unacceptable.

Accordingly, improved methods of using immune checkpoint inhibitors in vaccination strategy that provide safe and effective immunization is needed.

### SUMMARY OF THE INVENTION

Provided herein are compositions comprising a recombinant modified vaccinia Ankara (rMVA) viral vector for use as an adjuvant or vaccine during an immunization protocol in a host such as a human. The rMVA are constructed to express high concentrations of peptides capable of inhibiting one or more immune checkpoint pathways (immune checkpoint inhibitor peptide). In some embodiments, the immune checkpoint inhibitor peptides are expressed from a polycistronic, multimeric nucleic acid insert and secreted from the cell.

It has previously been shown that the use of a PD-1 inhibitor peptide (LD01-SEQ ID NO.: 1), when administered in combination with an adenovirus-based or irradiated sporozoite-based prophylactic malaria vaccine, enhances antigen-specific CD8+ T-cell expansion in immune-competent mice (see Phares et al. A peptide-based PD1 antagonist enhances T-cell priming and efficacy of a prophylactic malaria vaccine and promotes survival in a lethal malaria model. Front. Immunol. 11, 1377 (2020), incorporated herein by reference). As shown herein, it has now been found that expressing immune checkpoint inhibitors using MVA as a delivery vehicle provides significant advantages during vaccination strategies, as the natural tropism of the MVA viral vector includes professional antigen presenting cells such as dendritic cells, which are capable of migrating to draining lymph nodes and spread systemically. It is believed that by expressing sufficient and high quantities of therapeutic levels of an immune checkpoint inhibitor, for example in a polycistronic, multimeric conformation, in the lymph node environment during host exposure to an antigen, CD8+ T-cell priming is significantly enhanced. As shown in the Examples below, when used in concert with the administration of an antigen during a vaccination strategy, the immune checkpoint expressing rMVA viral construct provides significantly improved antigen-specific CD8+ T cell expansion, increased antigenic responses, and improved vaccination efficacy compared to, for example, the naked administration of such immune checkpoint inhibitor peptides, and provides a significant improvement over prior art adjuvant strategies.

In one aspect, provided herein is an rMVA viral vector comprising a heterologous polycistronic nucleic acid insert encoding one or more chimeric polypeptides, for example 1, 2, 3, 4, 5, 6, 7, 8, 9, or 10 or more chimeric polypeptides, each chimeric polypeptide comprising a secretion signal peptide and an immune checkpoint inhibitor peptide. In some embodiments, the rMVA viral vector comprises a heterologous nucleic acid insert encoding two or more chimeric polypeptides, wherein the two or more chimeric polypeptides are expressed from a single heterologous polycistronic nucleic acid insert, wherein each of the nucleic acid sequences encoding the two or more chimeric polypeptides are operably linked in the polycistronic nucleic acid sequence. In some embodiments, the rMVA comprises two or more heterologous polycistronic inserts, for example, 2, 3, or 4, or more polycistronic inserts. In some embodiments, the population of chimeric polypeptides expressed from the rMVA are comprised of two or more different immune checkpoint inhibitor peptides. In some embodiments, the rMVA further encodes one or more antigenic peptides, which when expressed by the rMVA, are capable of inducing sufficient immunogenicity to provide or enhance protective immunity to an infectious agent. In some embodiments, the rMVA further encodes one or more antigenic peptides, which when expressed by the rMVA, are capable of inducing an immune response in the host which ameliorates one or more symptoms or conditions of a disorder, e.g., an infectious disease or cancer.

In some aspects, each of the chimeric polypeptides comprising a secretion signal peptide and an immune checkpoint inhibitor peptide encoded by the polycistronic nucleic acid insert includes a peptide sequence capable of being cleaved during or following translation linked to the C-terminus of the immune checkpoint inhibitor peptide. Where the secretable immune checkpoint inhibitor peptides are inserted in a multimeric conformation, inclusion of a cleavable peptide allows each chimeric polypeptide of the multimer to be expressed as a monomer during translation (e.g., through a translational nascent chain separation event) or, in an alternative embodiment, cleaved into monomers following translation, or a combination of both. In some embodiments, the chimeric polypeptide encoded by the most 3′ nucleic acid lacks a cleavable peptide sequence.

In some embodiments, provided herein is an rMVA viral vector comprising a heterologous nucleic acid insert encoding a polypeptide wherein the polypeptide comprises a sequence (M)(Secretion Signal Peptide-Immune Checkpoint Inhibitor Peptide)ₓ, wherein x=2, 3, 4, 5, 6, 7, 8, 9, 10, or more than 10, and M=methionine.

In some embodiments, provided herein is an rMVA viral vector comprising a heterologous polycistronic nucleic acid insert encoding a polypeptide wherein the polypeptide comprises a tandem repeat sequence (M)(Secretion Signal Peptide-Immune Checkpoint Inhibitor Peptide-Cleavable Peptide)ₓ, wherein x=2, 3, 4, 5, 6, 7, 8, 9, 10, or more than 10, and M=methionine (see, e.g., FIGS. 1A-1B). In some embodiments, provided herein is an rMVA viral vector comprising a heterologous polycistronic nucleic acid insert encoding two or more polypeptides in a tandem repeat sequence and an additional polypeptide fused to the C-terminus of the last polypeptide in the tandem repeat sequence ((M)(Secretion Signal Peptide-Immune Checkpoint Inhibitor Peptide-Cleavable Peptide)ₓ(Secretion Signal Peptide-Immune Checkpoint Inhibitor Peptide)), wherein x=1, 2, 3, 4, 5, 6, 7, 8, 9, 10, or more than 10, and M-methionine (see, e.g., FIGS. 2A-2B).

In some embodiments, the rMVA viral vector comprises a polycistronic nucleic acid insert encoding two or more polypeptides, wherein the polypeptides comprise tandem repeat sequences as described herein, for example a first polypeptide tandem repeat sequence comprising ((M)(Secretion Signal Peptide-Immune Checkpoint Inhibitor Peptide-Cleavable Peptide)ₓ(Secretion Signal Peptide-Immune Checkpoint Inhibitor Peptide)), wherein x=1, 2, 3, 4, 5, 6, 7, 8, 9, 10, or more than 10, wherein M=methionine, wherein the first polypeptide encoding sequence is oriented in a 5′→3′ direction, and a second polypeptide tandem repeat sequence comprising ((M)(Secretion Signal Peptide-Immune Checkpoint Inhibitor Peptide-Cleavable Peptide)ₓ(Secretion Signal Peptide-Immune Checkpoint Inhibitor Peptide)), wherein x=1, 2, 3, 4, 5, 6, 7, 8, 9, 10, or more than 10, wherein M=methionine, wherein the second polypeptide encoding sequence is oriented in a 3′→5′ direction, wherein each cistron includes a poxvirus promoter capable of initiating transcription. In some embodiments, x=3, 4, 5, or 6.

As provided herein, the rMVA is used as an adjuvant to increase the immunogenicity of one or more co-administered antigens during a vaccination protocol. By expressing localized, high quantities of one or more immune checkpoint inhibitor peptides capable of downregulating one or more checkpoint inhibitor pathways, immune modulating activities which typically hinder the development of sufficient antigenicity to induce immunity can be downregulated. In certain aspects, the immune checkpoint inhibitor peptide is capable of inhibiting the activity of an immune checkpoint pathway mediated by a receptor protein select from, but not limited to, programmed cell death protein-1 (PD-1), programmed death-ligand 1 (PD-L1), programmed death-ligand 2 (PD-L2), cytotoxic T-lymphocyte-associated protein 4 (CTLA-4), lymphocyte-activation gene 3 (LAG-3), T-cell immunoglobulin and mucin domain-3 (TIM-3), V-domain Ig suppressor of T-cell activation (VISTA), a B7 homolog protein (B7), B7 homolog 3 protein (B7-H3), B7 homolog 4 protein (B7-H4), B7 homolog 5 protein (B7-H5), OX-40 (OX-40), OX-40 ligand (OX-40L), glucocorticoid-induced TNFR-related protein (GITR), CD137, CD40, B and T lymphocyte attenuator (BTLA), Herpes Virus Entry Mediator (HVEM), galactin-9 (GAL9), killer cell immunoglobulin-like receptor (KIR), Natural Killer Cell Receptor 2B4 (2B4), CD160, checkpoint kinase 1 (CHK1), checkpoint kinase 2 (CHK2), adenosine A2a receptor (A2aR), T cell immunoreceptor with Ig and ITIM domains (TIGIT), inducible T cell co-stimulator (ICOS), inducible T cell co-stimulator ligand (ICOS-L), or combinations thereof. In some embodiments, the immune checkpoint inhibitor peptide is capable of inhibiting PD-1. In some embodiments, the immune checkpoint inhibitor peptide is capable of inhibiting PD-L1. In some embodiments, the immune checkpoint inhibitor peptide is capable of inhibiting CTLA-4. In some embodiments, the immune checkpoint inhibitor peptide is capable of inhibiting PD-1, PD-L1, or CTLA-4, or a combination thereof. In some embodiments, the immune checkpoint inhibitor peptide is capable of inhibiting both PD-1 and CTLA-4.

In some embodiments, the immune checkpoint inhibitor peptide is selected from a peptide described in Table 1, or a homolog, derivative, or fragment thereof. In some embodiments, the immune checkpoint inhibitor peptide is selected from a peptide having an amino acid sequence selected from the group consisting of SEQ ID NO: 1-56, or peptide having an amino acid sequence at least 85%, 90%, 95%, 97%, or 99% identical thereto. In some embodiments, the immune checkpoint inhibitor peptide is selected from a peptide having an amino acid sequence selected from the group consisting of SEQ ID NO:1-5, or a peptide having an amino acid sequence at least 85%, 90%, 95%, 97%, or 99% identical thereto. In some embodiments, the immune checkpoint inhibitor peptide is selected from a peptide having an amino acid sequence of SEQ ID NO: 1 (CRRTSTGQISTLRVNITAPLSQ), or peptide having an amino acid sequence at least 85%, 90%, 95%, 97%, or 99% identical thereto. In some embodiments, the immune checkpoint inhibitor peptide is selected from a peptide having an amino acid sequence of SEQ ID NO: 5 (STGQISTLRVNITAPLSQ), or an amino acid having an amino acid sequence at least 85%, 90%, 95%, 97%, or 99% identical thereto. In some embodiments, the immune checkpoint inhibitor peptide is selected from a peptide having an amino acid sequence of SEQ ID NO: 6 (STGQISTLAVNITAPLSQ), or an amino acid having an amino acid sequence at least 85%, 90%, 95%, 97%, or 99% identical thereto.

In some aspects as provided herein, each of the immune checkpoint inhibitor peptides expressed by the rMVA is fused to a secretion signal peptide on its N-terminus and, wherein the rMVA expresses two or more immune checkpoint inhibitor peptides, to one or more cleavable peptides on its C-terminus. The secretion signal peptide allows the immune checkpoint inhibitor peptide to be translocated into the endoplasmic reticulum (ER). Following co-translational insertion of the growing peptide chain into the ER lumen, a signal peptidase cleaves the signal peptide from the immune checkpoint inhibitor peptide, and the immune checkpoint inhibitor is secreted (see, e.g., FIG. 3A, FIGS. 3B, and 3C). The secretion signal peptides for use herein can be any suitable signal peptide that allows for the secretion of the immune checkpoint inhibitor peptide. Secretion signal peptide for use in the present invention are known in the art (see, e.g., Kober et al., Optimized signal peptides for the development of high expressing CHO cell lines. Biotechnol Bioengin. 2013; 110:1164-1173, incorporated herein by reference). In some embodiments, the secretion signal peptide is a short peptide having a length of between about 15-30 amino acids derived from a natural human excretory protein. In some embodiments, the secretion signal is a secretion signal selected from those of Table 2 (SEQ ID NO: 57-90), or a homolog, derivative, or fragment thereof. In some embodiments, the secretion signal peptide is, or is derived from, for example, but not limited to a human growth factor, a human cytokine, interleukin-1, interleukin-2, human immunoglobulin kappa light chain, trypsinogen, serum albumin, prolactin, tissue plasminogen activator, alkaline phosphatase, or other appropriate secretion signal sequence as described herein. In some embodiments, the secretion signal peptide is derived from human tissue plasminogen activator. In some embodiments, the secretion signal peptide is derived from human tissue plasminogen activator comprising an amino acid sequence DAMKRGLCCVLLLCGAVFVSPSQ (SEQ ID NO: 65), or peptide having an amino acid sequence at least 85%, 90%, 95%, 97%, or 99% identical thereto. In some embodiments, the secretion signal peptide is derived from human tissue plasminogen activator comprising an amino acid sequence DAMKRGLCCVLLLCGAVFVSPSQEIHARFRRGAR (SEQ ID NO: 66), or peptide having an amino acid sequence at least 85%, 90%, 95%, 97%, or 99% identical thereto. In some embodiments, the Secretion Signal Peptide of the first polypeptide encoded by the polycistronic nucleic acid insert further comprises the initiation amino acid methionine (M).

In some embodiments, one or more of the immune checkpoint inhibitor chimeric polypeptides includes one or more peptide sequences fused to the C-terminus of the immune checkpoint inhibitor peptide which is capable of being cleaved during or following, or a combination thereof, the translation of the polycistronic nucleic acid (see, e.g., FIGS. 3A, 3B, and 3C). In some embodiments, the most C-terminus immune checkpoint inhibitor chimeric polypeptide does not include a cleavable peptide. In some embodiments, the cleavable peptide is capable of being cleaved by a proprotein convertase enzyme including, for example, but not limited to furin or a furin-like proprotein convertase. In some embodiments, the cleavable peptide sequence comprises a basic amino acid target sequence (canonically, RX(R/K)R), wherein X=any amino acid (SEQ ID NO: 91). In some embodiments, the cleavable peptide sequence comprises a basic amino acid target sequence (canonically, RX(R/K)R), wherein X=R, K, or H (SEQ ID NO: 92). In some embodiments, the cleavable peptide sequence is RAKR (SEQ ID NO: 93). In some embodiments, the cleavable peptide sequence is RRRR (SEQ ID NO: 94). In some embodiments, the cleavable peptide is RKRR (SEQ ID NO: 95). In some embodiments, the cleavable peptide is RRKR (SEQ ID NO: 96). In some embodiments, the cleavable peptide is RKKR (SEQ ID NO: 97). By including a cleavable peptide sequence on each of the covalently linked chimeric polypeptides, the multimeric polypeptide expressed during translation of the polycistronic nucleic acid insert can be processed through a cleaving mechanism into monomeric chimeric polypeptides following translation. This allows each chimeric polypeptide comprising the immune checkpoint inhibitor peptide to be secreted from the cell and function to downregulate an undesirable immune checkpoint pathway (see, e.g., FIG. 3A).

In some embodiments, each chimeric polypeptide includes one or more peptide sequences fused to the C-terminus of the immune checkpoint inhibitor peptide which is capable of inducing ribozyme skipping during translation of the polycistronic nucleic acid. Ribosomal “skipping” is an alternate mechanism of translation in which a specific peptide sequence prevents the ribosome from covalently linking a new inserted amino acid, but nonetheless continues translation. This results in a “cleavage” of the polyprotein through the induced ribosomal skipping. In some embodiments, the peptide capable of inducing ribosomal skipping is a cis-acting hydrolase element peptide (CHYSEL). In some embodiments, the CHYSEL sequence comprises a non-conserved sequence of amino-acids with a strong alpha-helical propensity followed by the consensus sequence D(V/I)EXNPGP, where X=any amino acid (SEQ ID NO: 98), wherein the ribosomal skipping cleavage occurs between the G and P sequence. In some embodiments, the CHYSEL sequence comprises DVEENPGP (SEQ ID NO: 99). In some embodiments, the CHYSEL peptide sequence is a sequence selected from those in Table 4, or a peptide having an amino acid sequence at least 85%, 90%, 95%, 97%, or 99% identical thereto. In some embodiments, the CHYSEL peptide sequence is an amino acid sequence selected from SEQ ID NOS: 100-122, or a peptide having an amino acid sequence at least 85%, 90%, 95%, 97%, or 99% identical thereto. In some embodiments, the CHYSEL peptide sequence is an amino acid sequence selected from SEQ ID NOS: 118-122, or a peptide having an amino acid sequence at least 85%, 90%, 95%, 97%, or 99% identical thereto. In some embodiments, the CHYSEL sequence comprises GSGATNFSLLKQAGDVEENPGP (SEQ ID NO: 120), or peptide having an amino acid sequence at least 85%, 90%, 95%, 97%, or 99% identical thereto. By including a peptide sequence which induces ribosomal skipping, multiple chimeric polypeptides encoded by the polycistronic nucleic acid insert are expressed as monomers, which are then secreted from the cell and function to downregulate an undesirable immune checkpoint pathway (see, e.g., FIG. 3B).

In some embodiments, the cleavable peptide sequence comprises two or more sequences which are capable of being cleaved by different mechanism, for example a cleavable peptide sequence which is capable of being cleaved following the translation of the polycistronic nucleic acid and a peptide sequence capable of inducing ribozyme skipping during translation of the polycistronic nucleic acid. By providing cleavable peptide sequences subject to multiple modes of cleaving, the efficiency of monomeric formation from the polycistronic nucleic acid can be improved. In some embodiments, the immune checkpoint inhibitor peptide has fused to its C-terminus a furin-cleavable peptide sequence, for example the peptide sequence RX(R/K)R), wherein X=any amino acid (SEQ ID NO: 91), and fused to the C-terminus of the furin-cleavable peptide sequence is a CHYSEL peptide sequence comprising, for example D(V/I)EXNPGP, where X=any amino acid (SEQ ID NO: 98). For example, by including a furin-cleavable peptide sequence, such as RAKR (SEQ ID NO: 93), fused to the N-terminus of a CHYSEL peptide sequence between each chimeric polypeptide, the transcribed polycistronic nucleic acid undergoes ribozyme skipping during translation, resulting in the production of monomeric chimeric polypeptides, and following post translational processing and the cleavage of the furin-peptide, all but the arginine (R) and alanine (A) residues of the furin cleavage sequence remains at the C-terminus of immune checkpoint inhibitor peptide, limiting the potential interference of the extra amino acid sequences on the function of the immune checkpoint inhibitor peptide (see e.g., FIG. 3C). In alternative embodiments, the use of the furin-cleavable peptide RRRR (SEQ ID NO: 94), RKRR (SEQ ID NO: 95), or RRKR (SEQ ID NO: 96) results in the complete furin cleavage sequence being removed from the C-terminus of the immune checkpoint inhibitor peptide, with no residual amino acids remaining. In some embodiments, the hybrid cleavage sequence is RAKRGSGATNFSLLKQAGDVEENPGP (SEQ ID NO: 123), or a peptide having an amino acid sequence at least 85%, 90%, 95%, 97%, or 99% identical thereto. In some embodiments, the hybrid cleavage sequence is RRRRGSGATNFSLLKQAGDVEENPGP (SEQ ID NO: 124), or a peptide having an amino acid sequence at least 85%, 90%, 95%, 97%, or 99% identical thereto. In some embodiments, the hybrid cleavage sequence is RKRRGSGATNFSLLKQAGDVEENPGP (SEQ ID NO: 125), or a peptide having an amino acid sequence at least 85%, 90%, 95%, 97%, or 99% identical thereto. In some embodiments, the hybrid cleavage sequence is RRKRGSGATNFSLLKQAGDVEENPGP (SEQ ID NO: 126), or a peptide having an amino acid sequence at least 85%, 90%, 95%, 97%, or 99% identical thereto. In some embodiments, the hybrid cleavage sequence is RKKRGSGATNFSLLKQAGDVEENPGP (SEQ ID NO: 127), or a peptide having an amino acid sequence at least 85%, 90%, 95%, 97%, or 99% identical thereto.

In some embodiments, the rMVA viral vector comprises a heterologous polycistronic nucleic acid insert encoding a polypeptide having an amino acid sequence selected from SEQ ID NOS: 309-340, or SEQ ID NOS: 341-348. In some embodiments, the rMVA viral vector comprises a heterologous polycistronic nucleic acid insert encoding a polypeptide having an amino acid sequence of SEQ ID NOS: 325-340, or SEQ ID NOS:345-348. In some embodiments, the rMVA viral vector comprises a heterologous polycistronic nucleic acid insert encoding a polypeptide having an amino acid sequence of SEQ ID NO: 325. In some embodiments, the rMVA viral vector comprises a heterologous polycistronic nucleic acid insert encoding a polypeptide having an amino acid sequence of SEQ ID NO: 329. In some embodiments, the rMVA viral vector comprises a heterologous polycistronic nucleic acid insert encoding a polypeptide having an amino acid sequence of SEQ ID NO: 333. In some embodiments, the rMVA viral vector comprises a heterologous polycistronic nucleic acid insert encoding a polypeptide having an amino acid sequence of SEQ ID NO: 337.

Transcription of the nucleic acid insert can be initiated by one or more promoters compatible with the MVA viral vector located 5′ of, and operably linked to, the initial start codon of the first coding sequence contained within the nucleic acid. Suitable promotors compatible with a poxviral expression vector are known in the art and include, but are not limited to, pmH5, p11, pSyn, pHyb, or any other suitable MVA promoter sequence. In some embodiments, the promoter is a natural promoter for an MVA ORF. In some embodiments, the promoter is selected from a promoter in Table 7, or a nucleic acid having a sequence at least 85%, 90%, 95%, 97%, or 99% identical thereto. In some embodiments, the promoter sequence is selected from SEQ ID NOS: 128-308. or a nucleic acid sequence at least 85%, 90%, 95%, 97%, or 99% identical thereto. In some embodiments, the promoter sequence is selected from SEQ ID NOS: 130-132, or a nucleic acid sequence at least 85%, 90%, 95%, 97%, or 99% identical thereto. In some embodiments, the promoter sequence is SEQ ID NO: 130, or a nucleic acid sequence at least 85%, 90%, 95%, 97%, or 99% identical thereto.

In some embodiments wherein multiple immune checkpoint inhibitor peptides are expressed, because the chimeric polypeptides are transcribed as a single transcript, the polycistronic nucleic acid insert includes one or more termination signals (for example, a stop codon such as TAA, TAG, or TGA or a combination or multiples thereof) only following the ORF sequence of the last chimeric polypeptide. When transcribed, the multiple chimeric polypeptides result in a single transcript which is then translated. Following post-translational processing, the multiple monomeric chimeric polypeptides are produced.

The provided rMVA viral constructs of the present invention can be used as an adjuvant for treating or preventing an infectious disease or cancer, or inducing an immune response against an infectious disease or cancer, in a subject. In some embodiments, the rMVA viral construct is administered to a subject in need thereof, for example a human, in a prophylactic vaccination protocol to prevent an infectious disease, for example at a priming stage, a boosting stage, or both a priming stage and bosting stage. In an alternative embodiment, the rMVA viral construct is administered to a subject in need thereof, for example a human, in a treatment modality incorporating a vaccination protocol, for example, to treat a cancer. Accordingly, the rMVA viral construct can be administered in concert with one or more antigens intended to induce an immune response against an antigenic target in order to induce partial or complete immunization in a subject in need thereof.

Thus, the rMVA of the present invention can be administered with one or more antigens targeting an infectious disease or cancer. Examples of antigens and antigen delivery vehicles that the rMVA can be used with as an adjuvant include: an antigenic protein, polypeptide, or peptide, or fragment thereof, a nucleic acid, for example mRNA or DNA, encoding one or more antigens; a polysaccharide or a conjugate of a polysaccharide to a protein; glycolipids, for example gangliosides; a toxoid; a subunit (e.g., of a virus, bacterium, fungi, amoeba, parasite, etc.); a virus like particle; a live virus; a split virus; an attenuated virus; an inactivated virus; an enveloped virus; a viral vector expressing one or more antigens; a tumor associated antigen; or any combination thereof.

In particular aspects, the present invention provides a method of preventing or treating, or inducing an immune response against, an infectious disease in a subject in need thereof, said method comprising administering an effective amount of the rMVA of the present invention in combination, alternation, or coordination with a prophylactically effective or therapeutically effective amount of one or more antigens, or antigen expressing vectors, wherein the rMVA enhances immunity directed against the targeted infectious diseases.

In some embodiments, the targeted infection is a viral infection, including but not limited to: a double-stranded DNA virus, including but not limited to Adenoviruses, Herpesviruses, and Poxviruses; a single stranded DNA, including but not limited to Parvoviruses; a double stranded RNA virus, including but not limited to Reoviruses; a positive-single stranded RNA virus, including but not limited to Coronaviruses, for example SARS-CoV2, Picornaviruses, and Togaviruses; a negative-single stranded RNA virus, including but not limited to Orthomyxoviruses, and Rhabdoviruses; a single-stranded RNA-Retrovirus, including but not limited to Retroviruses; or a double-stranded DNA-Retrovirus, including but not limited to Hepadnaviruses. In some embodiments, the targeted virus is adenovirus, avian influenza, coxsackievirus, cytomegalovirus, dengue fever virus, ebola virus, Epstein-Barr virus, equine encephalitis virus, flavivirus, hepadnavirus, hepatitis A virus, hepatitis B virus, hepatitis C virus, hepatitis D virus, hepatitis E virus, herpes simplex virus, human immunodeficiency virus, human papillomavirus, influenza virus, Japanese encephalitis virus, JC virus, measles morbillivirus, marburg virus, Middle Eastern respiratory syndrome-coronavirus, mumps rubulavirus, orthomyxovirus, papillomavirus, parainfluenza virus, parvovirus, picornavirus, poliovirus, pox virus, rabies virus, reovirus, respiratory syncytial virus, retrovirus, rhabdovirus, rhinovirus, Rift Valley fever virus, rotavirus, rubella virus, rubeola virus, severe acute respiratory syndrome-coronavirus 1, severe acute respiratory syndrome coronavirus 2, smallpox virus, togavirus, swine influenza virus, varicella-zoster virus, variola major, variola minor, and yellow fever virus.

In some embodiments, the targeted infection is a bacterium, including but not limited to a Borrelia species, Bacillus anthraces, Borrelia burgdorferi, Bordetella pertussis, Camphylobacter jejuni, Chlamydia species, Chlamydial psittaci, Chlamydial trachomatis, Clostridium species, Clostridium tetani, Clostridium botulinum, Clostridium perfringens, Corynebacterium diphtheriae, Coxiella species, an Enterococcus species, Erlichia species, Escherichia coli, Francisella tularensis, Haemophilus species, Haemophilus influenzae, Haemophilus parainjluenzae, Lactobacillus species, a Legionella species, Legionella pneumophila, Leptospirosis interrogans, Listeria species, Listeria monocytogenes, Mycobacterium species, Mycobacterium tuberculosis, Mycobacterium leprae, Mycoplasma species, Mycoplasma pneumoniae, Neisseria species, Neisseria meningitidis, Neisseria gonorrhoeae, Pneumococcus species, Pseudomonas species, Pseudomonas aeruginosa, Salmonella species, Salmonella typhi, Salmonella enterica, Streptococcus species, Rickettsia species, Rickettsia ricketsii, Rickettsia typhi, Shigella species, Staphylococcus species, Staphylococcus aureus, Streptococcus species, Streptococcus pneumoniae, Streptococcus pyrogenes, Streptococcus mutans, Treponema species, Treponema pallidum, a Vibrio species, Vibrio cholerae and Yersinia pestis.

In some embodiments, the targeted infection is a fungal infection, including but not limited to a fungus from an Aspergillus species, Candida species, Candida albicans, Candida tropicalis, Cryptococcus species, Cryptococcus neoformans, Entamoeba histolytica, Histoplasma capsulatum, Leishmania species, Nocardia asteroides, Plasmodium falciparum, Toxoplasma gondii, Trichomonas vaginalis, Toxoplasma species, Trypanosoma brucei, Schistosoma mansoni, Fusarium species and Trichophyton species.

In some embodiments, the targeted infection is a parasite, including but not limited to a parasite from Plasmodium species, Toxoplasma species, Entamoeba species, Babesia species, Trypanosoma species, Leishmania species, Pneumocystis species, Trichomonas species, Giardia species and Schisostoma species.

In some embodiments, a method of preventing or treating, or inducing an immune response to, a cancer in a subject in need thereof, said method comprising administering an effective amount of the rMVA of the present invention in combination, alternation, or coordination with a prophylactically effective or therapeutically effective amount of one or more tumor associated antigens, or tumor associated antigen expressing vectors, wherein the rMVA enhances immunity directed against the cancer. In some embodiments, the tumor associated antigen (TAA) is, but is not limited to: an oncofetal TAA, which is typically only expressed in fetal tissues and in cancerous somatic cells; an oncoviral TAA, which is typically encoded by tumorigenic transforming viruses; an overexpressed/accumulated TAA, which is typically expressed by both normal and neoplastic tissue, with the level of expression highly elevated in neoplasia; a cancer-testis TAA, which is typically expressed only by cancer cells and adult reproductive tissues such as testis and placenta; a lineage-restricted TAA, which is typically expressed largely by a single cancer histotype; a mutated TAA, which is typically only expressed by cancer as a result of genetic mutation or alteration in transcription; a post-translationally altered TAA, which typically has tumor-associated alterations in glycosylation, etc.; and an idiotypic TAA, which is typically highly polymorphic genes where a tumor cell expresses a specific “clonotype”, i.e., as in B cell, T cell lymphoma/leukemia resulting from clonal aberrancies. In some embodiments, the TAA is selected from: Wilm's tumor protein (WT1); melanoma antigen preferentially expressed in tumors (PRAME); survivin; cancer/testis antigen 1 (NY-ESO-1); melanoma-associated antigen 3 (MAGE-A3); melanoma-associated antigen 4 (MAGE-A4); proteinase 3 (Pr3); Cyclin A1; highly homologous synovial sarcoma X 2 (SSX2); Neutrophil Elastase (NE); mucin 1 (MUC1); alphafetoprotein (AFP); carcinoembryonic antigen (CEA); cancer antigen 125 (CA-125); epithelial tumor antigen (ETA); tyrosinase; abnormal products of ras; abnormal products of p53; Epstein Bar Virus early antigen (EA), latent membrane protein 1 (LMP1), and latent membrane protein 2 (LMP2); a gangliosides for example, GM1b, GD1c, GM3, GM2, GM1a, GD1a, GT1a, GD3, GD2, GD1b, GT1b, GQ1b, GT3, GT2, GT1c, GQ1c, and GP1c; and a ganglioside derivative for example, 9-O—Ac-GD3, 9-O—Ac-GD2, 5-N-de-GM3, N-glycolyl GM3, NeuGcGM3, and fucosyl-GM1; or a combination thereof.

In some embodiments, the antigen is derived from an amino acid sequence of SEQ ID NOS:349-394.

In alternative embodiments, the rMVA viral vectors of the present invention, in addition to the ability to express multiple immune checkpoint inhibitor peptides, may further be constructed to encode and express one or more antigenic peptides. The one or more antigenic peptides can be encoded on one or more separate nucleic acid inserts, or in an alternative embodiment, the one or more antigenic peptides are encoded on the same polycistronic nucleic acid insert as the multiple immune checkpoint inhibitor peptides. In some embodiments, provided herein is an rMVA viral vector comprising a heterologous polycistronic nucleic acid insert encoding a polypeptide wherein the polypeptide comprises ((M)(Secretion Signal Peptide-Immune Checkpoint Inhibitor Peptide-Cleavable Peptide)ₓ(Antigenic Peptide)), wherein x=1, 2, 3, 4, 5, 6, 7, 8, 9, 10, or more than 10, and M=methionine. In some embodiments, the antigenic peptide is contained in a chimeric polypeptide comprising a secretion signal peptide fused to the N-terminus of the antigenic peptide, for example ((M)(Secretion Signal Peptide-Immune Checkpoint Inhibitor Peptide-Cleavable Peptide)ₓ(Secretion Signal Peptide-Antigenic Peptide)), wherein x=1, 2, 3, 4, 5, 6, 7, 8, 9, 10, or more than 10 and M=methionine (see, e.g., FIGS. 4A-4B). In some embodiments, the antigenic peptide is also provided so that 2 or more antigenic peptides are encoded in the polycistronic nucleic acid insert, with each chimeric polypeptide separated by a cleavable peptide described herein. In some embodiments, the antigenic peptide is contained in a chimeric polypeptide comprising a secretion signal peptide fused to the N-terminus of the antigenic peptide, and a cleavable peptide fused to the C-terminus of the antigenic peptide, for example ((M)(Secretion Signal Peptide-Immune Checkpoint Inhibitor Peptide-Cleavable Peptide)ₓ(Secretion Signal Peptide-Antigenic Peptide-Cleavable Peptide)_{y}), wherein x=1, 2, 3, 4, 5, 6, 7, 8, 9, 10, or more than 10, wherein y=1, 2, 3, 4, 5, 6, 7, 8, 9, 10, or more than 10, and M=methionine. In some embodiments, the antigen containing chimeric polypeptide fused to the C-terminus of the last antigen containing chimeric polypeptide does not include a cleavable sequence, for example ((M)(Secretion Signal Peptide-Immune Checkpoint Inhibitor Peptide-Cleavable Peptide)ₓ(Secretion Signal Peptide-Antigenic Peptide-Cleavable Peptide)ₓ(Secretion Signal Peptide-Antigenic Peptide)), wherein x=1, 2, 3, 4, 5, 6, 7, 8, 9, 10, or more than 10, and M=methionine. In some embodiments, the antigenic peptide contained in the chimeric polypeptide comprising a secretion signal peptide fused to the N-terminus of the antigenic peptide, and a cleavable peptide fused to the C-terminus of the antigenic peptide can be oriented in the polycistronic nucleic acid insert so that the antigen containing chimeric polypeptide encoding nucleic acid is located 5′ of the immune checkpoint inhibitor peptide containing chimeric polypeptides, for example ((M)(Secretion Signal Peptide-Antigenic Peptide-Cleavable Peptide)_{y}(Secretion Signal Peptide-Immune Checkpoint Inhibitor Peptide-Cleavable Peptide)ₓ) or, alternatively ((M)(Secretion Signal Peptide-Antigenic Peptide-Cleavable Peptide)_{y}(Secretion Signal Peptide-Immune Checkpoint Inhibitor Peptide-Cleavable Peptide)ₓ(Secretion Signal Peptide-Immune Checkpoint Inhibitor Peptide)), wherein y=1, 2, 3, 4, 5, 6, 7, 8, 9, 10, or more than 10, and wherein x=1, 2, 3, 4, 5, 6, 7, 8, 9, 10, or more than 10, and wherein M=methionine. In some embodiments, the antigenic peptide includes its natural secretion signal peptide. In alternative peptides, the Secretion Signal Peptide is not derived from the antigen, but rather derived from a different protein, synthetic secretion signal, or a consensus secretion signal peptide. In some embodiments, the antigenic peptide is selected from SEQ ID NOS. 349-394.

In some embodiments, the antigenic peptide encoded by the polycistronic nucleic acid insert in the rMVA is contained in a chimeric polypeptide that includes a viral glycoprotein signal sequence fused to the N-terminus of the antigenic peptide, and a viral glycoprotein transmembrane domain fused to the C-terminus of the antigenic peptide, and the rMVA is further constructed to encode a viral matrix protein, wherein upon translational cleavage of the antigenic containing chimeric peptide, the viral matrix protein and antigen-viral glycoprotein chimeric polypeptide are capable of forming a non-infectious virus-like particle (VLP). In some embodiments, provided herein is an rMVA viral vector comprising a heterologous polycistronic nucleic acid insert encoding a polypeptide wherein the polypeptide comprises ((M)(Secretion Signal Peptide-Immune Checkpoint Inhibitor Peptide-Cleavable Peptide)ₓ(Glycoprotein Signal Peptide-Antigenic Peptide-Glycoprotein Transmembrane Domain)), wherein x=1, 2, 3, 4, 5, 6, 7, 8, 9, 10, or more than 10, and wherein M=methionine (see, e.g., FIGS. 5A & 5B). In some embodiments, the antigenic peptide is contained in a chimeric polypeptide comprising a viral glycoprotein signal sequence fused to the N-terminus of the antigenic peptide, and a viral glycoprotein transmembrane domain fused to the C-terminus of the antigenic peptide, and a cleavable peptide fused to the C-terminus of the viral glycoprotein transmembrane domain, for example ((M)(Secretion Signal Peptide-Immune Checkpoint Inhibitor Peptide-Cleavable Peptide)ₓ(Glycoprotein Signal Peptide-Antigenic Peptide-Glycoprotein Transmembrane Domain-Cleavable Peptide)ₓ), wherein x=1, 2, 3, 4, 5, 6, 7, 8, 9, 10, or more than 10, and wherein M=methionine. In some embodiments, the antigen containing chimeric polypeptide fused to the C-terminus of the last antigen containing chimeric polypeptide does not include a cleavable sequence, for example ((M)(Secretion Signal Peptide-Immune Checkpoint Inhibitor Peptide-Cleavable Peptide)ₓ(Glycoprotein Signal Peptide-Antigenic Peptide-Glycoprotein Transmembrane Domain-Cleavable Peptide)_{y}(Glycoprotein Signal Peptide-Antigenic Peptide-Glycoprotein Transmembrane Domain)), wherein x=1 2, 3, 4, 5, 6, 7, 8, 9, 10, or more than 10, wherein y=1, 2, 3, 4, 5, 6, 7, 8, 9, 10, or more than 10, and M=methionine. In some embodiments, the (Glycoprotein Signal Peptide-Antigenic Peptide-Glycoprotein Transmembrane Domain-Cleavable Peptide)_{y}, wherein y=1, 2, 3, 4, 5, 6, 7, 8, 9, 10, or more than 10, can be oriented in the polycistronic nucleic acid insert so that the antigen containing chimeric polypeptide encoding nucleic acid is located 5′ of the immune checkpoint inhibitor peptide containing chimeric polypeptides, for example ((M)(Glycoprotein Signal Peptide-Antigenic Peptide-Glycoprotein Transmembrane Domain-Cleavable Peptide)_{y}(Secretion Signal Peptide-Immune Checkpoint Inhibitor Peptide-Cleavable Peptide)ₓ) or, alternatively ((M)(Glycoprotein Signal Peptide-Antigenic Peptide-Glycoprotein Transmembrane Domain-Cleavable Peptide)_{y}(Secretion Signal Peptide-Immune Checkpoint Inhibitor Peptide-Cleavable Peptide)ₓ(Secretion Signal Peptide-Immune Checkpoint Inhibitor Peptide)), wherein x=1 2, 3, 4, 5, 6, 7, 8, 9, 10, or more than 10, y=1, 2, 3, 4, 5, 6, 7, 8, 9, 10, or more than 10, and wherein M=methionine. In yet a further embodiment, the polycistronic nucleic acid insert of the rMVA further encodes the viral matrix protein, for example, ((M)(Secretion Signal Peptide-Immune Checkpoint Inhibitor Peptide-Cleavable Peptide)ₓ(Glycoprotein Signal Peptide-Antigenic Peptide-Glycoprotein Transmembrane Domain-Cleavable Peptide)(Viral Matrix Protein)), wherein x=1, 2, 3, 4, 5, 6, 7, 8, 9, 10, or more than 10, and wherein M=methionine (see, e.g., FIGS. 6A & 6B). In alternative embodiments, the coding sequences for both the antigen containing chimeric polypeptide and the viral matrix protein are contained in the polycistronic nucleic acid in one or more copies, for example, ((M)(Secretion Signal Peptide-Immune Checkpoint Inhibitor Peptide-Cleavable Peptide)ₓ(Glycoprotein Signal Peptide-Antigenic Peptide-Glycoprotein Transmembrane Domain-Cleavable Peptide)_{y}(Viral Matrix Protein-Cleavable Peptide)_{y}), wherein x=1, 2, 3, 4, 5, 6, 7, 8, 9, 10, or more than 10, y=1, 2, 3, 4, 5, 6, 7, 8, 9, 10, or more than 10, and M=methionine. In some embodiments, the most C-terminus viral matrix protein lacks a cleavable peptide, for example, ((M)(Secretion Signal Peptide-Immune Checkpoint Inhibitor Peptide-Cleavable Peptide)ₓ(Glycoprotein Signal Peptide-Antigenic Peptide-Glycoprotein Transmembrane Domain-Cleavable Peptide)ₓ(Viral Matrix Protein-Cleavable Peptide)_{y}(Viral Matrix Protein)), wherein x=1, 2, 3, 4, 5, 6, 7, 8, 9, 10, or more than 10, y=1, 2, 3, 4, 5, 6, 7, 8, 9, 10, or more than 10, and wherein M=methionine. In some embodiments, the ((M)(Glycoprotein Signal Peptide-Antigenic Peptide-Glycoprotein Transmembrane Domain-Cleavable Peptide)_{y}(Viral Matrix Protein-Cleavable Peptide)_{y}), wherein y=1, 2, 3, 4, 5, 6, 7, 8, 9, 10, or more than 10, and wherein M=methionine, can be oriented in the polycistronic nucleic acid insert so that the sequences are located 5′ of the immune checkpoint inhibitor peptide containing chimeric polypeptides, for example ((M)(Glycoprotein Signal Peptide-Antigenic Peptide-Glycoprotein Transmembrane Domain-Cleavable Peptide)_{y}(Viral Matrix Protein-Cleavable Peptide)_{y}(Secretion Signal Peptide-Immune Checkpoint Inhibitor Peptide-Cleavable Peptide)ₓ) or, alternatively ((M)(Glycoprotein Signal Peptide-Antigenic Peptide-Glycoprotein Transmembrane Domain-Cleavable Peptide)_{y}(Viral Matrix Protein-Cleavable Peptide)_{y}(Secretion Signal Peptide-Immune Checkpoint Inhibitor Peptide-Cleavable Peptide)ₓ(Secretion Signal Peptide-Immune Checkpoint Inhibitor Peptide)), wherein x=1, 2, 3, 4, 5, 6, 7, 8, 9, 10, or more than 10, y=1, 2, 3, 4, 5, 6, 7, 8, 9, 10, or more than 10, and wherein M=methionine. In some embodiments, the natural secretion signal from the antigen is replaced with a viral Glycoprotein Signal Peptide. In some embodiments, the antigenic peptide is selected from SEQ ID NOS: 349-394.

The production of virus-like particles containing a target antigen are particularly suitable for use in vaccine strategies against enveloped viruses, as they are capable of inducing both strong and durable humoral and cellular immune responses. See, e.g., Salvato et al., A Single Dose of Modified Vaccinia Ankara Expressing Lassa Virus-like Particles Protects Mice from Lethal Intra-cerebral Virus Challenge. Pathogens (2019) 8:133. Suitable glycoproteins and matrix proteins for use to produce the antigen containing VLPs include, but are not limited to, those derived from: a Filoviridae, for example Marburg virus, Ebola virus, or Sudan virus; a Retroviridae, for example human immunodeficiency virus type 1 (HIV-1); an Arenaviridaea, for example Lassa virus; a Flaviviridae, for example Dengue virus and Zika virus. In particular embodiments, the glycoprotein and matrix proteins are derived from Marburg virus (MARV). In particular embodiments, the glycoprotein is derived from the MARV GP protein (Genbank accession number AFV31202.1). The amino acid sequence of the MARV GP protein is provided as SEQ ID NO: 395 in Table 10 below. In particular embodiments, the MARV GPS domain comprises amino acids 2 to 19 of the glycoprotein (WTTCFFISLILIQGIKTL) (SEQ ID NO: 396, which can be encoded by, for example the MVA optimized nucleic acid sequence of SEQ ID NO: 397), the GPTM domain comprises amino acid sequences 644-673 of the glycoprotein (WWTSDWGVLTNLGILLLLSIAVLIALSCICRIFTKYIG) (SEQ ID NO: 398, which can be encoded by, for example the MVA optimized nucleic acid sequence of SEQ ID NO: 399). In some embodiments, the MARV GPS signal further comprises a methionine as the first amino acid.

The MARV VP40 amino acid sequence is available at GenBank accession number JX458834, and provided below in Table 10 as SEQ ID NO: 400, or a nucleic acid sequence 70%, 75%, 80%, 85%, 90%, 95% or more identical thereto. In some embodiments, the MARV VP40 signal further comprises a methionine as the first amino acid.

In some embodiments, the rMVA antigenic peptide encoded by the polycistronic nucleic acid insert in the rMVA is contained in a chimeric polypeptide that includes a viral glycoprotein signal sequence fused to the N-terminus of the antigenic peptide, and a viral glycoprotein transmembrane domain fused to the C-terminus of the antigenic peptide, and the rMVA is further constructed to encode a viral matrix protein, wherein upon translational cleavage of the antigenic containing chimeric peptide, the viral matrix protein and antigen-viral glycoprotein chimeric polypeptide are capable of forming a non-infectious virus-like particle (VLP).

In alternative embodiments, the rMVA viral vectors of the present invention, in addition to the ability to express multiple immune checkpoint inhibitor peptides, are further constructed to encode and express one or more antigenic peptides, wherein the one or more antigenic peptides are encoded on one or more separate nucleic acid inserts.

In some aspects, provided herein is a recombinant modified vaccinia ankara (rMVA) viral vector comprising one or more heterologous nucleic acid inserts encoding one or more chimeric polypeptides, each chimeric polypeptide comprising ((M)(Immune Checkpoint Inhibitor Peptide)x), wherein x=1-10, and M is methionine, wherein the heterologous nucleic acid inserts are under the control of a vaccinia virus promoter. In particular aspects, provided herein is a recombinant modified vaccinia ankara (rMVA) viral vector comprising one or more heterologous nucleic acid inserts encoding one or more chimeric polypeptides, each chimeric polypeptide comprising ((M)(Immune Checkpoint Inhibitor Peptide)x), wherein x=1-10, the Immune Checkpoint Inhibitor comprises SEQ ID NO:1, and M is methionine, wherein the heterologous nucleic acid inserts are under the control of a vaccinia virus promoter. In particular aspects, provided herein is a recombinant modified vaccinia ankara (rMVA) viral vector comprising one or more heterologous nucleic acid inserts encoding one or more chimeric polypeptides, each chimeric polypeptide comprising ((M)(Immune Checkpoint Inhibitor Peptide)x), wherein x=1-10, the Immune Checkpoint Inhibitor comprises SEQ ID NO:5, and M is methionine, wherein the heterologous nucleic acid inserts are under the control of a vaccinia virus promoter.

In some aspects, provided herein is a recombinant modified vaccinia ankara (rMVA) viral vector comprising i) a first nucleic acid sequence encoding a chimeric amino acid sequence comprising (a) an extracellular fragment of MUC-1, (b) a transmembrane domain of a glycoprotein (GP) of Marburg virus (MARV), and (c) an intracellular fragment of MUC-1; ii) a second nucleic acid sequence encoding a MARV VP40 matrix protein; iii) a third nucleic acid sequence encoding one or more immune checkpoint inhibitor peptides; and wherein the first nucleic acid sequence, the second nucleic acid sequence, and the third nucleic acid sequence are under the control of a vaccinia virus promoter; and wherein upon expression, the chimeric amino acid sequence and VP40 matrix protein are capable of assembling together to form virus-like particles (VLPs). In particular aspects, provided herein is a recombinant modified vaccinia ankara (rMVA) viral vector comprising i) a first nucleic acid sequence comprising the nucleic acid sequence of SEQ ID NO: 402; ii) a second nucleic acid sequence comprising the nucleic acid sequence of SEQ ID NO: 404; iii) a third nucleic acid sequence encoding one or more immune checkpoint inhibitor peptides; and wherein the first nucleic acid sequence, the second nucleic acid sequence, and the third nucleic acid sequence are under the control of a vaccinia virus promoter; and wherein upon expression, the chimeric amino acid sequence and VP40 matrix protein are capable of assembling together to form virus-like particles (VLPs). In particular aspects, provided herein is a recombinant modified vaccinia ankara (rMVA) viral vector comprising i) a first nucleic acid sequence encoding a chimeric amino acid sequence comprising the amino acid sequence of SEQ ID NO: 403; ii) a second nucleic acid sequence encoding a MARV VP40 matrix protein comprising the amino acid sequence of SEQ ID NO: 405; iii) a third nucleic acid sequence encoding one or more immune checkpoint inhibitor peptides; and wherein the first nucleic acid sequence, the second nucleic acid sequence, and the third nucleic acid sequence are under the control of a vaccinia virus promoter; and wherein upon expression, the chimeric amino acid sequence and VP40 matrix protein are capable of assembling together to form virus-like particles (VLPs).

In one embodiment, the first nucleic acid sequence, the second nucleic acid sequence, and the third nucleic acid sequence are inserted into one or more deletion sites of the MVA selected from I, II, III, IV, V or VI.

In another embodiment, the first nucleic acid sequence, the second nucleic acid sequence, and the third nucleic acid sequence are inserted into the MVA in a natural deletion site, a modified natural deletion site, or between essential or non-essential MVA genes.

In another embodiment, the first nucleic acid sequence, the second nucleic acid sequence, and the third nucleic acid sequence are inserted into the same natural deletion site, a modified natural deletion site, or between the same essential or non-essential MVA genes.

In another embodiment, the first nucleic acid sequence, the second nucleic acid sequence, and the third nucleic acid sequence are inserted into different natural deletion sites, different modified deletion sites, or between different essential or non-essential MVA genes.

In another embodiment, wherein the first nucleic acid sequence, the second nucleic acid sequence, and the third nucleic acid sequence are inserted between two essential and highly conserved MVA genes; and the matrix protein sequence is inserted into a restructured and modified deletion III.

In another embodiment, wherein the first nucleic acid sequence is inserted between MVA genes I8R and GIL, the second nucleic acid sequence is inserted between MVA genes A50R and B1R in the restructured and modified deletion site III, and the third nucleic acid sequence is inserted between the two essential MVA genes A5R and A6L.

In another embodiment, wherein the vaccinia virus promoter is a nucleic acid sequence selected from SEQ ID NOS: 128-308.

In another embodiment, wherein the vaccinia virus promoter is SEQ ID NO:130, or a nucleic acid sequence 95% identical thereto.

In some embodiments, the MUC-1 nucleic acid sequence is provided as SEQ ID NO:403, or a nucleic acid sequence 70%, 75%, 80%, 85%, 90%, 95% or more identical thereto. In some embodiments, the Marburg VP40 nucleic acid sequence is provided as SEQ ID NO:404, or a nucleic acid sequence 70%, 75%, 80%, 85%, 90%, 95% or more identical thereto. In some embodiments, the 5×LD01 nucleic acid sequence is provided as SEQ ID NO:408, or a nucleic acid sequence 70%, 75%, 80%, 85%, 90%, 95% or more identical thereto. In some embodiments, the 5×LD10 nucleic acid sequence is provided as SEQ ID NO:409, or a nucleic acid sequence 70%, 75%, 80%, 85%, 90%, 95% or more identical thereto.

Also provided herein are shuttle vectors comprising the polycistronic nucleic acid sequences to be inserted into the MVA as described herein, as well as isolated nucleic acid sequences comprising the polycistronic nucleic acid sequence inserts described herein. Further provided herein are cells comprising the rMVA viral vectors described herein.

### BRIEF DESCRIPTION OF THE DRAWINGS

FIG. 1A provides an exemplary linear schematic of an exemplary recombinant MVA viral vector polycistronic nucleic acid insert open reading frame (ORF) encoding multiple chimeric polypeptides, wherein each chimeric polypeptide comprises a secretion signal peptide, an immune checkpoint inhibitor peptide fused to the C-terminus of the signal peptide, and a cleavable peptide fused to the C-terminus of the immune checkpoint inhibitor peptide. The polycistronic nucleic acid insert can encode from 2 to 10 or more chimeric polypeptides, and includes a methionine as its first amino acid.

FIG. 1B provides an exemplary linear schematic of an exemplary recombinant MVA viral vector comprising a polycistronic nucleic acid insert encoding multiple chimeric polypeptides, wherein each chimeric polypeptide comprises a secretion signal peptide (SP), an immune checkpoint inhibitor peptide (ICIP) fused to the C-terminus of the secretion signal peptide, and a cleavable peptide (cleavage sequence) fused to the C-terminus of the immune checkpoint inhibitor peptide. As exemplified, a promoter capable of initiating transcription of an MVA ORF (e.g., mH5 promoter (pmH5)) is operably linked to a nucleic acid encoding multiple chimeric polypeptides. The insert may include a translation initiation sequence, for example a Kozak sequence, prior to the start codon of the most 5′ chimeric polypeptide ORF. As exemplified, a stop codon is present 3′ of the last chimeric polypeptide ORF.

FIG. 2A provides an exemplary linear schematic of an exemplary recombinant MVA viral vector polycistronic nucleic acid insert open reading frame (ORF) encoding multiple chimeric polypeptides, wherein all of the chimeric polypeptides comprise a secretion signal peptide (SP), an immune checkpoint inhibitor peptide fused to the C-terminus of the signal peptide, and a cleavable peptide fused to the C-terminus of the immune checkpoint inhibitor peptide, except for the most C-terminus chimeric polypeptide, which lacks a cleavable peptide. The polycistronic nucleic acid insert can encode from 2 to 10 or more chimeric polypeptides, and includes a methionine as its first amino acid.

FIG. 2B provides an exemplary linear schematic of an exemplary recombinant MVA viral vector comprising a polycistronic nucleic acid insert encoding multiple chimeric polypeptides, wherein each chimeric polypeptide comprises a secretion signal peptide (SP), an immune checkpoint inhibitor peptide (ICIP) fused to the C-terminus of the secretion signal peptide, and a cleavable peptide (cleavage sequence) fused to the C-terminus of the immune checkpoint inhibitor peptide, except for the most C-terminus chimeric polypeptide, which lacks a cleavable peptide. As exemplified, a promoter capable of initiating transcription of an MVA ORF (e.g., mH5 promoter (pmH5)) is operably linked to a nucleic acid encoding multiple chimeric polypeptides. The insert may include a translation initiation sequence, for example a Kozak sequence, prior to the start codon of the most 5′ chimeric polypeptide ORF. As exemplified, a stop codon is present 3′ of the last chimeric polypeptide ORF.

FIGS. 3A, 3B, and 3C provide exemplary schematics of the translational processing of the various expressed chimeric polypeptides encoded by the polycistronic nucleic acid inserts of the present invention. In FIG. 3A, the chimeric polypeptides encode a cleavable peptide sequence, for example a furin or furin-like cleavage sequence, which is cleaved following translation of the polycistronic nucleic acid transcript. In addition, during or following translation, the secretion signal peptide fused to the immune checkpoint inhibitor peptide is also cleaved, and the resultant monomeric immune checkpoint inhibitor peptides are subsequently secreted from the cell. In FIG. 3B, the chimeric polypeptides encode a cleavable peptide sequence, for example a CHYSEL cleavage sequence, that induces ribosomal skipping, wherein the polyprotein undergoes a co-translational cleavage, resulting in the production of monomeric immune checkpoint inhibitor peptides during translation. Following or during translation, the chimeric polypeptide undergoes further cleavage of the secreted signal peptide, and the resultant monomeric immune checkpoint inhibitor peptides are subsequently secreted from the cell. In FIG. 3C, the chimeric polypeptides encode multiple cleavable peptide sequences, for example both a furin or furin-like cleavage sequence and a CHYSEL sequence, for example, RAKRGSGATNFSLLKQAGDVEENPGP (SEQ ID NO: 123). During translation, induces ribosomal skipping at glycine (G) and proline (P) amino acids at the C-terminus of the CHYSEL sequence, wherein the polyprotein undergoes a co-translational cleavage, resulting in the production of monomeric immune checkpoint inhibitor peptides during translation. The monomeric immune checkpoint inhibitor peptides undergo further processing during or after translation, wherein the secreted signal peptide is cleaved. In addition, following translation, the furin or furin-like peptide sequence is cleaved, resulting in monomeric immune checkpoint inhibitor peptides containing only the arginine (R) and alanine (A) residues of the furin or furin like cleavage sequence, reducing the potential for interference with the immune checkpoint inhibitor peptides.

FIG. 4A provides an exemplary linear schematic of an exemplary recombinant MVA viral vector polycistronic nucleic acid insert open reading frame (ORF) encoding multiple chimeric polypeptides comprising tandem repeats of a secretion signal peptide, an immune checkpoint inhibitor peptide fused to the C-terminus of the signal peptide, and a cleavable peptide fused to the C-terminus of the immune checkpoint inhibitor peptide, and a chimeric polypeptide comprising a signal peptide fused to an antigenic peptide, the antigenic containing chimeric polypeptide fused to the most C-terminus immune checkpoint inhibitor containing chimeric peptide. The polycistronic nucleic acid insert can encode from 1 to 10 or more immune checkpoint inhibitor containing chimeric peptides, and includes a methionine as its first amino acid. This same general concept described above is applicable to any of the constructs provided herein which include cleavable sequences.

FIG. 4B provides an exemplary linear schematic of an exemplary recombinant MVA viral vector comprising a polycistronic nucleic acid insert encoding multiple chimeric polypeptides comprising tandem repeats of a secretion signal peptide (SP), an immune checkpoint inhibitor peptide (ICIP) fused to the C-terminus of the signal peptide, and a cleavable peptide (cleavage sequence) fused to the C-terminus of the immune checkpoint inhibitor peptide, and a antigen containing chimeric polypeptide comprising a secretion signal peptide (SP) fused to an antigenic peptide (Antigen), the antigen containing chimeric polypeptide fused to the most C-terminus immune checkpoint inhibitor containing chimeric peptide. As exemplified, a promoter capable of initiating transcription of an MVA ORF (e.g., mH5 promoter (pmH5)) is operably linked to a nucleic acid encoding the multiple chimeric polypeptides. The insert may include a translation initiation sequence, for example a Kozak sequence, prior to the start codon of the most 5′ chimeric polypeptide ORF. As exemplified, a stop codon is present 3′ of the last chimeric polypeptide ORF.

FIG. 5A provides an exemplary linear schematic of an exemplary recombinant MVA viral vector polycistronic nucleic acid insert open reading frame (ORF) encoding multiple chimeric polypeptides comprising tandem repeats of a secretion signal peptide, an immune checkpoint inhibitor peptide fused to the C-terminus of the signal peptide, and a cleavable peptide fused to the C-terminus of the immune checkpoint inhibitor peptide, and an antigen containing chimeric polypeptide comprising a viral glycoprotein signal peptide fused to an antigenic peptide, which is fused to the transmembrane domain of a viral glycoprotein, wherein the antigen containing chimeric polypeptide is fused to the most C-terminus immune checkpoint inhibitor containing chimeric peptide. The polycistronic nucleic acid insert can encode from 1 to 10 or more immune checkpoint inhibitor containing chimeric polypeptides, and includes a methionine as its first amino acid.

FIG. 5B provides an exemplary linear schematic of an exemplary recombinant MVA viral vector comprising a polycistronic nucleic acid insert encoding multiple chimeric polypeptides comprising tandem repeats of a secretion signal peptide (SP), an immune checkpoint inhibitor peptide (ICIP) fused to the C-terminus of the signal peptide, and a cleavable peptide (Cleavage sequence) fused to the C-terminus of the immune checkpoint inhibitor peptide, and an antigen containing chimeric polypeptide comprising a viral glycoprotein signal peptide (GPSP) fused to an antigenic peptide (Antigen), which is fused to the transmembrane domain of a viral glycoprotein transmembrane domain (GPTM), fused to the most C-terminus immune checkpoint inhibitor containing chimeric peptide. As exemplified, a promoter capable of initiating transcription of an MVA ORF (e.g., mH5 promoter (pmH5)) is operably linked to the polycistronic nucleic acid encoding the multiple chimeric polypeptides. The insert may include a translation initiation sequence, for example a Kozak sequence, prior to the start codon of the most 5′ chimeric polypeptide ORF. As exemplified, a stop codon is present 3′ of the last polypeptide ORF.

FIG. 6A provides an exemplary linear schematic of an exemplary recombinant MVA viral vector polycistronic nucleic acid insert open reading frame (ORF) encoding multiple chimeric polypeptides comprising tandem repeats of a secretion signal peptide, an immune checkpoint inhibitor peptide fused to the C-terminus of the signal peptide, and a cleavable peptide fused to the C-terminus of the immune checkpoint inhibitor peptide, and an antigen containing chimeric polypeptide comprising a viral glycoprotein signal peptide fused to an antigenic peptide, which is fused to the transmembrane domain of a viral glycoprotein and further fused to a cleavable peptide, wherein the antigen containing chimeric polypeptide is fused to the most C-terminus immune checkpoint inhibitor containing chimeric peptide, and further comprising a viral matrix protein, wherein the viral matrix protein is fused to the C-terminus of the cleavable peptide of the antigen containing chimeric polypeptide. The polycistronic nucleic acid insert can encode from 1 to 10 or more immune checkpoint inhibitor containing chimeric polypeptides, and includes a methionine as its first amino acid.

FIG. 6B provides an exemplary linear schematic of an exemplary recombinant MVA viral vector comprising a polycistronic nucleic acid insert encoding multiple chimeric polypeptides comprising a secretion signal peptide (SP), an immune checkpoint inhibitor peptide (ICIP) fused to the C-terminus of the signal peptide, and a cleavable peptide (Cleavage sequence) fused to the C-terminus of the immune checkpoint inhibitor peptide, and an antigen containing chimeric polypeptide comprising a viral glycoprotein signal peptide (GPSP) fused to an antigenic peptide (Antigen), which is fused to the transmembrane domain of a viral glycoprotein transmembrane domain (GPTM) fused to a cleavable peptide, wherein the antigen containing chimeric polypeptide is fused to the most C-terminus immune checkpoint inhibitor containing chimeric peptide, and further comprising a viral matrix protein, wherein the viral matrix protein is fused to the C-terminus of the cleavable peptide of the antigen containing chimeric polypeptide. As exemplified, a promoter capable of initiating transcription of an MVA ORF (e.g., mH5 promoter (pmH5)) is operably linked to the polycistronic nucleic acid encoding the multiple chimeric polypeptides. The insert may include a translation initiation sequence, for example a Kozak sequence, prior to the start codon of the most 5′ chimeric polypeptide ORF. As exemplified, a stop codon is present 3′ of the viral matrix protein ORF.

FIG. 7 is a schematic of MVA-5X.LD01 and MVA-5X.LD10 vectors illustrating the design of peptide sequences inserted into the MVA genome between two essential genes under control of an MVA specific promoter. LD01 and LD10 sequences are preceded by a signal sequence routing peptide for secretion and followed by a cleavage site to separate duplicated peptides. The secretion signal, peptide sequence and cleavage site are repeated 5 times and then transcription is terminated with a stop codon.

FIG. 8 shows the production of LD01 and LD10 by MVA-infected cells. (FIG. 8A) DF-1 cells were infected with MVA-5X.LD01, MVA-5X.LD10 or parental MVA. Two days following infection cells were fixed, permeabilized and stained with an antibody specific for LD01 and LD10. Results show the peptides are detected intracellularly. LD01- and LD10-positive cells were stained as shown. Photomicrographs are presented at a magnification of 20×. (FIG. 8B) DF-1 cells were infected with MVA-5X.LD01, MVA-5X.LD10 or parental MVA. Two days following infection, supernatant was harvested, concentrated and dotted onto membrane along with chemically synthesized peptide (LD01) and probed with an antibody specific for LD01 and LD10. Results indicate that the peptides are secreted from the infected cells.

FIG. 9 shows the delivery of LD01 or LD10 via a viral vector enhances expansion of vaccine-induced, antigen-specific CD8⁺ T cells. (FIG. 9A and FIG. 9B). At day 12 post-AdPyCS immunization, immunogenicity was assessed by measuring the number of splenic PyCS-specific, IFN-7-secreting CD8⁺ T cells using the ELISpot assay (FIG. 9A) and flow cytometry (FIG. 9B) after stimulation with the H-2kd restricted CD8 epitope SYVPSAEQI (SEQ ID NO: 406). A 100 μg dose of LD01 or LD10da was given SC immediately following vaccination. For viral vectors, 10⁷ TCID₅₀ of MVA-5X.LD01, MVA-5X.LD10 or parental MVA was injected SC subsequent to vaccination. Data are expressed as the mean±SEM. Data from one of two independent experiments are shown. Significant differences between AdPyCS alone and treated mice were determined using a two-tailed Unpaired t-test and denoted by ** (p<0.001), *** (p<0.0005) and **** (p<0.0001). For FIG. 9A, the x axis is AdPyCS alone and treated mice and the y axis is number of IFN-γ spots per 1×10⁶ splenocytes measured in counts. For FIG. 9B, the x axis is AdPyCS alone and treated mice and the y axis is number of IFN-γ CB8 T cells within total CB8 T cells measured in percentage.

FIG. 10 shows a PCR gel of LD10, MUC-1, and VP40 inserts amplified from MVA-VLP-MUC-1-LD10 virus infected DF-1 cell DNA samples. DF1 cells infected with parental MVA (negative control), plasmids carrying LD10, MUC-1, or VP40 inserts (positive controls), or MVA-VLP-MUC-1-LD10 recombinant virus were harvested for viral DNA. PC′R analysis confirmed insert integrity.

FIG. 11 shows the expected PCR fragment sizes of LD10, MUC-1, and VP40 insert sizes collected from DF-1 cells infected with MVA-VLP-MUC-1-LD10 virus. The expected fragment sizes matched the band sizes of the PCR gel.

FIG. 12 shows the expression of recombinant MUC-1 protein in DF-1 cells infected with MVA-VLP-MUC-1-LD10. DF1 cells were infected with parental modified vaccinia Ankara (pMVA) or MVA encoding VLP-MUC-1-LD10. Uninfected cells were included as negative controls. Cellular lysate and supernatant were harvested for protein and analyzed by immunoblotting. Membranes were probed with MUC-1 antibody (mouse monoclonal VU4H5, Santa Cruz #sc-7313, 1:200), labeling a protein band of approximately 63 kDa in the MVS-VLP-MUC-1-LD10 lysate sample.

FIG. 13 shows the expression of recombinant VP40 protein in DF-1 cells infected with MVA-VLP-MUC-1-LD10. DF1 cells were infected with parental modified vaccinia Ankara (pMVA) or MVA encoding VLP-MUC-1-LD10. Uninfected cells were included as negative controls. Cellular lysate and supernatant were harvested for protein and analyzed by immunoblotting. Membranes were probed with VP40 antibody, labeling a protein band of approximately 32 kDa in the MVS-VLP-MUC-1-LD10 supernatant and lysate samples.

FIG. 14 shows the expression of recombinant LD10 protein in DF-1 cells infected with MVA-VLP-MUC-1-LD10. DF1 cells were transfected with parental modified vaccinia Ankara (pMVA) or MVA encoding VLP-MUC-1-LD10. Uninfected cells were included as negative controls. Cellular lysates were harvested for protein and applied to nitrocellulose membrane using a dot blot apparatus. Twenty micrograms of LD10 peptide was also loaded onto the membrane as a positive control of the LD10 antibody. The membrane was probed with LD10 antibody, demonstrating signal in the MVA-VLP-MUC-1-LD10 and LD10 peptide samples.

FIG. 15 shows the percentages of MUC-1-positive plaques following infection of DF-1 cells with different amounts of recombinant MVA-VLP-MUC-1-LD10 virus. DF1 cells were infected in 3 wells each of 30 plaque forming units (PFU) and 60 PFU of MVA-VLP-MUC-1-LD10 virus in a 6 well plate. All wells were probed with MUC-1 antibody and the number of MUC-1-positive plaques were counted. The wells were then washed before being probed again with MVA antibody and the number of MVA-positive plaques were counted. To calculate the purity of the vaccine, the percentage of MUC-1-positive plaques versus the number of MVA-positive plaques is shown. The number of positive plaques for each individual replicate are shown at the bottom of the figure.

FIG. 16 shows the percentages of VP40-positive plaques following infection of DF-1 cells with different amounts of recombinant MVA-VLP-MUC-1-LD10 virus. DF1 cells were infected in 3 wells each of 30 plaque forming units (PFU) and 60 PFU of MVA-VLP-MUC-1-LD10 virus in a 6 well plate. All wells were probed with MUC-1 antibody and the number of VP40-positive plaques were counted. The wells were then washed before being probed again with MVA antibody and the number of MVA-positive plaques were counted. To calculate the purity of the vaccine, the percentage of VP40-positive plaques versus the number of MVA-positive plaques is shown. The number of positive plaques for each individual replicate are shown at the bottom of the figure.

### DETAILED DESCRIPTION OF THE INVENTION

### Definitions

Where a term is provided in the singular, the inventors also contemplate aspects of the invention described by the plural of that term. As used in this specification and in the appended claims, the singular forms “a”, “an”, and “the” include plural references unless the context clearly dictates otherwise, e.g., “a peptide” or a “chimeric polypeptide” includes a plurality of peptides or chimeric polypeptides. Thus, for example, a reference to “a method” includes one or more methods, and/or steps of the type described herein, and/or which will become apparent to those persons skilled in the art upon reading this disclosure.

The term “adjuvant” as used herein means the use of the rMVA as described herein to enhance the immunogenicity of one or more antigens.

The term “antigen” refers to a substance or molecule, such as a protein, or fragment thereof, e.g., a peptide, that is capable of inducing an immune response.

“Chimeric” or “fused” as used herein indicates the covalent joining of peptides or proteins that do not naturally exist, resulting in a hybrid polypeptide. Translation of the chimeric or fused polypeptides described herein provide functional properties derived from each of the respective fused peptides or proteins.

“Coding sequence” or “encoding nucleic acid” or “nucleic acid sequence encoding” or the like, as used herein means the nucleic acids (RNA or DNA molecule) that comprise a nucleotide sequence which encodes an amino acid sequence, for example, a polyprotein, polypeptide, protein, peptide, or fragment thereof. The coding sequence can further include initiation and termination signals operably linked to regulatory elements including a promoter and polyadenylation signal capable of directing expression in the cells of human or mammal to which the nucleic acid is administered.

The term “conservative amino acid substitution” refers to substitution of a native amino acid residue with a non-native residue such that there is little or no effect on the size, polarity, charge, hydrophobicity, or hydrophilicity of the amino acid residue at that position, and without resulting in substantially altered immunogenicity. For example, these may be substitutions within the following groups: valine; glycine, alanine; valine, isoleucine, leucine; aspartic acid, glutamic acid; asparagine, glutamine; serine, threonine; lysine, arginine; and phenylalanine, tyrosine. Conservative amino acid modifications to the sequence of a polypeptide (and the corresponding modifications to the encoding nucleotides) may produce polypeptides having functional and chemical characteristics similar to those of a parental polypeptide.

The term “deletion” in the context of a polypeptide or protein refers to removal of codons for one or more amino acid residues from the polypeptide or protein sequence, wherein the regions on either side are joined together. The term deletion in the context of a nucleic acid refers to removal of one or more bases from a nucleic acid sequence, wherein the regions on either side are joined together.

The term “fragment” in the context of a proteinaceous agent refers to a peptide or polypeptide comprising an amino acid sequence of at least 2 contiguous amino acid residues, at least 5 contiguous amino acid residues, at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least 80 contiguous amino acid residues, at least 90 contiguous amino acid residues, at least 100 contiguous amino acid residues, at least 125 contiguous amino acid residues, at least 150 contiguous amino acid residues, at least 175 contiguous amino acid residues, at least 200 contiguous amino acid residues, or at least 250 contiguous amino acid residues of the amino acid sequence of a peptide, polypeptide, or protein. In one embodiment, the fragment constitutes at least 10%, 20%, 30%, 40%, 50%, 60%, 70%, 80%, or 90% of the entire length of the reference polypeptide. In one embodiment, a fragment of a full-length protein retains activity of the full-length protein. In another embodiment, the fragment of the full-length protein does not retain the activity of the full-length protein.

The term “fragment” in the context of a nucleic acid refers to a nucleic acid comprising an nucleic acid sequence of at least 2 contiguous nucleotides, at least 5 contiguous nucleotides, at least 10 contiguous nucleotides, at least 15 contiguous nucleotides, at least 20 contiguous nucleotides, at least 25 contiguous nucleotides, at least 30 contiguous nucleotides, at least 35 contiguous nucleotides, at least 40 contiguous nucleotides, at least 50 contiguous nucleotides, at least 60 contiguous nucleotides, at least 70 contiguous nucleotides, at least contiguous 80 nucleotides, at least 90 contiguous nucleotides, at least 100 contiguous nucleotides, at least 125 contiguous nucleotides, at least 150 contiguous nucleotides, at least 175 contiguous nucleotides, at least 200 contiguous nucleotides, at least 250 contiguous nucleotides, at least 300 contiguous nucleotides, at least 350 contiguous nucleotides, or at least 380 contiguous nucleotides of the nucleic acid sequence encoding a peptide, polypeptide or protein. In one embodiment the fragment constitutes at least 10%, 20%, 30%, 40%, 50%, 60%, 70%, 80%, or 90% of the entire length of the reference nucleic acid sequence. In a preferred embodiment, a fragment of a nucleic acid encodes a peptide or polypeptide that retains activity of the full-length protein. In another embodiment, the fragment encodes a peptide or polypeptide that of the full-length protein does not retain the activity of the full-length protein.

As used herein, the phrase “heterologous sequence” refers to any nucleic acid, protein, polypeptide, or peptide sequence which is not normally associated in nature with another nucleic acid or protein, polypeptide, or peptide sequence of interest.

As used herein, the phrase “heterologous nucleic acid insert” refers to any nucleic acid sequence that has been, or is to be inserted into the recombinant vectors described herein. The heterologous nucleic acid insert may refer to only the gene product encoding sequence or may refer to a sequence comprising a promoter, a gene product encoding sequence (for example secretion signal peptide-immune checkpoint inhibitor peptide chimeric polypeptides) and any regulatory sequences associated or operably linked therewith.

The term “homopolymer stretch” refers to a sequence comprising at least four of the same nucleotides uninterrupted by any other nucleotide, e.g., GGGG or TTTTTTT.

The terms “percent identical,” “percent homologous,” or “percent similarity”, and the like, when used in the context of nucleic acid sequences refers to the residues in the two sequences being compared which are the same when aligned for maximum correspondence. The length of sequence identity comparison may be over the full-length of the sequence, or, or alternatively a fragment of at least about 50 to 2500 nucleotides. Similarly, the terms “percent identical,” “percent homologous,” or “percent similarity”, may be readily determined for amino acid sequences, over the full-length of a protein, or a fragment thereof. Suitably, a fragment is at least about 8 amino acids in length and may be up to about 7500 amino acids. Examples of suitable fragments are described herein. Generally, “identity”, “homology” or “similarity” is determined in reference to “aligned” sequences. “Aligned” sequences or “alignments” refer to multiple nucleic acid sequences or protein (amino acids) sequences, often containing corrections for missing or additional bases or amino acids as compared to a reference sequence. Alignments can be performed using any of a variety of publicly or commercially available Multiple Sequence Alignment Programs. Examples of such programs include, “Clustal Omega”, “Clustal W”, “CAP Sequence Assembly”, “MAP”, and “MEME”, which are accessible through Web Servers on the internet. Other sources for such programs are known to those of skill in the art. Alternatively, Vector NTI utilities are also used. There are also a number of algorithms known in the art that can be used to measure nucleotide sequence identity, including those contained in the programs described above. As another example, polynucleotide sequences can be compared using Fasta™ a program in GCG Version 6.1. Fasta™ provides alignments and percent sequence identity of the regions of the best overlap between the query and search sequences. For instance, percent sequence identity between nucleic acid sequences can be determined using Fasta™ with its default parameters (a word size of 6 and the NOPAM factor for the scoring matrix) as provided in GCG Version 6.1, herein incorporated by reference. Multiple sequence alignment programs are also available for amino acid sequences, e.g., the “Clustal Omega”, “Clustal X”, “MAP”, “PIMA”, “MSA”, “BLOCKMAKER”, “MEME”, and “Match-Box” programs. Generally, any of these programs are used at default settings, although one of skill in the art can alter these settings as needed. Alternatively, one of skill in the art can utilize another algorithm or computer program which provides at least the level of identity or alignment as that provided by the referenced algorithms and programs. See, e.g., J. D. Thomson et al, Nucl. Acids. Res., “A comprehensive comparison of multiple sequence alignments”, 27(13):2682-2690 (1999).

The term “inducing an immune response” means eliciting a humoral response (e.g., the production of antibodies) or a cellular response (e.g., the activation of T cells), or both a humoral and a cellular response, directed against one or more antigenic proteins or fragments thereof expressed by the rMVA in a subject to which the rMVA has been administered.

The term “modified vaccinia Ankara,” “modified vaccinia ankara,” “Modified Vaccinia Ankara,” or “MVA” generally refers to a highly attenuated strain of vaccinia virus developed by Dr. Anton Mayr by serial passage on chick embryo fibroblast cells; or variants or derivatives thereof. MVA is reviewed in Mayr, A. et al. 1975 Infection 3:6-14. The genomic sequence of MVA and various variants is described, for example, at GenBank Accession Numbers AY603355, U94848, and DQ983238. In some embodiments, the MVA as provided herein can be derived synthetically, for example, through chemically synthesized plasmids and reconstituted to the full length genomic MVA sequence in a host cell, for example, as described in US2018/0251736, US2021/0230560, and WO2021/158565, each incorporated herein by reference.

“Nucleic acid” or “oligonucleotide” or “polynucleotide” as used herein means at least two nucleotides covalently linked together. The depiction of a single strand also defines the sequence of the complementary strand. Thus, a nucleic acid also encompasses the complementary strand of a depicted single strand. Many variants of a nucleic acid can be used for the same purpose as a given nucleic acid. Thus, a nucleic acid also encompasses substantially identical nucleic acids and complements thereof. A single strand provides a probe that can hybridize to a target sequence under stringent hybridization conditions. Thus, a nucleic acid also encompasses a probe that hybridizes under stringent hybridization conditions.

Nucleic acids can be single stranded or double stranded, or can contain portions of both double stranded and single stranded sequence. The nucleic acid can be DNA, both genomic and cDNA, RNA, or a hybrid, where the nucleic acid can contain combinations of deoxyribo- and ribo-nucleotides, and combinations of bases including uracil, adenine, thymine, cytosine, guanine, inosine, xanthine hypoxanthine, isocytosine and isoguanine. Nucleic acids can be obtained by chemical synthesis methods or by recombinant methods.

“Operably linked” as used herein means that expression of a gene is under the control of a promoter with which it is spatially connected. A promoter can be positioned 5′ (upstream) or 3′ (downstream) of a gene under its control. The distance between the promoter and a gene can be approximately the same as the distance between that promoter and the gene it controls in the gene from which the promoter is derived. As is known in the art, variation in this distance can be accommodated without loss of promoter function.

A “peptide,” “protein,” “polypeptide,” or “polyprotein” as used herein can mean a linked sequence of amino acids and can be natural, synthetic, or a modification or combination of natural and synthetic.

“Promoter” as used herein means a synthetic or naturally-derived molecule which is capable of conferring, activating, or enhancing the transcription of a nucleic acid in a cell. A promoter can comprise one or more specific transcriptional regulatory sequences to further enhance expression and/or to alter the spatial expression and/or temporal expression of same. A promoter can also comprise distal enhancer or repressor elements, which can be located as much as several thousand base pairs from the start site of transcription.

The term “prevent,” “preventing,” and “prevention” refers to the inhibition of the development or onset of a condition (e.g., an infection), or the prevention of the recurrence, onset, or development of one or more symptoms of a condition in a subject resulting from the administration of a therapy or the administration of a combination of therapies.

The term “prophylactically effective amount” refers to the amount of a composition (e.g., the target antigenic composition and/or rMVA described herein) which is sufficient to result in the prevention of the development, recurrence, or onset of a condition or a symptom thereof (e.g., a viral infection) or symptom associated therewith or to enhance or improve the prophylactic effect(s) of another therapy.

The term “recombinant,” with respect to a viral vector, means a vector (e.g., a viral genome) that has been manipulated in vitro, e.g., using recombinant nucleic acid techniques to express heterologous viral nucleic acid sequences.

The term “regulatory sequence” and “regulatory sequences” refers collectively to promoter sequences, polyadenylation signals, transcription termination sequences, upstream regulatory domains, origins of replication, internal ribosome entry sites (“IRES”), enhancers, and the like, which collectively provide for the transcription and translation of a coding sequence. Not all of these control sequences need always be present so long as the selected gene is capable of being transcribed and translated.

The term “shuttle vector” refers to a genetic vector (e.g., a DNA plasmid) that is useful for transferring genetic material from one host system into another. A shuttle vector can replicate alone (without the presence of any other vector) in at least one host (e.g., E. coli). In the context of MVA vector construction, shuttle vectors are usually DNA plasmids that can be manipulated in E. coli and then introduced into cultured cells infected with MVA vectors, resulting in the generation of new recombinant MVA vectors via, for example, homologous recombination.

The term “silent mutation” means a change in a nucleotide sequence that does not cause a change in the primary structure of the protein encoded by the nucleotide sequence, e.g., a change from AAA (encoding lysine) to AAG (also encoding lysine).

The “host,” “patient,” or “subject” treated is typically a human patient, although it is to be understood the methods described herein are effective with respect to other animals, such as mammals. More particularly, the term patient can include animals used in assays such as those used in preclinical testing including but not limited to mice, rats, monkeys, dogs, pigs and rabbits; as well as domesticated swine (pigs and hogs), ruminants, equine, poultry, felines, bovines, murines, canines, and the like. Determination of those subjects “at risk” can be made by any objective or subjective determination by a diagnostic test or opinion of a subject or health care provider (e.g., genetic test, enzyme or protein marker, marker history, and the like).

The term “synonymous codon” refers to the use of a codon with a different nucleic acid sequence to encode the same amino acid, e.g., AAA and AAG (both of which encode lysine). Codon optimization changes the codons for a protein to the synonymous codons that are most frequently used by a vector or a host cell.

The term “therapeutically effective amount” means the amount of the composition (e.g., the antigenic composition and/or recombinant MVA vector or pharmaceutical composition) that, when administered to a subject for treating or preventing a disorder, e.g., an infection or cancer, is sufficient to affect such treatment or prevention for the disorder.

The term “treating” or “treat” refer to the eradication or control of a disorder, the reduction or amelioration of the progression, severity, and/or duration of a disorder or one or more symptoms caused by the disorder resulting from the administration of one or more therapies.

The term “vaccine” means material used to provoke an immune response and confer immunity after administration of the material to a subject. Such immunity may include a cellular or humoral immune response that occurs when the subject is exposed to the immunogen after vaccine administration.

The term “virus-like particles” or “VLP” refers to a structure which resembles a virus but is not infectious because it does not contain viral genetic material.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

### Modified Vaccinia Ankara (MVA) Viral Vectors

Modified vaccinia Ankara (MVA) in particular has been employed as a safe and potent viral vector vaccine against infectious diseases. MVA is a highly attenuated strain of vaccinia virus derived by extensive serial passages in chicken embryo fibroblasts (CEF) (Sutter G, Staib C. Vaccinia vectors as candidate vaccines: the development of modified vaccinia virus Ankara for antigen delivery. Current Drug Targets-Infectious Disorders. 2003; 3:263-71). MVA is distinguished by its great attenuation, as demonstrated by diminished virulence and reduced ability to replicate in primate cells, while maintaining good immunogenicity. The MVA virus has been analyzed to determine alterations in the genome relative to the parental strain chorioallantois vaccinia virus Ankara (CVA) strain. Six major deletions of genomic DNA (deletion I, II, III, IV, V, and VI) totaling 31,000 base pairs have been identified (Meyer, H. et al. 1991 J Gen Virol 72: 1031-1038). The resulting MVA virus is host cell restricted to avian cells. Accordingly, MVA vaccines can be produced in large scale in chicken cell lines.

The viral vector compositions provided herein comprise the vaccinia virus strain modified vaccinia Ankara (MVA). Modified vaccinia Ankara (MVA) has been generated by long-term serial passages of the Ankara strain of vaccinia virus (CVA) on chicken embryo fibroblasts (for review see Mayr A, et al. Abstammung, eigenschafter und verwendung des attenuierten vaccinia-stammes. Infection 3: 6-14, 1975; Swiss Patent No. 568,392). The MVA virus is publicly available from American Type Culture Collection as ATCC No. VR-1508. MVA is distinguished by its great attenuation, as demonstrated by diminished virulence and reduced ability to replicate in primate cells, while maintaining good immunogenicity. The MVA virus has been analyzed to determine alterations in the genome relative to the parental CVA strain. Six major deletions of genomic DNA (deletion I, II, III, IV, V, and VI) totaling 31,000 base pairs have been identified (Meyer, H. et al. 1991 J Gen Virol 72: 1031-1038). The resulting MVA virus is host cell replication restricted to avian cells.

In particular embodiments, the MVA for use is the MVA is the MVA available as ATCC VR-1566, a virus isolated by serial passage of CVA (Ankara) strain in chick embryo fibroblasts (CEF) in the laboratory of Professor Anton Mayr, then given to the National Institutes of Health, where it was plaque purified three times in CEF cells. VR-1566 was derived by limited further passage of stock received from the NIH in the SL-29 chicken embryo fibroblast cell line [ATCC CRL-1590].

In alternative embodiments, the MVA is derived from an MVA having the genomic sequence as described in at GenBank Accession Numbers AY603355, U94848, and DQ983238. In some embodiments, the MVA as provided herein can be derived synthetically, for example, through chemically synthesized plasmids and reconstituted to the full length genomic MVA sequence in a host cell, for example, as described in US2018/0251736, US2021/0230560, and WO2021/158565, each incorporated herein by reference.

The construction of the recombinant MVA (rMVA) viral vectors of the present invention can be prepared by methods known in the art. For example, a DNA-construct which contains the heterologous polycistronic nucleic acid sequence described herein can be flanked by MVA DNA sequences adjacent to a predetermined insertion site (e.g. between two conserved essential MVA genes such as I8R/G1L (see, e.g., U.S. Pat. No. 9,133,478, incorporated herein by reference in its entirety); in restructured and modified deletion III (see, e.g., U.S. Pat. No. 9,133,480, incorporated herein by reference in its entirety); or at other non-essential sites within the MVA genome) is introduced into cells infected with MVA, to allow homologous recombination. Once the DNA-construct has been introduced into the eukaryotic cell and the foreign DNA has recombined with the viral DNA, it is possible to isolate the desired rMVA in a manner known per se, preferably with the aid of a marker. The DNA-construct to be inserted can be linear or circular. A plasmid or polymerase chain reaction product is preferred. Such methods of making recombinant MVA vectors are described in, e.g., U.S. Pat. No. 9,133,478, incorporated by reference herein. For the expression of a DNA sequence or gene, it is necessary for regulatory sequences, which are required for the transcription of the polycistronic nucleic acid sequence, to be present on the DNA. Such regulatory sequences (called promoters) are known to those skilled in the art, and include for example those described further below. The DNA-construct can be introduced into the MVA infected cells by transfection, for example by means of calcium phosphate precipitation (Graham et al. 1973 Virol 52:456-467; Wigler et al. 1979 Cell 16:777-785), by means of electroporation (Neumann et al. 1982 EMBO J. 1:841-845), by microinjection (Graessmann et al. 1983 Meth Enzymol 101:482-492), by means of liposomes (Straubinger et al. 1983 Meth Enzymol 101:512-527), by means of spheroplasts (Schaffher 1980 PNAS USA 77:2163-2167) or by other methods known to those skilled in the art.

In some embodiments, the rMVA as provided herein can be derived synthetically, for example, through chemically synthesized plasmids and reconstituted to the full length genomic MVA sequence in a host cell, for example, as described in US2018/0251736, US2021/0230560, and WO2021/158565, each incorporated herein by reference.

As described above, the heterologous polycistronic nucleic acid sequence of the present invention can be inserted into any suitable site within the rMVA genomic sequence. In some embodiments, the polycistronic nucleic acid sequence is inserted into the MVA vector in a natural deletion site, a modified natural deletion site, or between essential or non-essential MVA genes.

### Immune Checkpoint Inhibitor Peptides

Provided herein are compositions comprising a recombinant modified vaccinia Ankara (rMVA) viral vector for use as an adjuvant or vaccine during an immunization protocol in a host such as a human, the rMVA constructed to express high concentrations of peptides capable of inhibiting one or more immune checkpoint pathways (immune checkpoint inhibitor peptide). In some embodiments, the immune checkpoint inhibitor peptides are expressed from a polycistronic nucleic acid sequence comprising tandem repeats of the immune checkpoint inhibitors capable of being processed into monomers and secreted from the cell to enhance the immunogenicity of a targeted antigen. In some embodiments, the rMVA is used as an adjuvant to increase the immunogenicity of one or more co-administered antigens during a vaccination protocol. In some embodiments, the rMVA further encodes one or more antigenic peptides and is used as an adjuvating vaccine. By expressing localized, high quantities of two or more immune checkpoint inhibitor peptides capable of downregulating one or more checkpoint inhibitor pathways, immune modulating activities which typically hinder the development of sufficient antigenicity to induce immunity can be downregulated.

In certain aspects, the immune checkpoint inhibitor peptide is capable of inhibiting the activity of an immune checkpoint pathway mediated by a receptor protein select from, but not limited to, programmed cell death protein-1 (PD-1), programmed death-ligand 1 (PD-L1), programmed death-ligand 2 (PD-L2), cytotoxic T-lymphocyte-associated protein 4 (CTLA-4), lymphocyte-activation gene 3 (LAG-3), T-cell immunoglobulin and mucin domain-3 (TIM-3), V-domain Ig suppressor of T-cell activation (VISTA), a B7 homolog protein (B7), B7 homolog 3 protein (B7-H3), B7 homolog 4 protein (B7-H4), B7 homolog 5 protein (B7-H5), OX-40 (OX-40), OX-40 ligand (OX-40L), glucocorticoid-induced TNFR-related protein (GITR), CD137, CD40, B and T lymphocyte attenuator (BTLA), Herpes Virus Entry Mediator (HVEM), galactin-9 (GAL9), killer cell immunoglobulin-like receptor (KIR), Natural Killer Cell Receptor 2B4 (2B4), CD160, checkpoint kinase 1 (CHK1), checkpoint kinase 2 (CHK2), adenosine A2a receptor (A2aR), T cell immunoreceptor with Ig and ITIM domains (TIGIT), inducible T cell co-stimulator (ICOS), inducible T cell co-stimulator ligand (ICOS-L), or combinations thereof. In some embodiments, the immune checkpoint inhibitor peptide is capable of inhibiting PD-1. In some embodiments, the immune checkpoint inhibitor peptide is capable of inhibiting PD-L1. In some embodiments, the immune checkpoint inhibitor peptide is capable of inhibiting CTLA-4. In some embodiments, the immune checkpoint inhibitor peptide is capable of inhibiting PD-1, PD-L1, or CTLA-4, or a combination thereof. In some embodiments, the immune checkpoint inhibitor peptide is capable of inhibiting both PD-1 and CTLA-4.

In some embodiments, the immune checkpoint inhibitor is an inhibitor capable of inhibiting PD-1, PD-L1, CTLA4, LAG-3, TIM3, OX40, or a combination thereof. In some embodiments, the immune checkpoint inhibitor is capable of inhibiting PD-1 and CTLA4.

In some embodiments, the immune checkpoint inhibitor peptide is selected from the peptide sequences disclosed in Table 1, or a fragment, homolog, or derivative thereof. In some embodiments, the immune checkpoint inhibitor peptide is selected from the peptide sequences of SEQ ID Nos: 1-56, or peptide having an amino acid sequence at least 85%, 90%, 95%, 97%, or 99% identical thereto. In some embodiments, the immune checkpoint inhibitor peptide is selected from the peptide sequences of SEQ ID Nos: 1-15, or peptide having an amino acid sequence at least 85%, 90%, 95%, 97%, or 99% identical thereto. In some embodiments, the immune checkpoint inhibitor peptide has the peptide sequences of SEQ ID No: 1, or peptide having an amino acid sequence at least 85%, 90%, 95%, 97%, or 99% identical thereto. In some embodiments, the immune checkpoint inhibitor peptide has the peptide sequences of SEQ ID No: 2, or peptide having an amino acid sequence at least 85%, 90%, 95%, 97%, or 99% identical thereto. In some embodiments, the immune checkpoint inhibitor peptide has the peptide sequences of SEQ ID No: 3, or peptide having an amino acid sequence at least 85%, 90%, 95%, 97%, or 99% identical thereto. In some embodiments, the immune checkpoint inhibitor peptide has the peptide sequences of SEQ ID No: 4, or peptide having an amino acid sequence at least 85%, 90%, 95%, 97%, or 99% identical thereto. In some embodiments, the immune checkpoint inhibitor peptide has the peptide sequences of SEQ ID No: 5, or peptide having an amino acid sequence at least 85%, 90%, 95%, 97%, or 99% identical thereto. In some embodiments, the immune checkpoint inhibitor peptide has the peptide sequences of SEQ ID No: 6, or peptide having an amino acid sequence at least 85%, 90%, 95%, 97%, or 99% identical thereto. In some embodiments, the immune checkpoint inhibitor peptide has the peptide sequences of SEQ ID No: 7, or peptide having an amino acid sequence at least 85%, 90%, 95%, 97%, or 99% identical thereto. In some embodiments, the immune checkpoint inhibitor peptide has the peptide sequences of SEQ ID No: 8, or peptide having an amino acid sequence at least 85%, 90%, 95%, 97%, or 99% identical thereto. In some embodiments, the immune checkpoint inhibitor peptide has the peptide sequences of SEQ ID No: 9, or peptide having an amino acid sequence at least 85%, 90%, 95%, 97%, or 99% identical thereto. In some embodiments, the immune checkpoint inhibitor peptide has the peptide sequences of SEQ ID No: 10, or peptide having an amino acid sequence at least 85%, 90%, 95%, 97%, or 99% identical thereto. In some embodiments, the immune checkpoint inhibitor peptide has the peptide sequences of SEQ ID No: 11, or peptide having an amino acid sequence at least 85%, 90%, 95%, 97%, or 99% identical thereto. In some embodiments, the immune checkpoint inhibitor peptide has the peptide sequences of SEQ ID No: 12, or peptide having an amino acid sequence at least 85%, 90%, 95%, 97%, or 99% identical thereto. In some embodiments, the immune checkpoint inhibitor peptide has the peptide sequences of SEQ ID No: 13, or peptide having an amino acid sequence at least 85%, 90%, 95%, 97%, or 99% identical thereto. In some embodiments, the immune checkpoint inhibitor peptide has the peptide sequences of SEQ ID No: 14, or peptide having an amino acid sequence at least 85%, 90%, 95%, 97%, or 99% identical thereto. In some embodiments, the immune checkpoint inhibitor peptide has the peptide sequences of SEQ ID No: 15, or peptide having an amino acid sequence at least 85%, 90%, 95%, 97%, or 99% identical thereto. In some embodiments, the immune checkpoint inhibitor peptide has the peptide sequences selected from SEQ ID NOS: 16-56, or peptide having an amino acid sequence at least 85%, 90%, 95%, 97%, or 99% identical thereto.

TABLE 1
Immune Checkpoint Inhibitor Peptides

| SEQ | | |
|---|---|---|
| ID | | |
| NO: | Peptide Identifier | Peptide Sequence |
| 1 | LD01 | CRRTSTGQISTLRVNITAPLSQ |
| 2 | LD01r | RTSTGDITSLRVITA |
| 3 | LD01 TQ19 | TSTGQISTLRVNITAPLSQ |
| 4 | LD04 | STLRVNITAPLSQRYRVRIR |
| 5 | LD10 | STGQISTLRVNITAPLSQ |
| 6 | LD10 R9A | STGQISTLAVNITAPLSQ |
| 7 | LD10 P15A | STGQISTLRVNITAALSQ |
| 8 | LD11 | CHHTSTGQISTLRVNITAPLSQ |
| 9 | LD17m | STGQISTARVNITAPLSQ |
| 10 | LD40 | QISTLRVNITA |
| 11 | QP20 | QTRTVPMPKIHHPPWQNVVP |
| 12 | HD20 | HHHQVYQVRSHWTGMHSGHD |
| 13 | WQ20 | WNLPASFHNHHIRPHEHEWIQ |
| 14 | SQ20 | SSYHHFKMPELHFGKNTFHQ |
| 15 | LD12 | CRRTSTGQISTARVNITAPLSQ |
| 16 | C1 | YSAYQCWCWRQQGTS |
| 17 | C2 | YHQYSCWCWRPPGPY |
| 18 | C3 | YASYHCWCWRDPGRS |
| 19 | C4 | TAYWNCWCWREKAGQ |
| 20 | C5 | TYGYSCWCWRQHPWT |
| 21 | C6 | STAYHCWCWREPYGN |
| 22 | C7 | TTRYSCWCWRDDAYA |
| 23 | CLP001 | HYPFRPHANQAS |
| 24 | CLP002 | WHRSYYTWNLNT |
| 25 | CLP003 | WHFSYNWRWLPP |
| 26 | CLP004 | DYHDPSLPTLRK |
| 27 | Human PD-L1 | FNWDYSWKSERLKEAYDL |
| | Inhibitor I | |
| 28 | Human PD-L1 | FNWDYSLEELREKAKYK |
| | Inhibitor II | |
| 29 | Human PD-L1 | TEKDYRHGNIRMKLAYDL |
| | Inhibitor III | |
| 30 | Human PD-L1 | GNWDYNSQRAQLYNQ |
| | Inhibitor IV | |
| 31 | Human PD-L1 | LDYVNRRKMYQ |
| | Inhibitor V | |
| 32 | PPA-1 | NYSKPTDRQYHF |
| 33 | PPA-2 | KHAHHTHNLRLP |
| 34 | PPA-3 | AAKMDGHLHGGQ |
| 35 | PPA-4 | MRNRERYPKPYY |
| 36 | PPA-5 | TYLQRPSTNLER |
| 37 | P1 | SCFPNWSLRPMNQM |
| 38 | P2 | MDEKAQKGPAKLVFFACEKG |
| 39 | TP | SCFPNWSLRPMNQM |
| 40 | AP | MDEKAQKGPAKLVFFACEKG |
| 41 | PD6478 | MLNWNRLSPSNQTEK |
| 42 | PL120131 | GADYKRITVKVN |
| 43 | C17 | CNMHTPMVC |
| 44 | C19 | CNWMINKEC |
| 45 | C25 | CVPMTYRAC |
| 46 | P29 | ADFR VMADMSMY |
| 47 | P1309 | SHTTTEHAKDKI |
| 48 | P26 | GLIPLTTHMHIGK |
| 49 | P1310 | TFLNSVPTYSYW |
| 50 | P1307 | SHDTRSPFTWGR |
| 51 | P24 | GSWNTFRAQPTI |
| 52 | P22 | SFAGVHQFRPAP |
| 53 | P1303 | ALWPSSSMSSTI |
| 54 | P1301 | HDAFPRPHLFRN |
| 55 | P20 | LTPHKHHKHLHA |
| 56 | GTX89379 | C-KWYSHSKEKIQN |

The immune checkpoint inhibitors of Table 1 have previously been described in, for example: SEQ ID NOS: 1-15 in U.S. Pat. Nos. 10,098,950, 10,799,555, and 10,799,581, and U.S. Pat. App. Nos. 2018/0071385, 2018/0185474, 2018/0200328, and 2018/0339044; SEQ ID NOS: 16-22 in Li et al., Peptide Blocking of PD-1/PD-L1 Interaction for Cancer Immunotherapy, Cancer Immunol Res Feb. 1, 2018 (6) (2) 178-188; SEQ ID NOS: 23-26 in Liu et al., Discovery of low-molecular weight anti-PD-L1 peptides for cancer immunotherapy. J. Immunotherapy Cancer 7, 270 (2019); SEQ ID NOS: 27-31 in Keir et al. D-1 and its ligands in T-cell immunity. Curr Opin Immunol. 2007; 19(3):309-14 and Li et al., Discovery of peptide inhibitors targeting human programmed death 1 (PD-1) receptor. Oncotarget. 2016; 7(40):64967-64976; SEQ ID NOS: 32-36 in Wang et al., Journal of Medicinal Chemistry 2019 62 (4), 1715-1730; SEQ ID NOS: 37-40 in Xiao et al., ACS Appl. Mater. Interfaces 2020, 12, 36, 40042-40051; SEQ ID NOS: 41-42 in Boohaker et al., Rational design and development of a peptide inhibitor for the PD-1/PD-L1 interaction, Cancer Letters, 2018, 434, Pages 11-21; SEQ ID NOS: 43-45 in Zhai et al., A novel cyclic peptide targeting LAG-3 for cancer immunotherapy by activating antigen-specific CD8+ T cell responses, Acta Pharmaceutica Sinica B, 2020, 10(6), Pages 1047-1060; 6, June 2020; SEQ ID NOS: 46-56 in Zhong et al., The biologically functional identification of a novel TIM3-binding peptide P26 in vitro and in vivo. Cancer Chemother Pharmacol. 2020; 86(6):783-792. All of the references are incorporated herein by reference.

### Secretion Signal Peptide

As provided herein, the immune checkpoint inhibitor peptides expressed by the rMVA are secreted from the cell. In some embodiments, secretion may be accomplished by including the natural secretion signal associated with the immune checkpoint inhibitor peptide, if applicable. In alternative embodiments, the immune checkpoint inhibitor peptide expressed by the rMVA may be heterologous to the host or may not have appropriate secretion signaling to ensure secretion from the host cell. Because of this, secretion of the immune checkpoint inhibitor peptide can be accomplished by expressing a chimeric polypeptide that includes a secretion signal peptide fused to the immune checkpoint inhibitor peptide.

During the translation of the chimeric polypeptide comprising the secretion signal peptide and immune checkpoint inhibitor peptide, the signal peptide is recognized as it emerges from the ribosome; it is bound by the signal recognition particle (SRP) and translation is halted. This entire complex is transported to the external face of the Endoplasmic Reticulum (ER) where it binds to the SRP receptor, and the signal sequence is transferred to a translocon. While bound to the translocon, translation is reinitiated and the protein passes through the ER membrane and into the lumen. As it does this, the signal peptide is recognized by a signal peptidase and is cleaved to generate the immune checkpoint inhibitor peptide, which is trafficked through the Golgi network before being secreted from the cell via the classical secretory pathway.

Secretion signals suitable for use in the present invention can be naturally occurring secretion signals, consensus secretion signals (see, e.g., US20100305002, incorporated herein by reference), or a synthetic secretion signal.

In some embodiments, the secretion signal is selected from a peptide sequence of Table 2, or a homolog, derivative, or fragment thereof. In some embodiments, the secretion signal has a peptide sequence selected from SEQ ID NOS: 57-90, or a or peptide having an amino acid sequence at least 85%, 90%, 95%, 97%, or 99% identical thereto.

In some embodiments, the secretion signal is derived from the human tissue plasminogen activator (tPA) secretion signal or a homolog, derivative, or fragment thereof. In some embodiments, the secretion signal peptide has the peptide sequence of SEQ ID NO: 65, or a peptide having an amino acid sequence at least 85%, 90%, 95%, 97%, or 99% identical thereto. In some embodiments, the secretion signal peptide has the peptide sequence of SEQ ID NO: 66, or a peptide having an amino acid sequence at least 85%, 90%, 95%, 97%, or 99% identical thereto. It has been found that the tPA secretion signal is a particularly suitable secretion signal for use in the present invention, as it further enhances expression of the immune checkpoint inhibitor peptides.

TABLE 2
Secretion Signal Peptides

| SEQ ID NO: | Secretion Signal | Peptide Sequence |
|---|---|---|
| 57 | Human OSM | GVLLTQRTLLSLVLALLFPSMASM |
| 58 | VSV-G | KCLLYLAFLFIGVNC |
| 59 | Mouse Ig Kappa | ETDTLLLWVLLLWVPGSTGD |
| 60 | Human IgG2 H | GWSCIILFLVATATGVHS |
| 61 | BM40 | RAWIFFLLCLAGRALA |
| 62 | Secrecon | WWRLWWLLLLLLLLWPMVWA |
| 63 | Human IgKVIII | DMRVPAQLLGLLLLWLRGARC |
| 64 | CD33 | PLLLLLPLLWAGALA |
| 65 | tissue plasminogen activator | DAMKRGLCCVLLLCGAVFVSPS |
| | (tPA) | |
| 66 | tissue plasminogen activator | DAMKRGLCCVLLLCGAVFVSPSQEIH |
| | (tPA) | ARFRRGAR |
| 67 | Human Chymotrypsinogen | AFLWLLSCWALLGTTFG |
| 68 | Human trypsinogen-2 | NLLLILTFVAAAVA |
| 69 | Human IL-2 | YRMQLLSCIALSLALVTNS |
| 70 | Gaussia luc | GVKVLFALICIAVAEA |
| 71 | Albumin (HSA) | KWVTFISLLFSSAYS |
| 72 | Influenza Haemagglutinin | KTIIALSYIFCLVLG |
| 73 | Human insulin | ALWMRLLPLLALLALWGPDPAAA |
| 74 | Silkworm Fibroin LC | KPIFLVLL |
| 75 | Alkaline phosphatase | LGPCMLLLLLLLGLRLQLSLG |
| 76 | Secron 2 | RPTWAWWLFLVLLLALWAPARG |
| 77 | Human cystatin s | AGPLRAPLLLLAILAVALAVSPAAGSS |
| 78 | Lactotransferrin | HKLVFLVLLFLGALGLCLA |
| 79 | Erythropoietin | GVHECPAWLWLLLSLLSLPLGLPVLG |
| 80 | Human a-1- | ERMLPLLALGLLAAGFCPAVLC |
| | antichymotrypsin | |
| 81 | TNF receptor superfamily- | HLGIWTLLPLVLTSVA |
| | member 6 isoform 4 | |
| 82 | Human prolactin | NIKGSPWKGSLLLLLVSNLLLCQSVAP |
| 83 | Osteopontin | RLAVVCLCLFGLASC |
| 84 | ATC | GRMLPLLALLLLAAGFCPAVLA |
| 85 | Consensus | RSLSVLALLLLLLLAPASAA |
| 86 | Consensus | KSLSALVLLLLLLLLPGALAA |
| 87 | Consensus | RGAALVLLLLLLLLLALALAAPVP |
| 88 | Consensus | RGAALVLLLLLLLLLAGVLAAP |
| 89 | Consensus | RGAALVLLLLLLLLLSPALA |
| 90 | Consensus | RSLSVLALLLLLLLAPASAA |

In some embodiments, the Secretion Signal Peptide of the first polypeptide encoded by the polycistronic nucleic acid insert further comprises the initiation amino acid methionine (M).

### Cleavable Sequences

In addition to the secretion signal peptide on the N-terminus of each immune checkpoint inhibitor peptide, the polypeptide may also include a self-cleaving peptide fused to the C-terminus of the immune checkpoint inhibitor peptide. By providing a self-cleaving peptide sequence fused to the C-terminus of the immune checkpoint inhibitor peptide, the multiple immune checkpoint inhibitor peptides can be cleaved into multiple monomers during or following translation. Suitable cleavage sequences are known in the art (see, e.g., Donnelly et al., Analysis of the aphthovirus 2A/2B polyprotein ‘cleavage’ mechanism indicates not a proteolytic reaction, but a novel translational effect: a putative ribosomal ‘skip’. J. Gen. Virol. 82, 1013-1025 (2001), incorporated by reference in its entirety herein).

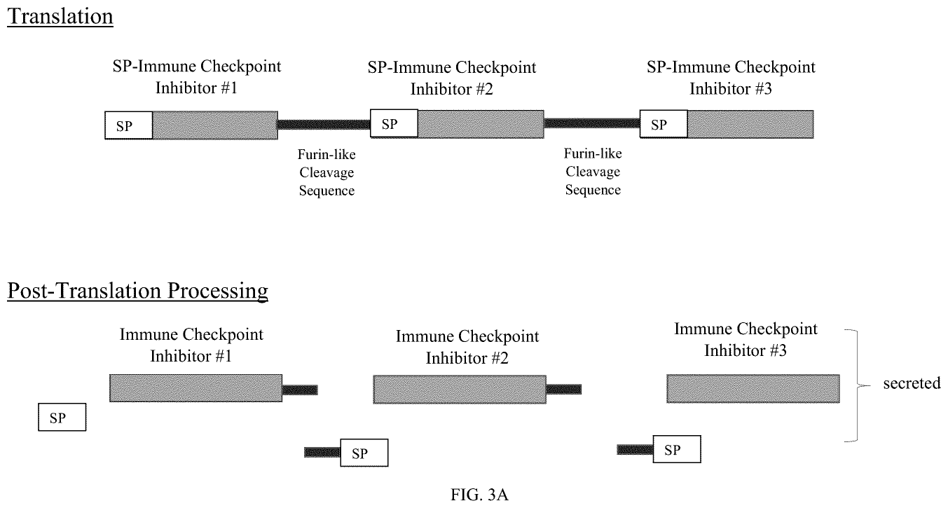

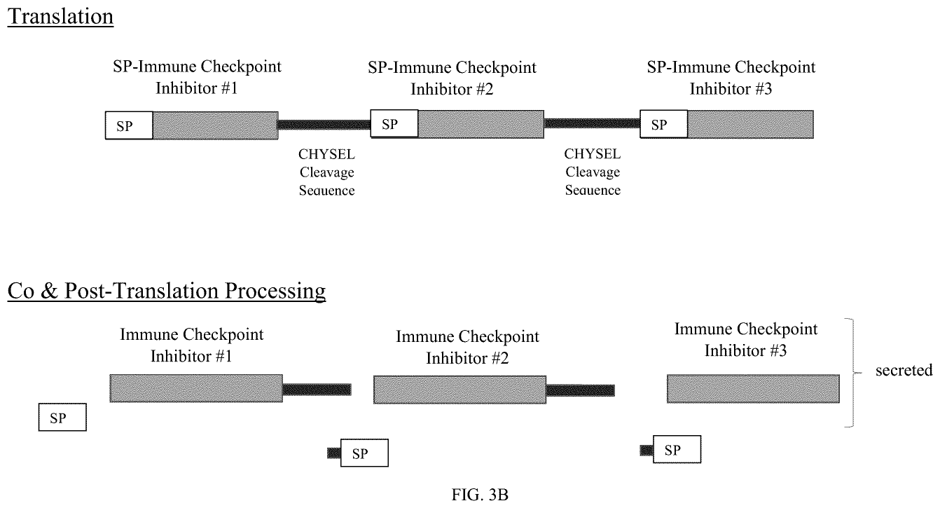

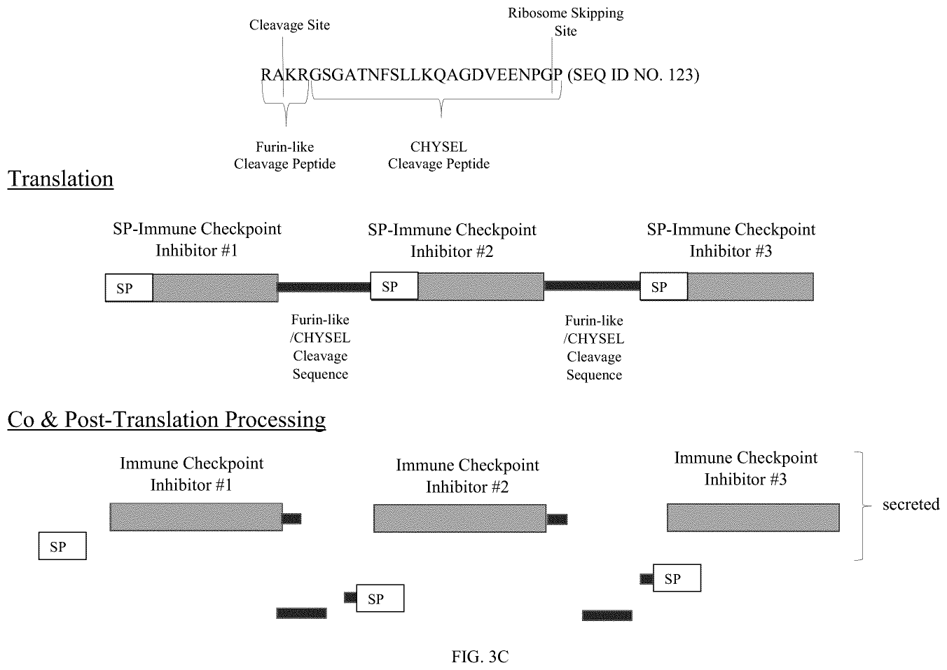

In some embodiments, one or more of the immune checkpoint inhibitor chimeric polypeptides includes one or more peptide sequences fused to the C-terminus of the immune checkpoint inhibitor peptide which is capable of being cleaved during or following, or a combination thereof, the translation of the polycistronic nucleic acid (see, e.g., FIGS. 3A, 3B, and 3C). In some embodiments, the most C-terminus immune checkpoint inhibitor chimeric polypeptide does not include a cleavable peptide.

In some embodiments, the cleavable peptide is capable of being cleaved by a proprotein convertase enzyme including, for example, but not limited to furin or a furin-like proprotein convertase (Table 3). In some embodiments, the cleavable peptide sequence comprises a basic amino acid target sequence (canonically, RX(R/K)R), wherein X=any amino acid (SEQ ID NO: 91). In some embodiments, the cleavable peptide sequence comprises a basic amino acid target sequence (canonically, RX(R/K)R), wherein X=R, K, or H (SEQ ID NO: 92). In some embodiments, the cleavable peptide sequence is RAKR (SEQ ID NO: 93). In some embodiments, the cleavable peptide sequence is RRRR (SEQ ID NO: 94). In some embodiments, the cleavable peptide is RKRR (SEQ ID NO: 95). In some embodiments, the cleavable peptide is RRKR (SEQ ID NO: 96). In some embodiments, the cleavable peptide is RKKR (SEQ ID NO: 97). By including a cleavable peptide sequence on each of the covalently linked chimeric polypeptides, the multimeric polypeptide expressed during translation of the polycistronic nucleic acid insert can be processed through a cleaving mechanism into monomeric chimeric polypeptides following translation. This allows each chimeric polypeptide comprising the immune checkpoint inhibitor peptide to be secreted from the cell and function to downregulate an undesirable immune checkpoint pathway (see, e.g., FIG. 3A).

TABLE 3
Cleavable Peptide Sequences

| SEQ ID NO: | Cleavable Peptide Sequence |
|---|---|
| 91 | RX(R/K)R |
| 92 | RX(R/K)R, X = R, K, or H |
| 93 | RAKR |
| 94 | RRRR |
| 95 | RKRR |
| 96 | RRKR |
| 97 | RKKR |

In some embodiments, each chimeric polypeptide includes one or more peptide sequences fused to the C-terminus of the immune checkpoint inhibitor peptide which is capable of inducing ribozyme skipping during translation of the polycistronic nucleic acid. Ribosomal “skipping” is an alternate mechanism of translation in which a specific peptide sequence prevents the ribosome from covalently linking a new inserted amino acid, but nonetheless continues translation. This results in a “cleavage” of the polyprotein through the induced ribosomal skipping (see, e.g., FIG. 3B).

In some embodiments, the peptide capable of inducing ribosomal skipping is a cis-acting hydrolase element peptide (CHYSEL). In some embodiments, the CHYSEL sequence comprises a non-conserved sequence of amino-acids with a strong alpha-helical propensity followed by the consensus sequence D(V/I)EXNPGP, where X=any amino acid (SEQ ID NO: 98), wherein the ribosomal skipping cleavage occurs between the G and P sequence. In some embodiments, the CHYSEL sequence comprises DVEENPGP (SEQ ID NO: 99).

In some embodiments, the CHYSEL cleavage sequence is derived from one or more 2A self-processing peptides. 2A sequences are oligopeptides located between the P1 and P2 proteins in some members of the viral families, for example the picornavirus family, and can undergo self-cleavage to generate the mature viral proteins P1 and P2 in eukaryotic cells (Ahier et al., Simultaneous expression of multiple proteins under a single promoter in Caenorhabditis elegans via a versatile 2A-based toolkit. Genetics. 2014; 196:605-613; Luke et al., Occurrence, function and evolutionary origins of ‘2A-like’ sequences in virus genomes. J Gen Virol. 2008 April; 89(Pt 4):1036-42; Doronina et al., Dissection of a co-translational nascent chain separation event. Biochem Soc Trans. 2008 August; 36(Pt 4):712-6; Martin et al., A Model for Nonstoichiometric, Cotranslational Protein Scission in Eukaryotic Ribosomes. Bioorganic Chemistry, Volume 27, Issue 1, February 1999, 55-79). The first discovered 2A was F2A (foot-and-mouth disease virus), after which E2A (equine rhinitis A virus), P2A (porcine teschovirus-1 2A), and T2A (thosea asigna virus 2A) were also identified (Ryan et al., Cleavage of foot-and-mouth disease virus polyprotein is mediated by residues located within a 19 amino acid sequence. The Journal of general virology. 1991; 72(Pt 11):2727-2732; Szymczak et al., Development of 2A peptide-based strategies in the design of multicistronic vectors. Expert opinion on biological therapy. 2005; 5:627-638).

In some embodiments, the CHYSEL cleavage sequence is derived from one or more 2A self-processing peptides provided for in Table 4, or peptide having an amino acid sequence at least 85%, 90%, 95%, 97%, or 99% identical thereto. In some embodiments, the CHYSEL cleavage sequence is derived from one or more 2A self-processing peptides having an amino acid sequence selected from SEQ ID NOS: 100-117, or peptide having an amino acid sequence at least 85%, 90%, 95%, 97%, or 99% identical thereto.

TABLE 4
CHYSEL Sequences

| SEQ ID NO: | Origin | Peptide Sequence |
|---|---|---|
| 98 | | D(V/I)EXNPGP |
| 99 | | DVEENPGP |
| 100 | Picornaviridae: | PSDARHKQRIVAPAKQLLNFDLLKLAGDVESNP |
| | Aphtovirus: Foot-and-mouth disease | GP |
| | virus | |
| 101 | Avisivirus: Avisivirua A | ARRTLEWARREVGAIDETDHKDILLGGDIEENP |
| | | GP |
| 102 | Avihepatovirus: Duck | RLKTLAFELNLEIESDQIRNKKDLTTEGVEPNPG |
| | hepatitis A virus | P |
| 103 | Cardiovirus: | VREENVFGLYRIFNAHYAGYFADLLIHDIETNPG |
| | Encephalomyocarditis: virus | P |
| 104 | Cosavirus: Cosavirus A | IMADSVLPRPLTRAERDVARDLLLIAGDIESNPG |
| | | P |
| 105 | Erbovirus: Equine | SEPIPEATLSTILSEGATNFSLLKLAGDVELNPGP |
| | rhinitis B virus | |
| 106 | Erbovirus: Seneca | RYKNARAWCPSMLPFRSYKQKMLMQSGDIETN |
| | Valley virus | PGP |
| 107 | Hunnivirus: | CPRPGMAIDPPAQSSGATNFSLLRLAGDVELNP |
| | Hunnivirus A | GP |
| 108 | Kunsagivirus: | SPRSLLHFLIGRPRPRVPPSPSLLLSGDVEPNPG\|P |
| | Kunsagivirus A | |
| 109 | Mischivirus: | DSYPASGEEEEDDFHDMEDHSDILLGGDVEENP |
| | Mischivirus A | GP |
| 110 | Mosavirus: Mosavirus A2 | TNSRAKLMVDEDYVIQRSAHRSVLLDGDVESN |
| | | PGP |
| 111 | Pasivirus: Pasivirus | DIPSFQRDFINWLGSKEELQNMILQCGDVEQNP |
| | A1 | GP |
| 112 | Teschovirus: Porcine | EGLSSAMTVMAFQGPGATNFSLLKQAGDVEEN |
| | teschovirus 1 | PGP |
| 113 | Iflaviridae: Iflavirus: | NYPLVPSIGNVARTLTRAEIEDELIRAGIESNPGP |
| | Infectious flacherie | |
| | virus | |
| 114 | Tetraviridae: Betatetra virus: | RSRRLRGPRPQNLGVRAEGRGSLLTCGDVEENP |
| | Thosea asigna | GP |
| | virus | |
| 115 | Dicistroviridae: | FQQWKLVSSNDECRAFLRKRTQLLMSGDVESN |
| | Cripavirus: Cricket | PGP |
| | paralysis virus | |
| 116 | Reoviridae: Rotavirus: | LKKHNGAGYPLIVANSKFQIDKILISGDIELNPGP |
| | Human rotavirus C | |
| 117 | Cypovirus: Lymantria: | TDFLSMTAFDFQQAVFRSNYDLLKLCGDVESNP |
| | Dispar cypovirus 1 | GP |

In some embodiments, the cleavage sequence is a 2A cleavage sequence derived from foot-and-mouth disease virus (FMDV), for example derived from the amino acid sequence comprising VKQTLNFDLLKLAGDVESNPGP (SEQ ID. No. 118), or peptide having an amino acid sequence at least 85%, 90%, 95%, 97%, or 99% identical thereto. In some embodiments, the 2A cleavage sequence is a 2A or 2A-like cleavage sequence selected from GSGEGRGSLLTCGDVEENPGP (SEQ ID NO: 119), GSGATNFSLLKQAGDVEENPGP (SEQ ID NO: 120), GSGQCTNYALLKLAGDVESNPGP (SEQ ID NO: 121), or GSGVKQTLNFDLLKLAGDVESNPGP (SEQ ID NO: 122), or peptide having an amino acid sequence at least 85%, 90%, 95%, 97%, or 99% identical thereto. In particular embodiments, the 2A-like cleavage sequence is GSGATNFSLLKQAGDVEENPGP (SEQ ID NO: 120), or peptide having an amino acid sequence at least 85%, 90%, 95%, 97%, or 99% identical thereto.

TABLE 5
2A/2A-like Cleavage Sequences

| SEQ ID NO: | Peptide Sequence |
|---|---|
| 118 | VKQTLNFDLLKLAGDVESNPGP |
| 119 | GSGEGRGSLLTCGDVEENPGP |
| 120 | GSGATNFSLLKQAGDVEENPGP |
| 121 | GSGQCTNYALLKLAGDVESNPGP |
| 122 | GSGVKQTLNFDLLKLAGDVESNPGP |

In some embodiments, the cleavable peptide sequence comprises two or more sequences which are capable of being cleaved by different mechanism, for example a cleavable peptide sequence which is capable of being cleaved following the translation of the polycistronic nucleic acid and a peptide sequence capable of inducing ribozyme skipping during translation of the polycistronic nucleic acid. By providing cleavable peptide sequences subject to multiple modes of cleaving, the efficiency of monomeric formation from the polycistronic nucleic acid can be improved. In some embodiments, the immune checkpoint inhibitor peptide has fused to its C-terminus a furin-cleavable peptide sequence, for example the peptide sequence RX(R/K)R, wherein X=any amino acid (SEQ ID NO: 91), and fused to the C-terminus of the furin-cleavable peptide sequence is a CHYSEL peptide sequence, for example a peptide comprising the amino acid sequence D(V/I)EXNPGP, where X=any amino acid (SEQ ID NO: 98). By including a furin-cleavable peptide sequence, such as RAKR (SEQ ID NO: 93), fused to the N-terminus of a CHYSEL peptide sequence between each chimeric polypeptide, the transcribed polycistronic nucleic acid undergoes ribozyme skipping during translation, resulting in the production of monomeric chimeric polypeptides, and all but the arginine (R) and alanine (A) residues of the furin cleavage sequence remains at the C-terminus of immune checkpoint inhibitor peptide, limiting the potential interference of the extra amino acid sequences on the function of the immune checkpoint inhibitor peptide (see e.g., FIG. 3C). In alternative embodiments, including a furin-cleavable peptide sequence, such as RRRR (SEQ ID NO: 94), RKRR (SEQ ID NO: 95), or RRKR (SEQ ID NO: 96), fused to the N-terminus of a CHYSEL peptide sequence between each chimeric polypeptide, the transcribed polycistronic nucleic acid undergoes ribozyme skipping during translation, resulting in the production of monomeric chimeric polypeptides, and the remaining furin cleavage sequence and CHYSEL peptide sequence are removed at the C-terminus of immune checkpoint inhibitor peptide.

In some embodiments, the hybrid cleavable peptide sequence comprises RAKR (SEQ ID NO: 93) fused to a CHYSEL containing amino acid sequence D(V/I)EXNPGP, where X=any amino acid (SEQ ID NO: 98). In some embodiments, the hybrid cleavable peptide sequence comprises RAKR (SEQ ID NO: 93) fused to a CHYSEL amino acid sequence selected from the group consisting of SEQ ID NOS: 100-122, or peptide having an amino acid sequence at least 85%, 90%, 95%, 97%, or 99% identical thereto. In some embodiments, the hybrid cleavable peptide sequence comprises RAKR (SEQ ID NO: 93) fused to a CHYSEL amino acid sequence selected from the group consisting of SEQ ID NOS: 118-122, or peptide having an amino acid sequence at least 85%, 90%, 95%, 97%, or 99% identical thereto. In some embodiments, the hybrid cleavable peptide sequence comprises RAKR (SEQ ID NO: 93) fused to a CHYSEL amino acid sequence of amino acid SEQ ID NO: 120, or peptide having an amino acid sequence at least 85%, 90%, 95%, 97%, or 99% identical thereto. In particular embodiments, the hybrid cleavable peptide is RAKRGSGATNFSLLKQAGDVEENPGP (SEQ ID NO: 123).

In some embodiments, the hybrid cleavable peptide sequence comprises RRRR (SEQ ID NO: 94) fused to a CHYSEL containing amino acid sequence D(V/I)EXNPGP, where X=any amino acid (SEQ ID NO: 98). In some embodiments, the hybrid cleavable peptide sequence comprises RRRR (SEQ ID NO: 94) fused to a CHYSEL amino acid sequence selected from the group consisting of SEQ ID NOS: 100-122, or peptide having an amino acid sequence at least 85%, 90%, 95%, 97%, or 99% identical thereto. In some embodiments, the hybrid cleavable peptide sequence comprises RRRR (SEQ ID NO: 93) fused to a CHYSEL amino acid sequence selected from the group consisting of SEQ ID NOS: 118-122, or peptide having an amino acid sequence at least 85%, 90%, 95%, 97%, or 99% identical thereto. In some embodiments, the hybrid cleavable peptide sequence comprises RRRR (SEQ ID NO: 94) fused to a CHYSEL amino acid sequence of amino acid SEQ ID NO: 120, or peptide having an amino acid sequence at least 85%, 90%, 95%, 97%, or 99% identical thereto. In particular embodiments, the hybrid cleavable peptide is RRRRGSGATNFSLLKQAGDVEENPGP (SEQ ID NO: 124).

In some embodiments, the hybrid cleavable peptide sequence comprises RKRR (SEQ ID NO: 95) fused to a CHYSEL containing amino acid sequence D(V/I)EXNPGP, where X=any amino acid (SEQ ID NO: 98). In some embodiments, the hybrid cleavable peptide sequence comprises RKRR (SEQ ID NO: 95) fused to a CHYSEL amino acid sequence selected from the group consisting of SEQ ID NOS: 100-122, or peptide having an amino acid sequence at least 85%, 90%, 95%, 97%, or 99% identical thereto. In some embodiments, the hybrid cleavable peptide sequence comprises RKRR (SEQ ID NO: 95) fused to a CHYSEL amino acid sequence selected from the group consisting of SEQ ID NOS: 118-122, or peptide having an amino acid sequence at least 85%, 90%, 95%, 97%, or 99% identical thereto. In some embodiments, the hybrid cleavable peptide sequence comprises RKRR (SEQ ID NO: 95) fused to a CHYSEL amino acid sequence of amino acid SEQ ID NO: 120, or peptide having an amino acid sequence at least 85%, 90%, 95%, 97%, or 99% identical thereto. In particular embodiments, the hybrid cleavable peptide is RKRRGSGATNFSLLKQAGDVEENPGP (SEQ ID NO: 125).

In some embodiments, the hybrid cleavable peptide sequence comprises RRKR (SEQ ID NO: 96) fused to a CHYSEL containing amino acid sequence D(V/I)EXNPGP, where X=any amino acid (SEQ ID NO: 98) (Table 6). In some embodiments, the hybrid cleavable peptide sequence comprises RRKR (SEQ ID NO: 96) fused to a CHYSEL amino acid sequence selected from the group consisting of SEQ ID NOS: 100-123, or peptide having an amino acid sequence at least 85%, 90%, 95%, 97%, or 99% identical thereto. In some embodiments, the hybrid cleavable peptide sequence comprises RRKR (SEQ ID NO: 96) fused to a CHYSEL amino acid sequence selected from the group consisting of SEQ ID NOS: 118-122, or peptide having an amino acid sequence at least 85%, 90%, 95%, 97%, or 99% identical thereto. In some embodiments, the hybrid cleavable peptide sequence comprises RRKR (SEQ ID NO: 96) fused to a CHYSEL amino acid sequence of amino acid SEQ ID NO: 120, or peptide having an amino acid sequence at least 85%, 90%, 95%, 97%, or 99% identical thereto. In particular embodiments, the hybrid cleavable peptide is RRKRGSGATNFSLLKQAGDVEENPGP (SEQ ID NO: 126).

In some embodiments, the hybrid cleavable peptide sequence comprises RKKR (SEQ ID NO: 97) fused to a CHYSEL containing amino acid sequence D(V/I)EXNPGP, where X=any amino acid (SEQ ID NO: 98). In some embodiments, the hybrid cleavable peptide sequence comprises RKKR (SEQ ID NO: 97) fused to a CHYSEL amino acid sequence selected from the group consisting of SEQ ID NOS: 100-123, or peptide having an amino acid sequence at least 85%, 90%, 95%, 97%, or 99% identical thereto. In some embodiments, the hybrid cleavable peptide sequence comprises RKKR (SEQ ID NO: 97) fused to a CHYSEL amino acid sequence selected from the group consisting of SEQ ID NOS: 118-122, or peptide having an amino acid sequence at least 85%, 90%, 95%, 97%, or 99% identical thereto. In some embodiments, the hybrid cleavable peptide sequence comprises RKKR (SEQ ID NO: 97) fused to a CHYSEL amino acid sequence of amino acid SEQ ID NO: 120, or peptide having an amino acid sequence at least 85%, 90%, 95%, 97%, or 99% identical thereto. In particular embodiments, the hybrid cleavable peptide is RKKRGSGATNFSLLKQAGDVEENPGP (SEQ ID NO: 127).

TABLE 6
Hybrid Cleavable Peptide Sequences

| SEQ ID NO: | Peptide Sequence |
|---|---|
| 123 | RAKRGSGATNFSLLKQAGDVEENPGP |
| 124 | RRRRGSGATNFSLLKQAGDVEENPGP |
| 125 | RKRRGSGATNFSLLKQAGDVEENPGP |
| 126 | RRKRGSGATNFSLLKQAGDVEENPGP |
| 127 | RKKRGSGATNFSLLKQAGDVEENPGP |

### Regulatory Sequences

As provided herein, the immune checkpoint inhibitor peptides are expressed from a nucleic acid sequence inserted into a suitable location within the MVA genomic sequence. For the expression of the nucleic acid insert within the rMVA genomic backbone, it is necessary for regulatory sequences such as promoters, which are required for the transcription of the polycistronic nucleic acid encoding the polyprotein, to be located in the 5′ region of the nucleic acid insert adjacent to the transcription start site in order to initiate transcription. Wherein the nucleic acid insert is a polycistronic nucleic acid encoding multiple proteins/peptides as a single polyprotein, one or more promoters can be located 5′ to the transcriptional start site of the ORF encoding the N-terminus most polypeptide of the polyprotein.

Because MVA is a cytoplasmic virus, suitable promoters, in some embodiments, include those derived from naturally occurring poxviral promoters. Poxviral genes, promoters, and transcription factors are divided into early, intermediate, and late classes, depending on their expression timing during poxvirus infections (see, e.g., Assarsson et al., Kinetic analysis of a complete poxvirus transcriptome reveals an immediate-early class of genes. PNAS 2008; 105(6):2140-2145; Yang Z et al., Genome-wide analysis of the 5′ and 3′ ends of vaccinia virus early mRNAs delineates regulatory sequences of annotated and anomalous transcripts. J Virol. 2011; 85(12):5897-5909). MVA replication in most mammalian cells (non-permissive cells) ceases during the assembly of progeny virions after all stages of expression occur. This supports the utility of all promoter classes, including late promoters, for controlling transgene expression (Sancho et al., The block in assembly of modified vaccinia virus Ankara in HeLa cells reveals new insights into vaccinia virus morphogenesis. J Virol. 2002; 76(16):8318-8334; Geiben-Lynn et al., Kinetics of recombinant adenovirus type 5, vaccinia virus, modified vaccinia ankara virus, and DNA antigen expression in vivo and the induction of memory T-lymphocyte responses. Clin Vaccine Immunol. 2008; 15(4):691-696). Some poxviral promoters have both early and late elements, allowing their open-reading frames (ORFs) or recombinant antigens to be expressed early in the virus infection and late after the viral genome replication, respectively (Broyles S S, Vaccinia virus transcription. J Gen Virol. 2003; 84(Pt 9):2293-2303). Poxviral promoters can be utilized cross-strain (see Prideaux et al., Comparative analysis of vaccinia virus promoter activity in fowlpox and vaccinia virus recombinants. Virus Res. 1990; 16(1):43-57; Tripathy et al., Regulation of foreign gene in fowlpox virus by a vaccinia virus promoter. Avian Dis. 1990; 34(1):218-220).

Such MVA promoter sequences are known to those skilled in the art, and include for example the p11 promoter, which drives expression of the ilk protein encoded by the F17R ORF (Wittek et al., Mapping of a gene coding for a major late structural polypeptide on the vaccinia virus genome. J Virol. 1984; 49(2):371-378); the p7.5 promoter (Cochran et al., In vitro mutagenesis of the promoter region for a vaccinia virus gene: evidence for tandem early and late regulatory signals. J Virol. 1985; 54(1):30-37); the pI1L promoter (Schmitt et al., Sequence and transcriptional analysis of the vaccinia virus HindIII I fragment. J Virol. 1988; 62(6):1889-1897); the pTK promoter (Weir and Moss, Determination of the promoter region of an early vaccinia virus gene encoding thymidine kinase. Virology. 1987; 158(1):206-210); the pF7L promoter (Coupar et al., Effect of in vitro mutations in a vaccinia virus early promoter region monitored by herpes simplex virus thymidine kinase expression in recombinant vaccinia virus. J Gen Virol. 1987; 68(Pt 9):2299-2309); the pH5 promoter (Perkus et al., Cloning and expression of foreign genes in vaccinia virus, using a host range selection system. J Virol. 1989; 63(9):3829-3836); the short synthetic promoter pSyn (Chakrabarti et al., Compact, synthetic, vaccinia virus early/late promoter for protein expression. Biotechniques. 1997; 23(6):1094-1097; Hammond et al., A synthetic vaccinia virus promoter with enhanced early and late activity. J Virol Methods. 1997; 66(1):135-1380); the pmH5 promoter (Wyatt et al., Development of a replication-deficient recombinant vaccinia virus vaccine effective against parainfluenza virus 3 infection in an animal model. Vaccine. 1996; 14(15):1451-1458); the pHyb promoter (Sancho et al., The block in assembly of modified vaccinia virus Ankara in HeLa cells reveals new insights into vaccinia virus morphogenesis. J Virol. 2002; 76(16):8318-8334); the LEO promoter (Wyatt et al., Correlation of immunogenicities and in vitro expression levels of recombinant modified vaccinia virus Ankara HIV vaccines. Vaccine. 2008; 26(4):486-493); the pB8 promoter (Orubu et al., Expression and cellular immunogenicity of a transgenic antigen driven by endogenous poxviral early promoters at their authentic loci in MVA. PLoS One. 2012; 7(6):e40167); the pFl1 promoter (Orubu et al., Expression and cellular immunogenicity of a transgenic antigen driven by endogenous poxviral early promoters at their authentic loci in MVA. PLoS One. 2012; 7(6):e40167). In some embodiments, the promoter is selected from one or more of pMH5, p11, pSyn, pHyb, or a combination thereof.

In some embodiments, the promoter is the pH5 promoter AAAAAATGAAAATAAATACAAAGGTTCTTGAGGGTTGTGTTAAATTGAAAGCGAGA AATAATCATAA (SEQ ID NO: 128), or a nucleic acid sequence at least 85%, 90%, 95%, 97%, or 99% identical thereto. In some embodiments, the promoter is the pH5 promoter AAAAAATGAAAATAAATACAAAGGTTCTTGAGGGTTGTGTTAAATTGAAAGCGAGA AATAATCATAAATT (SEQ ID NO: 129), or a nucleic acid sequence at least 85%, 90%, 95%, 97%, or 99% identical thereto.

In some embodiments, the promoter is the modified pH5 promoter (pmH5) AAAAATTGAAAATAAATACAAAGGTTCTTGAGGGTTGTGTTAAATTGAAAGCGAGA AATAATCATAA (SEQ ID NO: 130), or a nucleic acid sequence at least 85%, 90%, 95%, 97%, or 99% identical thereto. In some embodiments, the promoter is the modified pH5 promoter (pmH5) AAAAATTGAAAATAAATACAAAGGTTCTTGAGGGTTGTGTTAAATTGAAAG CGAGAAATAATCATAAATA (SEQ ID NO: 131), or a nucleic acid sequence at least 85%, 90%, 95%, 97%, or 99% identical thereto. In some embodiments, the promoter is the modified pH5 promoter (pmH5) AAAAAATGAAAATAAATACAAAGGTTCTTGAGGGTTGTGTTA AATTGAAAGCGAGAAATAATCATAAATA (SEQ ID NO: 132), or a nucleic acid sequence at least 85%, 90%, 95%, 97%, or 99% identical thereto.

Additional vaccinia virus promoters that may be particularly suitable as promoters in the present invention include those derived from natural promoter sequences, for example, as provided in Table 7 below, or a nucleic acid sequence at least 85%, 90%, 95%, 97%, or 99% identical thereto, wherein the nomenclature for the gene locus is based on the ORF nomenclatures originally used for the WR and Copenhagen strains of vaccinia virus. In some embodiments, the promoter is selected from one or more of SEQ ID. No. 133-308, or a combination thereof, or a nucleic acid sequence at least 85%, 90%, 95%, 97%, or 99% identical thereto.

TABLE 7
Additional Vaccinia Virus Promoters

| SEQ ID. | | |
|---|---|---|
| No. | Gene Locus | Promoter Sequence |
| 128 | pH5 promoter | AAAAAATGAAAATAAATACAAAGGTTCTTGAGGGTTGTGTTA |
| | | AATTGAAAGCGAGAAATAATCATAA |
| 129 | pH5 promoter | AAAAAATGAAAATAAATACAAAGGTTCTTGAGGGTTGTGTTA |
| | | AATTGAAAGCGAGAAATAATCATAAATT |
| 130 | modified pH5 | AAAAATTGAAAATAAATACAAAGGTTCTTGAGGGTTGTGTTA |
| | promoter (pmH5) | AATTGAAAGCGAGAAATAATCATAA |
| 131 | modified pH5 | AAAAATTGAAAATAAATACAAAGGTTCTTGAGGGTTGTGTTA |
| | promoter (pmH5) | AATTGAAAGCGAGAAATAATCATAAATA |
| 132 | modified pH5 | AAAAAATGAAAATAAATACAAAGGTTCTTGAGGGTTGTGTTA |
| | promoter (pmH5) | AATTGAAAGCGAGAAATAATCATAAATA |
| 133 | C23L | AAAGTAGAAAATATA |
| 134 | Pseudogene | TATCCGGAGACGTCA |
| 135 | C11R | ATTACTGAATTAATA |
| 136 | C10L | GCAACGTAAAACACA |
| 137 | no ortholog | AAAAAATAAAAAAAA |
| 138 | no ortholog | AGTAAAGAAAAAGAA |
| 139 | no ortholog | AAAATTGATAAATAA |
| 140 | no ortholog | AAATTAGACATTTGA |
| 141 | C9L | ATAACTGAAATGAAA |
| 142 | C7L | AAAGATGAAAAAGTA |
| 143 | C6L | ATTAATGAAATAATA |
| 144 | C5L | AAAAATGAAAATGGA |
| 145 | C4L | AAAACATAAAAATTA |
| 146 | N2L | ATAACATAAAAATAA |
| 147 | M2L | AAGATAGATTTCCTA |
| 148 | K1L | AAAAATGAAAAAATA |
| 149 | K3L | GAAAAAGAAATTCCT |
| 150 | K5L | AATGGTGAAAAAATG |
| 151 | K6L | AAAACATAAAAATAA |
| 152 | K7R | ATAATTGTAAAAACA |
| 153 | F7L | ATAATTGAAAATGGA |
| 154 | F8L | AAAAATTTAATTACA |
| 155 | F11L | AAAAGTGAAAAACAA |
| 156 | F12L | AAAAAAGAAAATAGA |
| 157 | F14L | GTAGAAGAAAATAAT |
| 158 | F15L | AAAAATGAAACGTAA |
| 159 | F16L | AAAAAACAAAATGAA |
| 160 | E1L | GAGACAGTAGTTTTA |
| 161 | E3L | AAAAATGATAAAATA |
| 162 | E4L | AATAATGAAAAAATA |
| 163 | E5R | ACAAAAGTGAATATA |
| 164 | E9L | TTAAATGAAAATATA |
| 165 | O1L | AATAATGAAAAAACA |
| 166 | I3L | TAAAGTGAAAATATA |
| 167 | I4L | ATTAATGAAAAGTTA |
| 168 | G2R | ATAACAAAAATAAAA |
| 169 | G5R | AAAAATGATAAGATA |
| 170 | G5.5R | AAAACTGTAACACGA |
| 171 | L2R | AAAACTGAAAATATA |
| 172 | J2R | TAAAGTGAACAATAA |
| 173 | J6R | AAAAGGGAAATTTGA |
| 174 | H3L | AGAATTGAAAACGAA |
| 175 | H5R | AAAAATGAAAATAAA |
| 176 | D1R | GTAAATGAAAAAAAA |
| 177 | D4R | GAAAATGAAAAGGTA |
| 178 | D7R | AAAACTGATGAAATA |
| 179 | D9R | AAAAATGAAATGATA |
| 180 | D12L | AATAATGAAAACAAA |
| 181 | A4L | AATTCTGAAACTAGA |
| 182 | A5R | AAAATTGAATTGCGA |
| 183 | A8R | TAAAGTGAAAATCTA |
| 184 | A18R | GCAATAGAAAAGATG |
| 185 | A20R | AAGAATGAAATAACA |
| 186 | A23R | AAAAATGTAATAACG |
| 187 | A29L | AAAGTCGAAAAAGAA |
| 188 | A31R | AAAACATAAATATAA |
| 189 | A33R | AATATGGAAAACTAA |
| 190 | A35R | AAAAATGAATTAATA |
| 191 | A37R | AAAATTGAAGTAATA |
| 192 | A40R | AATACTTAAAATGTA |
| 193 | A41L | AAAATATAAAATAAA |
| 194 | 268 | AAAAATGAACTCTTA |
| 195 | A44L | AAAATAGAATAAGTA |
| 196 | A46R | ATAAATGAAAAGATA |
| 197 | A47L | AAAACTGAAAATAAA |
| 198 | A48R | AAATTGTAAAAAATA |
| 199 | A50R | AAATATTAAAAAAAA |
| 200 | A52R | GAAATAAAAAACATA |
| 201 | A55R | AAAAATAAAAATATA |
| 202 | A56R | AATTTTGTAAAAATA |
| 203 | | ATTACATATTATATA |
| 204 | B1R | AAAACTTAAAATTTA |
| 205 | B2R | ATAAAAATTAAAAAA |
| 206 | B5R | ATATCTAAAAATCTT |
| 207 | B6R | AAAAATAATGACCAA |
| 208 | B8R | ATTATTCAAAATATG |
| 209 | B11R | GAAAATGAAAATATA |
| 210 | B12R | AAAACATAAAAAACA |
| 211 | B13R | AAGATTGAAATTATA |
| 212 | B17L | AAATATGTAAATATG |
| 213 | B19R | AAAACTGATATTATA |
| 214 | Pseudogene | ATAAATGTAGACTCT |
| 215 | C12L | TAAACTGAAGTTTAA |
| 216 | K2L | ATTTTTATACCGAACATAAAAATAAGGTTAATTATTAATACCA |
| | | TAAAATC |
| 217 | K4L | GGATTTTTAATAGAGTGAAGTGATATAGGATTATTCTTTTAAC |
| | | AAATAAA |
| 218 | F13L | ATTCTAGAATCGTTGATAGAACAGGATGTATAAGTTTTTATGT |
| | | TAACTAA |
| 219 | E11L | TTTGTATCATTTGTCCATCAACGTCATTTCAATAATATTGGATG |
| | | ATATAA |
| 220 | O2L | ACTAAAGAGTTAAATAAGTCGAGATAGTTTTATATCACTTAAA |
| | | TATTAAA |
| 221 | 03L | GTGCCTAATATTACTATATCAAGTAATGCTGAATAAAAATATT |
| | | TATAAAT |
| 222 | I1L | TTCTACTACTATTGATATATTTGTATTTAAAAGTTGTTTGGTGA |
| | | ACTTAA |
| 223 | I5L | ATACAACTAGGACTTTGTCACATATTCTTTGATCTAATTTTTAG |
| | | ATATAA |
| 224 | I6L | TGTGATATGTGATAAATTAACTACAAAATTAAATAGAATAGTA |
| | | AACGACG |
| 225 | G4L | CAGTGATTTATTTTCCAGCAGTAACGATTTTAAGTTTTTGATAC |
| | | CCATAA |
| 226 | H1L | AATTACACGCGTTTACCGATAAAGTAGTTTTATCCATTTGTAC |
| | | GTTATAA |
| 227 | H3L | AAAATATAACTCGTATTAAAGAGTTGTATATGATTAATTTCAA |
| | | TAACTAA |
| 228 | D8L | AATTCCCATACTAAGAGCTATTTTTAAACAGTTATCATTTCATT |
| | | TTTACT |
| 229 | D11L | TAAACTACTGCTGTGATTTTTAAAACATAGTTATTACTTATCAC |
| | | TCATAA |
| 230 | D13L | GATATTTCTCTACGGAGTTTATTGTAAGCTTTTTCCATTTTAAA |
| | | TAGAAA |
| 231 | A1L | AGGTTTTCTACTTGCTCATTAGAAGTATAAAAAAATAGTTCCG |
| | | TAATTAA |
| 232 | A2L | AAAATGTTTTTATATAAAATATTGGACGACGAGATACGTAGAG |
| | | TGTTAAC |
| 233 | A3L | AGATTGGATATTAAAATCACGCTTTCGAGTAAAAACTACGAAT |
| | | ATAAATA |
| 234 | A6L | AACTCTGGAAGAGCACAAATAAATTAAACAACTAAATCTGTA |
| | | AATAAATA |
| 235 | A12L | TATAATCTAGTTAAATCTTCTGTATAAATAAAAATATTTTTAGC |
| | | TTCTAA |
| 236 | A15L | CTATTTTATATCTATTTATTCGCGTCCTAAAATTAAAACAAATG |
| | | ATATAA |
| 237 | A16L | GATGTTGATATACCAACATTTAACAGTTTAAATACTGACGATT |
| | | ATTAAGA |
| 238 | A19L | TTGCACGATCGTGTTATAGGGCATATTCTGACTTATTTTTTACT |
| | | ACCTAA |
| 239 | | AATTCGAAAGAAAAAGAATCACAGTCCTAAAAGCTGAACTTC |
| | | GGAAATCT |
| 240 | | ATCTAGAATATCAGATCTTGAAAGACAGTTGAACGACTGTAG |
| | | ACGTAATA |
| 241 | A25L | TTATAATTACCCGATTGTAGTTAAGTTTTGAATAAAATTTTTTA |
| | | TAATAA |
| 242 | A27L | TACCAAATATAAATAACGCAGAGTGTCAGTTTCTAAAATCTGT |
| | | ACTTTAA |
| 243 | A30L | TCCATAAAAGACGAATAAGATACAAACACAAATGTTTATATA |
| | | ATATTTAA |
| 244 | A30.5L | ATGTTTTTTCCAAAAACCTAAGTGTATTTAAAATAGATGCCAT |
| | | GTTAAAA |
| 245 | A32L | TCCATATTTTGATTTATTATCAAATTAATTTAGTAACTGTAAAT |
| | | ATAATT |
| 246 | A38L | CAAAATAGAATAAAATAAATAACAAAGGTATCATTTTAAATA |
| | | AATAAAAA |
| 247 | | GATATCCATGGTATAGACCAAACAATAACGATATATATCATAA |
| | | ATAAATAA |
| 248 | E6R | TAATTATTAGAATAAGAGTGTAGTATCATAGATAACTCTCTTC |
| | | TATAAAA |
| 249 | E7R | TATACATAGATATAATTATCACATATTAAAAATTCACACATTT |
| | | TTGATAA |
| 250 | E8R | ACATAAAAACTCATTACATAGTTGATAAAAAGCGGTAGGATA |
| | | TAAATATT |
| 251 | I8R | TAGTTCTGGTATTTTACTAATTACTAAATCTGTATATCTTTCCA |
| | | TTTATC |
| 252 | G8R | CGACGCTGTTCTGCAGCCATTTAACTTTAAATAATTTACAAAA |
| | | ATTTAAA |
| 253 | L4R | TTTGTAACATCGGTACGGGTATTCATTTATCACAAAAAAAACT |
| | | TCTCTAA |
| 254 | J1R | TAGTAAACCGATAGTGTATAAAGATTGTGCAAAGCTTTTGCGA |
| | | TCAATAA |
| 255 | H7R | CTACGGATGGATGATATAGATCTTTACACAAATAATTACAAAA |
| | | CCGATAA |
| 256 | D6R | ATCTCCGTAAATATATGCTCATATATTTATAGAAGATATCACA |
| | | TATCTAA |
| 257 | D10R | GATAAATACGAATATCTGTCTTATATTTATAATATGCTAGTTA |
| | | ATAGTAA |
| 258 | A22R | CAATATTGAAAATACTAATTGTTTAAATAACCCGAGTATTGAA |
| | | ACTATAT |
| 259 | A34R | TATTTTTGTGTTAAAACAATGAACTAATATTTATTTTTGTACAT |
| | | TAATAA |
| 260 | | GATACGATACTATATGTATTCTTCGATAGTCCGCATTATGTAC |
| | | CTATTCT |
| 261 | A42R | CAAGTTTATTCCAATAGATGTCTTATTAAAAACATATATAATA |
| | | AATAACA |
| 262 | A43R | AACTGGTAATTAAAATAAAAAGTAATATTCATATGTAGTGTCA |
| | | ATTTTAA |
| 263 | A53R | TTTTTGATGGTGGTTTAACGTTTTAAAAAAAGATTTTGTTATTG |
| | | TAGTAT |
| 264 | B4R | TAACATTGTTAATTGAAAAGGGATAACATGTTACAGAATATAA |
| | | ATTATAT |
| 265 | B9R | TGCATATTATACACTGGTTAACGCCCTTATAGGCTCTAACCAT |
| | | TTTCAAG |
| 266 | | TTGCAGTGTTCATCTCCCAACTGCAAGTGAAGGATTGATAACT |
| | | GAAGGCA |
| 267 | | CTCTTCTCCCTTTCCCAGAAACAAACTTTTTTTACCCACTATAA |
| | | AATAAA |
| 268 | C13L | AATAGTATAAACTAAAAATTAAACAAATCGTTATTATAAGTAA |
| | | TATCAAA |
| 269 | C19L | TTCTGTTTTTCTTTCACATCTTTAATTATGAAAAAGTAAATCAT |
| | | TATGAG |
| 270 | C8L | CACTTACTAAATAGCCAAGGTGATTATTCGTATTTTTTTAAGG |
| | | AGTAACC |
| 271 | C3L | TTTTATTATTTGTACGATGTCCAGGATAACATTTTTACGGATAA |
| | | ATAAAT |
| 272 | F9L | TAGTTTCTTGGAAAAATTTATTATGAGAGACATTTTCTCAGAC |
| | | TGGATAA |
| 273 | F10L | TCTATCAAACCTGGACTTTCGTTTGTAAATTGGGGCTTTTTGTA |
| | | CAATAA |
| 274 | I2L | ATGAATATGATGAAGATAGCGATAAAGAAAAGCCAATATTCA |
| | | ATGTATAA |
| 275 | I7L | AACGCAGTTTGGAAAAAAGAAGATATCTGGTAAATTCTTTTCC |
| | | ATGATAA |
| 276 | G1L | TACGATGATAACGACATACGAACATTACTTCCTATTTTACTCC |
| | | TTAGTAA |
| 277 | G3L | ATCTTCTGTAAGTAGGAATTTGGACAAGTTGAACAAAATTAGA |
| | | TCTCTAA |
| 278 | G7L | ATTTTTATACGGATGCTCATTTTAAATTTTTGTAAATTATTTAA |
| | | AGTTAA |
| 279 | L3L | ATGAGGTTTTCTAGCAGTAGACTCATTTAGAGAAGTTTTTTTTG |
| | | TGATAA |
| 280 | J5L | TTATTACAACTATAAAAATAATAGTTATATTTACACTTTAAATT |
| | | TTTATC |
| 281 | H4L | TAAAAAAATTATACATCATAAACCAATTTCCTAGTTGTTTGTA |
| | | ACTTTAA |
| 282 | D2L | CGTTATCGTCGTTATCTACTTTGGGATACTTATTATCCTTAACT |
| | | ATAAAA |
| 283 | A2.5L | TATATTAGCGCTAGACATATTACAGAACTATTTTAGATTATGA |
| | | TATTTAA |
| 284 | A7L | AAGACTTACATCATCGGTAGTAGATTTTCACTTTACCCCACGA |
| | | TATAAAT |
| 285 | A9L | AAAATCTAAATATGACAGATGGTGACTCTGTCTCTTTTGATGA |
| | | TGAATAA |
| 286 | A10L | ATCGTTTTGTATATCCGTCACTGGTACGGTCGTCATTTAATACT |
| | | AAATAA |
| 287 | A13L | AAAAGATGATATATTGCATACTTGATCAATAGTGAAGTTATTG |
| | | TCAATAA |
| 288 | A14L | GTTTATATTCCACTTTGTTCATTCGGCGATTTAAAATTTTTATT |
| | | AGTTAA |
| 289 | A14.5L | ATTCGTATTATTTGAGCAAGAAAATATCCCACCACCTTTTCGT |
| | | CTAGTAA |
| 290 | A17L | GGCATAAAGATTATACTCCATCTTTAATAGTGACATTTTTTAAT |
| | | ATATAA |
| 291 | A21L | TGTACAGACTAAGTAATTCTTTTAAGTTAGTTAAATCAGCGCT |
| | | AGAAGTC |
| 292 | A26L | ACTTAACTCTTTTGTTAATTAAAAGTATATTCAAAAAATGAGT |
| | | TATATAA |
| 293 | A28L | CATTGTCTGATGCGTGTAAAAAAATTTTGTCAGCTTCTAATAG |
| | | ATTATAA |
| 294 | F17R | TGTATGTAAAAATATAGTAGAATTTCATTTTGTTTTTTTCTATG |
| | | CTATAA |
| 295 | E10R | TAATGCACCGAACATCCATTTATAGAATTTAGAAATATATTTT |
| | | CATTTAA |
| 296 | G6R | AGAACCTCAACGTAACTTAACAGTGCAACCTCTATTGGATATA |
| | | AACTAAT |
| 297 | G9R | GATCAACATCTTTATGGCGTTTTTAGATTAATACTTTCAATGAG |
| | | ATAAAT |
| 298 | L1R | TCAGTTTATTATCTCTCTTGGTAATATGGATACTAATTGTAGCT |
| | | ATTTAA |
| 299 | L5R | AAAAGAATATTCCTCTAACAGATATTCCGACAAAGGATTGATT |
| | | ACTATAA |
| 300 | H2R | GTAGTAGTAAGTATTTATACAAACTTTTCTTATCCATTTATAAC |
| | | GTACAA |
| 301 | H6R | AGGGAAAATCTAAAGTTGTTCGTAAAAAAGTTAAAACTTGTA |
| | | AGAAGTAA |
| 302 | D3R | ATAAAATACTACTGTTGAGTAAATCAGTTATTTTTTTTATATCG |
| | | ATATTG |
| 303 | A11L | TTGATCAAGAGTAACTATTGACTTAATAGGCATCATTTATTTA |
| | | GTATTAA |
| 304 | A39R | CCAATTTCCATCTAATATACTTTGTCGGATTATCTATAGTACAC |
| | | GGAATA |
| 305 | A45R | CCATTGCTGCCACTCATAATATCAGACTACTTATTCTATTTTAC |
| | | TAAATA |
| 306 | B7R | TTTGTATAAATAATTATTTCAATATACTAGTTAAAATTTTAAGA |
| | | TTTTAA |
| 307 | | TCCATCCACAGACGTTACCGAACCGATTAGTGATGTGACACCA |
| | | TCGGTGG |
| 308 | | ATACGAGGACGTGTATAGAGTAAGTAAAGAAAAAGAATGTGG |
| | | AATTTGCT |

In addition, the nucleic acid sequence for insertion may further include suitable translation initiation sequences, such as for example, a Kozak consensus sequence (GCCACC/ATG).

In addition, the polycistronic nucleic acid sequence for insertion can include appropriate stop codons, for example TAA, TAG, or TGA, or combinations or multiples thereof, at the 3′end of the nucleic acid sequence following the last amino acid encoding sequence of the polypeptide. Furthermore, the nucleic acid sequence can include a vaccinia virus termination sequence 3′ of the last stop codon of polyprotein. In addition, the nucleic acid sequence for insertion may further include restriction enzyme sites useful for generating shuttle vectors for ease of insertion of the immune checkpoint inhibitor encoding sequences.

### Antigenic Targets

The provided rMVA viral constructs of the present invention can be used as an adjuvant for treating or preventing an infectious disease or cancer in a subject. In some embodiments, the rMVA viral construct is administered to a subject in need thereof, for example a human, in a prophylactic vaccination protocol to prevent an infectious disease, for example at a priming stage, a boosting stage, or both a priming stage and bosting stage. In an alternative embodiment, the rMVA viral construct is administered to a subject in need thereof, for example a human, in a treatment modality incorporating a vaccination protocol, for example, to treat a cancer. Accordingly, the rMVA viral construct can be administered in concert with one or more antigens intended to induce an immune response against an antigenic target in order to induce partial or complete immunization in a subject in need thereof.

Thus, the rMVA of the present invention can be administered with one or more antigens targeting an infectious disease or cancer. Examples of antigens and antigen delivery vehicles that the rMVA can be used with as an adjuvant include: an antigenic protein, polypeptide, or peptide, or fragment thereof, a nucleic acid, for example mRNA or DNA, encoding one or more antigens; a polysaccharide or a conjugate of a polysaccharide to a protein; glycolipids, for example gangliosides; a toxoid; a subunit (e.g., of a virus, bacterium, fungi, amoeba, parasite, etc.); a virus like particle; a live virus; a split virus; an attenuated virus; an inactivated virus; an enveloped virus; a viral vector expressing one or more antigens; a tumor associated antigen; or any combination thereof.

In particular aspects, the present invention provides a method of preventing or treating an infectious disease in a subject in need thereof, said method comprising administering an effective amount of the rMVA of the present invention in combination, alternation, or coordination with a prophylactically effective or therapeutically effective amount of one or more antigens, or antigen expressing vectors, wherein the rMVA enhances immunity directed against the targeted infectious diseases.

In some embodiments, the targeted infection is a viral infection, including but not limited to: a double-stranded DNA virus, including but not limited to Adenoviruses, Herpesviruses, and Poxviruses; a single stranded DNA, including but not limited to Parvoviruses; a double stranded RNA virus, including but not limited to Reoviruses; a positive-single stranded RNA virus, including but not limited to Coronaviruses, Picornaviruses, and Togaviruses; a negative-single stranded RNA virus, including but not limited to Orthomyxoviruses, and Rhabdoviruses; a single-stranded RNA-Retrovirus, including but not limited to Retroviruses; or a double-stranded DNA-Retrovirus, including but not limited to Hepadnaviruses. In some embodiments, the targeted virus is adenovirus, avian influenza, coxsackievirus, cytomegalovirus, dengue fever virus, ebola virus, Epstein-Barr virus, equine encephalitis virus, flavivirus, hepadnavirus, hepatitis A virus, hepatitis B virus, hepatitis C virus, hepatitis D virus, hepatitis E virus, herpes simplex virus, human immunodeficiency virus, human papillomavirus, influenza virus, Japanese encephalitis virus, JC virus, measles morbillivirus, marburg virus, Middle Eastern respiratory syndrome (MERS-CoV)-coronavirus, mumps rubulavirus, orthomyxovirus, papillomavirus, parainfluenza virus, parvovirus, picornavirus, poliovirus, pox virus, rabies virus, reovirus, respiratory syncytial virus, retrovirus, rhabdovirus, rhinovirus, Rift Valley fever virus, rotavirus, rubella virus, rubeola virus, severe acute respiratory syndrome-coronavirus 1 (SARS-CoV), severe acute respiratory syndrome coronavirus 2 (SARS-CoV2), smallpox virus, togavirus, swine influenza virus, varicella-zoster virus, variola major, variola minor, and yellow fever virus. Examples of viruses that may be used as antigens also include measles virus, mumps virus (Mumps rubulavirus), Rubella virus, varicella zoster virus or a combination of all four or three thereof (e.g., measles, mumps, and rubella).

In some embodiments, the targeted infectious agent is a Flaviviridae virus, including infections with viruses of the genera Flavivirus and Pestivirus. Flavivirus infections include Dengue fever, Kyasanur Forest disease, Powassan disease, Wesselsbron disease, West Nile fever, yellow fever, Zika virus, Rio bravo, Rocio, Negishi, and the encephalitises including: California encephalitis, central European encephalitis, Ilheus virus, Murray Valley encephalitis, St. Louis encephalitis, Japanese B encephalitis, Louping ill, and Russian spring-rodents summer encephalitis. Pestivirus infections include primarily livestock diseases, including swine fever in pigs, BVDV (bovine viral diarrhea virus) in cattle, or Border Disease virus infections.

In some embodiments, the targeted infectious agent is an Alphavirus virus, for example, Eastern equine encephalitis (EEE) virus, Venezuelan equine encephalitis (VEE) virus, Western equine encephalitis (WEE) virus, the Everglades virus, Chikungunya virus, Mayaro virus, Ockelbo virus, O'nyong-nyong virus, Ross River virus, Semliki Forest virus or Sindbis virus (SINV).

In some embodiments, the targeted infectious agent is the equine arteritis virus, bovine viral diarrhea virus (BVDV), hog cholera virus or border disease virus. The only member of the Rubivirus genus is the rubella virus.

In some embodiments, the targeted infectious agent a Filoviridae virus such as the Ebola virus and Marburg virus; a Paramyxoviridae virus such as Measles virus, Mumps virus, Nipah virus, Hendra virus, respiratory syncytial virus (RSV) and Newcastle disease virus (NDV); Rhabdoviridae virus such as Rabies virus; a Nyamiviridae virus such as Nyavirus, an Arenaviridae virus such as Lassa virus, a Bunyaviridae virus such as Hantavirus, Crimean-Congo hemorrhagic fever; or Ophioviridae and Orthomyxoviridae viruses such as influenza virus.

In one embodiment, an antigen is taken from one or more bacteria selected from Borrelia species, Bacillus anthraces, Borrelia burgdorferi, Bordetella pertussis, Camphylobacter jejuni, Chlamydia species, Chlamydial psittaci, Chlamydial trachomatis, Clostridium species, Clostridium tetani, Clostridium botulinum, Clostridium perfringens, Corynebacterium diphtheriae, Coxiella species, an Enterococcus species, Erlichia species, Escherichia coli, Francisella tularensis, Haemophilus species, Haemophilus influenzae, Haemophilus parainjluenzae, Lactobacillus species, a Legionella species, Legionella pneumophila, Leptospirosis interrogans, Listeria species, Listeria monocytogenes, Mycobacterium species, Mycobacterium tuberculosis, Mycobacterium leprae, Mycoplasma species, Mycoplasma pneumoniae, Neisseria species, Neisseria meningitidis, Neisseria gonorrhoeae, Pneumococcus species, Pseudomonas species, Pseudomonas aeruginosa, Salmonella species, Salmonella typhi, Salmonella enterica, Streptococcus species, Rickettsia species, Rickettsia ricketsii, Rickettsia typhi, Shigella species, Staphylococcus species, Staphylococcus aureus, Streptococcus species, Streptococcus pneumoniae, Streptococcus pyrogenes, Streptococcus mutans, Treponema species, Treponema pallidum, a Vibrio species, Vibrio cholerae and Yersinia pestis. Such bacteria may be a whole cell (e.g., live, attenuated or inactivated) or a polypeptide or polysaccharide of such a bacterium.

In some embodiments, the targeted infectious agent is a bacterium. The antigenic bacterial agent for targeting can be a polysaccharide-polypeptide antigen such as a pneumococcal (e.g., S. pneumonia) polysaccharide (e.g., a cell capsule sugar)-protein (e.g., diphtheria protein) conjugate. In some embodiments, the conjugate comprises cell capture sugars of S. pneumonia conjugated to a protein (e.g., diphtheria protein), e.g., wherein the cell capsule sugars are of seven serotypes of the bacteria S. pneumoniae (4, 6B, 9V, 14, 18C, 19F and 23F), conjugated with diphtheria proteins. In some embodiments, the conjugate comprises Pneumococcal polysaccharide serotype 1, 4, 5, 6B, 7F, 9V, 14, 18C, 19F and 23F conjugated to a protein such as protein D derived from non-typeable Haemophilus influenza, tetanus toxoid carrier protein and/or diphtheria toxoid carrier protein. In some embodiments, the conjugate comprises Streptococcus pneumonia capsular polysaccharide conjugated to a diphtheria protein, e.g., Streptococcus pneumoniae type 1, 3, 4, 5, 6a, 6b, 7f, 9v, 14, 18c, 23f, 19a and 19f capsular polysaccharide conjugated to a protein such as diphtheria crm197 protein. In some embodiments, one or more of the polysaccharide-protein conjugates comprising capsular polysaccharides from at least one of serotypes 1, 2, 3, 4, 5, 6A, 6B, 6C, 6D, 6E, 6G, 6H, 7F, 7A, 7B, 7C, 8, 9A, 9L, 9N, 9V, 1° F., 10A, 10B, 10C, 11F, 11A, 11B, 11C, 11D, 11E, 12F, 12A, 12B, 13, 14, 15F, 15A, 15B, 15C, 16F, 16A, 17F, 17A, 18F, 18A, 18B, 18C, 19F, 19A, 19B, 19C, 20A, 20B, 21, 22F, 22A, 23F, 23A, 23B, 24F, 24A, 24B, 25F, 25A, 27, 28F, 28A, 29, 31, 32F, 32A, 33F, 33A, 33B, 33C, 33D, 33E, 34, 35F, 35A, 35B, 35C, 36, 37, 38, 39, 40, 41F, 41A, 42, 43, 44, 45, 46, 47F, 47A, 48, CWPS1, CWPS2, CWPS3 of Streptococcus pneumoniae conjugated to one or more carrier proteins.

In some embodiments, the targeted infectious agent is a fungus, for example, but not limited to one or more fungus selected from an Aspergillus species, Candida species, Candida albicans, Candida tropicalis, Cryptococcus species, Cryptococcus neoformans, Entamoeba histolytica, Histoplasma capsulatum, Leishmania species, Nocardia asteroides, Plasmodium falciparum, Toxoplasma gondii, Trichomonas vaginalis, Toxoplasma species, Trypanosoma brucei, Schistosoma mansoni, Fusarium species, and/or Trichophyton species. Such fungi may be a whole cell (e.g., live, attenuated or inactivated) or a polypeptide or polysaccharide of such a fungus.

In some embodiments, the targeted infectious agent is one or more parasites selected from Plasmodium species, Toxoplasma species, Entamoeba species, Babesia species, Trypanosoma species, Leishmania species, Pneumocystis species, Trichomonas species, Giardia species, and/or Schisostoma species. Such parasite antigens may be a whole cell (e.g., live, attenuated, or inactivated) or a polypeptide or polysaccharide of such a parasite.

In some embodiments, the antigenic agent is encoded by a nucleic acid. For example, in some embodiments, the antigenic agent is encoded by a nucleic acid is selected form DNA, RNA, mRNA, etc.

In some embodiments, the antigen is a toxoid. In some embodiments, the toxoid is diphtheria toxoid or tetanus toxoid or toxoids from C. Difficile.

In particular embodiments, the targeted antigen is derived from: the Ebola virus, for example, the envelope glycoprotein of Ebola virus Zaire strain (e.g., UniProtKB—P87671 (VGP_EBOEC)), the matrix protein VP40 of Ebola virus Zaire strain (e.g., UniProtKB—Q05128 (VP40_EBOZM)), or the matrix protein of Ebola virus Sudan strain (e.g., UniProtKB—Q7T9D9 (VGP_EBOSU)); the Lassa virus, for example, protein Z (e.g., UniProtKB—073557 (Z_LASSJ)); the Zika virus, for example, non-structural protein 1 (NSP-1); the Marburg virus, for example, the Marburg virus glycoprotein (GenBank accession number AFV31202.1), the Marburg VP40 matrix protein (GenBank accession number JX458834); the Plasmodium sp. parasite, for example Plasmodium falciparum, for example, circumsporozoite protein (CSP), the Male gametocyte surface protein P230p (Pfs230 antigen), sporozoite micronemal protein essential for cell traversal (SPECT2), or GTP-binding protein, putative antigen (GenBank accession number PF3D7_1462300); the human immunodeficiency virus, for example an Env protein, for example gp41, gp120, gp160, a Gag protein, MA, CA, SP1, NC, SP2, P6, or a Pol protein RT, RNase H, IN, PR.

In an alternative embodiment, the rMVA viral construct is administered to a subject in need thereof, for example a human, in a treatment modality incorporating a vaccination protocol, for example, to treat a cancer. Accordingly, the rMVA viral construct can be administered in concert with one or more antigens intended to induce an immune response against an antigenic target in order to induce partial or complete immunization in a subject in need thereof.

Antigens used for cancer immunotherapy are generally intentionally selected based on either uniqueness to tumor cells, greater expression in tumor cells as compared to normal cells, or ability of normal cells with antigen expression to be adversely affected without significant compromise to normal cells or tissue. Tumor-associated antigens (TAA) can be loosely categorized as oncofetal (typically only expressed in fetal tissues and in cancerous somatic cells), oncoviral (encoded by tumorigenic transforming viruses), overexpressed/accumulated (expressed by both normal and neoplastic tissue, with the level of expression highly elevated in neoplasia), cancer-testis (expressed only by cancer cells and adult reproductive tissues such as testis and placenta), lineage-restricted (expressed largely by a single cancer histotype), mutated (only expressed by cancer as a result of genetic mutation or alteration in transcription), post-translationally altered (tumor-associated alterations in glycosylation, etc.), or idiotypic (highly polymorphic genes where a tumor cell expresses a specific “clonotype”, i.e., as in B cell, T cell lymphoma/leukemia resulting from clonal aberrancies). Although they are preferentially expressed by tumor cells, TAAs are oftentimes found in normal tissues. However, their expression differs from that of normal tissues by their degree of expression in the tumor, alterations in their protein structure in comparison with their normal counterparts or by their aberrant subcellular localization within malignant or tumor cells.

Examples of oncofetal tumor associated antigens include Carcinoembryonic antigen (CEA), immature laminin receptor, and tumor-associated glycoprotein (TAG) 72. Examples of overexpressed/accumulated include BING-4, calcium-activated chloride channel (CLCA) 2, Cyclin A₁, Cyclin B₁, 9D7, epithelial cell adhesion molecule (Ep-Cam), EphA3, Her2/neu, telomerase, mesothelin, orphan tyrosine kinase receptor (ROR1), stomach cancer-associated protein tyrosine phosphatase 1 (SAP-1), and survivin.

Examples of cancer-testis antigens include the b melanoma antigen (BAGE) family, cancer-associated gene (CAGE) family, G antigen (GAGE) family, melanoma antigen (MAGE) family, sarcoma antigen (SAGE) family and X antigen (XAGE) family, CT9, CT10, NY-ESO-1, L antigen (LAGE) 1, Melanoma antigen preferentially expressed in tumors (PRAME), and synovial sarcoma X (SSX) 2. Examples of lineage restricted tumor antigens include melanoma antigen recognized by T cells-1/2 (Melan-A/MART-1/2), Gp100/pmel17, tyrosine-related protein (TRP) 1 and 2, P. polypeptide, melanocortin 1 receptor (MC1R), and prostate-specific antigen. Examples of mutated tumor antigens include β-catenin, breast cancer antigen (BRCA) 1/2, cyclin-dependent kinase (CDK) 4, chronic myelogenous leukemia antigen (CML) 66, fibronectin, p53, Ras, and TGF-βRII. An example of a post-translationally altered tumor antigen is mucin (MUC) 1. Examples of idiotypic tumor antigens include immunoglobulin (Ig) and T cell receptor (TCR).

In some embodiments, the antigen associated with the disease or disorder is selected from the group consisting of CD19, CD20, CD22, hepatitis B surface antigen, anti-folate receptor, CD23, CD24, CD30, CD33, CD38, CD44, EGFR, EGP-2, EGP-4, OEPHa2, ErbB2, 3, or 4, FBP, fetal acetylcholine receptor, HMW-MAA, IL-22R-alpha, IL-13R-alpha, kdr, kappa light chain, Lewis Y, MUC16 (CA-125), PSCA, NKG2D Ligands, oncofetal antigen, VEGF-R2, PSMA, estrogen receptor, progesterone receptor, ephrinB2, CD123, CS-1, c-Met and/or biotinylated molecules, and/or molecules expressed by HIV, HCV, HBV or other pathogens.

Exemplary tumor antigens include at least the following: carcinoembryonic antigen (CEA) for bowel cancers; CA-125 for ovarian cancer; MUC1 or epithelial tumor antigen (ETA) or CA15-3 for breast cancer; tyrosinase or melanoma-associated antigen (MAGE) for malignant melanoma; and abnormal products of ras, p53 for a variety of types of tumors; alphafetoprotein for hepatoma, ovarian, or testicular cancer; beta subunit of hCG for men with testicular cancer; prostate specific antigen for prostate cancer; beta 2 microglobulin for multiple myeloma and in some lymphomas; CA19-9 for colorectal, bile duct, and pancreatic cancer; chromogranin A for lung and prostate cancer; TA90 for melanoma, soft tissue sarcomas, and breast, colon, and lung cancer. Examples of TAAs are known in the art, for example in N. Vigneron, “Human Tumor Antigens and Cancer Immunotherapy,” BioMed Research International, vol. 2015, Article ID 948501, 17 pages, 2015. doi:10.1155/2015/948501; Ilyas et al., J Immunol. (2015) Dec. 1; 195(11): 5117-5122; Coulie et al., Nature Reviews Cancer (2014) volume 14, pages 135-146; Cheever et al., Clin Cancer Res. (2009) Sep. 1; 15(17):5323-37, which are incorporated by reference herein in its entirety.

Examples of oncoviral TAAs include human papilloma virus (HPV) L1, E6 and E7, Epstein-Barr Virus (EBV) Epstein-Barr nuclear antigen (EBNA) 1 and 2, EBV viral capsid antigen (VCA) Igm or IgG, EBV early antigen (EA), latent membrane protein (LMP) 1 and 2, hepatitis B surface antigen (HBsAg), hepatitis B e antigen (HBeAg), hepatitis B core antigen (HBcAg), hepatitis B x antigen (HBxAg), hepatitis C core antigen (HCV core Ag), Human T-Lymphotropic Virus Type 1 core antigen (HTLV-1 core antigen), HTLV-1 Tax antigen, HTLV-1 Group specific (Gag) antigens, HTLV-1 envelope (Env), HTLV-1 protease antigens (Pro), HTLV-1 Tof, HTLV-1 Rof, HTLV-1 polymerase (Pro) antigen, Human T-Lymphotropic Virus Type 2 core antigen (HTLV-2 core antigen), HTLV-2 Tax antigen, HTLV-2 Group specific (Gag) antigens, HTLV-2 envelope (Env), HTLV-2 protease antigens (Pro), HTLV-2 Tof, HTLV-2 Rof, HTLV-2 polymerase (Pro) antigen, latency-associated nuclear antigen (LANA), human herpesvirus-8 (HHV-8) K8.1, Merkel cell polyomavirus large T antigen (LTAg), and Merkel cell polyomavirus small T antigen (sTAg).

Elevated expression of certain types of glycolipids, for example gangliosides, is associated with the promotion of tumor survival in certain types of cancers. Examples of gangliosides include, for example, GM1b, GD1c, GM3, GM2, GM1a, GD1a, GT1a, GD3, GD2, GD1b, GT1b, GQlb, GT3, GT2, GT1c, GQ1c, and GPlc. Examples of ganglioside derivatives include, for example, 9-O—Ac-GD3, 9-O—Ac-GD2, 5-N-de-GM3, N-glycolyl GM3, NeuGcGM3, and fucosyl-GM1. Exemplary gangliosides that are often present in higher levels in tumors, for example melanoma, small-cell lung cancer, sarcoma, and neuroblastoma, include GD3, GM2, and GD2.

In addition to the TAAs described above, another class of TAAs is tumor-specific neoantigens, which arise via mutations that alter amino acid coding sequences (non-synonymous somatic mutations). Some of these mutated peptides can be expressed, processed and presented on the cell surface, and subsequently recognized by T cells. Because normal tissues do not possess these somatic mutations, neoantigen-specific T cells are not subject to central and peripheral tolerance, and also lack the ability to induce normal tissue destruction. See, e.g., Lu & Robins, Cancer Immunotherapy Targeting Neoantigens, Seminars in Immunology, Volume 28, Issue 1, February 2016, Pages 22-27, incorporated herein by reference.

In some embodiments, the TAA is specific to an oncofetal TAA selected from a group consisting of Carcinoembryonic antigen (CEA), immature laminin receptor, orphan tyrosine kinase receptor (ROR1), and tumor-associated glycoprotein (TAG) 72.

In some embodiments, a TAA is specific to an oncoviral TAA selected from a group consisting of human papilloma virus (HPV) E6 and E7, Epstein-Barr Virus (EBV) Epstein-Barr nuclear antigen (EBNA) 1 and 2, latent membrane protein (LMP) 1, and LMP2.

In some embodiments, the TAA is specific to an overexpressed/accumulated TAA selected from a group consisting of BING-4, calcium-activated chloride channel (CLCA) 2, CyclinAi, Cyclin B₁, 9D7, epithelial cell adhesion molecule (Ep-Cam), EphA3, Her2/neu, L1 cell adhesion molecule (L1-Cam), telomerase, mesothelin, stomach cancer-associated protein tyrosine phosphatase 1 (SAP-1), and survivin.

In some embodiments, the TAA is specific to a cancer-testis antigen selected from the group consisting of the b melanoma antigen (BAGE) family, cancer-associated gene (CAGE) family, G antigen (GAGE) family, melanoma antigen (MAGE) family, sarcoma antigen (SAGE) family and X antigen (XAGE) family, cutaneous T cell lymphoma associated antigen family (cTAGE), Interleukin-13 receptor subunit alpha-1 (IL13RA), CT9, Putative tumor antigen NA88-A, leucine zipper protein 4 (LUZP4), NY-ESO-1, L antigen (LAGE) 1, helicase antigen (HAGE), lipase I (LIPI), Melanoma antigen preferentially expressed in tumors (PRAME), synovial sarcoma X (SSX) family, sperm protein associated with the nucleus on the chromosome X (SPANX) family, cancer/testis antigen 2 (CTAG2), calcium-binding tyrosine phosphorylation-regulated fibrous sheath protein (CABYR), acrosin binding protein (ACRBP), centrosomal protein 55 (CEP55) and Synaptonemal Complex Protein 1 (SYCPi.

In some embodiments, the TAA is specific to a lineage restricted tumor antigen selected from the group consisting of melanoma antigen recognized by T cells-1/2 (Melan-A/MART-1/2), Gp100/pmel17, tyrosinase, tyrosine-related protein (TRP) 1 and 2, P. polypeptide, melanocortin 1 receptor (MC1R), and prostate-specific antigen.

In some embodiments, the TAA is specific to a mutated TAA selected from a group consisting of 0-catenin, breast cancer antigen (BRCA) 1/2, cyclin-dependent kinase (CDK) 4, chronic myelogenous leukemia antigen (CML) 66, fibronectin, MART-2, p53, Ras, TGF-βRII, and truncated epithelial growth factor (tEGFR).

In some embodiments, the TAA is specific to the post-translationally altered TAA mucin (MUC) 1.

In some embodiments, the TAA is specific to an idiotypic TAA selected from a group consisting of immunoglobulin (Ig) and T cell receptor (TCR).

In some embodiments, the TAA is specific to BCMA. In some embodiments, at least one T-cell subpopulation is specific to BCMA.

In some embodiments, the TAA is specific to CS1.

In some embodiments, the TAA is specific to XBP-1

In some embodiments, the TAA is specific to CD138.

In some embodiments, the TAA is specific to WT1, PRAME, Survivin, NY-ESO-1, MAGE-A3, MAGE-A4, Pr3, Cyclin A1, SSX2, Neutrophil Elastase (NE), HPV E6. HPV E7, EBV LMP1, EBV LMP2, EBV EBNA1, or EBV EBNA2.

In addition to the TAAs described above, another class of TAAs is tumor-specific neoantigens, which arise via mutations that alter amino acid coding sequences (non-synonymous somatic mutations). Some of these mutated peptides can be expressed, processed and presented on the cell surface, and subsequently recognized by T cells. Because normal tissues do not possess these somatic mutations, neoantigen-specific T cells are not subject to central and peripheral tolerance, and also lack the ability to induce normal tissue destruction. See, e.g., Lu & Robins, Cancer Immunotherapy Targeting Neoantigens, Seminars in Immunology, Volume 28, Issue 1, February 2016, Pages 22-27, incorporated herein by reference.

In specific embodiments, the TAA is derived from Mucin 1 (MUC1)(UniProtKB—P15941 (MUC1_HUMAN)). In some embodiments, the TAA is derived from Cyclin B1 (UniProtKB—P14635 (CCNB1_HUMAN)).

rMVA Viral Vectors

As provided herein is an rMVA viral vector comprising a heterologous nucleic acid insert encoding an immune checkpoint inhibitor capable of being secreted from the cell.

In some embodiments, the rMVA viral vector comprises a heterologous nucleic acid insert encoding a polypeptide wherein the polypeptide comprises (M)(Secretion Signal Peptide-Immune Checkpoint Inhibitor)x, wherein x=1, 2, 3, 4, 5, 6, 7, 8, 9, 10, or more than 10, wherein M=methionine.

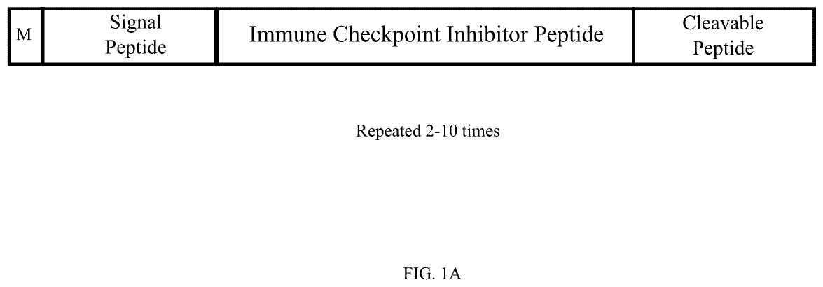

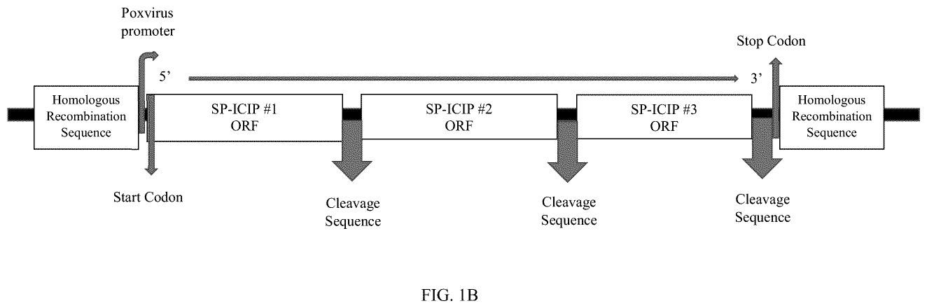

In some embodiments, the rMVA viral vector comprises a heterologous polycistronic nucleic acid insert encoding a polypeptide wherein the polypeptide comprises a tandem repeat sequence (M)(Secretion Signal Peptide-Immune Checkpoint Inhibitor Peptide-Cleavable Peptide)ₓ, wherein x=2, 3, 4, 5, 6, 7, 8, 9, 10, or more than 10, and wherein M=methionine (see, e.g., FIGS. 1A-1B).

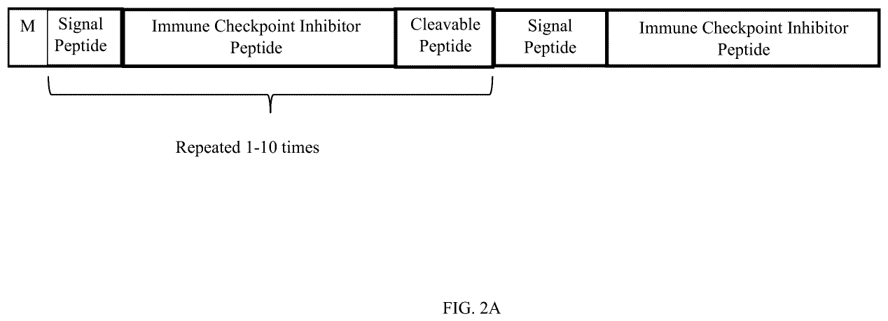

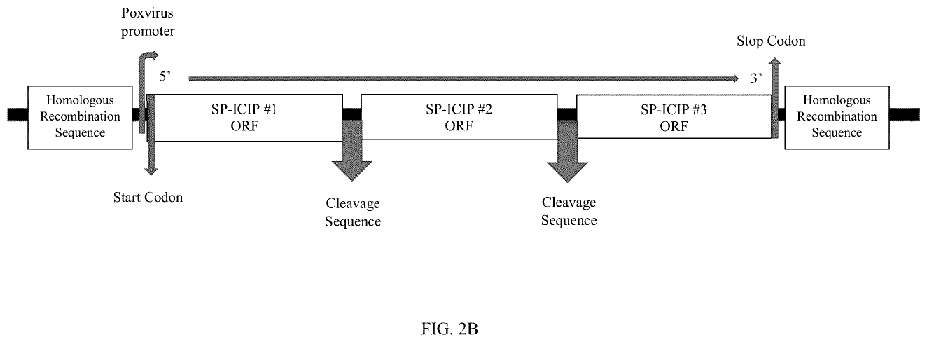

In some embodiments, provided herein is an rMVA viral vector comprising a heterologous polycistronic nucleic acid insert encoding one or more polypeptides in a tandem repeat sequence and an additional polypeptide fused to the C-terminus of the last polypeptide in the tandem repeat sequence ((M)(Secretion Signal Peptide-Immune Checkpoint Inhibitor Peptide-Cleavable Peptide)ₓ(Secretion Signal Peptide-Immune Checkpoint Inhibitor Peptide)), wherein x=1, 2, 3, 4, 5, 6, 7, 8, 9, 10, or more than 10, and wherein M=methionine (see, e.g., FIGS. 2A-2B). In particular embodiments, the encoded polypeptide comprises (M)(Secretion Signal Peptide-Immune Checkpoint Inhibitor Peptide-Cleavable Peptide)ₓ, wherein x=2, 3, 4, 5, 6, 7, 8, 9, 10, or more than 10, or in an alternative embodiment ((M)(Secretion Signal Peptide-Immune Checkpoint Inhibitor Peptide-Cleavable Peptide)ₓ(Secretion Signal Peptide-Immune Checkpoint Inhibitor Peptide)), wherein x=1, 2, 3, 4, 5, 6, 7, 8, 9, 10, or more than 10, wherein M=methionine, and wherein the Secretion Signal Peptide is selected from a peptide having an amino acid sequence selected from SEQ ID NOS: 57-90, the Immune Checkpoint Inhibitor Peptide is selected from a peptide having an amino acid sequence selected from SEQ ID NOS: 1-56, and the Cleavable Peptide is selected from a peptide having an amino acid sequence selected from SEQ ID NOS: 91-127. In some embodiments, the Secretion Signal Peptide is selected from a peptide having an amino acid sequence selected from SEQ ID NOS: 65 and 66, the Immune Checkpoint Inhibitor Peptide is selected from a peptide having an amino acid sequence selected from SEQ ID NOS: 1 and 5, and the Cleavable Peptide is selected from a peptide having an amino acid sequence selected from SEQ ID NOS: 93-97, 120, and 123-127.

In some embodiments, the Secretion Signal Peptide is a peptide having an amino acid sequence of SEQ ID NO: 66, the Immune Checkpoint Inhibitor Peptide is a peptide having an amino acid sequence of SEQ ID NO: 1.

In some embodiments, the Secretion Signal Peptide is a peptide having an amino acid sequence of SEQ ID NO: 66, the Immune Checkpoint Inhibitor Peptide is a peptide having an amino acid sequence of SEQ ID NO: 1, and the Cleavable Peptide is a peptide having an amino acid sequence of SEQ ID NO: 123, wherein x=2-10. In some embodiments, the Secretion Signal Peptide is a peptide having an amino acid sequence of SEQ ID NO: 66, the Immune Checkpoint Inhibitor Peptide is a peptide having an amino acid sequence of SEQ ID NO: 1, and the Cleavable Peptide is a peptide having an amino acid sequence of SEQ ID NO: 123, wherein x≥4. In some embodiments, the Secretion Signal Peptide is a peptide having an amino acid sequence of SEQ ID NO: 66, the Immune Checkpoint Inhibitor Peptide is a peptide having an amino acid sequence of SEQ ID NO: 1, and the Cleavable Peptide is a peptide having an amino acid sequence of SEQ ID NO: 123, wherein x=4, 5, or 6.

In some embodiments, the Secretion Signal Peptide is a peptide having an amino acid sequence of SEQ ID NO: 66, the Immune Checkpoint Inhibitor Peptide is a peptide having an amino acid sequence of SEQ ID NO: 5.

In some embodiments, the Secretion Signal Peptide is a peptide having an amino acid sequence of SEQ ID NO: 66, the Immune Checkpoint Inhibitor Peptide is a peptide having an amino acid sequence of SEQ ID NO: 5, and the Cleavable Peptide is a peptide having an amino acid sequence of SEQ ID NO: 123, wherein x=2-10. In some embodiments, the Secretion Signal Peptide is a peptide having an amino acid sequence of SEQ ID NO: 66, the Immune Checkpoint Inhibitor Peptide is a peptide having an amino acid sequence of SEQ ID NO: 5, and the Cleavable Peptide is a peptide having an amino acid sequence of SEQ ID NO: 123, wherein x≥4. In some embodiments, the Secretion Signal Peptide is a peptide having an amino acid sequence of SEQ ID NO: 66, the Immune Checkpoint Inhibitor Peptide is a peptide having an amino acid sequence of SEQ ID NO: 5, and the Cleavable Peptide is a peptide having an amino acid sequence of SEQ ID NO: 123, wherein x=4, 5, or 6.

In some embodiments, the polycistronic nucleic acid insert encodes a polypeptide comprising an amino acid of Table 8 below, or polypeptide having an amino acid sequence at least 85%, 90%, 95%, 97%, or 99% identical thereto. In some embodiments, the polycistronic nucleic acid insert encodes a polypeptide comprising an amino acid selected from the amino acid sequences of SEQ ID NOS: 309-340 or SEQ ID NOS: 341-348, or polypeptide having an amino acid sequence at least 85%, 90%, 95%, 97%, or 99% identical thereto. In some embodiments, the polycistronic nucleic acid insert encodes a polypeptide comprising an amino acid selected from the amino acid sequences of SEQ ID NO: 309, or polypeptide having an amino acid sequence at least 85%, 90%, 95%, 97%, or 99% identical thereto. In some embodiments, the polycistronic nucleic acid insert encodes a polypeptide comprising an amino acid selected from the amino acid sequences of SEQ ID NO: 310, or polypeptide having an amino acid sequence at least 85%, 90%, 95%, 97%, or 99% identical thereto. In some embodiments, the polycistronic nucleic acid insert encodes a polypeptide comprising an amino acid selected from the amino acid sequences of SEQ ID NO: 3110, or polypeptide having an amino acid sequence at least 85%, 90%, 95%, 97%, or 99% identical thereto. In some embodiments, the polycistronic nucleic acid insert encodes a polypeptide comprising an amino acid selected from the amino acid sequences of SEQ ID NO: 312, or polypeptide having an amino acid sequence at least 85%, 90%, 95%, 97%, or 99% identical thereto. In some embodiments, the polycistronic nucleic acid insert encodes a polypeptide comprising an amino acid selected from the amino acid sequences of SEQ ID NO: 313, or polypeptide having an amino acid sequence at least 85%, 90%, 95%, 97%, or 99% identical thereto. In some embodiments, the polycistronic nucleic acid insert encodes a polypeptide comprising an amino acid selected from the amino acid sequences of SEQ ID NO: 314, or polypeptide having an amino acid sequence at least 85%, 90%, 95%, 97%, or 99% identical thereto. In some embodiments, the polycistronic nucleic acid insert encodes a polypeptide comprising an amino acid selected from the amino acid sequences of SEQ ID NO: 315, or polypeptide having an amino acid sequence at least 85%, 90%, 95%, 97%, or 99% identical thereto. In some embodiments, the polycistronic nucleic acid insert encodes a polypeptide comprising an amino acid selected from the amino acid sequences of SEQ ID NO: 316, or polypeptide having an amino acid sequence at least 85%, 90%, 95%, 97%, or 99% identical thereto. In some embodiments, the polycistronic nucleic acid insert encodes a polypeptide comprising an amino acid selected from the amino acid sequences of SEQ ID NO: 317, or polypeptide having an amino acid sequence at least 85%, 90%, 95%, 97%, or 99% identical thereto. In some embodiments, the polycistronic nucleic acid insert encodes a polypeptide comprising an amino acid selected from the amino acid sequences of SEQ ID NO: 318, or polypeptide having an amino acid sequence at least 85%, 90%, 95%, 97%, or 99% identical thereto. In some embodiments, the polycistronic nucleic acid insert encodes a polypeptide comprising an amino acid selected from the amino acid sequences of SEQ ID NO: 319, or polypeptide having an amino acid sequence at least 85%, 90%, 95%, 97%, or 99% identical thereto. In some embodiments, the polycistronic nucleic acid insert encodes a polypeptide comprising an amino acid selected from the amino acid sequences of SEQ ID NO: 320, or polypeptide having an amino acid sequence at least 85%, 90%, 95%, 97%, or 99% identical thereto. In some embodiments, the polycistronic nucleic acid insert encodes a polypeptide comprising an amino acid selected from the amino acid sequences of SEQ ID NO: 321, or polypeptide having an amino acid sequence at least 85%, 90%, 95%, 97%, or 99% identical thereto. In some embodiments, the polycistronic nucleic acid insert encodes a polypeptide comprising an amino acid selected from the amino acid sequences of SEQ ID NO: 322, or polypeptide having an amino acid sequence at least 85%, 90%, 95%, 97%, or 99% identical thereto. In some embodiments, the polycistronic nucleic acid insert encodes a polypeptide comprising an amino acid selected from the amino acid sequences of SEQ ID NO: 323, or polypeptide having an amino acid sequence at least 85%, 90%, 95%, 97%, or 99% identical thereto. In some embodiments, the polycistronic nucleic acid insert encodes a polypeptide comprising an amino acid selected from the amino acid sequences of SEQ ID NO: 324, or polypeptide having an amino acid sequence at least 85%, 90%, 95%, 97%, or 99% identical thereto. In some embodiments, the polycistronic nucleic acid insert encodes a polypeptide comprising an amino acid selected from the amino acid sequences of SEQ ID NO: 325, or polypeptide having an amino acid sequence at least 85%, 90%, 95%, 97%, or 99% identical thereto. In some embodiments, the polycistronic nucleic acid insert encodes a polypeptide comprising an amino acid selected from the amino acid sequences of SEQ ID NO: 326, or polypeptide having an amino acid sequence at least 85%, 90%, 95%, 97%, or 99% identical thereto. In some embodiments, the polycistronic nucleic acid insert encodes a polypeptide comprising an amino acid selected from the amino acid sequences of SEQ ID NO: 327, or polypeptide having an amino acid sequence at least 85%, 90%, 95%, 97%, or 99% identical thereto. In some embodiments, the polycistronic nucleic acid insert encodes a polypeptide comprising an amino acid selected from the amino acid sequences of SEQ ID NO: 328, or polypeptide having an amino acid sequence at least 85%, 90%, 95%, 97%, or 99% identical thereto. In some embodiments, the polycistronic nucleic acid insert encodes a polypeptide comprising an amino acid selected from the amino acid sequences of SEQ ID NO: 329, or polypeptide having an amino acid sequence at least 85%, 90%, 95%, 97%, or 99% identical thereto. In some embodiments, the polycistronic nucleic acid insert encodes a polypeptide comprising an amino acid selected from the amino acid sequences of SEQ ID NO: 330, or polypeptide having an amino acid sequence at least 85%, 90%, 95%, 97%, or 99% identical thereto. In some embodiments, the polycistronic nucleic acid insert encodes a polypeptide comprising an amino acid selected from the amino acid sequences of SEQ ID NO: 331, or polypeptide having an amino acid sequence at least 85%, 90%, 95%, 97%, or 99% identical thereto. In some embodiments, the polycistronic nucleic acid insert encodes a polypeptide comprising an amino acid selected from the amino acid sequences of SEQ ID NO: 332, or polypeptide having an amino acid sequence at least 85%, 90%, 95%, 97%, or 99% identical thereto. In some embodiments, the polycistronic nucleic acid insert encodes a polypeptide comprising an amino acid selected from the amino acid sequences of SEQ ID NO: 333, or polypeptide having an amino acid sequence at least 85%, 90%, 95%, 97%, or 99% identical thereto. In some embodiments, the polycistronic nucleic acid insert encodes a polypeptide comprising an amino acid selected from the amino acid sequences of SEQ ID NO: 334, or polypeptide having an amino acid sequence at least 85%, 90%, 95%, 97%, or 99% identical thereto. In some embodiments, the polycistronic nucleic acid insert encodes a polypeptide comprising an amino acid selected from the amino acid sequences of SEQ ID NO: 335, or polypeptide having an amino acid sequence at least 85%, 90%, 95%, 97%, or 99% identical thereto. In some embodiments, the polycistronic nucleic acid insert encodes a polypeptide comprising an amino acid selected from the amino acid sequences of SEQ ID NO: 336, or polypeptide having an amino acid sequence at least 85%, 90%, 95%, 97%, or 99% identical thereto. In some embodiments, the polycistronic nucleic acid insert encodes a polypeptide comprising an amino acid selected from the amino acid sequences of SEQ ID NO: 337, or polypeptide having an amino acid sequence at least 85%, 90%, 95%, 97%, or 99% identical thereto. In some embodiments, the polycistronic nucleic acid insert encodes a polypeptide comprising an amino acid selected from the amino acid sequences of SEQ ID NO: 338, or polypeptide having an amino acid sequence at least 85%, 90%, 95%, 97%, or 99% identical thereto. In some embodiments, the polycistronic nucleic acid insert encodes a polypeptide comprising an amino acid selected from the amino acid sequences of SEQ ID NO: 339, or polypeptide having an amino acid sequence at least 85%, 90%, 95%, 97%, or 99% identical thereto. In some embodiments, the polycistronic nucleic acid insert encodes a polypeptide comprising an amino acid selected from the amino acid sequences of SEQ ID NO: 340, or polypeptide having an amino acid sequence at least 85%, 90%, 95%, 97%, or 99% identical thereto. In some embodiments, the polycistronic nucleic acid insert encodes a polypeptide comprising an amino acid selected from the amino acid sequences of SEQ ID NO: 341, or polypeptide having an amino acid sequence at least 85%, 90%, 95%, 97%, or 99% identical thereto. In some embodiments, the polycistronic nucleic acid insert encodes a polypeptide comprising an amino acid selected from the amino acid sequences of SEQ ID NO: 342, or polypeptide having an amino acid sequence at least 85%, 90%, 95%, 97%, or 99% identical thereto. In some embodiments, the polycistronic nucleic acid insert encodes a polypeptide comprising an amino acid selected from the amino acid sequences of SEQ ID NO: 343, or polypeptide having an amino acid sequence at least 85%, 90%, 95%, 97%, or 99% identical thereto. In some embodiments, the polycistronic nucleic acid insert encodes a polypeptide comprising an amino acid selected from the amino acid sequences of SEQ ID NO: 344, or polypeptide having an amino acid sequence at least 85%, 90%, 95%, 97%, or 99% identical thereto. In some embodiments, the polycistronic nucleic acid insert encodes a polypeptide comprising an amino acid selected from the amino acid sequences of SEQ ID NO: 345, or polypeptide having an amino acid sequence at least 85%, 90%, 95%, 97%, or 99% identical thereto. In some embodiments, the polycistronic nucleic acid insert encodes a polypeptide comprising an amino acid selected from the amino acid sequences of SEQ ID NO: 346, or polypeptide having an amino acid sequence at least 85%, 90%, 95%, 97%, or 99% identical thereto. In some embodiments, the polycistronic nucleic acid insert encodes a polypeptide comprising an amino acid selected from the amino acid sequences of SEQ ID NO: 347, or polypeptide having an amino acid sequence at least 85%, 90%, 95%, 97%, or 99% identical thereto. In some embodiments, the polycistronic nucleic acid insert encodes a polypeptide comprising an amino acid selected from the amino acid sequences of SEQ ID NO: 348, or polypeptide having an amino acid sequence at least 8500, 900%, 9500, 9700 or 9900 identical thereto.

TABLE 8
rMVA Viral Vectors

| SEQ ID | Sequence | |
|---|---|---|
| NO: | Description | Encoded Polypeptide |
| 309 | (M)(tPA + LD01 + | (M)(DAMKRGLCCVLLLCGAVFVSPSQEIHARFRRGARCRRTSTGQISTL |
| | RAKR cleavable | RVNITAPLSQRAKRGSGATNFSLLKQAGDVEENPGP)x, |
| | sequence + | wherein x = 2, 3, 4, 5, 6, 7, 8, 9, 10, or more. |
| | 2A/2A-like | |
| | cleavage | |
| | sequence)x | |
| 310 | (M)(tPA + LD01 + | (M)(DAMKRGLCCVLLLCGAVFVSPSQEIHARFRRGARCRRTSTGQISTL |
| | RRRR cleavable | RVNITAPLSQRRRRGSGATNFSLLKQAGDVEENPGP)x, |
| | sequence + | wherein x = 2, 3, 4, 5, 6, 7, 8, 9, 10, or more. |
| | 2A/2A-like | |
| | cleavage | |
| | sequence)x | |
| 311 | (M)(tPA + LD01 + | (M)(DAMKRGLCCVLLLCGAVFVSPSQEIHARFRRGARCRRTSTGQISTL |
| | RKRR cleavable | RVNITAPLSQRKRRGSGATNFSLLKQAGDVEENPGP)x, |
| | sequence 1 | wherein x = 2, 3, 4, 5, 6, 7, 8, 9, 10, or more. |
| | 2A/2A-like | |
| | cleavage | |
| | sequence)x | |
| 312 | (M)(tPA + LD01 + | (M)(DAMKRGLCCVLLLCGAVFVSPSQEIHARFRRGARCRRTSTGQISTL |
| | RRKR cleavable | RVNITAPLSQRRKRGSGATNFSLLKQAGDVEENPGP)x, |
| | sequence + | wherein x = 2, 3, 4, 5, 6, 7, 8, 9, 10, or more. |
| | 2A/2A-like | |
| | cleavage | |
| | sequence)x | |
| 313 | (M)(tPA + LD10 + | (M)(DAMKRGLCCVLLLCGAVFVSPSQEIHARFRRGARSTGQISTLRVNIT |
| | RAKR cleavable | APLSQRAKRGSGATNFSLLKQAGDVEENPGP)x, |
| | sequence + | wherein x = 2, 3, 4, 5, 6, 7, 8, 9, 10, or more. |
| | 2A/2A-like | |
| | cleavage | |
| | sequence)x | |
| 314 | (M)(tPA + LD10 + | (M)(DAMKRGLCCVLLLCGAVFVSPSQEIHARFRRGARSTGQISTLRVNIT |
| | RRRR cleavable | APLSQRRRRGSGATNFSLLKQAGDVEENPGP)x, |
| | sequence + | wherein x = 2, 3, 4, 5, 6, 7, 8, 9, 10, or more. |
| | 2A/2A-like | |
| | cleavage | |
| | sequence)x | |
| 315 | (M)(tPA + LD10 + | (M)(DAMKRGLCCVLLLCGAVFVSPSQEIHARFRRGARSTGQISTLRVNIT |
| | RKRR cleavable | APLSQRKRRGSGATNFSLLKQAGDVEENPGP)x, |
| | sequence + | wherein x = 2, 3, 4, 5, 6, 7, 8, 9, 10, or more. |
| | 2A/2A-like | |
| | cleavage | |
| | sequence)x | |
| 316 | (M)(tPA + LD10 + | (M)(DAMKRGLCCVLLLCGAVFVSPSQEIHARFRRGARSTGQISTLRVNIT |
| | RRKR cleavable | APLSQRRKRGSGATNFSLLKQAGDVEENPGP)x, |
| | sequence + | wherein x = 2, 3, 4, 5, 6, 7, 8, 9, 10, or more. |
| | 2A/2A-like | |
| | cleavage | |
| | sequence)x | |
| 317 | (M)(tPA + LD01 + | (M)(DAMKRGLCCVLLLCGAVFVSPSQEIHARFRRGARCRRTSTGQISTL |
| | RAKR cleavable | RVNITAPLSQRAKRGSGATNFSLLKQAGDVEENPGP)x(DAMKRGLCCV |
| | sequence + | LLLCGAVFVSPSQEIHARFRRGARCRRTSTGQISTLRVNITAPLSQ), |
| | 2A/2A-like | wherein x = 2, 3, 4, 5, 6, 7, 8, 9, 10, or more. |
| | cleavage | |
| | sequence)x(tPA + | |
| | LD01) | |
| 318 | (M)(tPA + LD01 + | (M)(DAMKRGLCCVLLLCGAVFVSPSQEIHARFRRGARCRRTSTGQISTL |
| | RRRR cleavable | RVNITAPLSQRRRRGSGATNFSLLKQAGDVEENPGP)x(DAMKRGLCCV |
| | sequence + | LLLCGAVFVSPSQEIHARFRRGARCRRTSTGQISTLRVNITAPLSQ), |
| | 2A/2A-like | wherein x = 2, 3, 4, 5, 6, 7, 8, 9, 10, or more. |
| | cleavage | |
| | sequence)x(tPA + | |
| | LD01) | |
| 319 | (M)(tPA + LD01 + | (M)(DAMKRGLCCVLLLCGAVFVSPSQEIHARFRRGARCRRTSTGQISTL |
| | RKRR cleavable | RVNITAPLSQRKRRGSGATNFSLLKQAGDVEENPGP)x(DAMKRGLCCV |
| | sequence + | LLLCGAVFVSPSQEIHARFRRGARCRRTSTGQISTLRVNITAPLSQ), |
| | 2A/2A-like | wherein x = 2, 3, 4, 5, 6, 7, 8, 9, 10, or more. |
| | cleavage | |
| | sequence)x(tPA + | |
| | LD01) | |
| 320 | (M)(tPA + LD01 + | (M)(DAMKRGLCCVLLLCGAVFVSPSQEIHARFRRGARCRRTSTGQISTL |
| | RRKR cleavable | RVNITAPLSQRRKRGSGATNFSLLKQAGDVEENPGP)x(DAMKRGLCCV |
| | sequence + | LLLCGAVFVSPSQEIHARFRRGARCRRTSTGQISTLRVNITAPLSQ), |
| | 2A/2A-like | wherein x = 2, 3, 4, 5, 6, 7, 8, 9, 10, or more. |
| | cleavage | |
| | sequence)x(tPA + | |
| | LD01) | |
| 321 | (M)(tPA + LD10 + | (M)(DAMKRGLCCVLLLCGAVFVSPSQEIHARFRRGARSTGQISTLRVNIT |
| | RAKR cleavable | APLSQRAKRGSGATNFSLLKQAGDVEENPGP)x(DAMKRGLCCVLLLCG |
| | sequence + | AVFVSPSQEIHARFRRGARSTGQISTLRVNITAPLSQ), |
| | 2A/2A-like | wherein x = 2, 3, 4, 5, 6, 7, 8, 9, 10, or more. |
| | cleavage | |
| | sequence)x(tPA + | |
| | LD10) | |
| 322 | (M)(tPA + LD10 + | (M)(DAMKRGLCCVLLLCGAVFVSPSQEIHARFRRGARSTGQISTLRVNIT |
| | RRRR cleavable | APLSQRRRRGSGATNFSLLKQAGDVEENPGP)x(DAMKRGLCCVLLLCG |
| | sequence + | AVFVSPSQEIHARFRRGARSTGQISTLRVNITAPLSQ), |
| | 2A/2A-like | wherein x = 2, 3, 4, 5, 6, 7, 8, 9, 10, or more. |
| | cleavage | |
| | sequence)x(tPA + | |
| | LD10) | |
| 323 | (M)(tPA + LD10 + | (M)(DAMKRGLCCVLLLCGAVFVSPSQEIHARFRRGARSTGQISTLRVNIT |
| | RKRR cleavable | APLSQRKRRGSGATNFSLLKQAGDVEENPGP)x(DAMKRGLCCVLLLCG |
| | sequence + | AVFVSPSQEIHARFRRGARSTGQISTLRVNITAPLSQ), |
| | 2A/2A-like | wherein x = 2, 3, 4, 5, 6, 7, 8, 9, 10, or more. |
| | cleavage | |
| | sequence)x(tPA + | |
| | LD10) | |
| 324 | (M)(tPA + LD10 + | (M)(DAMKRGLCCVLLLCGAVFVSPSQEIHARFRRGARSTGQISTLRVNIT |
| | RRKR cleavable | APLSQRRKRGSGATNFSLLKQAGDVEENPGP)x(DAMKRGLCCVLLLCG |
| | sequence + | AVFVSPSQEIHARFRRGARSTGQISTLRVNITAPLSQ), |
| | 2A/2A-like | wherein x = 2, 3, 4, 5, 6, 7, 8, 9, 10, or more. |
| | cleavage | |
| | sequence)x(tPA + | |
| | LD10) | |
| 325 | (M)(tPA + LD01 + | MDAMKRGLCCVLLLCGAVFVSPSQEIHARFRRGARCRRTSTGQISTLRV |
| | RAKR cleavable | NITAPLSQRAKRGSGATNFSLLKQAGDVEENPGPDAMKRGLCCVLLLC |
| | sequence + | GAVFVSPSQEIHARFRRGARCRRTSTGQISTLRVNITAPLSQRAKRGSGA |
| | 2A/2A-like | TNFSLLKQAGDVEENPGPDAMKRGLCCVLLLCGAVFVSPSQEIHARFRR |
| | cleavage | GARCRRTSTGQISTLRVNITAPLSQRAKRGSGATNFSLLKQAGDVEENP |
| | sequence)₅ | GPDAMKRGLCCVLLLCGAVFVSPSQEIHARFRRGARCRRTSTGQISTLR |
| | | VNITAPLSQRAKRGSGATNFSLLKQAGDVEENPGPDAMKRGLCCVLLL |
| | | CGAVFVSPSQEIHARFRRGARCRRTSTGQISTLRVNITAPLSQRAKRGSG |
| | | ATNFSLLKQAGDVEENPGP |
| 326 | (M)(tPA + LD01 + | MDAMKRGLCCVLLLCGAVFVSPSQEIHARFRRGARCRRTSTGQISTLRV |
| | RRRR | NITAPLSQRRRRGSGATNFSLLKQAGDVEENPGPDAMKRGLCCVLLLC |
| | cleavable | GAVFVSPSQEIHARFRRGARCRRTSTGQISTLRVNITAPLSQRRRRGSGA |
| | sequence + | TNFSLLKQAGDVEENPGPDAMKRGLCCVLLLCGAVFVSPSQEIHARFRR |
| | 2A/2A-like | GARCRRTSTGQISTLRVNITAPLSQRRRRGSGATNFSLLKQAGDVEENP |
| | cleavage | GPDAMKRGLCCVLLLCGAVFVSPSQEIHARFRRGARCRRTSTGQISTLR |
| | sequence)₅ | VNITAPLSQRRRRGSGATNFSLLKQAGDVEENPGPDAMKRGLCCVLLL |
| | | CGAVFVSPSQEIHARFRRGARCRRTSTGQISTLRVNITAPLSQRRRRGSG |
| | | ATNFSLLKQAGDVEENPGP |
| 327 | (M)(tPA + LD01 + | MDAMKRGLCCVLLLCGAVFVSPSQEIHARFRRGARCRRTSTGQISTLRV |
| | RKRR | NITAPLSQRKRRGSGATNFSLLKQAGDVEENPGPDAMKRGLCCVLLLC |
| | cleavable | GAVFVSPSQEIHARFRRGARCRRTSTGQISTLRVNITAPLSQRKRRGSGA |
| | sequence + | TNFSLLKQAGDVEENPGPDAMKRGLCCVLLLCGAVFVSPSQEIHARFRR |
| | 2A/2A-like | GARCRRTSTGQISTLRVNITAPLSQRKRRGSGATNFSLLKQAGDVEENP |
| | cleavage | GPDAMKRGLCCVLLLCGAVFVSPSQEIHARFRRGARCRRTSTGQISTLR |
| | sequence)₅ | VNITAPLSQRKRRGSGATNFSLLKQAGDVEENPGPDAMKRGLCCVLLL |
| | | CGAVFVSPSQEIHARFRRGARCRRTSTGQISTLRVNITAPLSQRKRRGSG |
| | | ATNFSLLKQAGDVEENPGP |
| 328 | (M)(tPA + LD01 + | MDAMKRGLCCVLLLCGAVFVSPSQEIHARFRRGARCRRTSTGQISTLRV |
| | RRKR | NITAPLSQRRKRGSGATNFSLLKQAGDVEENPGPDAMKRGLCCVLLLC |
| | cleavable | GAVFVSPSQEIHARFRRGARCRRTSTGQISTLRVNITAPLSQRRKRGSGA |
| | sequence + | TNFSLLKQAGDVEENPGPDAMKRGLCCVLLLCGAVFVSPSQEIHARFRR |
| | 2A/2A-like | GARCRRTSTGQISTLRVNITAPLSQRRKRGSGATNFSLLKQAGDVEENP |
| | cleavage | GPDAMKRGLCCVLLLCGAVFVSPSQEIHARFRRGARCRRTSTGQISTLR |
| | sequence)₅ | VNITAPLSQRRKRGSGATNFSLLKQAGDVEENPGPDAMKRGLCCVLLL |
| | | CGAVFVSPSQEIHARFRRGARCRRTSTGQISTLRVNITAPLSQRRKRGSG |
| | | ATNFSLLKQAGDVEENPGP |
| 329 | (M)(tPA + LD01 + | MDAMKRGLCCVLLLCGAVFVSPSQEIHARFRRGARCRRTSTGQISTLRV |
| | RAKR cleavable | NITAPLSQRAKRGSGATNFSLLKQAGDVEENPGPDAMKRGLCCVLLLC |
| | sequence + | GAVFVSPSQEIHARFRRGARCRRTSTGQISTLRVNITAPLSQRAKRGSGA |
| | 2A/2A-like | TNFSLLKQAGDVEENPGPDAMKRGLCCVLLLCGAVFVSPSQEIHARFRR |
| | cleavage | GARCRRTSTGQISTLRVNITAPLSQRAKRGSGATNFSLLKQAGDVEENP |
| | sequence)₄(tPA + | GPDAMKRGLCCVLLLCGAVFVSPSQEIHARFRRGARCRRTSTGQISTLR |
| | LD01) | VNITAPLSQRAKRGSGATNFSLLKQAGDVEENPGPDAMKRGLCCVLLL |
| | | CGAVFVSPSQEIHARFRRGARCRRTSTGQISTLRVNITAPLSQ |
| 330 | (M)(tPA + LD01 + | MDAMKRGLCCVLLLCGAVFVSPSQEIHARFRRGARCRRTSTGQISTLRV |
| | RRRR cleavable | NITAPLSQRRRRGSGATNFSLLKQAGDVEENPGPDAMKRGLCCVLLLC |
| | sequence + | GAVFVSPSQEIHARFRRGARCRRTSTGQISTLRVNITAPLSQRRRRGSGA |
| | 2A/2A-like | TNFSLLKQAGDVEENPGPDAMKRGLCCVLLLCGAVFVSPSQEIHARFRR |
| | cleavage | GARCRRTSTGQISTLRVNITAPLSQRRRRGSGATNFSLLKQAGDVEENP |
| | sequence)₄(tPA + | GPDAMKRGLCCVLLLCGAVFVSPSQEIHARFRRGARCRRTSTGQISTLR |
| | LD01) | VNITAPLSQRRRRGSGATNFSLLKQAGDVEENPGPDAMKRGLCCVLLL |
| | | CGAVFVSPSQEIHARFRRGARCRRTSTGQISTLRVNITAPLSQ |
| 331 | (M)(tPA + LD01 + | MDAMKRGLCCVLLLCGAVFVSPSQEIHARFRRGARCRRTSTGQISTLRV |
| | RKRR cleavable | NITAPLSQRKRRGSGATNFSLLKQAGDVEENPGPDAMKRGLCCVLLLC |
| | sequence + | GAVFVSPSQEIHARFRRGARCRRTSTGQISTLRVNITAPLSQRKRRGSGA |
| | 2A/2A-like | TNFSLLKQAGDVEENPGPDAMKRGLCCVLLLCGAVFVSPSQEIHARFRR |
| | cleavage | GARCRRTSTGQISTLRVNITAPLSQRKRRGSGATNFSLLKQAGDVEENP |
| | sequence)₄(tPA + | GPDAMKRGLCCVLLLCGAVFVSPSQEIHARFRRGARCRRTSTGQISTLR |
| | LD01) | VNITAPLSQRKRRGSGATNFSLLKQAGDVEENPGPDAMKRGLCCVLLL |
| | | CGAVFVSPSQEIHARFRRGARCRRTSTGQISTLRVNITAPLSQ |
| 332 | (M)(tPA + LD01 + | MDAMKRGLCCVLLLCGAVFVSPSQEIHARFRRGARCRRTSTGQISTLRV |
| | RRKR cleavable | NITAPLSQRRKRGSGATNFSLLKQAGDVEENPGPDAMKRGLCCVLLLC |
| | sequence + | GAVFVSPSQEIHARFRRGARCRRTSTGQISTLRVNITAPLSQRRKRGSGA |
| | 2A/2A-like | TNFSLLKQAGDVEENPGPDAMKRGLCCVLLLCGAVFVSPSQEIHARFRR |
| | cleavage | GARCRRTSTGQISTLRVNITAPLSQRRKRGSGATNFSLLKQAGDVEENP |
| | sequence)₄(tPA + | GPDAMKRGLCCVLLLCGAVFVSPSQEIHARFRRGARCRRTSTGQISTLR |
| | LD01) | VNITAPLSQRRKRGSGATNFSLLKQAGDVEENPGPDAMKRGLCCVLLL |
| | | CGAVFVSPSQEIHARFRRGARCRRTSTGQISTLRVNITAPLSQ |
| 333 | (M)(tPA + LD10 + | MDAMKRGLCCVLLLCGAVFVSPSQEIHARFRRGARSTGQISTLRVNITA |
| | RAKR cleavable | PLSQRAKRGSGATNFSLLKQAGDVEENPGPDAMKRGLCCVLLLCGAVF |
| | sequence + | VSPSQEIHARFRRGARSTGQISTLRVNITAPLSQRAKRGSGATNFSLLKQ |
| | 2A/2A-like | AGDVEENPGPDAMKRGLCCVLLLCGAVFVSPSQEIHARFRRGARSTGQI |
| | cleavage | STLRVNITAPLSQRAKRGSGATNFSLLKQAGDVEENPGPDAMKRGLCC |
| | sequence)₅ | VLLLCGAVFVSPSQEIHARFRRGARSTGQISTLRVNITAPLSQRAKRGSG |
| | | ATNFSLLKQAGDVEENPGPDAMKRGLCCVLLLCGAVFVSPSQEIHARF |
| | | RRGARSTGQISTLRVNITAPLSQRAKRGSGATNFSLLKQAGDVEENPGP |
| 334 | (M)(tPA + LD10 + | MDAMKRGLCCVLLLCGAVFVSPSQEIHARFRRGARSTGQISTLRVNITA |
| | RRRR cleavable | PLSQRRRRGSGATNFSLLKQAGDVEENPGPDAMKRGLCCVLLLCGAVF |
| | sequence + | VSPSQEIHARFRRGARSTGQISTLRVNITAPLSQRRRRGSGATNFSLLKQ |
| | 2A/2A-like | AGDVEENPGPDAMKRGLCCVLLLCGAVFVSPSQEIHARFRRGARSTGQI |
| | cleavage | STLRVNITAPLSQRRRRGSGATNFSLLKQAGDVEENPGPDAMKRGLCC |
| | sequence)₅ | VLLLCGAVFVSPSQEIHARFRRGARSTGQISTLRVNITAPLSQRRRRGSG |
| | | ATNFSLLKQAGDVEENPGPDAMKRGLCCVLLLCGAVFVSPSQEIHARF |
| | | RRGARSTGQISTLRVNITAPLSQRRRRGSGATNFSLLKQAGDVEENPGP |
| 335 | (M)(tPA + LD10 + | MDAMKRGLCCVLLLCGAVFVSPSQEIHARFRRGARSTGQISTLRVNITA |
| | RKRR cleavable | PLSQRKRRGSGATNFSLLKQAGDVEENPGPDAMKRGLCCVLLLCGAVF |
| | sequence + | VSPSQEIHARFRRGARSTGQISTLRVNITAPLSQRKRRGSGATNFSLLKQ |
| | 2A/2A-like | AGDVEENPGPDAMKRGLCCVLLLCGAVFVSPSQEIHARFRRGARSTGQI |
| | cleavage | STLRVNITAPLSQRKRRGSGATNFSLLKQAGDVEENPGPDAMKRGLCC |
| | sequence)₅ | VLLLCGAVFVSPSQEIHARFRRGARSTGQISTLRVNITAPLSQRKRRGSG |
| | | ATNFSLLKQAGDVEENPGPDAMKRGLCCVLLLCGAVFVSPSQEIHARF |
| | | RRGARSTGQISTLRVNITAPLSQRKRRGSGATNFSLLKQAGDVEENPGP |
| 336 | (M)(tPA + LD10 + | MDAMKRGLCCVLLLCGAVFVSPSQEIHARFRRGARSTGQISTLRVNITA |
| | RRKR cleavable | PLSQRRKRGSGATNFSLLKQAGDVEENPGPDAMKRGLCCVLLLCGAVF |
| | sequence + | VSPSQEIHARFRRGARSTGQISTLRVNITAPLSQRRKRGSGATNFSLLKQ |
| | 2A/2A-like | AGDVEENPGPDAMKRGLCCVLLLCGAVFVSPSQEIHARFRRGARSTGQI |
| | cleavage | STLRVNITAPLSQRRKRGSGATNFSLLKQAGDVEENPGPDAMKRGLCC |
| | sequence)₅ | VLLLCGAVFVSPSQEIHARFRRGARSTGQISTLRVNITAPLSQRRKRGSG |
| | | ATNFSLLKQAGDVEENPGPDAMKRGLCCVLLLCGAVFVSPSQEIHARF |
| | | RRGARSTGQISTLRVNITAPLSQRRKRGSGATNFSLLKQAGDVEENPGP |
| 337 | (M)(tPA + LD10 + | MDAMKRGLCCVLLLCGAVFVSPSQEIHARFRRGARSTGQISTLRVNITA |
| | RAKR cleavable | PLSQRAKRGSGATNFSLLKQAGDVEENPGPDAMKRGLCCVLLLCGAVF |
| | sequence + | VSPSQEIHARFRRGARSTGQISTLRVNITAPLSQRAKRGSGATNFSLLKQ |
| | 2A/2A-like | AGDVEENPGPDAMKRGLCCVLLLCGAVFVSPSQEIHARFRRGARSTGQI |
| | cleavage | STLRVNITAPLSQRAKRGSGATNFSLLKQAGDVEENPGPDAMKRGLCC |
| | sequence)₄(tPA + | VLLLCGAVFVSPSQEIHARFRRGARSTGQISTLRVNITAPLSQRAKRGSG |
| | LD10) | ATNFSLLKQAGDVEENPGPDAMKRGLCCVLLLCGAVFVSPSQEIHARF |
| | | RRGARSTGQISTLRVNITAPLSQ |
| 338 | (M)(tPA + LD10 + | MDAMKRGLCCVLLLCGAVFVSPSQEIHARFRRGARSTGQISTLRVNITA |
| | RRRR cleavable | PLSQRRRRGSGATNFSLLKQAGDVEENPGPDAMKRGLCCVLLLCGAVF |
| | sequence + | VSPSQEIHARFRRGARSTGQISTLRVNITAPLSQRRRRGSGATNFSLLKQ |
| | 2A/2A-like | AGDVEENPGPDAMKRGLCCVLLLCGAVFVSPSQEIHARFRRGARSTGQI |
| | cleavage | STLRVNITAPLSQRRRRGSGATNFSLLKQAGDVEENPGPDAMKRGLCC |
| | sequence)₄(tPA + | VLLLCGAVFVSPSQEIHARFRRGARSTGQISTLRVNITAPLSQRRRRGSG |
| | LD10) | ATNFSLLKQAGDVEENPGPDAMKRGLCCVLLLCGAVFVSPSQEIHARF |
| | | RRGARSTGQISTLRVNITAPLSQ |
| 339 | (M)(PA + LD10 + | MDAMKRGLCCVLLLCGAVFVSPSQEIHARFRRGARSTGQISTLRVNITA |
| | RKRR cleavable | PLSQRKRRGSGATNFSLLKQAGDVEENPGPDAMKRGLCCVLLLCGAVF |
| | sequence + | VSPSQEIHARFRRGARSTGQISTLRVNITAPLSQRKRRGSGATNFSLLKQ |
| | 2A/2A-like | AGDVEENPGPDAMKRGLCCVLLLCGAVFVSPSQEIHARFRRGARSTGQI |
| | cleavage | STLRVNITAPLSQRKRRGSGATNFSLLKQAGDVEENPGPDAMKRGLCC |
| | sequence)₄(tPA + | VLLLCGAVFVSPSQEIHARFRRGARSTGQISTLRVNITAPLSQRKRRGSG |
| | LD10) | ATNFSLLKQAGDVEENPGPDAMKRGLCCVLLLCGAVFVSPSQEIHARF |
| | | RRGARSTGQISTLRVNITAPLSQ |
| 340 | (M)(tPA + LD10 + | MDAMKRGLCCVLLLCGAVFVSPSQEIHARFRRGARSTGQISTLRVNITA |
| | RRKR cleavable | PLSQRRKRGSGATNFSLLKQAGDVEENPGPDAMKRGLCCVLLLCGAVF |
| | sequence + | VSPSQEIHARFRRGARSTGQISTLRVNITAPLSQRRKRGSGATNFSLLKQ |
| | 2A/2A-like | AGDVEENPGPDAMKRGLCCVLLLCGAVFVSPSQEIHARFRRGARSTGQI |
| | cleavage | STLRVNITAPLSQRRKRGSGATNFSLLKQAGDVEENPGPDAMKRGLCC |
| | sequence)₄(tPA + | VLLLCGAVFVSPSQEIHARFRRGARSTGQISTLRVNITAPLSQRRKRGSG |
| | LD10) | ATNFSLLKQAGDVEENPGPDAMKRGLCCVLLLCGAVFVSPSQEIHARF |
| | | RRGARSTGQISTLRVNITAPLSQ |
| 341 | (M)(tPA + LD01 + | (M)(DAMKRGLCCVLLLCGAVFVSPSQEIHARFRRGARCRRTSTGQISTL |
| | RKKR cleavable | RVNITAPLSQRKKRGSGATNFSLLKQAGDVEENPGP)x, |
| | sequence + | wherein x = 2, 3, 4, 5, 6, 7, 8, 9, 10, or more. |
| | 2A/2A-like | |
| | cleavage | |
| | sequence)x | |
| 342 | (M)(tPA + LD10 + | (M)(DAMKRGLCCVLLLCGAVFVSPSQEIHARFRRGARSTGQISTLRVNIT |
| | RKKR cleavable | APLSQRKKRGSGATNFSLLKQAGDVEENPGP)x, |
| | sequence + | wherein x = 2, 3, 4, 5, 6, 7, 8, 9, 10, or more. |
| | 2A/2A-like | |
| | cleavage | |
| | sequence)x | |
| 343 | (M)(tPA + LD01 + | (M)(DAMKRGLCCVLLLCGAVFVSPSQEIHARFRRGARCRRTSTGQISTL |
| | RKKR cleavable | RVNITAPLSQRKKRGSGATNFSLLKQAGDVEENPGP)x(DAMKRGLCCV |
| | sequence + | LLLCGAVFVSPSQEIHARFRRGARCRRTSTGQISTLRVNITAPLSQ), |
| | 2A/2A-like | wherein x = 2, 3, 4, 5, 6, 7, 8, 9, 10, or more. |
| | cleavage | |
| | sequence)x(tPA + | |
| | LD01) | |
| 344 | (M)(tPA + LD10 + | (M)(DAMKRGLCCVLLLCGAVFVSPSQEIHARFRRGARSTGQISTLRVNIT |
| | RKKR cleavable | APLSQRKKRGSGATNFSLLKQAGDVEENPGP)x(DAMKRGLCCVLLLCG |
| | sequence + | AVFVSPSQEIHARFRRGARSTGQISTLRVNITAPLSQ), |
| | 2A/2A-like | wherein x = 2, 3, 4, 5, 6, 7, 8, 9, 10, or more. |
| | cleavage | |
| | sequence)x(tPA + | |
| | LD10) | |
| 345 | (M)(tPA + LD01 + | MDAMKRGLCCVLLLCGAVFVSPSQEIHARFRRGARCRRTSTGQISTLRV |
| | RKKR cleavable | NITAPLSQRKKRGSGATNFSLLKQAGDVEENPGPDAMKRGLCCVLLLC |
| | sequence + | GAVFVSPSQEIHARFRRGARCRRTSTGQISTLRVNITAPLSQRKKRGSGA |
| | 2A/2A-like | TNFSLLKQAGDVEENPGPDAMKRGLCCVLLLCGAVFVSPSQEIHARFRR |
| | cleavage | GARCRRTSTGQISTLRVNITAPLSQRKKRGSGATNFSLLKQAGDVEENP |
| | sequence)₅ | GPDAMKRGLCCVLLLCGAVFVSPSQEIHARFRRGARCRRTSTGQISTLR |
| | | VNITAPLSQRKKRGSGATNFSLLKQAGDVEENPGPDAMKRGLCCVLLL |
| | | CGAVFVSPSQEIHARFRRGARCRRTSTGQISTLRVNITAPLSQRKKRGSG |
| | | ATNFSLLKQAGDVEENPGP |
| 346 | (M)(tPA + LD10 + | MDAMKRGLCCVLLLCGAVFVSPSQEIHARFRRGARSTGQISTLRVNITA |
| | RKKR cleavable | PLSQRKKRGSGATNFSLLKQAGDVEENPGPDAMKRGLCCVLLLCGAVF |
| | sequence + | VSPSQEIHARFRRGARSTGQISTLRVNITAPLSQRKKRGSGATNFSLLKQ |
| | 2A/2A-like | AGDVEENPGPDAMKRGLCCVLLLCGAVFVSPSQEIHARFRRGARSTGQI |
| | cleavage | STLRVNITAPLSQRKKRGSGATNFSLLKQAGDVEENPGPDAMKRGLCC |
| | sequence)₅ | VLLLCGAVFVSPSQEIHARFRRGARSTGQISTLRVNITAPLSQRKKRGSG |
| | | ATNFSLLKQAGDVEENPGPDAMKRGLCCVLLLCGAVFVSPSQEIHARF |
| | | RRGARSTGQISTLRVNITAPLSQRKKRGSGATNFSLLKQAGDVEENPGP |
| 347 | (M)(tPA + LD01 + | MDAMKRGLCCVLLLCGAVFVSPSQEIHARFRRGARCRRTSTGQISTLRV |
| | RKKR cleavable | NITAPLSQRKKRGSGATNFSLLKQAGDVEENPGPDAMKRGLCCVLLLC |
| | sequence + | GAVFVSPSQEIHARFRRGARCRRTSTGQISTLRVNITAPLSQRKKRGSGA |
| | 2A/2A-like | TNFSLLKQAGDVEENPGPDAMKRGLCCVLLLCGAVFVSPSQEIHARFRR |
| | cleavage | GARCRRTSTGQISTLRVNITAPLSQRKKRGSGATNFSLLKQAGDVEENP |
| | sequence)₄(tPA + | GPDAMKRGLCCVLLLCGAVFVSPSQEIHARFRRGARCRRTSTGQISTLR |
| | LD01) | VNITAPLSQRKKRGSGATNFSLLKQAGDVEENPGPDAMKRGLCCVLLL |
| | | CGAVFVSPSQEIHARFRRGARCRRTSTGQISTLRVNITAPLSQ |
| 348 | (M)(PA + LD10 + | MDAMKRGLCCVLLLCGAVFVSPSQEIHARFRRGARSTGQISTLRVNITA |
| | RKKR cleavable | PLSQRKKRGSGATNFSLLKQAGDVEENPGPDAMKRGLCCVLLLCGAVF |
| | sequence + | VSPSQEIHARFRRGARSTGQISTLRVNITAPLSQRKKRGSGATNFSLLKQ |
| | 2A/2A-like | AGDVEENPGPDAMKRGLCCVLLLCGAVFVSPSQEIHARFRRGARSTGQI |
| | cleavage | STLRVNITAPLSQRKKRGSGATNFSLLKQAGDVEENPGPDAMKRGLCC |
| | sequence)₄(tPA + | VLLLCGAVFVSPSQEIHARFRRGARSTGQISTLRVNITAPLSQRKKRGSG |
| | LD10) | ATNFSLLKQAGDVEENPGPDAMKRGLCCVLLLCGAVFVSPSQEIHARF |
| | | RRGARSTGQISTLRVNITAPLSQ |

As provided herein, the polycistronic nucleic acid insert encoding the immune checkpoint inhibitor polypeptide as described herein can be inserted into the MVA genome at any suitable location, for example, a natural deletion site, a modified natural deletion site, in a non-essential MVA gene, for example the MVA thymidine kinase locus, or in an intergenic region between essential or non-essential MVA genes. Suitable insertion sites have been described, for example, in U.S. Pat. Nos. 6,998,252, 9,133,478, Ober et al., Immunogenicity and safety of defective vaccinia virus lister: comparison with modified vaccinia virus Ankara. J. Virol., August 2002 (pg. 7713-7723), U.S. Pat. Nos. 9,133,480, 8,288,125, each of which is incorporated herein by reference.

In some embodiments, the polycistronic nucleic acid insert encoding the immune checkpoint inhibitor polypeptide as described herein is inserted into a natural deletion site, for example a deletion site selected from the natural deletion sites I, II, III, IV, V or VI, a modified natural deletion site, for example the restructured and modified deletion III site between the MVA genes A50R and B1R (see, e.g., U.S. Pat. No. 9,133,480), between non-essential MVA genes, between essential MVA genes, for example I8R and G1L or A5R and A6L or other suitable insertion site, in a non-essential locus, for example in the MVA TK locus, or a combination thereof.

In alternative embodiments, the rMVA viral vectors of the present invention, in addition to the ability to express multiple immune checkpoint inhibitor peptides, may further be constructed to encode and express one or more antigen peptides. The one or more antigenic peptides can be encoded on one or more separate nucleic acid inserts, or in an alternative embodiment, the one or more antigenic peptides are encoded on the same polycistronic nucleic acid insert as the multiple immune checkpoint inhibitor peptides.

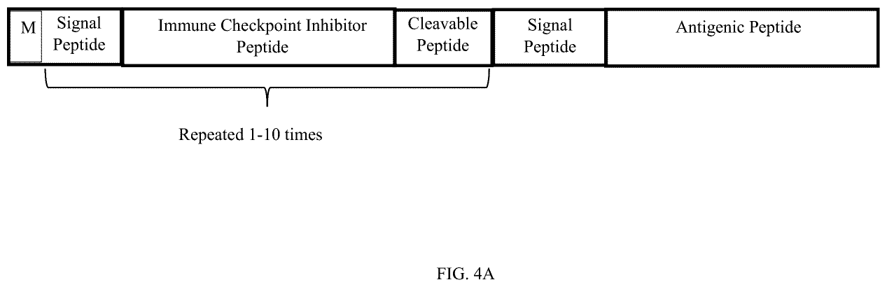

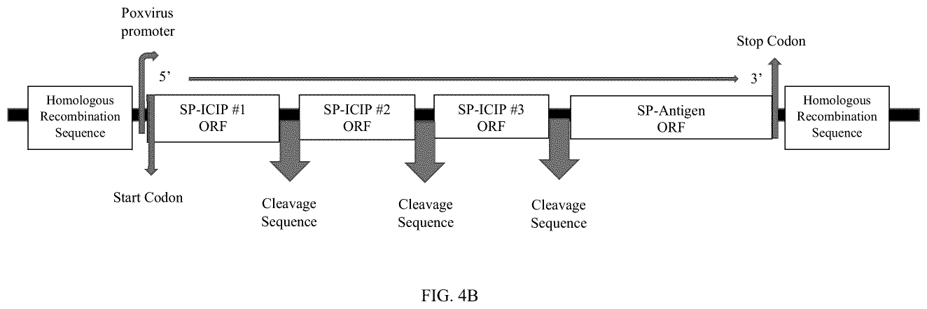

In some embodiments, provided herein is an rMVA viral vector comprising a heterologous polycistronic nucleic acid insert encoding a polypeptide wherein the polypeptide comprises ((M)(Secretion Signal Peptide-Immune Checkpoint Inhibitor Peptide-Cleavable Peptide)ₓ(Antigenic Peptide)), wherein x=1, 2, 3, 4, 5, 6, 7, 8, 9, 10, or more than 10, and wherein M=methionine. In some embodiments, the antigenic peptide is contained in a chimeric polypeptide comprising a secretion signal peptide fused to the N-terminus of the antigenic peptide, for example ((M)(Secretion Signal Peptide-Immune Checkpoint Inhibitor Peptide-Cleavable Peptide)ₓ(Secretion Signal Peptide-Antigenic Peptide)), wherein x=1, 2, 3, 4, 5, 6, 7, 8, 9, 10, or more than 10, and wherein M=methionine (see, e.g., FIGS. 4A-4B). In some embodiments, the antigenic peptide is also provided so that 2 or more antigenic peptides are encoded in the polycistronic nucleic acid insert, with each chimeric polypeptide separated by a cleavable peptide described herein. In some embodiments, the antigenic peptide is contained in a chimeric polypeptide comprising a secretion signal peptide fused to the N-terminus of the antigenic peptide, and a cleavable peptide fused to the C-terminus of the antigenic peptide, for example ((M)(Secretion Signal Peptide-Immune Checkpoint Inhibitor Peptide-Cleavable Peptide)ₓ(Secretion Signal Peptide-Antigenic Peptide-Cleavable Peptide)_{y}), wherein x=1, 2, 3, 4, 5, 6, 7, 8, 9, 10, or more than 10, wherein y=1, 2, 3, 4, 5, 6, 7, 8, 9, 10, or more than 10, and wherein M=methionine. In some embodiments, the antigen containing chimeric polypeptide fused to the C-terminus of the last antigen containing chimeric polypeptide does not include a cleavable sequence, for example ((M)(Secretion Signal Peptide-Immune Checkpoint Inhibitor Peptide-Cleavable Peptide)x(Secretion Signal Peptide-Antigenic Peptide-Cleavable Peptide)ₓ(Secretion Signal Peptide-Antigenic Peptide)), wherein x=1, 2, 3, 4, 5, 6, 7, 8, 9, 10, or more than 10, and wherein M=methionine. In some embodiments, the antigenic peptide contained in the chimeric polypeptide comprising a secretion signal peptide fused to the N-terminus of the antigenic peptide, and a cleavable peptide fused to the C-terminus of the antigenic peptide can be oriented in the polycistronic nucleic acid insert so that the antigen containing chimeric polypeptide encoding nucleic acid is located 5′ of the immune checkpoint inhibitor peptide containing chimeric polypeptides, for example ((M)(Secretion Signal Peptide-Antigenic Peptide-Cleavable Peptide)_{y}(Secretion Signal Peptide-Immune Checkpoint Inhibitor Peptide-Cleavable Peptide)ₓ) or, alternatively ((M)(Secretion Signal Peptide-Antigenic Peptide-Cleavable Peptide)_{y}(Secretion Signal Peptide-Immune Checkpoint Inhibitor Peptide-Cleavable Peptide)ₓ(Secretion Signal Peptide-Immune Checkpoint Inhibitor Peptide)), wherein y=1, 2, 3, 4, 5, 6, 7, 8, 9, 10, or more than 10, wherein x=1, 2, 3, 4, 5, 6, 7, 8, 9, 10, or more than 10, and wherein M=methionine.

In some embodiments, the antigenic peptide is a peptide derived from an infectious agent, for example a virus, bacteria, parasite, fungus, or toxoid, or alternatively, a tumor associated antigen, or an antigen derived from an agent described in the section titled Antigenic Targets above, which is expressly incorporated into this section.

In some embodiments, the polycistronic nucleic acid insert encodes a polypeptide comprising an antigenic amino acid of Table 9 below, or polypeptide having an amino acid sequence at least 85%, 90%, 95%, 97%, or 99% identical thereto. In some embodiments, the polycistronic nucleic acid insert encodes a antigen comprising an amino acid derived from an amino acid sequence selected from SEQ ID NOS: 349-396, 398, 400, 402, or 405, or a fragment thereof, or a polypeptide having an amino acid sequence at least 85%, 90%, 95%, 97%, or 99%

TABLE 9
Antigenic Peptides

| SEQ ID | | |
|---|---|---|
| NO: | Antigen | Amino Acid Sequence |
| 349 | Human Mucin 1 | TPGTQSPFFLLLLLTVLTVVTGSGHASSTPGGEKETSATQRSSVPSSTEK |
| | | NAVSMTSSVLSSHSPGSGSSTTQGQDVTLAPATEPASGSAATWGQDVT |
| | | SVPVTRPALGSTTPPAHDVTSAPDNKPAPGSTAPPAHGVTSAPDTRPAP |
| | | GSTAPPAHGVTSAPDTRPAPGSTAPPAHGVTSAPDTRPAPGSTAPPAHG |
| | | VTSAPDTRPAPGSTAPPAHGVTSAPDNRPALGSTAPPVHNVTSASGSAS |
| | | GSASTLVHNGTSARATTTPASKSTPFSIPSHHSDTPTTLASHSTKTDASS |
| | | THHSTVPPLTSSNHSTSPQLSTGVSFFFLSFHISNLQFNSSLEDPSTDYYQ |
| | | ELQRDISEMFLQIYKQGGFLGLSNIKFRPGSVVVQLTLAFREGTINVHD |
| | | VETQFNQYKTEAASRYNLTISDVSVSDVPFPFSAQSGAGV |
| 350 | Cyclin B1 | LPGMALRVTRNSKINAENKAKINMAGAKRVPTAPAATSKPGLRPRTA |
| | | LGDIGNKVSEQLQAKMPMKKEAKPSATGKVIDKKLPKPLEKVPMLVP |
| | | VPVSEPVPEPEPEPEPEPVKEEKLSPEPILVDTASPSPMETSGCAPAEEDL |
| | | CQAFSDVILAVNDVDAEDGADPNLCSEYVKDIYAYLRQLEEEQAVRP |
| | | KYLLGREVTGNMRAILIDWLVQVQMKFRLLQETMYMTVSIIDRFMQN |
| | | NCVPKKMLQLVGVTAMFIASKYEEMYPPEIGDFAFVTDNTYTKHQIRQ |
| | | MEMKILRALNFGLGRPLPLHFLRRASKIGEVDVEQHTLAKYLMELTML |
| | | DYDMVHFPPSQIAAGAFCLALKILDNGEWTPTLQHYLSYTEESLLPVM |
| | | QHLAKNVVMVNQGLTKHMTVKNKYATSKHAKISTLPQLNSALVQDL |
| | | AKAVAKV |
| 351 | HBV PresS2 | QWNSTTFHQTLQDPRVRGLYFPAGGSSSGAVNPVPTTASPLSSIFSRIG |
| | | DPALNMENITSGFLGPLLVLQAGFFLLTRILTIPQSLDSWWTSLNFLGG |
| | | TTVCLGQNSQSPTSNHSPTSCPPTCPGYRWMCLRRFIIFLFILLLCLIFLL |
| | | VLLDYQGMLPVCPLIPGSSTTSTGPCRTCMTTAQGTSMYPSCCCTKPS |
| | | DGNCTCIPIPSSWAFGKFLWEWASARFSWLSLLVPFVQWFVGLSPTVW |
| | | LSVIWMMWYWGPSLYSILSPFLPLLPIFFCLWVYI |
| 352 | HBV | HLFHLCLIISCSCPTVQASKLCLGWLWGMDIDPYKEFGATVELLSFLPS |
| | PreCore/Core | DFFPSVRDLLDTASALYREALESPEHCSPHHTALRQAILCWGELMTLA |
| | | TWVGVNLEDPASRDLVVSYVNTNMGLKFRQLLWFHISCLTFGRETVIE |
| | | YLVSFGVWIRTPPAYRPPNAPILSTLPETTVVRRRGRSPRRRTPSPRRRR |
| | | SQSPRRRRSQSREPQC |
| 353 | HBV Truncated | AARLCCQLDPARDVLCLRPVGAESCGRPFSGSLETLSSPSPSAVPTDHG |
| | X Gene Product | AHLSLRGLPAMSTTDLEAYFKDCLFKDWEELGEEIRLKVFVLGGCRHK |
| | | LVCAPAPCTFFTSA |
| 354 | HBV PreS, HA | EAKLFVLFCAFTALKAMGTNLSVPNPLGFFPDHQLDPAFGANSNNPD |
| | (chimeric fusion | WDFNPIKDHWPAANQVGVGAFGPGLTPPHGGILGWSPQAQGILTTVST |
| | including the | IPPPASTNRQSGRQPTPISPPLRDSHPQAMQWNSTAFHQALQDPRVRGL |
| | signal peptide of | YLPAGGSSSGTVNPAPNIASHISSISARTGDPVTNKLESVGVHQILAIYS |
| | influenza HA, | TVASSLVLLVSLGAISFWMCSNGSLQCRICI |
| | preS, and the | |
| | transmembrane/ | |
| | cytoplasmic | |
| | domains of | |
| | influenza HA) | |
| 355 | Plasmodium sp. | ARPGMMRKLAILSVSSFLFVEALFQEYQCYGSSSNTRVLNELNYDNAG |
| | CSP | TNLYNELEMNYYGKQENWYSLKKNSRSLGENDDGNNEDNEKLRKPK |
| | | HKKLKQPADGNPDPGGGSNKNNQGNGQGHNMPNDPNRNVDENANA |
| | | NSAVKNNNNEEPSDKHIKEYLNKIQNSLSTEWSPCSVTCGNGIQVRIKP |
| | | GSANKPKDELDYANDIEKKICKMEKCSSVFNVVNS |
| 356 | Plasmodium sp. | PIPGMMRKLAILSVSSFLFVEALFQEYQCYGSSSNTRVLNELNYDNAGT |
| | CSP CSP21R (21 | NLYNELEMNYYGKQENWYSLKKNSRSLGENDDGNNEDNEKLRKPKH |
| | Repeats) | KKLKQPADGNPDPNANPNVDPNANPNVDPNANPNVDPNANPNANPN |
| | | ANPNANPNANPNANPNANPNANPNANPNANPNANPNANPNANPNAN |
| | | PNANPNANPNANPNVDPNANPNKNNQGNGQGHNMPNDPNRNVDEN |
| | | ANANSAVKNNNNEEPSDKHIKEYLNKIQNSLSTEWSPCSVTCGNGIQV |
| | | RIKPGSANKPKDELDYANDIEKKICKMEKCSSVFNVVNS |
| 357 | Plasmodium sp. | PGATMSVLQSGALPSVGVDELDKIDLSYETTESGDTAVSEDSYDKYAS |
| | Pfs230 | NNTNKEYVCDFTDQLKPTESGPKVKKCEVKVNEPLIKVKIICPLKGSVE |
| | | KLYDNIEYVPKKSPYVVLTKEETKLKEKLLSKLIYGLLISPTVNEKENN |
| | | FKEGVIEFTLPPVVHKATVFYFICDNSKTEDDNKKGNRGIVEVYVEPY |
| | | GNKING |
| 358 | Human Mucin-I | AHGVTSAPDTRPAPGSTAPP |
| | extracellular | |
| | domain fragment | |
| 359 | Human Mucin-I | AHGVTSAPDNRPALGSTAPP |
| | extracellular | |
| | domain fragment | |
| 360 | Human Mucin-I | AHGVTSAPDTRPAPGSTAPPAHGVTSAPDNRPALGSTAPP |
| | extracellular | |
| | domain fragment | |
| 361 | Human Mucin-I | AHGVTSAPDTRPAPGSTAPPAHGVTSAPDTRPAPGSTAPPAHGVTSAP |
| | extracellular | DTRPAPGSTAPPAHGVTSAPDTRPAPGSTAPPAHGVTSAPDNRPALGST |
| | domain fragment | APP |
| 362 | Human Mucin-I | RRKNYGQLDIFPARDTYHPMSEYPTYHTHGRYVPPSSTDRSPYEKVSA |
| | intracellular | GNGGSSLSYTNPAVAATSANL |
| | domain fragment | |
| 363 | Human Mucin-I | TPGTQSPFFLLLLLTVLTVVTGSGHASSTPGGEKETSATQRSSVPSSTEK |
| | 1 tandem repeat | NAVSMTSSVLSSHSPGSGSSTTQGQDVTLAPATEPASGSAATWGQDVT |
| | | SVPVTRPALGSTTPPAHDVTSAPDNKPAPGSTAPPIAHGVTSAPDTRPA |
| | | PGSTAPAAHGVTSAPDNRPALGSTAPPVHNVTSASGSASGSASTLVHN |
| | | GTSARATTTPASKSTPFSIPSHHSDTPTTLASHSTKTDASSTHHSTVPPLT |
| | | SSNHSTSPQLSTGVSFFFLSFHISNLQFNSSLEDPSTDYYQELQRDISEMF |
| | | LQIYKQGGFLGLSNIKFRPGSVVVQLTLAFREGTINVHDVETQFNQYKT |
| | | EAASRYNLTISDVSVSDVPFPFSAQSGAGVPGWGIALLVLVCVLVALAI |
| | | VYLIALAVCQCRRKNYGQLDIFPARDTYHPMSEYPTYHTHGRYVPPSS |
| | | TDRSPYEKVSAGNGGSSLSYTNPAVAATSANL |
| 364 | Human Mucin-I | TPGTQSPFFLLLLLTVLTVVTGSGHASSTPGGEKETSATQRSSVPSSTEK |
| | 4 tandem repeat | NAVSMTSSVLSSHSPGSGSSTTQGQDVTLAPATEPASGSAATWGQDVT |
| | | SVPVTRPALGSTTPPAHDVTSAPDNKPAPGSTAPPAHGVTSAPDTRPAP |
| | | GSTPPAHGVTSAPDTRPAPGSTAPPAHGVTSAPDTRPAPGSTAPPAHGV |
| | | TSAPDTRPAPGSTAPPHGVTSAPDNRPALGSTAPPVHNVTSASGSASGS |
| | | ASTLVHNGTSARATTTPASKSTPFSIPSHHSDTPTTLASHSTKTDASSTH |
| | | HSTVPPLTSSNHSTSPQLSTGVSFFFLSFHISNLQFNSSLEDPSTDYYQEL |
| | | QRDISEMFLQIYKQGGFLGLSNIKFRPGSVVVQLTLAFREGTINVHDVE |
| | | TQFNQYKTEAASRYNLTISDVSVSDVPFPFSAQSGAGVPGWGIALLVL |
| | | VCVLVALAIVYLIALAVCQCRRKNYGQLDIFPARDTYHPMSEYPTYHT |
| | | HGRYVPPSSTDRSPYEKVSAGNGGSSLSYTNPAVAATSANL |
| 365 | Lassa virus | GQIVTFFQEVPHVIEEVMNIVLIALSVLAVLKGLYNFATCGLVGLVTFL |
| | Glycoprotein | LLCGRSCTTSLYKGVYELQTLELNMETLNMTMPLSCTKNNSHHYIMV |
| | | GNETGLELTLTNTSIINHKFCNLSDAHKKNLYDHALMSIISTFHLSIPNF |
| | | NQYEAMSCDFNGGKISVQYNLSHSYAGDAANHCGTVANGVLQTFMR |
| | | MAWGGSYIALDSGRGNWDCIMTSYQYLIIQNTTWEDHCQFSRPSPIGY |
| | | LGLLSQRTRDIYISRRLLGTFTWTLSDSEGKDTPGGYCLTRWMLIEAEL |
| | | KCFGNTAVAKCNEKHDEEFCDMLRLFDFNKQAIQRLKAEAQMSIQLIN |
| | | KAVNALINDQLIMKNHLRDIMGIPYCNYSKYWYLNHTTTGRTSLPKC |
| | | WLVSNGSYLNETHFSDDIEQQADNMITEMLQKEYMERQGKTPLGLVD |
| | | LFVFSTSFYLISIFLHLVKIPTHRHIVGKSCPKPHRLNHMGICSCGLYKQP |
| | | GVPVKWKR |
| 366 | Lassa virus Z | GNKQAKAPESKDSPRASLIPDATHLGPQFCKSCWFENKGLVECNNHYL |
| | protein | CLNCLTLLLSVSNRCPICKMPLPTKLRPSAAPTAPPTGAADSIRPPPYSP |
| 367 | Ebola virus | GVTGILQLPRDRFKRTSFFLWVIILFQRTFSIPLGVIHNSTLQVSDVDKL |
| | Glycoprotein | VCRDKLSSTNQLRSVGLNLEGNGVATDVPSVTKRWGFRSGVPPKVVN |
| | | YEAGEWAENCYNLEIKKPDGSECLPAAPDGIRGFPRCRYVHKVSGTGP |
| | | CAGDFAFHKEGAFFLYDRLASTVIYRGTTFAEGVVAFLILPQAKKDFFS |
| | | SHPLREPVNATEDPSSGYYSTTIRYQATGFGTNETEYLFEVDNLTYVQL |
| | | ESRFTPQFLLQLNETIYASGKRSNTTGKLIWKVNPEIDTTIGEWAFWET |
| | | KKNLTRKIRSEELSFTAVSNGPKNISGQSPARTSSDPETNTTNEDHKIM |
| | | ASENSSAMVQVHSQGRKAAVSHLTTLATISTSPQPPTTKTGPDNSTHN |
| | | TPVYKLDISEATQVGQHHRRADNDSTASDTPPATTAAGPLKAENTNTS |
| | | KSADSLDLATTTSPQNYSETAGNNNTHHQDTGEESASSGKLGLITNTIA |
| | | GVAGLITGGRRTRREVIVNAQPKCNPNLHYWTTQDEGAAIGLAWIPYF |
| | | GPAAEGIYTEGLMHNQDGLICGLRQLANETTQALQLFLRATTELRTFSI |
| | | LNRKAIDFLLQRWGGTCHILGPDCCIEPHDWTKNITDKIDQIIHDFVDK |
| | | TLPDQGDNDNWWTGWRQWIPAGIGVTGVIIAVIALFCICKFVF |
| 368 | Ebola Virus | RRVILPTAPPEYMEAIYPARSNSTIARGGNSNTGFLTPESVNGDTPSNPL |
| | VP40 protein | RPIADDTIDHASHTPGSVSSAFILEAMVNVISGPKVLMKQIPIWLPLGVA |
| | | DQKTYSFDSTTAAIMLASYTITHFGKATNPLVRVNRLGPGIPDHPLRLL |
| | | RIGNQAFLQEFVLPPVQLPQYFTFDLTALKLITQPLPAATWTDDTPTGS |
| | | NGALRPGISFHPKLRPILLPNKSGKKGNSADLTSPEKIQAIMTSLQDFKI |
| | | VPIDPTKNIMGIEVPETLVHKLTGKKVTSKNGQPIIPVLLPKYIGLDPVA |
| | | PGDLTMVITQDCDTCHSPASLPAVVEK |
| 369 | Zika virus- | KNPKKKSGGFRIVNMLKRGVARVSPFGGLKRLPAGLLLGHGPIRMVL |
| | native | AILAFLRFTAIKPSLGLINRWGSVGKKEAMEIIKKFKKDLAAMLRIINAR |
| | polyprotein | KEKKRRGADTSVGIVGLLLTTAMAAEVTRRGSAYYMYLDRNDAGEAI |
| | sequence for | SFPTTLGMNKCYIQIMDLGHTCDATMSYECPMLDEGVEPDDVDCWCN |
| | Zika, from | TTSTWVVYGTCHHKKGEARRSRRAVTLPSHSTRKLQTRSQTWLESRE |
| | GenBank | YTKHLIRVENWIFRNPGFALAAAAIAWLLGSSTSQKVIYLVMILLIAPA |
| | (ALX35659) | YSIRCIGVSNRDFVEGMSGGTWVDVVLEHGGCVTVMAQDKPTVDIEL |
| | | VTTTVSNMAEVRSYCYEASISDMASDSRCPTQGEAYLDKQSDTQYVC |
| | | KRTLVDRGWGNGCGLFGKGSLVTCAKFACSKKMTGKSIQPENLEYRI |
| | | MLSVHGSQHSGMIVNDTGHETDENRAKVEITPNSPRAEATLGGFGSLG |
| | | LDCEPRTGLDFSDLYYLTMNNKHWLVHKEWFHDIPLPWHAGADTGT |
| | | PHWNNKEALVEFKDAHAKRQTVVVLGSQEGAVHTALAGALEAEMD |
| | | GAKGRLSSGHLKCRLKMDKLRLKGVSYSLCTAAFTFTKIPAETLHGTV |
| | | TVEVQYAGTDGPCKVPAQMAVDMQTLTPVGRLITANPVITESTENSK |
| | | MMLELDPPFGDSYIVIGVGEKKITHHWHRSGSTIGKAFEATVRGAKRM |
| | | AVLGDTAWDFGSVGGALNSLGKGIHQIFGAAFKSLFGGMSWFSQILIG |
| | | TLLMWLGLNAKNGSISLMCLALGGVLIFLSTAVSADVGCSVDFSKKET |
| | | RCGTGVFVYNDVEAWRDRYKYHPDSPRRLAAAVKQAWEDGICGISS |
| | | VSRMENIMWRSVEGELNAILEENGVQLTVVVGSVKNPMWRGPQRLP |
| | | VPVNELPHGWKAWGKSYFVRAAKTNNSFVVDGDTLKECPLKHRAW |
| | | NSFLVEDHGFGVFHTSVWLKVREDYSLECDPAVIGTAVKGKEAVHSD |
| | | LGYWIESEKNDTWRLKRAHLIEMKTCEWPKSHTLWTDGIEESDLIIPKS |
| | | LAGPLSHHNTREGYRTQMKGPWHSEELEIRFEECPGTKVHVEETCGTR |
| | | GPSLRSTTASGRVIEEWCCRECTMPPLSFRAKDGCWYGMEIRPRKEPE |
| | | SNLVRSMVTAGSTDHMDHFSLGVLVILLMVQEGLKKRMTTKIIISTSM |
| | | AVLVAMILGGFSMSDLAKLAILMGATFAEMNTGGDVAHLALIAAFKV |
| | | RPALLVSFIFRANWTPRESMLLALASCLLQTAISALEGDLMVLINGFAL |
| | | AWLAIRAMVVPRTDNITLAILAALTPLARGTLLVAWRAGLATCGGFM |
| | | LLSLKGKGSVKKNLPFVMALGLTAVRLVDPINVVGLLLLTRSGKRSWP |
| | | PSEVLTAVGLICALAGGFAKADIEMAGPMAAVGLLIVSYVVSGKSVD |
| | | MYIERAGDITWEKDAEVTGNSPRLDVALDESGDFSLVEDDGPPMREIIL |
| | | KVVLMTICGMNPIAIPFAAGAWYVYVKTGKRSGALWDVPAPKEVKK |
| | | GETTDGVYRVMTRRLLGSTQVGVGVMQEGVFHTMWHVTKGSALRS |
| | | GEGRLDPYWGDVKQDLVSYCGPWKLDAAWDGHSEVQLLAVPPGER |
| | | ARNIQTLPGIFKTKDGDIGAVALDYPAGTSGSPILDKCGRVIGLYGNGV |
| | | VIKNGSYVSAITQGRREEETPVECFEPSMLKKKQLTVLDLHPGAGKTR |
| | | RVLPEIVREAIKTRLRTVILAPTRVVAAEMEEALRGLPVRYMTTAVNV |
| | | THSGTEIVDLMCHATFTSRLLQPIRVPNYNLYIMDEAHFTDPSSIAARG |
| | | YISTRVEMGEAAAIFMTATPPGTRDAFPDSNSPIMDTEVEVPERAWSSG |
| | | FDWVTDHSGKTVWFVPSVRNGNEIAACLTKAGKRVIQLSRKTFETEFQ |
| | | KTKHQEWDFVVTTDISEMGANFKADRVIDSRRCLKPVILDGERVILAG |
| | | PMPVTHASAAQRRGRIGRNPNKPGDEYLYGGGCAETDEDHAHWLEA |
| | | RMLLDNIYLQDGLIASLYRPEADKVAAIEGEFKLRTEQRKTFVELMKR |
| | | GDLPVWLAYQVASAGITYTDRRWCFDGTTNNTIMEDSVPAEVWTRH |
| | | GEKRVLKPRWMDARVCSDHAALKSFKEFAAGKRGAAFGVMEALGTL |
| | | PGHMTERFQEAIDNLAVLMRAETGSRPYKAAAAQLPETLETIMLLGLL |
| | | GTVSLGIFFVLMRNKGIGKMGFGMVTLGASAWLMWLSEIEPARIACV |
| | | LIVVFLLLVVLIPEPEKQRSPQDNQMAIIIMVAVGLLGLITANELGWLER |
| | | TKSDLSHLMGRREEGATIGFSMDIDLRPASAWAIYAALTTFITPAVQHA |
| | | VTTSYNNYSLMAMATQAGVLFGMGKGMPFYAWDFGVPLLMIGCYSQ |
| | | LTPLTLIVAIILLVAHYMYLIPGLQAAAARAAQKRTAAGIMKNPVVDGI |
| | | VVTDIDTMTIDPQVEKKMGQVLLIAVAVSSAILSRTAWGWGEAGALIT |
| | | AATSTLWEGSPNKYWNSSTATSLCNIFRGSYLAGASLIYTVTRNAGLV |
| | | KRRGGGTGETLGEKWKARLNQMSALEFYSYKKSGITEVCREEARRAL |
| | | KDGVATGGHAVSRGSAKLRWLVERGYLQPYGKVIDLGCGRGGWSYY |
| | | AATIRKVQEVKGYTKGGPGHEEPVLVQSYGWNIVRLKSGVDVFHMA |
| | | AEPCDTLLCDIGESSSSPEVEEARTLRVLSMVGDWLEKRPGAFCIKVLC |
| | | PYTSTMMETLERLQRRYGGGLVRVPLSRNSTHEMYWVSGAKSNTIKS |
| | | VSTTSQLLLGRMDGPRRPVKYEEDVNLGSGTRAVVSCAEAPNMKIIGN |
| | | RIERIRSEHAETWFFDENHPYRTWAYHGSYEAPTQGSASSLINGVVRLL |
| | | SKPWDVVTGVTGIAMTDTTPYGQQRVFKEKVDTRVPDPQEGTRQVM |
| | | SMVSSWLWKELGKHKRPRVCTKEEFINKVRSNAALGAIFEEEKEWKT |
| | | AVEAVNDPRFWALVDKEREHHLRGECQSCVYNMMGKREKKQGEFG |
| | | KAKGSRAIWYMWLGARFLEFEALGFLNEDHWMGRENSGGGVEGLGL |
| | | QRLGYVLEEMSRIPGGRMYADDTAGWDTRISRFDLENEALITNQMEK |
| | | GHRALALAIIKYTYQNKVVKVLRPAEKGKTVMDIISRQDQRGSGQVV |
| | | TYALNTFTNLVVQLIRNMEAEEVLEMQDLWLLRRSEKVINWLQSNG |
| | | WDRLKRMAVSGDDCVVKPIDDRFAHALRFLNDMGKVRKDTQEWKPS |
| | | TGWDNWEEVPFCSHHFNKLHLKDGRSIVVPCRHQDELIGRARVSPGA |
| | | GWSIRETACLAKSYAQMWQLLYFHRRDLRLMANAICSSVPVDWVPT |
| | | GRTTWSIHGKGEWMTTEDMLVVWNRVWIEENDHMEDKTPVTKWTD |
| | | IPYLGKREDLWCGSLIGHRPRTTWAENIKNTVNMVRRIIGDEEKYMDY |
| | | LSTQVRYLGEEGSTPGVL |
| 370 | Zika virus-PrM + | TRRGSAYYMYLDRNDAGEAISFPTTLGMNKCYIQIMDLGHTCDATMS |
| | E | YECPMLDEGVEPDDVDCWCNTTSTWVVYGTCHHKKGEARRSRRAVT |
| | | LPSHSTRKLQTRSQTWLESREYTKHLIRVENWIFRNPGFALAAAAIAW |
| | | LLGSSTSQKVIYLVMILLIAPAYSIRCIGVSNRDFVEGMSGGTWVDVVL |
| | | EHGGCVTVMAQDKPTVDIELVTTTVSNMAEVRSYCYEASISDMASDS |
| | | RCPTQGEAYLDKQSDTQYVCKRTLVDRGWGNGCGLFGKGSLVTCAK |
| | | FACSKKMTGKSIQPENLEYRIMLSVHGSQHSGMIVNDTGHETDENRAK |
| | | VEITPNSPRAEATLGGFGSLGLDCEPRTGLDFSDLYYLTMNNKHWLVH |
| | | KEWFHDIPLPWHAGADTGTPHWNNKEALVEFKDAHAKRQTVVVLGS |
| | | QEGAVHTALAGALEAEMDGAKGRLSSGHLKCRLKMDKLRLKGVSYS |
| | | LCTAAFTFTKIPAETLHGTVTVEVQYAGTDGPCKVPAQMAVDMQTLT |
| | | PVGRLITANPVITESTENSKMMLELDPPFGDSYIVIGVGEKKITHHWHR |
| | | SGSTIGKAFEATVRGAKRMAVLGDTAWDFGSVGGALNSLGKGIHQIF |
| | | GAAFKSLFGGMSWFSQILIGTLLMWLGLNAKNGSISLMCLALGGVLIF |
| | | LSTAVSA |
| 371 | Zika virus-JEV | GKRSAGSIMWLASLAVVIACAGA |
| | signal | |
| 372 | Zika virus-JEV | GKRSAGSIMWLASLAVVIACAGATRRGSAYYMYLDRNDAGEAISFPT |
| | signal + PrM + E | TLGMNKCYIQIMDLGHTCDATMSYECPMLDEGVEPDDVDCWCNTTS |
| | | TWVVYGTCHHKKGEARRSRRAVTLPSHSTRKLQTRSQTWLESREYTK |
| | | HLIRVENWIFRNPGFALAAAAIAWLLGSSTSQKVIYLVMILLIAPAYSIR |
| | | CIGVSNRDFVEGMSGGTWVDVVLEHGGCVTVMAQDKPTVDIELVTTT |
| | | VSNMAEVRSYCYEASISDMASDSRCPTQGEAYLDKQSDTQYVCKRTL |
| | | VDRGWGNGCGLFGKGSLVTCAKFACSKKMTGKSIQPENLEYRIMLSV |
| | | HGSQHSGMIVNDTGHETDENRAKVEITPNSPRAEATLGGFGSLGLDCE |
| | | PRTGLDFSDLYYLTMNNKHWLVHKEWFHDIPLPWHAGADTGTPHWN |
| | | NKEALVEFKDAHAKRQTVVVLGSQEGAVHTALAGALEAEMDGAKGR |
| | | LSSGHLKCRLKMDKLRLKGVSYSLCTAAFTFTKIPAETLHGTVTVEVQ |
| | | YAGTDGPCKVPAQMAVDMQTLTPVGRLITANPVITESTENSKMMLEL |
| | | DPPFGDSYIVIGVGEKKITHHWHRSGSTIGKAFEATVRGAKRMAVLGD |
| | | TAWDFGSVGGALNSLGKGIHQIFGAAFKSLFGGMSWFSQILIGTLLMW |
| | | LGLNAKNGSISLMCLALGGVLIFLSTAVSA |
| 373 | Zika virus-full- | VGCSVDFSKKETRCGTGVFVYNDVEAWRDRYKYHPDSPRRLAAAVK |
| | length Zika virus | QAWEDGICGISSVSRMENIMWRSVEGELNAILEENGVQLTVVVGSVK |
| | NS1 protein | NPMWRGPQRLPVPVNELPHGWKAWGKSYFVRAAKTNNSFVVDGDTL |
| | sequence | KECPLKHRAWNSFLVEDHGFGVFHTSVWLKVREDYSLECDPAVIGTA |
| | | VKGKEAVHSDLGYWIESEKNDTWRLKRAHLIEMKTCEWPKSHTLWT |
| | | DGIEESDLIIPKSLAGPLSHHNTREGYRTQMKGPWHSEELEIRFEECPGT |
| | | KVHVEETCGTRGPSLRSTTASGRVIEEWCCRECTMPPLSFRAKDGCWY |
| | | GMEIRPRKEPESNLVRSMVTAG |
| 374 | Zika virus-Zika | GKRSAGSIMWLASLAVVIACAGATRRGSAYYMYLDRNDAGEAISFPT |
| | virus polyprotein | TLGMNKCYIQIMDLGHTCDATMSYECPMLDEGVEPDDVDCWCNTTS |
| | JEV signal + prM + | TWVVYGTCHHKKGEARRSRRAVTLPSHSTRKLQTRSQTWLESREYTK |
| | E + K643-S644 | HLIRVENWIFRNPGFALAAAAIAWLLGSSTSQKVIYLVMILLIAPAYSIR |
| | | CIGVSNRDFVEGMSGGTWVDVVLEHGGCVTVMAQDKPTVDIELVTTT |
| | | VSNMAEVRSYCYEASISDMASDSRCPTQGEAYLDKQSDTQYVCKRTL |
| | | VDRGWGNGCGLFGKGSLVTCAKFACSKKMTGKSIQPENLEYRIMLSV |
| | | HGSQHSGMIVNDTGHETDENRAKVEITPNSPRAEATLGGFGSLGLDCE |
| | | PRTGLDFSDLYYLTMNNKHWLVHKEWFHDIPLPWHAGADTGTPHWN |
| | | NKEALVEFKDAHAKRQTVVVLGSQEGAVHTALAGALEAEMDGAKGR |
| | | LSSGHLKCRLKMDKLRLKGVSYSLCTAAFTFTKIPAETLHGTVTVEVQ |
| | | YAGTDGPCKVPAQMAVDMQTLTPVGRLITANPVITESTENSKMMLEL |
| | | DPPFGDSYIVIGVGEKKITHHWHRSGSTIGKAFEATVRGAKRMAVLGD |
| | | TAWDFGSVGGALNSLGKGIHQIFGAAFKSLFGGMSWFSQILIGTLLMW |
| | | LGLNAKNGSISLMCLALGGVLIFLSTAVSA |
| 375 | Zika virus-gene | GKRSAGSIMWLASLAVVIACAGATRRGSAYYMYLDRNDAGEAISFPT |
| | product | TLGMNKCYIQIMDLGHTCDATMSYECPMLDEGVEPDDVDCWCNTTS |
| | | TWVVYGTCHHKKGEARRSRRAVTLPSHSTRKLQTRSQTWLESREYTK |
| | | HLIRVENWIFRNPGFALAAAAIAWLLGSSTSQKVIYLVMILLIAPAYSIR |
| | | CIGVSNRDFVEGMSGGTWVDVVLEHGGCVTVMAQDKPTVDIELVTTT |
| | | VSNMAEVRSYCYEASISDMASDSRCPTQGEAYLDKQSDTQYVCKRTL |
| | | VDRGWGNGCGLFGKGSLVTCAKFACSKKMTGKSIQPENLEYRIMLSV |
| | | HGSQHSGMIVNDTGHETDENRAKVEITPNSPRAEATLGGFGSLGLDCE |
| | | PRTGLDFSDLYYLTMNNKHWLVHKEWFHDIPLPWHAGADTGTPHWN |
| | | NKEALVEFKDAHAKRQTVVVLGSQEGAVHTALAGALEAEMDGAKGR |
| | | LSSGHLKCRLKMDKLRLKGVSYSLCTAAFTFTKIPAETLHGTVTVEVQ |
| | | YAGTDGPCKVPAQMAVDMQTLTPVGRLITANPVITESTENSKMMLEL |
| | | DPPFGDSYIVIGVGEKKITHHWHRSGSTIGKAFEATVRGAKRMAVLGD |
| | | TAWDFGSVGGALNSLGKGIHQIFGAAFK |
| 376 | Zika virus- | TRRGSAYYMYLDRNDAGEAISFPTTLGMNKCYIQIMDLGHTCDATMS |
| | prMsE | YECPMLDEGVEPDDVDCWCNTTSTWVVYGTCHHKKGEARRSRRAVT |
| | | LPSHSTRKLQTRSQTWLESREYTKHLIRVENWIFRNPGFALAAAAIAW |
| | | LLGSSTSQKVIYLVMILLIAPAYSIRCIGVSNRDFVEGMSGGTWVDVVL |
| | | EHGGCVTVMAQDKPTVDIELVTTTVSNMAEVRSYCYEASISDMASDS |
| | | RCPTQGEAYLDKQSDTQYVCKRTLVDRGWGNGCGLFGKGSLVTCAK |
| | | FACSKKMTGKSIQPENLEYRIMLSVHGSQHSGMIVNDTGHETDENRAK |
| | | VEITPNSPRAEATLGGFGSLGLDCEPRTGLDFSDLYYLTMNNKHWLVH |
| | | KEWFHDIPLPWHAGADTGTPHWNNKEALVEFKDAHAKRQTVVVLGS |
| | | QEGAVHTALAGALEAEMDGAKGRLSSGHLKCRLKMDKLRLKGVSYS |
| | | LCTAAFTFTKIPAETLHGTVTVEVQYAGTDGPCKVPAQMAVDMQTLT |
| | | PVGRLITANPVITESTENSKMMLELDPPFGDSYIVIGVGEKKITHHWHR |
| | | SGSTIGKAFEATVRGAKRMAVLGDTAWDFGSVGGALNSLGKGIHQIF |
| | | GAAFK |
| 377 | SARS-CoV2 | FVFLVLLPLVSSQCVNLTTRTQLPPAYTNSFTRGVYYPDKVFRSSVLHS |
| | full-length S | TQDLFLPFFSNVTWFHAIHVSGTNGTKRFDNPVLPFNDGVYFASTEKS |
| | protein-Wuhan | NIIRGWIFGTTLDSKTQSLLIVNNATNVVIKVCEFQFCNDPFLGVYYHK |
| | Strain | NNKSWMESEFRVYSSANNCTFEYVSQPFLMDLEGKQGNFKNLREFVF |
| | | KNIDGYFKIYSKHTPINLVRDLPQGFSALEPLVDLPIGINITRFQTLLALH |
| | | RSYLTPGDSSSGWTAGAAAYYVGYLQPRTFLLKYNENGTITDAVDCA |
| | | LDPLSETKCTLKSFTVEKGIYQTSNFRVQPTESIVRFPNITNLCPFGEVEN |
| | | ATRFASVYAWNRKRISNCVADYSVLYNSASFSTFKCYGVSPTKLNDLC |
| | | FTNVYADSFVIRGDEVRQIAPGQTGKIADYNYKLPDDFTGCVIAWNSN |
| | | NLDSKVGGNYNYLYRLFRKSNLKPFERDISTEIYQAGSTPCNGVEGEN |
| | | CYFPLQSYGFQPTNGVGYQPYRVVVLSFELLHAPATVCGPKKSTNLVK |
| | | NKCVNFNFNGLTGTGVLTESNKKFLPFQQFGRDIADTTDAVRDPQTLE |
| | | ILDITPCSFGGVSVITPGTNTSNQVAVLYQDVNCTEVPVAIHADQLTPT |
| | | WRVYSTGSNVFQTRAGCLIGAEHVNNSYECDIPIGAGICASYQTQTNSP |
| | | RRARSVASQSIIAYTMSLGAENSVAYSNNSIAIPTNFTISVTTEILPVSMT |
| | | KTSVDCTMYICGDSTECSNLLLQYGSFCTQLNRALTGIAVEQDKNTQE |
| | | VFAQVKQIYKTPPIKDFGGFNFSQILPDPSKPSKRSFIEDLLFNKVTLAD |
| | | AGFIKQYGDCLGDIAARDLICAQKFNGLTVLPPLLTDEMIAQYTSALLA |
| | | GTITSGWTFGAGAALQIPFAMQMAYRFNGIGVTQNVLYENQKLIANQF |
| | | NSAIGKIQDSLSSTASALGKLQDVVNQNAQALNTLVKQLSSNFGAISSV |
| | | LNDILSRLDKVEAEVQIDRLITGRLQSLQTYVTQQLIRAAEIRASANLA |
| | | ATKMSECVLGQSKRVDFCGKGYHLMSFPQSAPHGVVFLHVTYVPAQE |
| | | KNFTTAPAICHDGKAHFPREGVFVSNGTHWFVTQRNFYEPQIITTDNTF |
| | | VSGNCDVVIGIVNNTVYDPLQPELDSFKEELDKYFKNHTSPDVDLGDIS |
| | | GINASVVNIQKEIDRLNEVAKNLNESLIDLQELGKYEQYIKWPWYIWL |
| | | GFIAGLIAIVMVTIMLCCMTSCCSCLKGCCSCGSCCKFDEDDSEPVLKG |
| | | VKLHYT |
| 378 | SARS-CoV2 | FVFLVLLPLVSSQCVNLTTRTQLPPAYTNSFTRGVYYPDKVFRSSVLHS |
| | full-length S | TQDLFLPFFSNVTWFHAIHVSGTNGTKRFDNPVLPFNDGVYFASTEKS |
| | protein-K417T, | NIIRGWIFGTTLDSKTQSLLIVNNATNVVIKVCEFQFCNDPFLGVYYHK |
| | E484K, and | NNKSWMESEFRVYSSANNCTFEYVSQPFLMDLEGKQGNFKNLREFVF |
| | N501Y | KNIDGYFKIYSKHTPINLVRDLPQGFSALEPLVDLPIGINITRFQTLLALH |
| | | RSYLTPGDSSSGWTAGAAAYYVGYLQPRTFLLKYNENGTITDAVDCA |
| | | LDPLSETKCTLKSFTVEKGIYQTSNFRVQPTESIVRFPNITNLCPFGEVEN |
| | | ATRFASVYAWNRKRISNCVADYSVLYNSASFSTFKCYGVSPTKLNDLC |
| | | FTNVYADSFVIRGDEVRQIAPGQTGTIADYNYKLPDDFTGCVIAWNSN |
| | | NLDSKVGGNYNYLYRLFRKSNLKPFERDISTEIYQAGSTPCNGVKGFN |
| | | CYFPLQSYGFQPTYGVGYQPYRVVVLSFELLHAPATVCGPKKSTNLVK |
| | | NKCVNFNFNGLTGTGVLTESNKKFLPFQQFGRDIADTTDAVRDPQTLE |
| | | ILDITPCSFGGVSVITPGTNTSNQVAVLYQDVNCTEVPVAIHADQLTPT |
| | | WRVYSTGSNVFQTRAGCLIGAEHVNNSYECDIPIGAGICASYQTQTNSP |
| | | RRARSVASQSIIAYTMSLGAENSVAYSNNSIAIPTNFTISVTTEILPVSMT |
| | | KTSVDCTMYICGDSTECSNLLLQYGSFCTQLNRALTGIAVEQDKNTQE |
| | | VFAQVKQIYKTPPIKDFGGFNFSQILPDPSKPSKRSFIEDLLFNKVTLAD |
| | | AGFIKQYGDCLGDIAARDLICAQKFNGLTVLPPLLTDEMIAQYTSALLA |
| | | GTITSGWTFGAGAALQIPFAMQMAYRFNGIGVTQNVLYENQKLIANQF |
| | | NSAIGKIQDSLSSTASALGKLQDVVNQNAQALNTLVKQLSSNFGAISSV |
| | | LNDILSRLDKVEAEVQIDRLITGRLQSLQTYVTQQLIRAAEIRASANLA |
| | | ATKMSECVLGQSKRVDFCGKGYHLMSFPQSAPHGVVFLHVTYVPAQE |
| | | KNFTTAPAICHDGKAHFPREGVFVSNGTHWFVTQRNFYEPQIITTDNTF |
| | | VSGNCDVVIGIVNNTVYDPLQPELDSFKEELDKYFKNHTSPDVDLGDIS |
| | | GINASVVNIQKEIDRLNEVAKNLNESLIDLQELGKYEQYIKWPWYIWL |
| | | GFIAGLIAIVMVTIMLCCMTSCCSCLKGCCSCGSCCKFDEDDSEPVLKG |
| | | VKLHYT |
| 379 | SARS-CoV2 | FVFLVLLPLVSSQCVNLRTRTQLPPAYTNSFTRGVYYPDKVFRSSVLHS |
| | full-length S | TQDLFLPFFSNVTWFHAIHVSGTNGTKRFDNPVLPFNDGVYFASIEKSN |
| | protein-delta | IIRGWIFGTTLDSKTQSLLIVNNATNVVIKVCEFQFCNDPFLDVYYHKN |
| | variant | NKSWMESGVYSSANNCTFEYVSQPFLMDLEGKQGNFKNLREFVFKNI |
| | | DGYFKIYSKHTPINLVRDLPQGFSALEPLVDLPIGINITRFQTLLALHRSY |
| | | LTPGDSSSGWTAGAAAYYVGYLQPRTFLLKYNENGTITDAVDCALDP |
| | | LSETKCTLKSFTVEKGIYQTSNFRVQPTESIVRFPNITNLCPFGEVENAT |
| | | RFASVYAWNRKRISNCVADYSVLYNSASFSTFKCYGVSPTKLNDLCFT |
| | | NVYADSFVIRGDEVRQIAPGQTGKIADYNYKLPDDFTGCVIAWNSNNL |
| | | DSKVGGNYNYRYRLFRKSNLKPFERDISTEIYQAGSKPCNGVEGENCY |
| | | FPLQSYGFQPTNGVGYQPYRVVVLSFELLHAPATVCGPKKSTNLVKNK |
| | | CVNFNFNGLTGTGVLTESNKKFLPFQQFGRDIADTTDAVRDPQTLEILD |
| | | ITPCSFGGVSVITPGTNTSNQVAVLYQGVNCTEVPVAIHADQLTPTWR |
| | | VYSTGSNVFQTRAGCLIGAEHVNNSYECDIPIGAGICASYQTQTNSRRR |
| | | ARSVASQSIIAYTMSLGAENSVAYSNNSIAIPTNFTISVTTEILPVSMTKT |
| | | SVDCTMYICGDSTECSNLLLQYGSFCTQLNRALTGIAVEQDKNTQEVF |
| | | AQVKQIYKTPPIKDFGGFNFSQILPDPSKPSKRSFIEDLLFNKVTLADAG |
| | | FIKQYGDCLGDIAARDLICAQKFNGLTVLPPLLTDEMIAQYTSALLAGT |
| | | ITSGWTFGAGAALQIPFAMQMAYRFNGIGVTQNVLYENQKLIANQFNS |
| | | AIGKIQDSLSSTASALGKLQNVVNQNAQALNTLVKQLSSNFGAISSVLN |
| | | DILSRLDKVEAEVQIDRLITGRLQSLQTYVTQQLIRAAEIRASANLAAT |
| | | KMSECVLGQSKRVDFCGKGYHLMSFPQSAPHGVVFLHVTYVPAQEK |
| | | NFTTAPAICHDGKAHFPREGVFVSNGTHWFVTQRNFYEPQIITTDNTFV |
| | | SGNCDVVIGIVNNTVYDPLQPELDSFKEELDKYFKNHTSPDVDLGDISG |
| | | INASVVNIQKEIDRLNEVAKNLNESLIDLQELGKYEQYIKWPWYIWLG |
| | | FIAGLIAIVMVTIMLCCMTSCCSCLKGCCSCGSCCKFDEDDSEPVLKGV |
| | | KLHYT |
| 380 | SARS-CoV2 | FVFLVLLPLVSSQCVNLRTRTQLPPAYTNSFTRGVYYPDKVFRSSVLHS |
| | full-length S | TQDLFLPFFSNVTWFHAIHFSGTNGTKRFDNPVLPFNDGVYFASIEKSNI |
| | protein-delta | IRGWIFGTTLDSKTQSLLIVNNATNVVIKVCEFQFCNDPFLDVYYHKNN |
| | variant plus | KSWMESGVYSSANNCTFEYVSQPFLMDLEGKQGNFKNLREFVFKNID |
| | | GYFKIYSKHTPINLVRDLPQGFSVLEPLVDLPIGINITRFQTLLALHRSYL |
| | | TPGDSSSGLTAGAAAYYVGYLQPRTFLLKYNENGTITDAVDCALDPLS |
| | | ETKCTLKSFTVEKGIYQTSNFRVQPTESIVRFPNITNLCPFGEVFNATRF |
| | | ASVYAWNRKRISNCVADYSVLYNSASFSTFKCYGVSPTKLNDLCFTNV |
| | | YADSFVIRGDEVRQIAPGQTGNIADYNYKLPDDFTGCVIAWNSNNLDS |
| | | KVGGNYNYRYRLFRKSNLKPFERDISTEIYQAGSKPCNGVEGENCYFP |
| | | LQSYGFQPTNGVGYQPYRVVVLSFELLHAPATVCGPKKSTNLVKNKC |
| | | VNFNFNGLTGTGVLTESNKKFLPFQQFGRDIADTTDAVRDPQTLEILDI |
| | | TPCSFGGVSVITPGTNTSNQVAVLYQGVNCTEVPVAIHADQLTPTWRV |
| | | YSTGSNVFQTRAGCLIGAEHVNNSYECDIPIGAGICASYQTQTNSRRRA |
| | | RSVASQSIIAYTMSLGAENSVAYSNNSIAIPTNFTISVTTEILPVSMTKTS |
| | | VDCTMYICGDSTECSNLLLQYGSFCTQLNRALTGIAVEQDKNTQEVFA |
| | | QVKQIYKTPPIKDFGGFNFSQILPDPSKPSKRSFIEDLLFNKVTLADAGFI |
| | | KQYGDCLGDIAARDLICAQKFNGLTVLPPLLTDEMIAQYTSALLAGTIT |
| | | SGWTFGAGAALQIPFAMQMAYRFNGIGVTQNVLYENQKLIANQFNSA |
| | | IGKIQDSLSSTASALGKLQNVVNQNAQALNTLVKQLSSNFGAISSVLND |
| | | ILSRLDKVEAEVQIDRLITGRLQSLQTYVTQQLIRAAEIRASANLAATK |
| | | MSECVLGQSKRVDFCGKGYHLMSFPQSAPHGVVFLHVTYVPAQEKNF |
| | | TTAPAICHDGKAHFPREGVFVSNGTHWFVTQRNFYEPQIITTDNTFVSG |
| | | NCDVVIGIVNNTVYDPLQPELDSFKEELDKYFKNHTSPDVDLGDISGIN |
| | | ASVVNIQKEIDRLNEVAKNLNESLIDLQELGKYEQYIKWPWYIWLGFI |
| | | AGLIAIVMVTIMLCCMTSCCSCLKGCCSCGSCCKFDEDDSEPVLKGVK |
| | | LHYT |
| 381 | SARS-CoV2 | FVFLVLLPLVSSQCVNLTTRTQLPPAYTNSFTRGVYYPDKVFRSSVLHS |
| | full-length S | TQDLFLPFFSNVTWFHAIHVSGTNGTKRFDNPVLPFNDGVYFASTEKS |
| | protein | NIIRGWIFGTTLDSKTQSLLIVNNATNVVIKVCEFQFCNDPFLGVYYHK |
| | stabilized with 2 | NNKSWMESEFRVYSSANNCTFEYVSQPFLMDLEGKQGNFKNLREFVF |
| | proline | KNIDGYFKIYSKHTPINLVRDLPQGFSALEPLVDLPIGINITRFQTLLALH |
| | substitutions | RSYLTPGDSSSGWTAGAAAYYVGYLQPRTFLLKYNENGTITDAVDCA |
| | | LDPLSETKCTLKSFTVEKGIYQTSNFRVQPTESIVRFPNITNLCPFGEVFN |
| | | ATRFASVYAWNRKRISNCVADYSVLYNSASFSTFKCYGVSPTKLNDLC |
| | | FTNVYADSFVIRGDEVRQIAPGQTGKIADYNYKLPDDFTGCVIAWNSN |
| | | NLDSKVGGNYNYLYRLFRKSNLKPFERDISTEIYQAGSTPCNGVEGEN |
| | | CYFPLQSYGFQPTNGVGYQPYRVVVLSFELLHAPATVCGPKKSTNLVK |
| | | NKCVNFNFNGLTGTGVLTESNKKFLPFQQFGRDIADTTDAVRDPQTLE |
| | | ILDITPCSFGGVSVITPGTNTSNQVAVLYQDVNCTEVPVAIHADQLTPT |
| | | WRVYSTGSNVFQTRAGCLIGAEHVNNSYECDIPIGAGICASYQTQTNSP |
| | | RRARSVASQSIIAYTMSLGAENSVAYSNNSIAIPTNFTISVTTEILPVSMT |
| | | KTSVDCTMYICGDSTECSNLLLQYGSFCTQLNRALTGIAVEQDKNTQE |
| | | VFAQVKQIYKTPPIKDFGGFNFSQILPDPSKPSKRSFIEDLLFNKVTLAD |
| | | AGFIKQYGDCLGDIAARDLICAQKFNGLTVLPPLLTDEMIAQYTSALLA |
| | | GTITSGWTFGAGAALQIPFAMQMAYRFNGIGVTQNVLYENQKLIANQF |
| | | NSAIGKIQDSLSSTASALGKLQDVVNQNAQALNTLVKQLSSNFGAISSV |
| | | LNDILSRLDPPEAEVQIDRLITGRLQSLQTYVTQQLIRAAEIRASANLAA |
| | | TKMSECVLGQSKRVDFCGKGYHLMSFPQSAPHGVVFLHVTYVPAQEK |
| | | NFTTAPAICHDGKAHFPREGVFVSNGTHWFVTQRNFYEPQIITTDNTFV |
| | | SGNCDVVIGIVNNTVYDPLQPELDSFKEELDKYFKNHTSPDVDLGDISG |
| | | INASVVNIQKEIDRLNEVAKNLNESLIDLQELGKYEQYIKWPWYIWLG |
| | | FIAGLIAIVMVTIMLCCMTSCCSCLKGCCSCGSCCKFDEDDSEPVLKGV |
| | | KLHYT |
| 382 | SARS-CoV2 | FVFLVLLPLVSSQCVNLTTRTQLPPAYTNSFTRGVYYPDKVFRSSVLHS |
| | full-length | TQDLFLPFFSNVTWFHAIHVSGTNGTKRFDNPVLPFNDGVYFASTEKS |
| | stabilized S | NIIRGWIFGTTLDSKTQSLLIVNNATNVVIKVCEFQFCNDPFLGVYYHK |
| | protein-K417T, | NNKSWMESEFRVYSSANNCTFEYVSQPFLMDLEGKQGNFKNLREFVF |
| | E484K, and | KNIDGYFKIYSKHTPINLVRDLPQGFSALEPLVDLPIGINITRFQTLLALH |
| | N501Y | RSYLTPGDSSSGWTAGAAAYYVGYLQPRTFLLKYNENGTITDAVDCA |
| | | LDPLSETKCTLKSFTVEKGIYQTSNFRVQPTESIVRFPNITNLCPFGEVEN |
| | | ATRFASVYAWNRKRISNCVADYSVLYNSASFSTFKCYGVSPTKLNDLC |
| | | FTNVYADSFVIRGDEVRQIAPGQTGTIADYNYKLPDDFTGCVIAWNSN |
| | | NLDSKVGGNYNYLYRLFRKSNLKPFERDISTEIYQAGSTPCNGVKGFN |
| | | CYFPLQSYGFQPTYGVGYQPYRVVVLSFELLHAPATVCGPKKSTNLVK |
| | | NKCVNFNFNGLTGTGVLTESNKKFLPFQQFGRDIADTTDAVRDPQTLE |
| | | ILDITPCSFGGVSVITPGTNTSNQVAVLYQDVNCTEVPVAIHADQLTPT |
| | | WRVYSTGSNVFQTRAGCLIGAEHVNNSYECDIPIGAGICASYQTQTNSP |
| | | RRARSVASQSIIAYTMSLGAENSVAYSNNSIAIPTNFTISVTTEILPVSMT |
| | | KTSVDCTMYICGDSTECSNLLLQYGSFCTQLNRALTGIAVEQDKNTQE |
| | | VFAQVKQIYKTPPIKDFGGFNFSQILPDPSKPSKRSFIEDLLFNKVTLAD |
| | | AGFIKQYGDCLGDIAARDLICAQKFNGLTVLPPLLTDEMIAQYTSALLA |
| | | GTITSGWTFGAGAALQIPFAMQMAYRFNGIGVTQNVLYENQKLIANQF |
| | | NSAIGKIQDSLSSTASALGKLQDVVNQNAQALNTLVKQLSSNFGAISSV |
| | | LNDILSRLDPPEAEVQIDRLITGRLQSLQTYVTQQLIRAAEIRASANLAA |
| | | TKMSECVLGQSKRVDFCGKGYHLMSFPQSAPHGVVFLHVTYVPAQEK |
| | | NFTTAPAICHDGKAHFPREGVFVSNGTHWFVTQRNFYEPQIITTDNTFV |
| | | SGNCDVVIGIVNNTVYDPLQPELDSFKEELDKYFKNHTSPDVDLGDISG |
| | | INASVVNIQKEIDRLNEVAKNLNESLIDLQELGKYEQYIKWPWYIWLG |
| | | FIAGLIAIVMVTIMLCCMTSCCSCLKGCCSCGSCCKFDEDDSEPVLKGV |
| | | KLHYT |
| 383 | SARS-CoV2 | FVFLVLLPLVSSQCVNLRTRTQLPPAYTNSFTRGVYYPDKVFRSSVLHS |
| | full-length | TQDLFLPFFSNVTWFHAIHVSGTNGTKRFDNPVLPFNDGVYFASIEKSN |
| | stabilized S | IIRGWIFGTTLDSKTQSLLIVNNATNVVIKVCEFQFCNDPFLDVYYHKN |
| | protein Delta | NKSWMESGVYSSANNCTFEYVSQPFLMDLEGKQGNFKNLREFVFKNI |
| | variant | DGYFKIYSKHTPINLVRDLPQGFSALEPLVDLPIGINITRFQTLLALHRSY |
| | | LTPGDSSSGWTAGAAAYYVGYLQPRTFLLKYNENGTITDAVDCALDP |
| | | LSETKCTLKSFTVEKGIYQTSNFRVQPTESIVRFPNITNLCPFGEVFNAT |
| | | RFASVYAWNRKRISNCVADYSVLYNSASFSTFKCYGVSPTKLNDLCFT |
| | | NVYADSFVIRGDEVRQIAPGQTGKIADYNYKLPDDFTGCVIAWNSNNL |
| | | DSKVGGNYNYRYRLFRKSNLKPFERDISTEIYQAGSKPCNGVEGENCY |
| | | FPLQSYGFQPTNGVGYQPYRVVVLSFELLHAPATVCGPKKSTNLVKNK |
| | | CVNFNFNGLTGTGVLTESNKKFLPFQQFGRDIADTTDAVRDPQTLEILD |
| | | ITPCSFGGVSVITPGTNTSNQVAVLYQGVNCTEVPVAIHADQLTPTWR |
| | | VYSTGSNVFQTRAGCLIGAEHVNNSYECDIPIGAGICASYQTQTNSRRR |
| | | ARSVASQSIIAYTMSLGAENSVAYSNNSIAIPTNFTISVTTEILPVSMTKT |
| | | SVDCTMYICGDSTECSNLLLQYGSFCTQLNRALTGIAVEQDKNTQEVF |
| | | AQVKQIYKTPPIKDFGGFNFSQILPDPSKPSKRSFIEDLLFNKVTLADAG |
| | | FIKQYGDCLGDIAARDLICAQKFNGLTVLPPLLTDEMIAQYTSALLAGT |
| | | ITSGWTFGAGAALQIPFAMQMAYRFNGIGVTQNVLYENQKLIANQFNS |
| | | AIGKIQDSLSSTASALGKLQNVVNQNAQALNTLVKQLSSNFGAISSVLN |
| | | DILSRLDPPEAEVQIDRLITGRLQSLQTYVTQQLIRAAEIRASANLAATK |
| | | MSECVLGQSKRVDFCGKGYHLMSFPQSAPHGVVFLHVTYVPAQEKNF |
| | | TTAPAICHDGKAHFPREGVFVSNGTHWFVTQRNFYEPQIITTDNTFVSG |
| | | NCDVVIGIVNNTVYDPLQPELDSFKEELDKYFKNHTSPDVDLGDISGIN |
| | | ASVVNIQKEIDRLNEVAKNLNESLIDLQELGKYEQYIKWPWYIWLGFI |
| | | AGLIAIVMVTIMLCCMTSCCSCLKGCCSCGSCCKFDEDDSEPVLKGVK |
| | | LHYT |
| 384 | SARS-CoV2 | FVFLVLLPLVSSQCVNLRTRTQLPPAYTNSFTRGVYYPDKVFRSSVLHS |
| | full-length | TQDLFLPFFSNVTWFHAIHFSGTNGTKRFDNPVLPFNDGVYFASIEKSNI |
| | stabilized S | IRGWIFGTTLDSKTQSLLIVNNATNVVIKVCEFQFCNDPFLDVYYHKNN |
| | protein Delta | KSWMESGVYSSANNCTFEYVSQPFLMDLEGKQGNFKNLREFVFKNID |
| | variant, plus | GYFKIYSKHTPINLVRDLPQGFSVLEPLVDLPIGINITRFQTLLALHRSYL |
| | | TPGDSSSGLTAGAAAYYVGYLQPRTFLLKYNENGTITDAVDCALDPLS |
| | | ETKCTLKSFTVEKGIYQTSNFRVQPTESIVRFPNITNLCPFGEVFNATRF |
| | | ASVYAWNRKRISNCVADYSVLYNSASFSTFKCYGVSPTKLNDLCFTNV |
| | | YADSFVIRGDEVRQIAPGQTGNIADYNYKLPDDFTGCVIAWNSNNLDS |
| | | KVGGNYNYRYRLFRKSNLKPFERDISTEIYQAGSKPCNGVEGFNCYFP |
| | | LQSYGFQPTNGVGYQPYRVVVLSFELLHAPATVCGPKKSTNLVKNKC |
| | | VNFNFNGLTGTGVLTESNKKFLPFQQFGRDIADTTDAVRDPQTLEILDI |
| | | TPCSFGGVSVITPGTNTSNQVAVLYQGVNCTEVPVAIHADQLTPTWRV |
| | | YSTGSNVFQTRAGCLIGAEHVNNSYECDIPIGAGICASYQTQTNSRRRA |
| | | RSVASQSIIAYTMSLGAENSVAYSNNSIAIPTNFTISVTTEILPVSMTKTS |
| | | VDCTMYICGDSTECSNLLLQYGSFCTQLNRALTGIAVEQDKNTQEVFA |
| | | QVKQIYKTPPIKDFGGFNFSQILPDPSKPSKRSFIEDLLFNKVTLADAGFI |
| | | KQYGDCLGDIAARDLICAQKFNGLTVLPPLLTDEMIAQYTSALLAGTIT |
| | | SGWTFGAGAALQIPFAMQMAYRFNGIGVTQNVLYENQKLIANQFNSA |
| | | IGKIQDSLSSTASALGKLQNVVNQNAQALNTLVKQLSSNFGAISSVLND |
| | | ILSRLDPPEAEVQIDRLITGRLQSLQTYVTQQLIRAAEIRASANLAATKM |
| | | SECVLGQSKRVDFCGKGYHLMSFPQSAPHGVVFLHVTYVPAQEKNFT |
| | | TAPAICHDGKAHFPREGVFVSNGTHWFVTQRNFYEPQIITTDNTFVSGN |
| | | CDVVIGIVNNTVYDPLQPELDSFKEELDKYFKNHTSPDVDLGDISGINA |
| | | SVVNIQKEIDRLNEVAKNLNESLIDLQELGKYEQYIKWPWYIWLGFIA |
| | | GLIAIVMVTIMLCCMTSCCSCLKGCCSCGSCCKFDEDDSEPVLKGVKL |
| | | HYT |
| 385 | SARS-CoV2 E | YSFVSEETGTLIVNSVLLFLAFVVFLLVTLAILTALRLCAYCCNIVNVSL |
| | protein amino | VKPSFYVYSRVKNLNSSRVPDLLV |
| | acid sequence | |
| 386 | SARS-CoV2 M | ADSNGTITVEELKKLLEQWNLVIGFLFLTWICLLQFAYANRNRFLYIIK |
| | protein amino | LIFLWLLWPVTLACFVLAAVYRINWITGGIAIAMACLVGLMWLSYFIA |
| | acid sequence | SFRLFARTRSMWSFNPETNILLNVPLHGTILTRPLLESELVIGAVILRGH |
| | | LRIAGHHLGRCDIKDLPKEITVATSRTLSYYKLGASQRVAGDSGFAAY |
| | | SRYRIGNYKLNTDHSSSSDNIALLVQ |
| 387 | SARS-CoV2 | ESLVPGFNEKTHVQLSLPVLQVRDVLVRGFGDSVEEVLSEARQHLKDG |
| | PP1ab | TCGLVEVEKGVLPQLEQPYVFIKRSDARTAPHGHVMVELVAELEGIQY |
| | polyprotein | GRSGETLGVLVPHVGEIPVAYRKVLLRKNGNKGAGGHSYGADLKSFD |
| | amino acid | LGDELGTDPYEDFQENWNTKHSSGVTRELMRELNGGAYTRYVDNNF |
| | sequence | CGPDGYPLECIKDLLARAGKASCTLSEQLDFIDTKRGVYCCREHEHEIA |
| | | WYTERSEKSYELQTPFEIKLAKKFDTFNGECPNFVFPLNSIIKTIQPRVE |
| | | KKKLDGFMGRIRSVYPVASPNECNQMCLSTLMKCDHCGETSWQTGDF |
| | | VKATCEFCGTENLTKEGATTCGYLPQNAVVKIYCPACHNSEVGPEHSL |
| | | AEYHNESGLKTILRKGGRTIAFGGCVFSYVGCHNKCAYWVPRASANIG |
| | | CNHTGVVGEGSEGLNDNLLEILQKEKVNINIVGDFKLNEEIAIILASFSA |
| | | STSAFVETVKGLDYKAFKQIVESCGNFKVTKGKAKKGAWNIGEQKSIL |
| | | SPLYAFASEAARVVRSIFSRTLETAQNSVRVLQKAAITILDGISQYSLRLI |
| | | DAMMFTSDLATNNLVVMAYITGGVVQLTSQWLTNIFGTVYEKLKPVL |
| | | DWLEEKFKEGVEFLRDGWEIVKFISTCACEIVGGQIVTCAKEIKESVQT |
| | | FFKLVNKFLALCADSIIIGGAKLKALNLGETFVTHSKGLYRKCVKSREE |
| | | TGLLMPLKAPKEIIFLEGETLPTEVLTEEVVLKTGDLQPLEQPTSEAVEA |
| | | PLVGTPVCINGLMLLEIKDTEKYCALAPNMMVTNNTFTLKGGAPTKV |
| | | TFGDDTVIEVQGYKSVNITFELDERIDKVLNEKCSAYTVELGTEVNEFA |
| | | CVVADAVIKTLQPVSELLTPLGIDLDEWSMATYYLFDESGEFKLASHM |
| | | YCSFYPPDEDEEEGDCEEEEFEPSTQYEYGTEDDYQGKPLEFGATSAAL |
| | | QPEEEQEEDWLDDDSQQTVGQQDGSEDNQTTTIQTIVEVQPQLEMELT |
| | | PVVQTIEVNSFSGYLKLTDNVYIKNADIVEEAKKVKPTVVVNAANVYL |
| | | KHGGGVAGALNKATNNAMQVESDDYIATNGPLKVGGSCVLSGHNLA |
| | | KHCLHVVGPNVNKGEDIQLLKSAYENFNQHEVLLAPLLSAGIFGADPI |
| | | HSLRVCVDTVRTNVYLAVFDKNLYDKLVSSFLEMKSEKQVEQKIAEIP |
| | | KEEVKPFITESKPSVEQRKQDDKKIKACVEEVTTTLEETKFLTENLLLYI |
| | | DINGNLHPDSATLVSDIDITFLKKDAPYIVGDVVQEGVLTAVVIPTKKA |
| | | GGTTEMLAKALRKVPTDNYITTYPGQGLNGYTVEEAKTVLKKCKSAF |
| | | YILPSIISNEKQEILGTVSWNLREMLAHAEETRKLMPVCVETKAIVSTIQ |
| | | RKYKGIKIQEGVVDYGARFYFYTSKTTVASLINTLNDLNETLVTMPLG |
| | | YVTHGLNLEEAARYMRSLKVPATVSVSSPDAVTAYNGYLTSSSKTPEE |
| | | HFIETISLAGSYKDWSYSGQSTQLGIEFLKRGDKSVYYTSNPTTFHLDG |
| | | EVITFDNLKTLLSLREVRTIKVFTTVDNINLHTQVVDMSMTYGQQFGP |
| | | TYLDGADVTKIKPHNSHEGKTFYVLPNDDTLRVEAFEYYHTTDPSFLG |
| | | RYMSALNHTKKWKYPQVNGLTSIKWADNNCYLATALLTLQQIELKFN |
| | | PPALQDAYYRARAGEAANFCALILAYCNKTVGELGDVRETMSYLFQH |
| | | ANLDSCKRVLNVVCKTCGQQQTTLKGVEAVMYMGTLSYEQFKKGVQ |
| | | IPCTCGKQATKYLVQQESPFVMMSAPPAQYELKHGTFTCASEYTGNY |
| | | QCGHYKHITSKETLYCIDGALLTKSSEYKGPITDVFYKENSYTTTIKPVT |
| | | YKLDGVVCTEIDPKLDNYYKKDNSYFTEQPIDLVPNQPYPNASFDNFK |
| | | FVCDNIKFADDLNQLTGYKKPASRELKVTFFPDLNGDVVAIDYKHYTP |
| | | SFKKGAKLLHKPIVWHVNNATNKATYKPNTWCIRCLWSTKPVETSNS |
| | | FDVLKSEDAQGMDNLACEDLKPVSEEVVENPTIQKDVLECNVKTTEV |
| | | VGDIILKPANNSLKITEEVGHTDLMAAYVDNSSLTIKKPNELSRVLGLK |
| | | TLATHGLAAVNSVPWDTIANYAKPFLNKVVSTTTNIVTRCLNRVCTNY |
| | | MPYFFTLLLQLCTFTRSTNSRIKASMPTTIAKNTVKSVGKFCLEASFNY |
| | | LKSPNFSKLINIIIWFLLLSVCLGSLIYSTAALGVLMSNLGMPSYCTGYR |
| | | EGYLNSTNVTIATYCTGSIPCSVCLSGLDSLDTYPSLETIQITISSFKWDL |
| | | TAFGLVAEWFLAYILFTRFFYVLGLAAIMQLFFSYFAVHFISNSWLMW |
| | | LIINLVQMAPISAMVRMYIFFASFYYVWKSYVHVVDGCNSSTCMMCY |
| | | KRNRATRVECTTIVNGVRRSFYVYANGGKGFCKLHNWNCVNCDTFC |
| | | AGSTFISDEVARDLSLQFKRPINPTDQSSYIVDSVTVKNGSIHLYFDKAG |
| | | QKTYERHSLSHFVNLDNLRANNTKGSLPINVIVFDGKSKCEESSAKSAS |
| | | VYYSQLMCQPILLLDQALVSDVGDSAEVAVKMFDAYVNTFSSTFNVP |
| | | MEKLKTLVATAEAELAKNVSLDNVLSTFISAARQGFVDSDVETKDVV |
| | | ECLKLSHQSDIEVTGDSCNNYMLTYNKVENMTPRDLGACIDCSARHIN |
| | | AQVAKSHNIALIWNVKDFMSLSEQLRKQIRSAAKKNNLPFKLTCATTR |
| | | QVVNVVTTKIALKGGKIVNNWLKQLIKVTLVFLFVAAIFYLITPVHVM |
| | | SKHTDFSSEIIGYKAIDGGVTRDIASTDTCFANKHADFDTWFSQRGGSY |
| | | TNDKACPLIAAVITREVGFVVPGLPGTILRTTNGDFLHFLPRVFSAVGNI |
| | | CYTPSKLIEYTDFATSACVLAAECTIFKDASGKPVPYCYDTNVLEGSVA |
| | | YESLRPDTRYVLMDGSIIQFPNTYLEGSVRVVTTFDSEYCRHGTCERSE |
| | | AGVCVSTSGRWVLNNDYYRSLPGVFCGVDAVNLLTNMFTPLIQPIGAL |
| | | DISASIVAGGIVAIVVTCLAYYFMRFRRAFGEYSHVVAFNTLLFLMSFT |
| | | VLCLTPVYSFLPGVYSVIYLYLTFYLTNDVSFLAHIQWMVMFTPLVPF |
| | | WITIAYIICISTKHFYWFFSNYLKRRVVFNGVSFSTFEEAALCTFLINKE |
| | | MYLKLRSDVLLPLTQYNRYLALYNKYKYFSGAMDTTSYREAACCHL |
| | | AKALNDFSNSGSDVLYQPPQTSITSAVLQSGFRKMAFPSGKVEGCMVQ |
| | | VTCGTTTLNGLWLDDVVYCPRHVICTSEDMLNPNYEDLLIRKSNHNFL |
| | | VQAGNVQLRVIGHSMQNCVLKLKVDTANPKTPKYKFVRIQPGQTFSV |
| | | LACYNGSPSGVYQCAMRPNFTIKGSFLNGSCGSVGFNIDYDCVSFCYM |
| | | HHMELPTGVHAGTDLEGNFYGPFVDRQTAQAAGTDTTITVNVLAWL |
| | | YAAVINGDRWFLNRFTTTLNDFNLVAMKYNYEPLTQDHVDILGPLSA |
| | | QTGIAVLDMCASLKELLQNGMNGRTILGSALLEDEFTPFDVVRQCSGV |
| | | TFQSAVKRTIKGTHHWLLLTILTSLLVLVQSTQWSLFFFLYENAFLPFA |
| | | MGIIAMSAFAMMFVKHKHAFLCLFLLPSLATVAYFNMVYMPASWVM |
| | | RIMTWLDMVDTSLSGFKLKDCVMYASAVVLLILMTARTVYDDGARR |
| | | VWTLMNVLTLVYKVYYGNALDQAISMWALIISVTSNYSGVVTTVMFL |
| | | ARGIVFMCVEYCPIFFITGNTLQCIMLVYCFLGYFCTCYFGLFCLLNRY |
| | | FRLTLGVYDYLVSTQEFRYMNSQGLLPPKNSIDAFKLNIKLLGVGGKP |
| | | CIKVATVQSKMSDVKCTSVVLLSVLQQLRVESSSKLWAQCVQLHNDI |
| | | LLAKDTTEAFEKMVSLLSVLLSMQGAVDINKLCEEMLDNRATLQAIAS |
| | | EFSSLPSYAAFATAQEAYEQAVANGDSEVVLKKLKKSLNVAKSEFDR |
| | | DAAMQRKLEKMADQAMTQMYKQARSEDKRAKVTSAMQTMLFTML |
| | | RKLDNDALNNIINNARDGCVPLNIIPLTTAAKLMVVIPDYNTYKNTCD |
| | | GTTFTYASALWEIQQVVDADSKIVQLSEISMDNSPNLAWPLIVTALRA |
| | | NSAVKLQNNELSPVALRQMSCAAGTTQTACTDDNALAYYNTTKGGR |
| | | FVLALLSDLQDLKWARFPKSDGTGTIYTELEPPCRFVTDTPKGPKVKY |
| | | LYFIKGLNNLNRGMVLGSLAATVRLQAGNATEVPANSTVLSFCAFAV |
| | | DAAKAYKDYLASGGQPITNCVKMLCTHTGTGQAITVTPEANMDQESF |
| | | GGASCCLYCRCHIDHPNPKGFCDLKGKYVQIPTTCANDPVGFTLKNTV |
| | | CTVCGMWKGYGCSCDQLREPMLQSADAQSFLNRVCGVSAARLTPCG |
| | | TGTSTDVVYRAFDIYNDKVAGFAKFLKTNCCRFQEKDEDDNLIDSYFV |
| | | VKRHTFSNYQHEETIYNLLKDCPAVAKHDFFKFRIDGDMVPHISRQRL |
| | | TKYTMADLVYALRHFDEGNCDTLKEILVTYNCCDDDYFNKKDWYDF |
| | | VENPDILRVYANLGERVRQALLKTVQFCDAMRNAGIVGVLTLDNQDL |
| | | NGNWYDFGDFIQTTPGSGVPVVDSYYSLLMPILTLTRALTAESHVDTD |
| | | LTKPYIKWDLLKYDFTEERLKLFDRYFKYWDQTYHPNCVNCLDDRCI |
| | | LHCANFNVLFSTVFPPTSFGPLVRKIFVDGVPFVVSTGYHFRELGVVHN |
| | | QDVNLHSSRLSFKELLVYAADPAMHAASGNLLLDKRTTCFSVAALTN |
| | | NVAFQTVKPGNFNKDFYDFAVSKGFFKEGSSVELKHFFFAQDGNAAIS |
| | | DYDYYRYNLPTMCDIRQLLFVVEVVDKYFDCYDGGCINANQVIVNNL |
| | | DKSAGFPFNKWGKARLYYDSMSYEDQDALFAYTKRNVIPTITQMNLK |
| | | YAISAKNRARTVAGVSICSTMTNRQFHQKLLKSIAATRGATVVIGTSKF |
| | | YGGWHNMLKTVYSDVENPHLMGWDYPKCDRAMPNMLRIMASLVLA |
| | | RKHTTCCSLSHRFYRLANECAQVLSEMVMCGGSLYVKPGGTSSGDAT |
| | | TAYANSVFNICQAVTANVNALLSTDGNKIADKYVRNLQHRLYECLYR |
| | | NRDVDTDFVNEFYAYLRKHFSMMILSDDAVVCFNSTYASQGLVASIK |
| | | NFKSVLYYQNNVFMSEAKCWTETDLTKGPHEFCSQHTMLVKQGDDY |
| | | VYLPYPDPSRILGAGCFVDDIVKTDGTLMIERFVSLAIDAYPLTKHPNQ |
| | | EYADVFHLYLQYIRKLHDELTGHMLDMYSVMLINDNTSRYWEPEFY |
| | | EAMYTPHTVLQAVGACVLCNSQTSLRCGACIRRPFLCCKCCYDHVIST |
| | | SHKLVLSVNPYVCNAPGCDVTDVTQLYLGGMSYYCKSHKPPISFPLCA |
| | | NGQVFGLYKNTCVGSDNVTDFNAIATCDWTNAGDYILANTCTERLKL |
| | | FAAETLKATEETFKLSYGIATVREVLSDRELHLSWEVGKPRPPLNRNY |
| | | VFTGYRVTKNSKVQIGEYTFEKGDYGDAVVYRGTTTYKLNVGDYFVL |
| | | TSHTVMPLSAPTLVPQEHYVRITGLYPTLNISDEFSSNVANYQKVGMQ |
| | | KYSTLQGPPGTGKSHFAIGLALYYPSARIVYTACSHAAVDALCEKALK |
| | | YLPIDKCSRIIPARARVECFDKFKVNSTLEQYVFCTVNALPETTADIVVF |
| | | DEISMATNYDLSVVNARLRAKHYVYIGDPAQLPAPRTLLTKGTLEPEY |
| | | FNSVCRLMKTIGPDMFLGTCRRCPAEIVDTVSALVYDNKLKAHKDKS |
| | | AQCFKMFYKGVITHDVSSAINRPQIGVVREFLTRNPAWRKAVFISPYNS |
| | | QNAVASKILGLPTQTVDSSQGSEYDYVIFTQTTETAHSCNVNRFNVAIT |
| | | RAKVGILCIMSDRDLYDKLQFTSLEIPRRNVATLQAENVTGLFKDCSK |
| | | VITGLHPTQAPTHLSVDTKFKTEGLCVDIPGIPKDMTYRRLISMMGFK |
| | | MNYQVNGYPNMFITREEAIRHVRAWIGFDVEGCHATREAVGTNLPLQ |
| | | LGFSTGVNLVAVPTGYVDTPNNTDFSRVSAKPPPGDQFKHLIPLMYKG |
| | | LPWNVVRIKIVQMLSDTLKNLSDRVVFVLWAHGFELTSMKYFVKIGPE |
| | | RTCCLCDRRATCFSTASDTYACWHHSIGFDYVYNPFMIDVQQWGFTG |
| | | NLQSNHDLYCQVHGNAHVASCDAIMTRCLAVHECFVKRVDWTIEYPII |
| | | GDELKINAACRKVQHMVVKAALLADKFPVLHDIGNPKAIKCVPQADV |
| | | EWKFYDAQPCSDKAYKIEELFYSYATHSDKFTDGVCLFWNCNVDRYP |
| | | ANSIVCRFDTRVLSNLNLPGCDGGSLYVNKHAFHTPAFDKSAFVNLKQ |
| | | LPFFYYSDSPCESHGKQVVSDIDYVPLKSATCITRQNLGGAVCRHHAN |
| | | EYRLYLDAYNMMISAGFSLWVYKQFDTYNLWNTFTRLQSLENVAFN |
| | | VVNKGHFDGQQGEVPVSIINNTVYTKVDGVDVELFENKTTLPVNVAF |
| | | ELWAKRNIKPVPEVKILNNLGVDIAANTVIWDYKRDAPAHISTIGVCS |
| | | MTDIAKKPTETICAPLTVFFDGRVDGQVDLFRNARNGVLITEGSVKGL |
| | | QPSVGPKQASLNGVTLIGEAVKTQFNYYKKVDGVVQQLPETYFTQSR |
| | | NLQEFKPRSQMEIDFLELAMDEFIERYKLEGYAFEHIVYGDFSHSQLGG |
| | | LHLLIGLAKRFKESPFELEDFIPMDSTVKNYFITDAQTGSSKCVCSVIDL |
| | | LLDDFVEIIKSQDLSVVSKVVKVTIDYTEISFMLWCKDGHVETFYPKLQ |
| | | SSQAWQPGVAMPNLYKMQRMLLEKCDLQNYGDSATLPKGIMMNVA |
| | | KYTQLCQYLNTLTLAVPYNMRVIHFGAGSDKGVAPGTAVLRQWLPTG |
| | | TLLVDSDLNDFVSDADSTLIGDCATVHTANKWDLIISDMYDPKTKNVT |
| | | KENDSKEGFFTYICGFIQQKLALGGSVAIKITEHSWNADLYKLMGHFA |
| | | WWTAFVTNVNASSSEAFLIGCNYLGKPREQIDGYVMHANYIFWRNTN |
| | | PIQLSSYSLFDMSKFPLKLRGTAVMSLKEGQINDMILSLLSKGRLIIREN |
| | | NRVVISSDVLVNN |
| 388 | SARS-CoV2 | ESLVPGFNEKTHVQLSLPVLQVRDVLVRGFGDSVEEVLSEARQHLKDG |
| | PPla polyprotein | TCGLVEVEKGVLPQLEQPYVFIKRSDARTAPHGHVMVELVAELEGIQY |
| | amino acid | GRSGETLGVLVPHVGEIPVAYRKVLLRKNGNKGAGGHSYGADLKSFD |
| | sequence. | LGDELGTDPYEDFQENWNTKHSSGVTRELMRELNGGAYTRYVDNNF |
| | (Wuhan-Hu-1) | CGPDGYPLECIKDLLARAGKASCTLSEQLDFIDTKRGVYCCREHEHEIA |
| | | WYTERSEKSYELQTPFEIKLAKKFDTFNGECPNFVFPLNSIIKTIQPRVE |
| | | KKKLDGFMGRIRSVYPVASPNECNQMCLSTLMKCDHCGETSWQTGDF |
| | | VKATCEFCGTENLTKEGATTCGYLPQNAVVKIYCPACHNSEVGPEHSL |
| | | AEYHNESGLKTILRKGGRTIAFGGCVFSYVGCHNKCAYWVPRASANIG |
| | | CNHTGVVGEGSEGLNDNLLEILQKEKVNINIVGDFKLNEEIAIILASFSA |
| | | STSAFVETVKGLDYKAFKQIVESCGNFKVTKGKAKKGAWNIGEQKSIL |
| | | SPLYAFASEAARVVRSIFSRTLETAQNSVRVLQKAAITILDGISQYSLRLI |
| | | DAMMFTSDLATNNLVVMAYITGGVVQLTSQWLTNIFGTVYEKLKPVL |
| | | DWLEEKFKEGVEFLRDGWEIVKFISTCACEIVGGQIVTCAKEIKESVQT |
| | | FFKLVNKFLALCADSIIIGGAKLKALNLGETFVTHSKGLYRKCVKSREE |
| | | TGLLMPLKAPKEIIFLEGETLPTEVLTEEVVLKTGDLQPLEQPTSEAVEA |
| | | PLVGTPVCINGLMLLEIKDTEKYCALAPNMMVTNNTFTLKGGAPTKV |
| | | TFGDDTVIEVQGYKSVNITFELDERIDKVLNEKCSAYTVELGTEVNEFA |
| | | CVVADAVIKTLQPVSELLTPLGIDLDEWSMATYYLFDESGEFKLASHM |
| | | YCSFYPPDEDEEEGDCEEEEFEPSTQYEYGTEDDYQGKPLEFGATSAAL |
| | | QPEEEQEEDWLDDDSQQTVGQQDGSEDNQTTTIQTIVEVQPQLEMELT |
| | | PVVQTIEVNSFSGYLKLTDNVYIKNADIVEEAKKVKPTVVVNAANVYL |
| | | KHGGGVAGALNKATNNAMQVESDDYIATNGPLKVGGSCVLSGHNLA |
| | | KHCLHVVGPNVNKGEDIQLLKSAYENFNQHEVLLAPLLSAGIFGADPI |
| | | HSLRVCVDTVRTNVYLAVFDKNLYDKLVSSFLEMKSEKQVEQKIAEIP |
| | | KEEVKPFITESKPSVEQRKQDDKKIKACVEEVTTTLEETKFLTENLLLYI |
| | | DINGNLHPDSATLVSDIDITFLKKDAPYIVGDVVQEGVLTAVVIPTKKA |
| | | GGTTEMLAKALRKVPTDNYITTYPGQGLNGYTVEEAKTVLKKCKSAF |
| | | YILPSIISNEKQEILGTVSWNLREMLAHAEETRKLMPVCVETKAIVSTIQ |
| | | RKYKGIKIQEGVVDYGARFYFYTSKTTVASLINTLNDLNETLVTMPLG |
| | | YVTHGLNLEEAARYMRSLKVPATVSVSSPDAVTAYNGYLTSSSKTPEE |
| | | HFIETISLAGSYKDWSYSGQSTQLGIEFLKRGDKSVYYTSNPTTFHLDG |
| | | EVITFDNLKTLLSLREVRTIKVFTTVDNINLHTQVVDMSMTYGQQFGP |
| | | TYLDGADVTKIKPHNSHEGKTFYVLPNDDTLRVEAFEYYHTTDPSFLG |
| | | RYMSALNHTKKWKYPQVNGLTSIKWADNNCYLATALLTLQQIELKFN |
| | | PPALQDAYYRARAGEAANFCALILAYCNKTVGELGDVRETMSYLFQH |
| | | ANLDSCKRVLNVVCKTCGQQQTTLKGVEAVMYMGTLSYEQFKKGVQ |
| | | IPCTCGKQATKYLVQQESPFVMMSAPPAQYELKHGTFTCASEYTGNY |
| | | QCGHYKHITSKETLYCIDGALLTKSSEYKGPITDVFYKENSYTTTIKPVT |
| | | YKLDGVVCTEIDPKLDNYYKKDNSYFTEQPIDLVPNQPYPNASFDNFK |
| | | FVCDNIKFADDLNQLTGYKKPASRELKVTFFPDLNGDVVAIDYKHYTP |
| | | SFKKGAKLLHKPIVWHVNNATNKATYKPNTWCIRCLWSTKPVETSNS |
| | | FDVLKSEDAQGMDNLACEDLKPVSEEVVENPTIQKDVLECNVKTTEV |
| | | VGDIILKPANNSLKITEEVGHTDLMAAYVDNSSLTIKKPNELSRVLGLK |
| | | TLATHGLAAVNSVPWDTIANYAKPFLNKVVSTTTNIVTRCLNRVCTNY |
| | | MPYFFTLLLQLCTFTRSTNSRIKASMPTTIAKNTVKSVGKFCLEASFNY |
| | | LKSPNFSKLINIIIWFLLLSVCLGSLIYSTAALGVLMSNLGMPSYCTGYR |
| | | EGYLNSTNVTIATYCTGSIPCSVCLSGLDSLDTYPSLETIQITISSFKWDL |
| | | TAFGLVAEWFLAYILFTRFFYVLGLAAIMQLFFSYFAVHFISNSWLMW |
| | | LIINLVQMAPISAMVRMYIFFASFYYVWKSYVHVVDGCNSSTCMMCY |
| | | KRNRATRVECTTIVNGVRRSFYVYANGGKGFCKLHNWNCVNCDTFC |
| | | AGSTFISDEVARDLSLQFKRPINPTDQSSYIVDSVTVKNGSIHLYFDKAG |
| | | QKTYERHSLSHFVNLDNLRANNTKGSLPINVIVFDGKSKCEESSAKSAS |
| | | VYYSQLMCQPILLLDQALVSDVGDSAEVAVKMFDAYVNTFSSTFNVP |
| | | MEKLKTLVATAEAELAKNVSLDNVLSTFISAARQGFVDSDVETKDVV |
| | | ECLKLSHQSDIEVTGDSCNNYMLTYNKVENMTPRDLGACIDCSARHIN |
| | | AQVAKSHNIALIWNVKDFMSLSEQLRKQIRSAAKKNNLPFKLTCATTR |
| | | QVVNVVTTKIALKGGKIVNNWLKQLIKVTLVFLFVAAIFYLITPVHVM |
| | | SKHTDFSSEIIGYKAIDGGVTRDIASTDTCFANKHADFDTWFSQRGGSY |
| | | TNDKACPLIAAVITREVGFVVPGLPGTILRTTNGDFLHFLPRVFSAVGNI |
| | | CYTPSKLIEYTDFATSACVLAAECTIFKDASGKPVPYCYDTNVLEGSVA |
| | | YESLRPDTRYVLMDGSIIQFPNTYLEGSVRVVTTFDSEYCRHGTCERSE |
| | | AGVCVSTSGRWVLNNDYYRSLPGVFCGVDAVNLLTNMFTPLIQPIGAL |
| | | DISASIVAGGIVAIVVTCLAYYFMRFRRAFGEYSHVVAFNTLLFLMSFT |
| | | VLCLTPVYSFLPGVYSVIYLYLTFYLTNDVSFLAHIQWMVMFTPLVPF |
| | | WITIAYIICISTKHFYWFFSNYLKRRVVFNGVSFSTFEEAALCTFLINKE |
| | | MYLKLRSDVLLPLTQYNRYLALYNKYKYFSGAMDTTSYREAACCHL |
| | | AKALNDFSNSGSDVLYQPPQTSITSAVLQSGFRKMAFPSGKVEGCMVQ |
| | | VTCGTTTLNGLWLDDVVYCPRHVICTSEDMLNPNYEDLLIRKSNHNFL |
| | | VQAGNVQLRVIGHSMQNCVLKLKVDTANPKTPKYKFVRIQPGQTFSV |
| | | LACYNGSPSGVYQCAMRPNFTIKGSFLNGSCGSVGFNIDYDCVSFCYM |
| | | HHMELPTGVHAGTDLEGNFYGPFVDRQTAQAAGTDTTITVNVLAWL |
| | | YAAVINGDRWFLNRFTTTLNDFNLVAMKYNYEPLTQDHVDILGPLSA |
| | | QTGIAVLDMCASLKELLQNGMNGRTILGSALLEDEFTPFDVVRQCSGV |
| | | TFQSAVKRTIKGTHHWLLLTILTSLLVLVQSTQWSLFFFLYENAFLPFA |
| | | MGIIAMSAFAMMFVKHKHAFLCLFLLPSLATVAYFNMVYMPASWVM |
| | | RIMTWLDMVDTSLSGFKLKDCVMYASAVVLLILMTARTVYDDGARR |
| | | VWTLMNVLTLVYKVYYGNALDQAISMWALIISVTSNYSGVVTTVMFL |
| | | ARGIVFMCVEYCPIFFITGNTLQCIMLVYCFLGYFCTCYFGLFCLLNRY |
| | | FRLTLGVYDYLVSTQEFRYMNSQGLLPPKNSIDAFKLNIKLLGVGGKP |
| | | CIKVATVQSKMSDVKCTSVVLLSVLQQLRVESSSKLWAQCVQLHNDI |
| | | LLAKDTTEAFEKMVSLLSVLLSMQGAVDINKLCEEMLDNRATLQAIAS |
| | | EFSSLPSYAAFATAQEAYEQAVANGDSEVVLKKLKKSLNVAKSEFDR |
| | | DAAMQRKLEKMADQAMTQMYKQARSEDKRAKVTSAMQTMLFTML |
| | | RKLDNDALNNIINNARDGCVPLNIIPLTTAAKLMVVIPDYNTYKNTCD |
| | | GTTFTYASALWEIQQVVDADSKIVQLSEISMDNSPNLAWPLIVTALRA |
| | | NSAVKLQNNELSPVALRQMSCAAGTTQTACTDDNALAYYNTTKGGR |
| | | FVLALLSDLQDLWARFPKSDGTGTIYTELEPPCRFVTDTPKGPKVKYL |
| | | YFIKGLNNLNRGMVLGSLAATVRLQAGNATEVPANSTVLSFCAFAVD |
| | | AAKAYKDYLASGGQPITNCVKMLCTHTGTGQAITVTPEANMDQESFG |
| | | GASCCLYCRCHIDHPNPKGFCDLKGKYVQIPTTCANDPVGFTLKNTVC |
| | | TVCGMWKGYGCSCDQLREPMLQSADAQSFLNGFAV |
| 389 | SARS-CoV2 | ESLVPGFNEKTHVQLSLPVLQVRDVLVRGFGDSVEEVLSEARQHLKDG |
| | NSP1-3 amino | TCGLVEVEKGVLPQLEQPYVFIKRSDARTAPHGHVMVELVAELEGIQY |
| | acid sequence | GRSGETLGVLVPHVGEIPVAYRKVLLRKNGNKGAGGHSYGADLKSFD |
| | (Wuhan Hu1) | LGDELGTDPYEDFQENWNTKHSSGVTRELMRELNGGAYTRYVDNNF |
| | | CGPDGYPLECIKDLLARAGKASCTLSEQLDFIDTKRGVYCCREHEHEIA |
| | | WYTERSEKSYELQTPFEIKLAKKFDTFNGECPNFVFPLNSIIKTIQPRVE |
| | | KKKLDGFMGRIRSVYPVASPNECNQMCLSTLMKCDHCGETSWQTGDF |
| | | VKATCEFCGTENLTKEGATTCGYLPQNAVVKIYCPACHNSEVGPEHSL |
| | | AEYHNESGLKTILRKGGRTIAFGGCVFSYVGCHNKCAYWVPRASANIG |
| | | CNHTGVVGEGSEGLNDNLLEILQKEKVNINIVGDFKLNEEIAIILASFSA |
| | | STSAFVETVKGLDYKAFKQIVESCGNFKVTKGKAKKGAWNIGEQKSIL |
| | | SPLYAFASEAARVVRSIFSRTLETAQNSVRVLQKAAITILDGISQYSLRLI |
| | | DAMMFTSDLATNNLVVMAYITGGVVQLTSQWLTNIFGTVYEKLKPVL |
| | | DWLEEKFKEGVEFLRDGWEIVKFISTCACEIVGGQIVTCAKEIKESVQT |
| | | FFKLVNKFLALCADSIIIGGAKLKALNLGETFVTHSKGLYRKCVKSREE |
| | | TGLLMPLKAPKEIIFLEGETLPTEVLTEEVVLKTGDLQPLEQPTSEAVEA |
| | | PLVGTPVCINGLMLLEIKDTEKYCALAPNMMVTNNTFTLKGGAPTKV |
| | | TFGDDTVIEVQGYKSVNITFELDERIDKVLNEKCSAYTVELGTEVNEFA |
| | | CVVADAVIKTLQPVSELLTPLGIDLDEWSMATYYLFDESGEFKLASHM |
| | | YCSFYPPDEDEEEGDCEEEEFEPSTQYEYGTEDDYQGKPLEFGATSAAL |
| | | QPEEEQEEDWLDDDSQQTVGQQDGSEDNQTTTIQTIVEVQPQLEMELT |
| | | PVVQTIEVNSFSGYLKLTDNVYIKNADIVEEAKKVKPTVVVNAANVYL |
| | | KHGGGVAGALNKATNNAMQVESDDYIATNGPLKVGGSCVLSGHNLA |
| | | KHCLHVVGPNVNKGEDIQLLKSAYENFNQHEVLLAPLLSAGIFGADPI |
| | | HSLRVCVDTVRTNVYLAVFDKNLYDKLVSSFLEMKSEKQVEQKIAEIP |
| | | KEEVKPFITESKPSVEQRKQDDKKIKACVEEVTTTLEETKFLTENLLLYI |
| | | DINGNLHPDSATLVSDIDITFLKKDAPYIVGDVVQEGVLTAVVIPTKKA |
| | | GGTTEMLAKALRKVPTDNYITTYPGQGLNGYTVEEAKTVLKKCKSAF |
| | | YILPSIISNEKQEILGTVSWNLREMLAHAEETRKLMPVCVETKAIVSTIQ |
| | | RKYKGIKIQEGVVDYGARFYFYTSKTTVASLINTLNDLNETLVTMPLG |
| | | YVTHGLNLEEAARYMRSLKVPATVSVSSPDAVTAYNGYLTSSSKTPEE |
| | | HFIETISLAGSYKDWSYSGQSTQLGIEFLKRGDKSVYYTSNPTTFHLDG |
| | | EVITFDNLKTLLSLREVRTIKVFTTVDNINLHTQVVDMSMTYGQQFGP |
| | | TYLDGADVTKIKPHNSHEGKTFYVLPNDDTLRVEAFEYYHTTDPSFLG |
| | | RYMSALNHTKKWKYPQVNGLTSIKWADNNCYLATALLTLQQIELKFN |
| | | PPALQDAYYRARAGEAANFCALILAYCNKTVGELGDVRETMSYLFQH |
| | | ANLDSCKRVLNVVCKTCGQQQTTLKGVEAVMYMGTLSYEQFKKGVQ |
| | | IPCTCGKQATKYLVQQESPFVMMSAPPAQYELKHGTFTCASEYTGNY |
| | | QCGHYKHITSKETLYCIDGALLTKSSEYKGPITDVFYKENSYTTTIKPVT |
| | | YKLDGVVCTEIDPKLDNYYKKDNSYFTEQPIDLVPNQPYPNASFDNFK |
| | | FVCDNIKFADDLNQLTGYKKPASRELKVTFFPDLNGDVVAIDYKHYTP |
| | | SFKKGAKLLHKPIVWHVNNATNKATYKPNTWCIRCLWSTKPVETSNS |
| | | FDVLKSEDAQGMDNLACEDLKPVSEEVVENPTIQKDVLECNVKTTEV |
| | | VGDIILKPANNSLKITEEVGHTDLMAAYVDNSSLTIKKPNELSRVLGLK |
| | | TLATHGLAAVNSVPWDTIANYAKPFLNKVVSTTTNIVTRCLNRVCTNY |
| | | MPYFFTLLLQLCTFTRSTNSRIKASMPTTIAKNTVKSVGKFCLEASFNY |
| | | LKSPNFSKLINIIIWFLLLSVCLGSLIYSTAALGVLMSNLGMPSYCTGYR |
| | | EGYLNSTNVTIATYCTGSIPCSVCLSGLDSLDTYPSLETIQITISSFKWDL |
| | | TAFGLVAEWFLAYILFTRFFYVLGLAAIMQLFFSYFAVHFISNSWLMW |
| | | LIINLVQMAPISAMVRMYIFFASFYYVWKSYVHVVDGCNSSTCMMCY |
| | | KRNRATRVECTTIVNGVRRSFYVYANGGKGFCKLHNWNCVNCDTFC |
| | | AGSTFISDEVARDLSLQFKRPINPTDQSSYIVDSVTVKNGSIHLYFDKAG |
| | | QKTYERHSLSHFVNLDNLRANNTKGSLPINVIVFDGKSKCEESSAKSAS |
| | | VYYSQLMCQPILLLDQALVSDVGDSAEVAVKMFDAYVNTFSSTFNVP |
| | | MEKLKTLVATAEAELAKNVSLDNVLSTFISAARQGFVDSDVETKDVV |
| | | ECLKLSHQSDIEVTGDSCNNYMLTYNKVENMTPRDLGACIDCSARHIN |
| | | AQVAKSHNIALIWNVKDFMSLSEQLRKQIRSAAKKNNLPFKLTCATTR |
| | | QVVNVVTTKIALKGG |
| 390 | SARS-CoV2 | KIVNNWLKQLIKVTLVFLFVAAIFYLITPVHVMSKHTDFSSEIIGYKAID |
| | NSP4-11 amino | GGVTRDIASTDTCFANKHADFDTWFSQRGGSYTNDKACPLIAAVITRE |
| | acid sequence | VGFVVPGLPGTILRTTNGDFLHFLPRVFSAVGNICYTPSKLIEYTDFATS |
| | (Wuhan Hu1) | ACVLAAECTIFKDASGKPVPYCYDTNVLEGSVAYESLRPDTRYVLMD |
| | | GSIIQFPNTYLEGSVRVVTTFDSEYCRHGTCERSEAGVCVSTSGRWVLN |
| | | NDYYRSLPGVFCGVDAVNLLTNMFTPLIQPIGALDISASIVAGGIVAIVV |
| | | TCLAYYFMRFRRAFGEYSHVVAFNTLLFLMSFTVLCLTPVYSFLPGVY |
| | | SVIYLYLTFYLTNDVSFLAHIQWMVMFTPLVPFWITIAYIICISTKHFYW |
| | | FFSNYLKRRVVFNGVSFSTFEEAALCTFLLNKEMYLKLRSDVLLPLTQ |
| | | YNRYLALYNKYKYFSGAMDTTSYREAACCHLAKALNDFSNSGSDVL |
| | | YQPPQTSITSAVLQSGFRKMAFPSGKVEGCMVQVTCGTTTLNGLWLD |
| | | DVVYCPRHVICTSEDMLNPNYEDLLIRKSNHNFLVQAGNVQLRVIGHS |
| | | MQNCVLKLKVDTANPKTPKYKFVRIQPGQTFSVLACYNGSPSGVYQC |
| | | AMRPNFTIKGSFLNGSCGSVGFNIDYDCVSFCYMHHMELPTGVHAGT |
| | | DLEGNFYGPFVDRQTAQAAGTDTTITVNVLAWLYAAVINGDRWFLNR |
| | | FTTTLNDFNLVAMKYNYEPLTQDHVDILGPLSAQTGIAVLDMCASLKE |
| | | LLQNGMNGRTILGSALLEDEFTPFDVVRQCSGVTFQSAVKRTIKGTHH |
| | | WLLLTILTSLLVLVQSTQWSLFFFLYENAFLPFAMGIIAMSAFAMMFV |
| | | KHKHAFLCLFLLPSLATVAYFNMVYMPASWVMRIMTWLDMVDTSLS |
| | | GFKLKDCVMYASAVVLLILMTARTVYDDGARRVWTLMNVLTLVYK |
| | | VYYGNALDQAISMWALIISVTSNYSGVVTTVMFLARGIVFMCVEYCPI |
| | | FFITGNTLQCIMLVYCFLGYFCTCYFGLFCLLNRYFRLTLGVYDYLVST |
| | | QEFRYMNSQGLLPPKNSIDAFKLNIKLLGVGGKPCIKVATVQSKMSDV |
| | | KCTSVVLLSVLQQLRVESSSKLWAQCVQLHNDILLAKDTTEAFEKMV |
| | | SLLSVLLSMQGAVDINKLCEEMLDNRATLQAIASEFSSLPSYAAFATAQ |
| | | EAYEQAVANGDSEVVLKKLKKSLNVAKSEFDRDAAMQRKLEKMAD |
| | | QAMTQMYKQARSEDKRAKVTSAMQTMLFTMLRKLDNDALNNIINNA |
| | | RDGCVPLNIIPLTTAAKLMVVIPDYNTYKNTCDGTTFTYASALWEIQQ |
| | | VVDADSKIVQLSEISMDNSPNLAWPLIVTALRANSAVKLQNNELSPVA |
| | | LRQMSCAAGTTQTACTDDNALAYYNTTKGGRFVLALLSDLQDLKWA |
| | | RFPKSDGTGTIYTELEPPCRFVTDTPKGPKVKYLYFIKGLNNLNRGMVL |
| | | GSLAATVRLQAGNATEVPANSTVLSFCAFAVDAAKAYKDYLASGGQP |
| | | ITNCVKMLCTHTGTGQAITVTPEANMDQESFGGASCCLYCRCHIDHPN |
| | | PKGFCDLKGKYVQIPTTCANDPVGFTLKNTVCTVCGMWKGYGCSCD |
| | | QLREPMLQSADAQSFLNGFAV |
| 391 | SARS-CiV2 | SADAQSFLNRVCGVSAARLTPCGTGTSTDVVYRAFDIYNDKVAGFAK |
| | ORF1b | FLKTNCCRFQEKDEDDNLIDSYFVVKRHTFSNYQHEETIYNLLKDCPA |
| | polyprotein | VAKHDFFKFRIDGDMVPHISRQRLTKYTMADLVYALRHFDEGNCDTL |
| | NSP12-16 amino | KEILVTYNCCDDDYFNKKDWYDFVENPDILRVYANLGERVRQALLKT |
| | acid sequence | VQFCDAMRNAGIVGVLTLDNQDLNGNWYDFGDFIQTTPGSGVPVVDS |
| | (Wuhan Hu1) | YYSLLMPILTLTRALTAESHVDTDLTKPYIKWDLLKYDFTEERLKLFDR |
| | | YFKYWDQTYHPNCVNCLDDRCILHCANFNVLFSTVFPPTSFGPLVRKIF |
| | | VDGVPFVVSTGYHFRELGVVHNQDVNLHSSRLSFKELLVYAADPAMH |
| | | AASGNLLLDKRTTCFSVAALTNNVAFQTVKPGNFNKDFYDFAVSKGF |
| | | FKEGSSVELKHFFFAQDGNAAISDYDYYRYNLPTMCDIRQLLFVVEVV |
| | | DKYFDCYDGGCINANQVIVNNLDKSAGFPFNKWGKARLYYDSMSYE |
| | | DQDALFAYTKRNVIPTITQMNLKYAISAKNRARTVAGVSICSTMTNRQ |
| | | FHQKLLKSIAATRGATVVIGTSKFYGGWHNMLKTVYSDVENPHLMG |
| | | WDYPKCDRAMPNMLRIMASLVLARKHTTCCSLSHRFYRLANECAQV |
| | | LSEMVMCGGSLYVKPGGTSSGDATTAYANSVFNICQAVTANVNALLS |
| | | TDGNKIADKYVRNLQHRLYECLYRNRDVDTDFVNEFYAYLRKHFSM |
| | | MILSDDAVVCFNSTYASQGLVASIKNFKSVLYYQNNVFMSEAKCWTE |
| | | TDLTKGPHEFCSQHTMLVKQGDDYVYLPYPDPSRILGAGCFVDDIVKT |
| | | DGTLMIERFVSLAIDAYPLTKHPNQEYADVFHLYLQYIRKLHDELTGH |
| | | MLDMYSVMLTNDNTSRYWEPEFYEAMYTPHTVLQAVGACVLCNSQT |
| | | SLRCGACIRRPFLCCKCCYDHVISTSHKLVLSVNPYVCNAPGCDVTDV |
| | | TQLYLGGMSYYCKSHKPPISFPLCANGQVFGLYKNTCVGSDNVTDFN |
| | | AIATCDWTNAGDYILANTCTERLKLFAAETLKATEETFKLSYGIATVRE |
| | | VLSDRELHLSWEVGKPRPPLNRNYVFTGYRVTKNSKVQIGEYTFEKGD |
| | | YGDAVVYRGTTTYKLNVGDYFVLTSHTVMPLSAPTLVPQEHYVRITG |
| | | LYPTLNISDEFSSNVANYQKVGMQKYSTLQGPPGTGKSHFAIGLALYY |
| | | PSARIVYTACSHAAVDALCEKALKYLPIDKCSRIIPARARVECFDKFKV |
| | | NSTLEQYVFCTVNALPETTADIVVFDEISMATNYDLSVVNARLRAKHY |
| | | VYIGDPAQLPAPRTLLTKGTLEPEYFNSVCRLMKTIGPDMFLGTCRRCP |
| | | AEIVDTVSALVYDNKLKAHKDKSAQCFKMFYKGVITHDVSSAINRPQI |
| | | GVVREFLTRNPAWRKAVFISPYNSQNAVASKILGLPTQTVDSSQGSEY |
| | | DYVIFTQTTETAHSCNVNRFNVAITRAKVGILCIMSDRDLYDKLQFTSL |
| | | EIPRRNVATLQAENVTGLFKDCSKVITGLHPTQAPTHLSVDTKFKTEGL |
| | | CVDIPGIPKDMTYRRLISMMGFKMNYQVNGYPNMFITREEAIRHVRA |
| | | WIGFDVEGCHATREAVGTNLPLQLGFSTGVNLVAVPTGYVDTPNNTD |
| | | FSRVSAKPPPGDQFKHLIPLMYKGLPWNVVRIKIVQMLSDTLKNLSDR |
| | | VVFVLWAHGFELTSMKYFVKIGPERTCCLCDRRATCFSTASDTYACW |
| | | HHSIGFDYVYNPFMIDVQQWGFTGNLQSNHDLYCQVHGNAHVASCD |
| | | AIMTRCLAVHECFVKRVDWTIEYPIIGDELKINAACRKVQHMVVKAAL |
| | | LADKFPVLHDIGNPKAIKCVPQADVEWKFYDAQPCSDKAYKIEELFYS |
| | | YATHSDKFTDGVCLFWNCNVDRYPANSIVCRFDTRVLSNLNLPGCDG |
| | | GSLYVNKHAFHTPAFDKSAFVNLKQLPFFYYSDSPCESHGKQVVSDID |
| | | YVPLKSATCITRQNLGGAVCRHHANEYRLYLDAYNMMISAGFSLWVY |
| | | KQFDTYNLWNTFTRLQSLENVAFNVVNKGHFDGQQGEVPVSIINNTV |
| | | YTKVDGVDVELFENKTTLPVNVAFELWAKRNIKPVPEVKILNNLGVDI |
| | | AANTVIWDYKRDAPAHISTIGVCSMTDIAKKPTETICAPLTVFFDGRVD |
| | | GQVDLFRNARNGVLITEGSVKGLQPSVGPKQASLNGVTLIGEAVKTQF |
| | | NYYKKVDGVVQQLPETYFTQSRNLQEFKPRSQMEIDFLELAMDEFIER |
| | | YKLEGYAFEHIVYGDFSHSQLGGLHLLIGLAKRFKESPFELEDFIPMDS |
| | | TVKNYFITDAQTGSSKCVCSVIDLLLDDFVEIIKSQDLSVVSKVVKVTID |
| | | YTEISFMLWCKDGHVETFYPKLQSSQAWQPGVAMPNLYKMQRMLLE |
| | | KCDLQNYGDSATLPKGIMMNVAKYTQLCQYLNTLTLAVPYNMRVIHF |
| | | GAGSDKGVAPGTAVLRQWLPTGTLLVDSDLNDFVSDADSTLIGDCAT |
| | | VHTANKWDLIISDMYDPKTKNVTKENDSKEGFFTYICGFIQQKLALGG |
| | | SVAIKITEHSWNADLYKLMGHFAWWTAFVTNVNASSSEAFLIGCNYL |
| | | GKPREQIDGYVMHANYIFWRNTNPIQLSSYSLFDMSKFPLKLRGTAVM |
| | | SLKEGQINDMILSLLSKGRLIIRENNRVVISSDVLVNN |
| 392 | SARS-CoV2 | SADAQSFLNRVCGVSAARLTPCGTGTSTDVVYRAFDIYNDKVAGFAK |
| | NSP12 amino | FLKTNCCRFQEKDEDDNLIDSYFVVKRHTFSNYQHEETIYNLLKDCPA |
| | acid | VAKHDFFKFRIDGDMVPHISRQRLTKYTMADLVYALRHFDEGNCDTL |
| | sequence | KEILVTYNCCDDDYFNKKDWYDFVENPDILRVYANLGERVRQALLKT |
| | (Wuhan Hu1) | VQFCDAMRNAGIVGVLTLDNQDLNGNWYDFGDFIQTTPGSGVPVVDS |
| | | YYSLLMPILTLTRALTAESHVDTDLTKPYIKWDLLKYDFTEERLKLFDR |
| | | YFKYWDQTYHPNCVNCLDDRCILHCANFNVLFSTVFPPTSFGPLVRKIF |
| | | VDGVPFVVSTGYHFRELGVVHNQDVNLHSSRLSFKELLVYAADPAMH |
| | | AASGNLLLDKRTTCFSVAALTNNVAFQTVKPGNFNKDFYDFAVSKGF |
| | | FKEGSSVELKHFFFAQDGNAAISDYDYYRYNLPTMCDIRQLLFVVEVV |
| | | DKYFDCYDGGCINANQVIVNNLDKSAGFPFNKWGKARLYYDSMSYE |
| | | DQDALFAYTKRNVIPTITQMNLKYAISAKNRARTVAGVSICSTMTNRQ |
| | | FHQKLLKSIAATRGATVVIGTSKFYGGWHNMLKTVYSDVENPHLMG |
| | | WDYPKCDRAMPNMLRIMASLVLARKHTTCCSLSHRFYRLANECAQV |
| | | LSEMVMCGGSLYVKPGGTSSGDATTAYANSVFNICQAVTANVNALLS |
| | | TDGNKIADKYVRNLQHRLYECLYRNRDVDTDFVNEFYAYLRKHFSM |
| | | MILSDDAVVCFNSTYASQGLVASIKNFKSVLYYQNNVFMSEAKCWTE |
| | | TDLTKGPHEFCSQHTMLVKQGDDYVYLPYPDPSRILGAGCFVDDIVKT |
| | | DGTLMIERFVSLAIDAYPLTKHPNQEYADVFHLYLQYIRKLHDELTGH |
| | | MLDMYSVMLTNDNTSRYWEPEFYEAMYTPHTVLQ |
| 393 | SARS-CoV2 | AVGACVLCNSQTSLRCGACIRRPFLCCKCCYDHVISTSHKLVLSVNPY |
| | NSP13-14 amino | VCNAPGCDVTDVTQLYLGGMSYYCKSHKPPISFPLCANGQVFGLYKN |
| | acid sequence | TCVGSDNVTDFNAIATCDWTNAGDYILANTCTERLKLFAAETLKATEE |
| | (Wuhan Hu1) | TFKLSYGIATVREVLSDRELHLSWEVGKPRPPLNRNYVFTGYRVTKNS |
| | | KVQIGEYTFEKGDYGDAVVYRGTTTYKLNVGDYFVLTSHTVMPLSAP |
| | | TLVPQEHYVRITGLYPTLNISDEFSSNVANYQKVGMQKYSTLQGPPGT |
| | | GKSHFAIGLALYYPSARIVYTACSHAAVDALCEKALKYLPIDKCSRIIP |
| | | ARARVECFDKFKVNSTLEQYVFCTVNALPETTADIVVFDEISMATNYD |
| | | LSVVNARLRAKHYVYIGDPAQLPAPRTLLTKGTLEPEYFNSVCRLMKT |
| | | IGPDMFLGTCRRCPAEIVDTVSALVYDNKLKAHKDKSAQCFKMFYKG |
| | | VITHDVSSAINRPQIGVVREFLTRNPAWRKAVFISPYNSQNAVASKILG |
| | | LPTQTVDSSQGSEYDYVIFTQTTETAHSCNVNRFNVAITRAKVGILCIM |
| | | SDRDLYDKLQFTSLEIPRRNVATLQAENVTGLFKDCSKVITGLHPTQAP |
| | | THLSVDTKFKTEGLCVDIPGIPKDMTYRRLISMMGFKMNYQVNGYPN |
| | | MFITREEAIRHVRAWIGFDVEGCHATREAVGTNLPLQLGFSTGVNLVA |
| | | VPTGYVDTPNNTDFSRVSAKPPPGDQFKHLIPLMYKGLPWNVVRIKIV |
| | | QMLSDTLKNLSDRVVFVLWAHGFELTSMKYFVKIGPERTCCLCDRRA |
| | | TCFSTASDTYACWHHSIGFDYVYNPFMIDVQQWGFTGNLQSNHDLYC |
| | | QVHGNAHVASCDAIMTRCLAVHECFVKRVDWTIEYPIIGDELKINAAC |
| | | RKVQHMVVKAALLADKFPVLHDIGNPKAIKCVPQADVEWKFYDAQP |
| | | CSDKAYKIEELFYSYATHSDKFTDGVCLFWNCNVDRYPANSIVCRFDT |
| | | RVLSNLNLPGCDGGSLYVNKHAFHTPAFDKSAFVNLKQLPFFYYSDSP |
| | | CESHGKQVVSDIDYVPLKSATCITRQNLGGAVCRHHANEYRLYLDAY |
| | | NMMISAGFSLWVYKQFDTYNLWNTFTRLQ |
| 394 | SARS-CoV2 | SLENVAFNVVNKGHFDGQQGEVPVSIINNTVYTKVDGVDVELFENKT |
| | NSP15-16 amino | TLPVNVAFELWAKRNIKPVPEVKILNNLGVDIAANTVIWDYKRDAPAH |
| | acid sequence | ISTIGVCSMTDIAKKPTETICAPLTVFFDGRVDGQVDLFRNARNGVLITE |
| | (Wuhan Hu1) | GSVKGLQPSVGPKQASLNGVTLIGEAVKTQFNYYKKVDGVVQQLPET |
| | | YFTQSRNLQEFKPRSQMEIDFLELAMDEFIERYKLEGYAFEHIVYGDFS |
| | | HSQLGGLHLLIGLAKRFKESPFELEDFIPMDSTVKNYFITDAQTGSSKC |
| | | VCSVIDLLLDDFVEIIKSQDLSVVSKVVKVTIDYTEISFMLWCKDGHVE |
| | | TFYPKLQSSQAWQPGVAMPNLYKMQRMLLEKCDLQNYGDSATLPKG |
| | | IMMNVAKYTQLCQYLNTLTLAVPYNMRVIHFGAGSDKGVAPGTAVL |
| | | RQWLPTGTLLVDSDLNDFVSDADSTLIGDCATVHTANKWDLIISDMY |
| | | DPKTKNVTKENDSKEGFFTYICGFIQQKLALGGSVAIKITEHSWNADLY |
| | | KLMGHFAWWTAFVTNVNASSSEAFLIGCNYLGKPREQIDGYVMHAN |
| | | YIFWRNTNPIQLSSYSLFDMSKFPLKLRGTAVMSLKEGQINDMILSLLS |
| | | KGRLIIRENNRVVISSDVLVNN |

In some embodiments, any of the above SEQ ID NOS:349-395 or 401, further includes the amino acid residue methionine (M) as the first amino acid residue.

In some embodiments, the antigenic insert is derived from a tumor associated antigen. In some embodiments, the antigenic insert is derived from human mucin-1, or a fragment thereof. In some embodiments, the antigenic insert is derived from an amino acid sequence selected from SEQ ID NO: 349, 358-364, or 403, or a fragment thereof, or an amino acid sequence at least 85%, 90%, 95%, 96%, 97%, 98%, or 99% identical thereto.

In some embodiments, the antigenic insert is derived from a human cyclin B1 protein, or a fragment thereof. In some embodiments, the antigenic insert is derived from an amino acid sequence selected from SEQ ID NO: 350, or a fragment thereof, or an amino acid sequence at least 85%, 90%, 95%, 96%, 97%, 98%, or 99% identical thereto.

In some embodiments, the antigenic insert is derived from a hepatitis B virus protein, or a fragment thereof. In some embodiments, the antigenic insert is derived from an amino acid sequence selected from SEQ ID NOS: 351-354, or a fragment thereof, or an amino acid sequence at least 85%, 90%, 95%, 96%, 97%, 98%, or 99% identical thereto.

In some embodiments, the antigenic insert is derived from a Plasmodium sp. protein, or a fragment thereof. In some embodiments, the antigenic insert is derived from an amino acid sequence selected from SEQ ID NOS: 355-357, or a fragment thereof, or an amino acid sequence at least 85%, 90%, 95%, 96%, 97%, 98%, or 99% identical thereto.

In some embodiments, the antigenic insert is derived from a Lassa virus protein, or a fragment thereof. In some embodiments, the antigenic insert is derived from an amino acid sequence selected from SEQ ID NOS: 365-366, or a fragment thereof, or an amino acid sequence at least 85%, 90%, 95%, 96%, 97%, 98%, or 99% identical thereto.

In some embodiments, the antigenic insert is derived from a ebola virus protein, or a fragment thereof. In some embodiments, the antigenic insert is derived from an amino acid sequence selected from SEQ ID NOS: 367-368, or a fragment thereof, or an amino acid sequence at least 85%, 90%, 95%, 96%, 97%, 98%, or 99% identical thereto.

In some embodiments, the antigenic insert is derived from a Zika virus protein, or a fragment thereof. In some embodiments, the antigenic insert is derived from an amino acid sequence selected from SEQ ID NOS: 369-376, or a fragment thereof, or an amino acid sequence at least 85%, 90%, 95%, 96%, 97%, 98%, or 99% identical thereto.

In some embodiments, the antigenic insert is derived from one or more SARS-CoV-2 proteins or polypeptides, for example, a protein or peptide derived from one or more of the spike (S) (NCBI Reference Sequence YP_009724390), membrane (M) (NCBI Reference Sequence YP_009724393), envelope (E) (NCBI Reference Sequence YP_009724392), nucleoside (N) (NCBI Reference Sequence YP_009724397), ORF1AB (NCBI Reference Sequence YP_009724389), ORF3a (NCBI Reference Sequence YP_009724391), ORF6 (NCBI Reference Sequence YP_009724394), ORF7a (NCBI Reference Sequence YP_009724395), ORF7b (NCBI Reference Sequence YP_009725318), ORF8 (NCBI Reference Sequence YP_009724396), or ORF10 (NCBI Reference Sequence YP_009725255), In certain embodiments, the antigenic insert is derived from SARS-CoV2 S protein, or a variant thereof. In some embodiments, the S protein is expressed as a full-length protein and contains one or more amino acid substitutions compared to NCBI Reference Sequence YP_009724390. In some embodiments, the S protein is derived from the amino acid sequence of SEQ ID NO:377, or fragment thereof, or amino acid sequence at least 85%, 90%, 95%, 96%, 97%, 98%, or 99% identical thereto. In some embodiments, the S protein is expressed as a full-length protein and contains one or more substitutions selected from K417T, E484K or N501Y of SEQ ID NO:377. In some embodiments, the S protein is expressed as a full-length protein and contains the following substitutions: K417T, E484K, and N501Y of SEQ ID NO:377. In some embodiments, the rMVA contains a nucleic acid sequence which encodes the S protein further comprising substitutions at L452R, T478K, or P681R, or a combination thereof of SEQ ID NO: 377. In some embodiments, the rMVA contains a nucleic acid sequence which encodes the S protein further comprising substitutions at L452R, T478K, and P681R of SEQ ID NO: 377. In some embodiments, the rMVA contains a nucleic acid sequence which encodes the S protein further comprising a substitution at N440K, S443A, G476S, E484R, and/or G502P, or combinations thereof of SEQ ID NO: 377. In some embodiments, the rMVA contains a nucleic acid sequence which encodes the S protein further comprising a substitution at one or more of T19R, G142D, R158G, K417N, L452R, T478K, E484Q, D614G, P681R, D950N, E156del, F157del, N501Y, spike deletion 69-70del, spike deletion 144del, A570D, T716I, S982A, D1118H, P681H, L18F, D80A, D215G, 242-244del, R246I, K471N, E484K, A701V, N440K, S443A, G476S, E484R, and G502P, or any combinations thereof of SEQ ID NO: 377. In some embodiments, the rMVA contains a nucleic acid sequence which encodes the S protein further comprising a substitution at T19R, T95I, G142D, E156del, F157del, R158G, L452R, T478K, D614G, P681R, and D950N of SEQ ID NO: 377. In some embodiments, the substitution is K417N. In some embodiments, the rMVA contains a nucleic acid sequence which encodes the S protein further comprising a substitution at T19R, V70F, T95I, G142D, E156del, F157del, R158G, A222V, W258L, K417N, L452R, T478K, D614G, P681R, and D950N of SEQ ID NO: 377. In some embodiments, the rMVA contains a nucleic acid sequence which encodes the S protein further comprising a substitution at N501Y, D614G, and P681H of SEQ ID NO: 377. In some embodiments, the rMVA contains a nucleic acid sequence which encodes the S protein further comprising a substitution at E484K, N501Y, D614G, and P681H of SEQ ID NO: 377. In some embodiments, the rMVA contains a nucleic acid sequence which encodes the S protein further comprising a substitution at K417N, E484K, N501Y, D614G, and A701V of SEQ ID NO: 377. In some embodiments, the rMVA contains a nucleic acid sequence which encodes the S protein further comprising a substitution at K417T, E484K, N501Y, D614G, and H655Y of SEQ ID NO: 377. In some embodiments, the rMVA contains a nucleic acid sequence which encodes the S protein further comprising a substitution at L452R, T478K, D614G, and P681R of SEQ ID NO: 377. In some embodiments, the rMVA contains a nucleic acid sequence which encodes the S protein further comprising a substitution at E484K, D614G, and Q677H of SEQ ID NO: 377. In some embodiments, the rMVA contains a nucleic acid sequence which encodes the S protein further comprising a substitution at E484K, N501Y, D614G, and P681H of SEQ ID NO: 377. In some embodiments, the rMVA contains a nucleic acid sequence which encodes the S protein further comprising a substitution at L452R, E484Q, D614G, and P681R of SEQ ID NO: 377. In some embodiments, the rMVA contains a nucleic acid sequence which encodes the S protein further comprising a substitution at S477N, E484K, D614G, and P681H of SEQ ID NO: 377. In some embodiments, the rMVA contains a nucleic acid sequence which encodes the S protein further comprising a substitution at R346K, E484K, N501Y, D614G, and P681H of SEQ ID NO: 377. In some embodiments, the rMVA contains a nucleic acid sequence which encodes the S protein further comprising a substitution at L452Q, F490S, and D614G of SEQ ID NO: 377. In some embodiments, the rMVA contains a nucleic acid sequence which encodes the S protein further comprising a substitution at L452R, E484Q, D614G, and P681R of SEQ ID NO: 377. In some embodiments, the rMVA contains a nucleic acid sequence which encodes the S protein further comprising a substitution at Q414K, N450K, ins214TDR, and D614G of SEQ ID NO: 377. In some embodiments, the rMVA contains a nucleic acid sequence which encodes the S protein further comprising a substitution at V367F, E484K, and Q613H of SEQ ID NO: 377. In some embodiments, the rMVA contains a nucleic acid sequence which encodes the S protein further comprising a substitution at L452R, N501Y, A653V, and H655Y of SEQ ID NO: 377. In some embodiments, the rMVA contains a nucleic acid sequence which encodes the S protein further comprising a substitution at E484K, N501T, and H655Y of SEQ ID NO: 377. In some embodiments, the rMVA contains a nucleic acid sequence which encodes the S protein further comprising a substitution at L452R, and D614G of SEQ ID NO: 377. In some embodiments, the rMVA contains a nucleic acid sequence which encodes the S protein further comprising a substitution at P384L, K417N, E484K, N501Y, D614G, and A701V of SEQ ID NO: 377. In some embodiments, the rMVA contains a nucleic acid sequence which encodes the S protein further comprising a substitution at K417N, E484K, N501Y, E516Q, D614G, and A701V of SEQ ID NO: 377. In some embodiments, the rMVA contains a nucleic acid sequence which encodes the S protein further comprising a substitution at L452R, N501Y, D614G, and P681H of SEQ ID NO: 377. In some embodiments, the rMVA contains a nucleic acid sequence which encodes the S protein further comprising a substitution at S494P, N501Y, D614G, and P681H of SEQ ID NO: 377. In some embodiments, the rMVA contains a nucleic acid sequence which encodes the S protein further comprising a substitution at L452R, D614G, and Q677H of SEQ ID NO: 377. In some embodiments, the rMVA contains a nucleic acid sequence which encodes the S protein further comprising a substitution at E484K, D614G, N679K, and ins679GIAL of SEQ ID NO: 377. In some embodiments, the rMVA contains a nucleic acid sequence which encodes the S protein further comprising a substitution at E484K, D614G, and A701V of SEQ ID NO: 377. In some embodiments, the rMVA contains a nucleic acid sequence which encodes the S protein further comprising a substitution at L452R, and D614G of SEQ ID NO: 377. In some embodiments, the rMVA contains a nucleic acid sequence which encodes the S protein further comprising a substitution at S477N, and D614G of SEQ ID NO: 377. In some embodiments, the rMVA contains a nucleic acid sequence which encodes the S protein further comprising a substitution at E484K, D614G, and P681H of SEQ ID NO: 377. In some embodiments, the rMVA contains a nucleic acid sequence which encodes the S protein further comprising a substitution at E484K, and D614G of SEQ ID NO: 377. In some embodiments, the rMVA contains a nucleic acid sequence which encodes the S protein further comprising a substitution at T478K, and D614G of SEQ ID NO: 377. In some embodiments, the rMVA contains a nucleic acid sequence which encodes the S protein further comprising a substitution at N439K, E484K, D614G, and P681H of SEQ ID NO: 377. In some embodiments, the rMVA contains a nucleic acid sequence which encodes the S protein further comprising a substitution at D614G, E484K, H655Y, K417T, N501Y, and P681H of SEQ ID NO: 377. In some embodiments, the rMVA contains a nucleic acid sequence which encodes the S protein further comprising a substitution at L452R, T478K, D614G, P681R, and K417N of SEQ ID NO: 377. In some embodiments, the rMVA contains a nucleic acid sequence which encodes the S protein further comprising a substitution at D614G, E484K, H655Y, N501Y, N679K, and Y449H of SEQ ID NO: 377.

In some embodiments, the S protein is expressed as a full-length protein and has a deletion of one or more spike protein amino acids H69, V70, or Y144, or combinations thereof, of SEQ ID NO: 377. In some embodiments, the S protein is expressed as a full-length protein and contains one or more substitutions selected from D614G, A570D, P681H, T716I, S982A, D1118H, K417N or K417T, D215G, A701V, L18F, R246I, Y453F, I692V, M1229I, N439K, A222V, S477N, or A376T, or combinations thereof, of SEQ ID NO:377. In some embodiments, the variant strain is a SARS-CoV2 virus which has a spike protein deletion at amino acids 242-244 of SEQ ID NO: 377. In some embodiments, the S protein is expressed as a full-length protein and contains the following deletions and substitutions: deletion of amino acids 69-70, deletion of amino acid Y144, amino acid substitution N501Y, amino acid substitution A570D, amino acid substitution D614G, amino acid substitution P681H, amino acid substitution T716I, amino acid substitution S982A, and amino acid substitution D1118H, or SEQ ID NO: 377. In some embodiments, the S protein is expressed as a full-length protein and contains the following deletions and substitutions: N501Y, K417N or K417T, E484K, D80A, A701V, L18F, and amino acid deletion at amino acids 242-244, of SEQ ID NO: 377. In some embodiments, the S protein is expressed as a full-length protein and contains one or more of the following substitutions: D614G; D936Y; P1263L; L5F; N439K; R21I; D839Y; L54F; A879S; L18F; F1121L; R847K; L452R; T478I; A829T; Q675H; S477N; H49Y; T29I; G769V; G1124V; V1176F; K1073N; P479S; S1252P; Y145 deletion; E583D; R214L; A1020V; Q1208H; D215G; H146Y; S98F; T95I; G1219C; A846V; I197V; R102I; V367F; T572I; A1078S; A831V; P1162L; T73I; A845S; G1219V; H245Y; L8V; Q675R; S254F; V483A; Q677H; D138H; D80Y; M1237T; D1146H; E654D; H655Y; S50L; S939F; S943P; G485R; Q613H; T76I; V341I; M153I; S221L; T859I; W258L; L242F; P681L; V289I; A520S; V1104L; V1228L; L176F; M1237I; T307I; T716I; L141; M1229I; A1087S; P26S; P330S; P384L; R765L; S940F; T323I; V826L; E1202Q; L1203F; L611F; V615I; A262S; A522V; A688V; A706V; A892S; E554D; Q836H; T1027I; T22I; A222V; A27S; A626V; C1247F; K1191N; M731I; P26L; S1147L; S1252F; S255F; V1264L; V308L; D80A; I670L; P251L; P631S; *1274Q; A344S; A771S; A879T; D1084Y; D253G; H1101Y; L1200F; Q14H; Q239K; A623V; D215Y; E1150D; G476S; K77M; M177I; P812S; S704L; T51I; T547I; T791I; V1122L; Y145H; D574Y; G142D; G181V; I834T; N370S; P812L; S12F; T791P; V90F; W152L; A292S; A570V; A647S; A845V; D1163Y; G181R; L84I; L938F; P1143L; P809S; R78M; T1160I; V1133F; V213L; V615F; A831V; D839Y; D839N; D839E; S943P; P1263L; S13I; or V622F; and combinations thereof, of SEQ ID NO: 377.

In some embodiments, the S protein is selected from SEQ ID NOS: 377-384, or a fragment thereof, or an amino acid sequence at least 85%, 90%, 95%, 96%, 97%, 98%, or 99% identical thereto.

In some embodiments, the Stabilized S protein is expressed as a full-length protein and contains one or more substitutions selected from K417T, E484K or N501Y of SEQ ID NO: 381. In some embodiments, the Stabilized S protein is expressed as a full-length protein and contains the following substitutions: K417T, E484K, and N501Y of SEQ ID NO:381. In some embodiments, the rMVA contains a nucleic acid sequence which encodes the Stabilized S protein further comprising substitutions at L452R, T478K, or P681R, or a combination thereof of SEQ ID NO: 381. In some embodiments, the rMVA contains a nucleic acid sequence which encodes the Stabilized S protein further comprising substitutions at L452R, T478K, and P681R of SEQ ID NO: 381. In some embodiments, the rMVA contains a nucleic acid sequence which encodes the Stabilized S protein further comprising a substitution at N440K, S443A, G476S, E484R, and/or G502P, or combinations thereof of SEQ ID NO: 381. In some embodiments, the rMVA contains a nucleic acid sequence which encodes the Stabilized S protein further comprising a substitution at one or more of T19R, G142D, R158G, K417N, L452R, T478K, E484Q, D614G, P681R, D950N, E156del, F157del, N501Y, spike deletion 69-70del, spike deletion 144del, A570D, T716I, S982A, D1118H, P681H, L18F, D80A, D215G, 242-244del, R246I, K471N, E484K, A701V, N440K, S443A, G476S, E484R, and G502P, or any combinations thereof of SEQ ID NO: 381. In some embodiments, the rMVA contains a nucleic acid sequence which encodes the Stabilized S protein further comprising a substitution at T19R, T95I, G142D, E156del, F157del, R158G, L452R, T478K, D614G, P681R, and D950N of SEQ ID NO: 381. In some embodiments, the substitution is K417N. In some embodiments, the rMVA contains a nucleic acid sequence which encodes the Stabilized S protein further comprising a substitution at T19R, V70F, T95I, G142D, E156del, F157del, R158G, A222V, W258L, K417N, L452R, T478K, D614G, P681R, and D950N of SEQ ID NO: 381. In some embodiments, the rMVA contains a nucleic acid sequence which encodes the Stabilized S protein further comprising a substitution at N501Y, D614G, and P681H of SEQ ID NO: 381. In some embodiments, the rMVA contains a nucleic acid sequence which encodes the Stabilized S protein further comprising a substitution at E484K, N501Y, D614G, and P681H of SEQ ID NO: 381. In some embodiments, the rMVA contains a nucleic acid sequence which encodes the Stabilized S protein further comprising a substitution at K417N, E484K, N501Y, D614G, and A701V of SEQ ID NO: 381. In some embodiments, the rMVA contains a nucleic acid sequence which encodes the Stabilized S protein further comprising a substitution at K417T, E484K, N501Y, D614G, and H655Y of SEQ ID NO: 381. In some embodiments, the rMVA contains a nucleic acid sequence which encodes the Stabilized S protein further comprising a substitution at L452R, T478K, D614G, and P681R of SEQ ID NO: 381. In some embodiments, the rMVA contains a nucleic acid sequence which encodes the Stabilized S protein further comprising a substitution at E484K, D614G, and Q677H of SEQ ID NO: 381. In some embodiments, the rMVA contains a nucleic acid sequence which encodes the Stabilized S protein further comprising a substitution at E484K, N501Y, D614G, and P681H of SEQ ID NO: 381. In some embodiments, the rMVA contains a nucleic acid sequence which encodes the Stabilized S protein further comprising a substitution at L452R, E484Q, D614G, and P681R of SEQ ID NO: 381. In some embodiments, the rMVA contains a nucleic acid sequence which encodes the Stabilized S protein further comprising a substitution at S477N, E484K, D614G, and P681H of SEQ ID NO: 381. In some embodiments, the rMVA contains a nucleic acid sequence which encodes the Stabilized S protein further comprising a substitution at R346K, E484K, N501Y, D614G, and P681H of SEQ ID NO: 381. In some embodiments, the rMVA contains a nucleic acid sequence which encodes the Stabilized S protein further comprising a substitution at L452Q, F490S, and D614G of SEQ ID NO: 381. In some embodiments, the rMVA contains a nucleic acid sequence which encodes the Stabilized S protein further comprising a substitution at L452R, E484Q, D614G, and P681R of SEQ ID NO: 8. In some embodiments, the rMVA contains a nucleic acid sequence which encodes the Stabilized S protein further comprising a substitution at Q414K, N450K, ins214TDR, and D614G of SEQ ID NO: 381. In some embodiments, the rMVA contains a nucleic acid sequence which encodes the Stabilized S protein further comprising a substitution at V367F, E484K, and Q613H of SEQ ID NO: 381. In some embodiments, the rMVA contains a nucleic acid sequence which encodes the Stabilized S protein further comprising a substitution at L452R, N501Y, A653V, and H655Y of SEQ ID NO: 381. In some embodiments, the rMVA contains a nucleic acid sequence which encodes the Stabilized S protein further comprising a substitution at E484K, N501T, and H655Y of SEQ ID NO: 381. In some embodiments, the rMVA contains a nucleic acid sequence which encodes the Stabilized S protein further comprising a substitution at L452R, and D614G of SEQ ID NO: 381. In some embodiments, the rMVA contains a nucleic acid sequence which encodes the Stabilized S protein further comprising a substitution at P384L, K417N, E484K, N501Y, D614G, and A701V of SEQ ID NO: 381. In some embodiments, the rMVA contains a nucleic acid sequence which encodes the Stabilized S protein further comprising a substitution at K417N, E484K, N501Y, E516Q, D614G, and A701V of SEQ ID NO: 381. In some embodiments, the rMVA contains a nucleic acid sequence which encodes the Stabilized S protein further comprising a substitution at L452R, N501Y, D614G, and P681H of SEQ ID NO: 381. In some embodiments, the rMVA contains a nucleic acid sequence which encodes the Stabilized S protein further comprising a substitution at S494P, N501Y, D614G, and P681H of SEQ ID NO: 381. In some embodiments, the rMVA contains a nucleic acid sequence which encodes the Stabilized S protein further comprising a substitution at L452R, D614G, and Q677H of SEQ ID NO: 381. In some embodiments, the rMVA contains a nucleic acid sequence which encodes the Stabilized S protein further comprising a substitution at E484K, D614G, N679K, and ins679GIAL of SEQ ID NO: 381. In some embodiments, the rMVA contains a nucleic acid sequence which encodes the Stabilized S protein further comprising a substitution at E484K, D614G, and A701V of SEQ ID NO: 381. In some embodiments, the rMVA contains a nucleic acid sequence which encodes the Stabilized S protein further comprising a substitution at L452R, and D614G of SEQ ID NO: 8. In some embodiments, the rMVA contains a nucleic acid sequence which encodes the Stabilized S protein further comprising a substitution at S477N, and D614G of SEQ ID NO: 381. In some embodiments, the rMVA contains a nucleic acid sequence which encodes the Stabilized S protein further comprising a substitution at E484K, D614G, and P681H of SEQ ID NO: 381. In some embodiments, the rMVA contains a nucleic acid sequence which encodes the Stabilized S protein further comprising a substitution at E484K, and D614G of SEQ ID NO: 381. In some embodiments, the rMVA contains a nucleic acid sequence which encodes the Stabilized S protein further comprising a substitution at T478K, and D614G of SEQ ID NO: 381. In some embodiments, the rMVA contains a nucleic acid sequence which encodes the Stabilized S protein further comprising a substitution at N439K, E484K, D614G, and P681H of SEQ ID NO: 381. In some embodiments, the rMVA contains a nucleic acid sequence which encodes the Stabilized S protein further comprising a substitution at D614G, E484K, H655Y, K417T, N501Y, and P681H of SEQ ID NO: 381. In some embodiments, the rMVA contains a nucleic acid sequence which encodes the Stabilized S protein further comprising a substitution at L452R, T478K, D614G, P681R, and K417N of SEQ ID NO: 381. In some embodiments, the rMVA contains a nucleic acid sequence which encodes the Stabilized S protein further comprising a substitution at D614G, E484K, H655Y, N501Y, N679K, and Y449H of SEQ ID NO: 381.

In some embodiments, the Stabilized S protein is expressed as a full-length protein and has a deletion of one or more spike protein amino acids H69, V70, or Y144, or combinations thereof, of SEQ ID NO: 381. In some embodiments, the Stabilized S protein is expressed as a full-length protein and contains one or more substitutions selected from D614G, A570D, P681H, T716I, S982A, D1118H, K417N or K417T, D215G, A701V, L18F, R246I, Y453F, 1692V, M1229I, N439K, A222V, S477N, or A376T, or combinations thereof, of SEQ ID NO:1. In some embodiments, the variant strain is a SARS-CoV2 virus which has a spike protein deletion at amino acids 242-244 of SEQ ID NO: 381. In some embodiments, the Stabilized S protein is expressed as a full-length protein and contains the following deletions and substitutions: deletion of amino acids 69-70, deletion of amino acid Y144, amino acid substitution N501Y, amino acid substitution A570D, amino acid substitution D614G, amino acid substitution P681H, amino acid substitution T716I, amino acid substitution S982A, and amino acid substitution D1118H, or SEQ ID NO: 381. In some embodiments, the Stabilized S protein is expressed as a full-length protein and contains the following deletions and substitutions: N501Y, K417N or K417T, E484K, D80A, A701V, L18F, and amino acid deletion at amino acids 242-244, of SEQ ID NO: 381. In some embodiments, the S protein is expressed as a full-length protein and has a deletion of one or more spike protein amino acids H69, V70, or Y144, or combinations thereof, of SEQ ID NO: 381. In some embodiments, the S protein is expressed as a full-length protein and contains one or more substitutions selected from D614G, A570D, P681H, T716I, S982A, D1118H, K417N, K417T, D215G, A701V, L18F, R246I, Y453F, I692V, M1229I, N439K, A222V, S477N, or A376T, or combinations thereof, of SEQ ID NO: 381. In some embodiments, the spike protein includes a deletion at amino acids 242-244 of SEQ ID NO: 381. In some embodiments, the S protein is expressed as a full-length protein and contains the following deletions and substitutions: deletion of amino acids 69-70, deletion of amino acid Y144, amino acid substitution N501Y, amino acid substitution A570D, amino acid substitution D614G, amino acid substitution P681H, amino acid substitution T716I, amino acid substitution S982A, and amino acid substitution D1118H, of SEQ ID NO: 381. In some embodiments, the S protein is expressed as a full-length protein and contains the following deletions and substitutions: N501Y, K417N or K417T, E484K, D80A, A701V, L18F, and amino acid deletion at amino acids 242-244, of SEQ ID NO: 381. encodes the stabilized S protein further comprising substitutions at L452R, T478K, and P681R of SEQ ID NO: 381. In some embodiments, the rMVA contains a nucleic acid sequence which encodes the stabilized S protein further comprising a substitution at N440K, S443A, G476S, E484R, and/or G502P, or combinations thereof of SEQ ID NO: 381. In some embodiments, the rMVA contains a nucleic acid sequence which encodes the stabilized S protein further comprising a substitution at one or more of T19R, G142D, R158G, K417N, L452R, T478K, E484Q, D614G, P681R, D950N, E156del, F157del, N501Y, spike deletion 69-70del, spike deletion 144del, A570D, T716I, S982A, D1118H, P681H, L18F, D80A, D215G, 242-244del, R246I, K471N, E484K, A701V, N440K, S443A, G476S, E484R, and G502P, or any combinations thereof of SEQ ID NO: 381.

In some embodiments, the Stabilized S protein is expressed as a full-length protein and contains one or more of the following substitutions: D614G; D936Y; P1263L; L5F; N439K; R21I; D839Y; L54F; A879S; L18F; F1121L; R847K; L452R; T478I; A829T; Q675H; S477N; H49Y; T29I; G769V; G1124V; V1176F; K1073N; P479S; S1252P; Y145 deletion; E583D; R214L; A1020V; Q1208H; D215G; H146Y; S98F; T95I; G1219C; A846V; I197V; R102I; V367F; T572I; A1078S; A831V; P1162L; T73I; A845S; G1219V; H245Y; L8V; Q675R; S254F; V483A; Q677H; D138H; D80Y; M1237T; D1146H; E654D; H655Y; S50L; S939F; S943P; G485R; Q613H; T76I; V341I; M153I; S221L; T859I; W258L; L242F; P681L; V289I; A520S; V1104L; V1228L; L176F; M1237I; T307I; T716I; L141; M1229I; A1087S; P26S; P330S; P384L; R765L; S940F; T323I; V826L; E1202Q; L1203F; L611F; V615I; A262S; A522V; A688V; A706V; A892S; E554D; Q836H; T1027I; T22I; A222V; A27S; A626V; C1247F; K1191N; M731I; P26L; S1147L; S1252F; S255F; V1264L; V308L; D80A; I670L; P251L; P631S; *1274Q; A344S; A771S; A879T; D1084Y; D253G; H1101Y; L1200F; Q14H; Q239K; A623V; D215Y; E1150D; G476S; K77M; M177I; P812S; S704L; T51I; T547I; T791I; V1122L; Y145H; D574Y; G142D; G181V; I834T; N370S; P812L; S12F; T791P; V90F; W152L; A292S; A570V; A647S; A845V; D1163Y; G181R; L84I; L938F; P1143L; P809S; R78M; T1160I; V1133F; V213L; V615F; A831V; D839Y; D839N; D839E; S943P; P1263L; S13I; or V622F; and combinations thereof, of SEQ ID NO: 381.

In some embodiments, the stabilized S protein is expressed as a full-length protein of SEQ ID NO: 378, 379, 380, 381, 382, 383, or 384, or an amino acid sequence 80%, 85%, 90%, 95%, 98%, or 99% homologous thereto.

SARS-CoV-2 is an enveloped, positive-sense, single-stranded RNA virus that causes coronavirus disease 2019 (COVID-19). Virus particles include the RNA genetic material and structural proteins needed for invasion of host cells. Once inside the cell the infecting RNA is used to encode structural proteins that make up virus particles, nonstructural proteins that direct virus assembly, transcription, replication and host control and accessory proteins whose function has not been determined. ORF1ab, the largest gene, contains overlapping open reading frames that encode polyproteins PP1ab and PP1a. The polyproteins are cleaved to yield 16 nonstructural proteins, NSP1-16. Production of the longer (PP1ab) or shorter protein (PP1a) depends on a −1 ribosomal frameshifting event. The proteins, based on similarity to other coronaviruses, include the papain-like proteinase protein (NSP3), 3C-like proteinase (NSP5), RNA-dependent RNA polymerase (NSP12, RdRp), helicase (NSP13, HEL), endoRNAse (NSP15), 2′-O-Ribose-Methyltransferase (NSP16) and other nonstructural proteins. A description of the various NSPs encoded by ORF1ab can be found, for example, in Arya et al., Structural insights into SARS-CoV-2 proteins. J Mol Biol. 2021 Jan. 22; 433(2): 166725, incorporated herein by reference. In some embodiments provided herein, the rMVA antigenic insert is derived from one or more SARS-CoV-2 proteins or polypeptides selected from SEQ ID NOS:377-394.

In some embodiments, the antigenic insert is derived from a Marburg virus protein, or fragment thereof. In some embodiments, the antigenic insert is derived from an amino acid sequence selected from SEQ ID NO: 395-396, 398, or 400, or a fragment thereof, or an amino acid sequence at least 85%, 90%, 95%, 96%, 97%, 98%, or 99% identical thereto.

In particular embodiments, the encoded polypeptide comprises, in various alternative embodiments, ((M)(Secretion Signal Peptide-Immune Checkpoint Inhibitor Peptide-Cleavable Peptide)ₓ(Antigenic Peptide)), ((M)(Secretion Signal Peptide-Immune Checkpoint Inhibitor Peptide-Cleavable Peptide)ₓ(Secretion Signal Peptide-Antigenic Peptide)), ((M)(Secretion Signal Peptide-Immune Checkpoint Inhibitor Peptide-Cleavable Peptide)ₓ(Secretion Signal Peptide-Antigenic Peptide-Cleavable Peptide)_{y}), ((M)(Secretion Signal Peptide-Immune Checkpoint Inhibitor Peptide-Cleavable Peptide)ₓ(Secretion Signal Peptide-Antigenic Peptide-Cleavable Peptide)ₓ(Secretion Signal Peptide-Antigenic Peptide)), wherein y=1, 2, 3, 4, 5, 6, 7, 8, 9, 10, or more than 10, wherein x=1, 2, 3, 4, 5, 6, 7, 8, 9, 10, or more than 10, wherein M=methionine, and wherein the Secretion Signal Peptide is selected from a peptide having an amino acid sequence selected from SEQ ID NOS: 57-90, the Immune Checkpoint Inhibitor Peptide is selected from a peptide having an amino acid sequence selected from SEQ ID NOS: 1-56, the Cleavable Peptide is selected from a peptide having an amino acid sequence selected from SEQ ID NOS: 91-127, and the antigenic peptide is a peptide derived from an infectious agent, for example a virus, bacteria, parasite, fungus, or toxoid, or alternatively, a tumor associated antigen. In some embodiments, the Secretion Signal Peptide is selected from a peptide having an amino acid sequence selected from SEQ ID NOS: 65 and 66, the Immune Checkpoint Inhibitor Peptide is selected from a peptide having an amino acid sequence selected from SEQ ID NOS: 1 and 5, and the Cleavable Peptide is selected from a peptide having an amino acid sequence selected from SEQ ID NOS: 93, 120, and 123. In some embodiments, the Secretion Signal Peptide is a peptide having an amino acid sequence of SEQ ID NO: 66, the Immune Checkpoint Inhibitor Peptide is a peptide having an amino acid sequence of SEQ ID NO: 1, and the Cleavable Peptide is a peptide having an amino acid sequence of SEQ ID NO: 123, wherein x=2-10. In some embodiments, the Secretion Signal Peptide is a peptide having an amino acid sequence of SEQ ID NO: 66, the Immune Checkpoint Inhibitor Peptide is a peptide having an amino acid sequence of SEQ ID NO: 1, and the Cleavable Peptide is a peptide having an amino acid sequence of SEQ ID NO: 123, wherein x≥4. In some embodiments, the Secretion Signal Peptide is a peptide having an amino acid sequence of SEQ ID NO: 66, the Immune Checkpoint Inhibitor Peptide is a peptide having an amino acid sequence of SEQ ID NO: 1, and the Cleavable Peptide is a peptide having an amino acid sequence of SEQ ID NO: 123, wherein x=4, 5, or 6. In some embodiments, the Secretion Signal Peptide is a peptide having an amino acid sequence of SEQ ID NO: 66, the Immune Checkpoint Inhibitor Peptide is a peptide having an amino acid sequence of SEQ ID NO: 5, and the Cleavable Peptide is a peptide having an amino acid sequence of SEQ ID NO: 123, wherein x=2-10. In some embodiments, the Secretion Signal Peptide is a peptide having an amino acid sequence of SEQ ID NO: 66, the Immune Checkpoint Inhibitor Peptide is a peptide having an amino acid sequence of SEQ ID NO: 5, and the Cleavable Peptide is a peptide having an amino acid sequence of SEQ ID NO: 123, wherein x≥4. In some embodiments, the Secretion Signal Peptide is a peptide having an amino acid sequence of SEQ ID NO: 66, the Immune Checkpoint Inhibitor Peptide is a peptide having an amino acid sequence of SEQ ID NO: 5, and the Cleavable Peptide is a peptide having an amino acid sequence of SEQ ID NO: 123, wherein x=4, 5, or 6. In some embodiments, the antigenic peptide is selected from SEQ ID NOS: 349-394.

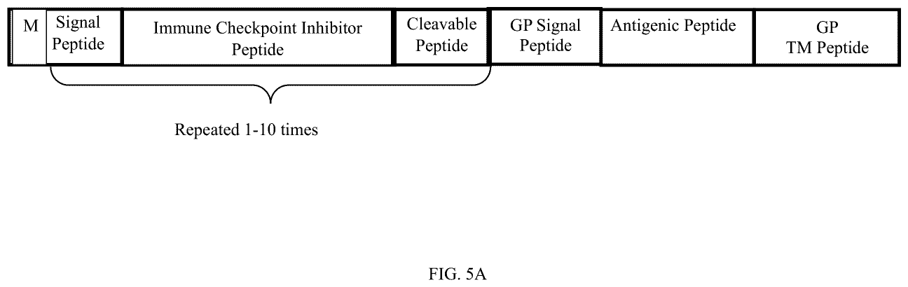

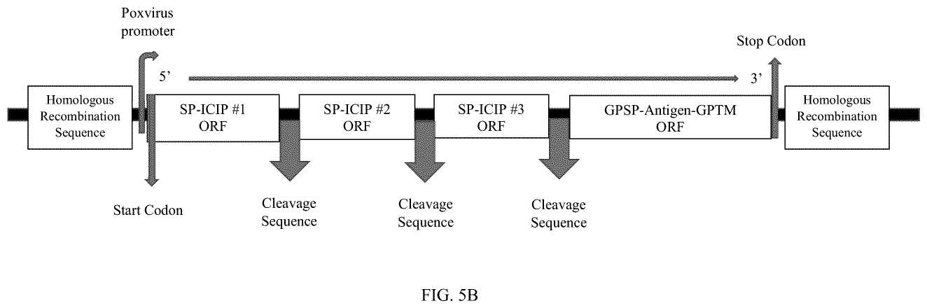

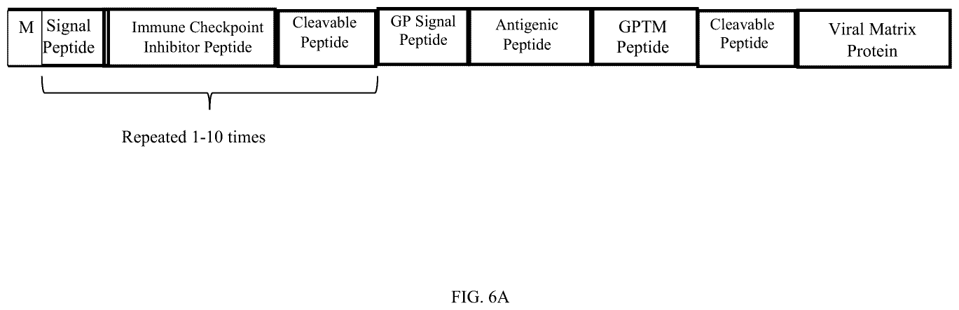

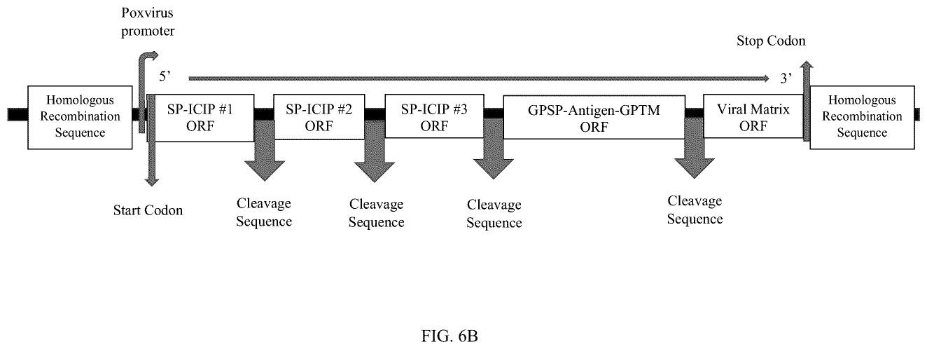

In some embodiments, the antigenic peptide encoded by the polycistronic nucleic acid insert in the rMVA is contained in a chimeric polypeptide that includes a viral glycoprotein signal sequence fused to the N-terminus of the antigenic peptide, and a viral glycoprotein transmembrane domain fused to the C-terminus of the antigenic peptide, and the rMVA is further constructed to encode a viral matrix protein, wherein upon translational cleavage of the antigenic containing chimeric peptide, the viral matrix protein and antigen-viral glycoprotein chimeric polypeptide are capable of forming a non-infectious virus-like particle (VLP). In some embodiments, provided herein is an rMVA viral vector comprising a heterologous polycistronic nucleic acid insert encoding a polypeptide wherein the polypeptide comprises ((M)(Secretion Signal Peptide-Immune Checkpoint Inhibitor Peptide-Cleavable Peptide)ₓ(Glycoprotein Signal Peptide-Antigenic Peptide-Glycoprotein Transmembrane Domain)), wherein x=1, 2, 3, 4, 5, 6, 7, 8, 9, 10, or more than 10, and wherein M=methionine (see, e.g., FIGS. 5A & 5B). In some embodiments, the antigenic peptide is contained in a chimeric polypeptide comprising a viral glycoprotein signal sequence fused to the N-terminus of the antigenic peptide, and a viral glycoprotein transmembrane domain fused to the C-terminus of the antigenic peptide, and a cleavable peptide fused to the C-terminus of the viral glycoprotein transmembrane domain, for example ((M)(Secretion Signal Peptide-Immune Checkpoint Inhibitor Peptide-Cleavable Peptide)ₓ(Glycoprotein Signal Peptide-Antigenic Peptide-Glycoprotein Transmembrane Domain-Cleavable Peptide)_{y}), wherein x=1, 2, 3, 4, 5, 6, 7, 8, 9, 10, or more than 10, y=1, 2, 3, 4, 5, 6, 7, 8, 9, 10, or more than 10, and wherein M=methionine. In some embodiments, the antigen containing chimeric polypeptide fused to the C-terminus of the last antigen containing chimeric polypeptide does not include a cleavable sequence, for example ((M)(Secretion Signal Peptide-Immune Checkpoint Inhibitor Peptide-Cleavable Peptide)ₓ(Glycoprotein Signal Peptide-Antigenic Peptide-Glycoprotein Transmembrane Domain-Cleavable Peptide)_{y}(Glycoprotein Signal Peptide-Antigenic Peptide-Glycoprotein Transmembrane Domain)), wherein x=1, 2, 3, 4, 5, 6, 7, 8, 9, 10, or more than 10, wherein y=1, 2, 3, 4, 5, 6, 7, 8, 9, 10, or more than 10, and wherein M=methionine. In some embodiments, the (Glycoprotein Signal Peptide-Antigenic Peptide-Glycoprotein Transmembrane Domain-Cleavable Peptide)y, wherein y=1, 2, 3, 4, 5, 6, 7, 8, 9, 10, or more than 10, can be oriented in the polycistronic nucleic acid insert so that the antigen containing chimeric polypeptide encoding nucleic acid is located 5′ of the immune checkpoint inhibitor peptide containing chimeric polypeptides, for example ((M)(Glycoprotein Signal Peptide-Antigenic Peptide-Glycoprotein Transmembrane Domain-Cleavable Peptide)_{y}(Secretion Signal Peptide-Immune Checkpoint Inhibitor Peptide-Cleavable Peptide)ₓ) or, alternatively ((M)(Glycoprotein Signal Peptide-Antigenic Peptide-Glycoprotein Transmembrane Domain-Cleavable Peptide)y(Secretion Signal Peptide-Immune Checkpoint Inhibitor Peptide-Cleavable Peptide)ₓ(Secretion Signal Peptide-Immune Checkpoint Inhibitor Peptide)), wherein x=1, 2, 3, 4, 5, 6, 7, 8, 9, 10, or more than 10, y=1, 2, 3, 4, 5, 6, 7, 8, 9, 10, or more than 10, and M=methionine. In yet a further embodiment, the polycistronic nucleic acid insert of the rMVA further encodes the viral matrix protein, for example, ((M)(Secretion Signal Peptide-Immune Checkpoint Inhibitor Peptide-Cleavable Peptide)ₓ(Glycoprotein Signal Peptide-Antigenic Peptide-Glycoprotein Transmembrane Domain-Cleavable Peptide)(Viral Matrix Protein)), wherein x=1, 2, 3, 4, 5, 6, 7, 8, 9, 10, or more than 10, and M=methionine (see, e.g., FIGS. 6A & 6B). In alternative embodiments, the coding sequences for both the antigen containing chimeric polypeptide and the viral matrix protein are contained in the polycistronic nucleic acid in one or more copies, for example, ((M)(Secretion Signal Peptide-Immune Checkpoint Inhibitor Peptide-Cleavable Peptide)ₓ(Glycoprotein Signal Peptide-Antigenic Peptide-Glycoprotein Transmembrane Domain-Cleavable Peptide)_{y}(Viral Matrix Protein-Cleavable Peptide)y), wherein x=1, 2, 3, 4, 5, 6, 7, 8, 9, 10, or more than 10, y=1, 2, 3, 4, 5, 6, 7, 8, 9, 10, or more than 10, and M=methionine. In some embodiments, the most C-terminus viral matrix protein lacks a cleavable peptide, for example, ((M)(Secretion Signal Peptide-Immune Checkpoint Inhibitor Peptide-Cleavable Peptide)ₓ(Glycoprotein Signal Peptide-Antigenic Peptide-Glycoprotein Transmembrane Domain-Cleavable Peptide)ₓ(Viral Matrix Protein-Cleavable Peptide)_{y}(Viral Matrix Protein)), wherein x=1, 2, 3, 4, 5, 6, 7, 8, 9, 10, or more than 10, y=1, 2, 3, 4, 5, 6, 7, 8, 9, 10, or more than 10, and M=methionine. In some embodiments, the ((Glycoprotein Signal Peptide-Antigenic Peptide-Glycoprotein Transmembrane Domain-Cleavable Peptide)_{y}(Viral Matrix Protein-Cleavable Peptide)_{y}), wherein y=1, 2, 3, 4, 5, 6, 7, 8, 9, 10, or more than 10, and M=methionine, can be oriented in the polycistronic nucleic acid insert so that the sequences are located 5′ of the immune checkpoint inhibitor peptide containing chimeric polypeptides, for example ((M)(Glycoprotein Signal Peptide-Antigenic Peptide-Glycoprotein Transmembrane Domain-Cleavable Peptide)_{y}(Viral Matrix Protein-Cleavable Peptide)_{y}(Secretion Signal Peptide-Immune Checkpoint Inhibitor Peptide-Cleavable Peptide)ₓ) or, alternatively ((M)(Glycoprotein Signal Peptide-Antigenic Peptide-Glycoprotein Transmembrane Domain-Cleavable Peptide)_{y}(Viral Matrix Protein-Cleavable Peptide)_{y}(Secretion Signal Peptide-Immune Checkpoint Inhibitor Peptide-Cleavable Peptide)ₓ(Secretion Signal Peptide-Immune Checkpoint Inhibitor Peptide)), wherein x=1, 2, 3, 4, 5, 6, 7, 8, 9, 10, or more than 10, y=1, 2, 3, 4, 5, 6, 7, 8, 9, 10, or more than 10, and M=methionine.

In particular embodiments, the glycoprotein and matrix proteins are derived from Marburg virus (MARV). In particular embodiments, the glycoprotein is derived from the MARV GP protein (Genbank accession number AFV31202.1). The amino acid sequence of the MARV GP protein is provided as SEQ TD. No. 395 in Table 10 below. In particular embodiments, the MARV GPS domain comprises amino acids 2 to 19 ofthe glycoprotein (WTTCFFISLIIQGIKTL) (SEQ ID. No. 396, which can be encoded by, for example the MVA optimized nucleic acid sequence of SEQ TD. No. 397), the GPTM domain comprises amino acid sequences 644-673 of the glycoprotein (WWTSDWGVLTNLGILLLLSIAVLIALSCICRIFTKYIG) (SEQ ID. No. 398, which can be encoded by, for example the MVA optimized nucleic acid sequence of SEQ TD. No. 399), or a nucleic acid sequence 70%, 75%, 80%, 85%, 90%, 95% or more identical thereto. In some embodiments, the MARV GPS signal further comprises a methionine as the first amino acid.

The MARV VP40 amino acid sequence is available at Genflank accession number JX458834, and provided below in Table 10 as SEQ ID. No. 400, which can be encoded by, for example, the MVA optimized nucleic acid sequence of SEQ ID. No. 401, or a nucleic acid sequence 7000, 7500, 8000, 850%, 900%, 9500 or more identical thereto. In some embodiments, the MARV VP40 amino acid sequence further comprises a methionine as the first amino acid.

TABLE 10
MARV Glycoprotein Domains and VP40 Protein

| SEQ ID NO: | Sequence |
|---|---|
| 395-GP MARV | WTTCFFISLILIQGIKTLPILEIASNDQPQNVDSVCSGTLQKTEDVHLMGFTLSGQKV |
| amino acid | ADSPLEASKRWAFRTGVPPKNVEYTEGEEAKTCYNISVTDPSGKSLLLDPPTNVRD |
| sequence | YPKCKTIHHIQGQNPHAQGIALHLWGAFFLYDRIASTTMYRGKVFTEGNIAAMIVN |
| | KTVHKMIFSRQGQGYRHMNLTSTNKYWTSSNGTQTNDTGCFGTLQEYNSTKNQT |
| | CAPSKTPPPPPTAHPEIKPTSTPTDATRLNTTNPNSDDEDLTTSGSGSGEQEPYTTSD |
| | AVTKQGLSSTMPPTLSPQPGTPQQGGNNTNHSQDAATELDNTNTTAQPPMPSHNT |
| | TTISTNNTSKHNLSTLSEPPQNTTNPNTQSMATENEKTSAPPKTTLPPTESPTTEKST |
| | NNTKSPTTMEPNTTNGHFTSPSSTPNSTTQHLIYFRRKRSILWREGDMFPFLDGLIN |
| | APIDFDPVPNTKTIFDESSSSGASAEEDQHASSNISLTLSYLPHTSENTAYSGENEND |
| | CDAELRIWSVQEDDLAAGLSWIPFFGPGIEGLYTAGLIKNQNNLVCRLRRLANQTA |
| | KSLELLLRVTTEERTFSLINRHAIDFLLTRWGGTCKVLGPDCCIGIEDLSRNISEQID |
| | QIKKDEQKEGTGWGLGGKWWTSDWGVLTNLGILLLLSIAVLIALSCICRIFTKYIG |
| 396-Signal | WTTCFFISLILIQGIKTL |
| peptide amino acid | |
| sequence of GP | |
| MARV | |
| 397-Signal | TGGACGACCTGCTTCTTCATCTCCCTAATCCTAATCCAGGGAATCAAGACCCTA |
| peptide nucleic acid | |
| sequence of GP | |
| MARV-optimized | |
| 398 | WWTSDWGVLTNLGILLLLSIAVLIALSCICRIFTKYIG |
| Transmembrane | |
| domain amino acid | |
| sequence of GP | |
| MARV | |
| 399 | TGGTGGACATCTGACTGGGGAGTCCTAACGAACCTAGGAATCCTACTACTATT |
| Transmembrane | GTCGATCGCGGTCCTAATCGCGCTATCCTGTATCTGTAGAATCTTCACCAAGTA |
| domain nucleic | CATCGGA |
| acid sequence of | |
| GP MARV- | |
| optimized | |
| 400-MARVVP 40 | ASSSNYNTYMQYLNPPPYADHGANQLIPADQLSNQHGITPNYVGDLNLDDQFKGN |
| amino acid | VCHAFTLEAIIDISAYNERTVKGVPAWLPLGIMSNFEYPLAHTVAALLTGSYTITQF |
| sequence | THNGQKFVRVNRLGTGIPAHPLRMLREGNQAFIQNMVIPRNFSTNQFTYNLTNLVL |
| | SVQKLPDDAWRPSKDKLIGNTMHPAISIHPNLPPIVLPTVKKQAYRQHKNPNNGPL |
| | LAISGILHQLRVEKVPEKTSLFRISLPADMFSVKEGMMKKRGESSPVVYFQAPENFP |
| | LNGFNNRQVVLAYANPTLSAI |
| 401-MARVVP 40 | GCGTCTAGTTCTAATTATAATACTTATATGCAATATCTAAATCCACCACCATAT |
| nucleic acid | GCGGATCATGGTGCTAATCAACTAATTCCAGCGGATCAACTATCTAATCAACA |
| sequence- | TGGAATTACACCAAATTATGTTGGAGATCTAAATCTAGATGATCAGTTTAAAG |
| optimized | GAAATGTTTGTCATGCGTTTACACTAGAAGCGATTATTGATATTTCTGCGTATA |
| | ATGAAAGAACAGTAAAAGGTGTACCAGCTTGGCTACCACTAGGAATTATGTCT |
| | AATTTTGAATATCCACTAGCGCATACAGTAGCGGCGCTATTGACAGGATCTTAT |
| | ACAATTACACAGTTTACACATAATGGACAAAAgTTTGTTAGAGTAAATAGACT |
| | AGGAACTGGAATACCAGCGCATCCACTAAGAATGCTAAGAGAAGGAAATCAA |
| | GCTTTTATTCAAAATATGGTTATTCCAAGAAATTTcTCTACAAATCAGTTTACTT |
| | ATAATCTAACTAATCTAGTACTATCTGTACAAAAGCTACCAGATGATGCTTGGA |
| | GACCATCTAAAGATAAACTAATTGGAAATACAATGCATCCAGCGATTTCTATT |
| | CATCCAAATCTACCACCAATAGTACTACCAACTGTAAAgAAACAAGCGTATAG |
| | ACAACATAAgAATCCAAATAATGGACCACTATTGGCGATTTCTGGAATTCTACA |
| | TCAACTAAGAGTAGAAAAgGTACCAGAAAAgACATCTTTGTTTAGAATTTCTCT |
| | ACCAGCGGATATGTTTTCTGTAAAAGAAGGAATGATGAAgAAAAGAGGAGAAT |
| | CTTCTCCAGTAGTATATTTTCAAGCGCCAGAAAATTTTCCATTGAATGGTTTTA |
| | ATAATAGACAAGTAGTACTAGCGTATGCGAATCCAACACTATCTGCGATATAA |
| | TAA |

In some embodiments, any of the above SEQ TD NOS:395-396 and 400, further includes the amino acid residue methionine (M) as the first amino acid residue. In some embodiments, any of the above SEQ TD NOS:397 ad 401, further includes the nucleic acid codon ATG as the first codon of the coding sequence. In particular embodiments, the encoded polypeptide comprises, in various alternative embodiments, ((M)(Secretion Signal Peptide-Immune Checkpoint Inhibitor Peptide-Cleavable Peptide)ₓ(Glycoprotein Signal Peptide-Antigenic Peptide-Glycoprotein Transmembrane Domain)), ((M)(Secretion Signal Peptide-Immune Checkpoint Inhibitor Peptide-Cleavable Peptide)ₓ(Glycoprotein Signal Peptide-Antigenic Peptide-Glycoprotein Transmembrane Domain-Cleavable Peptide)ₓ), ((M)(Secretion Signal Peptide-Immune Checkpoint Inhibitor Peptide-Cleavable Peptide)ₓ(Glycoprotein Signal Peptide-Antigenic Peptide-Glycoprotein Transmembrane Domain-Cleavable Peptide)_{y}(Glycoprotein Signal Peptide-Antigenic Peptide-Glycoprotein Transmembrane Domain)), ((M)(Glycoprotein Signal Peptide-Antigenic Peptide-Glycoprotein Transmembrane Domain-Cleavable Peptide)_{y}(Secretion Signal Peptide-Immune Checkpoint Inhibitor Peptide-Cleavable Peptide)ₓ), ((M)(Glycoprotein Signal Peptide-Antigenic Peptide-Glycoprotein Transmembrane Domain-Cleavable Peptide)_{y}(Secretion Signal Peptide-Immune Checkpoint Inhibitor Peptide-Cleavable Peptide)ₓ(Secretion Signal Peptide-Immune Checkpoint Inhibitor Peptide)), ((M)(Secretion Signal Peptide-Immune Checkpoint Inhibitor Peptide-Cleavable Peptide)ₓ(Glycoprotein Signal Peptide-Antigenic Peptide-Glycoprotein Transmembrane Domain-Cleavable Peptide)(Viral Matrix Protein)), ((M)(Secretion Signal Peptide-Immune Checkpoint Inhibitor Peptide-Cleavable Peptide)ₓ(Glycoprotein Signal Peptide-Antigenic Peptide-Glycoprotein Transmembrane Domain-Cleavable Peptide)_{y}(Viral Matrix Protein-Cleavable Peptide)_{y}), ((M)(Secretion Signal Peptide-Immune Checkpoint Inhibitor Peptide-Cleavable Peptide)ₓ(Glycoprotein Signal Peptide-Antigenic Peptide-Glycoprotein Transmembrane Domain-Cleavable Peptide)ₓ(Viral Matrix Protein-Cleavable Peptide)_{y}(Viral Matrix Protein)), ((M)(Glycoprotein Signal Peptide-Antigenic Peptide-Glycoprotein Transmembrane Domain-Cleavable Peptide)_{y}(Viral Matrix Protein-Cleavable Peptide)_{y}), ((M)(Glycoprotein Signal Peptide-Antigenic Peptide-Glycoprotein Transmembrane Domain-Cleavable Peptide)_{y}(Viral Matrix Protein-Cleavable Peptide)_{y}(Secretion Signal Peptide-Immune Checkpoint Inhibitor Peptide-Cleavable Peptide)ₓ), or ((M)(Glycoprotein Signal Peptide-Antigenic Peptide-Glycoprotein Transmembrane Domain-Cleavable Peptide)_{y}(Viral Matrix Protein-Cleavable Peptide)_{y}(Secretion Signal Peptide-Immune Checkpoint Inhibitor Peptide-Cleavable Peptide)ₓ(Secretion Signal Peptide-Immune Checkpoint Inhibitor Peptide)), wherein x=1, 2, 3, 4, 5, 6, 7, 8, 9, 10, or more than 10, y=1, 2, 3, 4, 5, 6, 7, 8, 9, 10, or more than 10, M=methionine, and wherein the Secretion Signal Peptide is selected from a peptide having an amino acid sequence selected from SEQ ID NOS: 57-90, the Immune Checkpoint Inhibitor Peptide is selected from a peptide having an amino acid sequence selected from SEQ ID NOS: 1-56, the Cleavable Peptide is selected from a peptide having an amino acid sequence selected from SEQ ID NOS: 91-127, the Glycoprotein Signal Peptide is a peptide having the amino acid sequence of SEQ ID NO. 396, the Glycoprotein Transmembrane Domain is a peptide having the amino acid sequence of SEQ ID NO. 398, the Viral Matrix Protein, when present, is a peptide having the amino acid sequence of SEQ ID NO: 400, and the antigenic peptide is a peptide derived from an infectious agent, for example a virus, bacteria, parasite, fungus, or toxoid, or alternatively, a tumor associated antigen. In some embodiments, the antigenic peptide is selected from SEQ ID NOS: 349-394. In some embodiments, the Secretion Signal Peptide is selected from a peptide having an amino acid sequence selected from SEQ ID NOS: 65 and 66, the Immune Checkpoint Inhibitor Peptide is selected from a peptide having an amino acid sequence selected from SEQ ID NOS: 1 and 5, the Cleavable Peptide is selected from a peptide having an amino acid sequence selected from SEQ ID NOS: 93, 120, and 123, the Glycoprotein Signal Peptide is a peptide having the amino acid sequence of SEQ ID NO. 396, the Glycoprotein Transmembrane Domain is a peptide having the amino acid sequence of SEQ ID NO. 398, and the Viral Matrix Protein, when present, is a peptide having the amino acid sequence of SEQ ID NO: 400, and the antigenic peptide is a peptide derived from an infectious agent, for example a virus, bacteria, parasite, fungus, or toxoid, or alternatively, a tumor associated antigen, or the antigenic peptide is selected from SEQ ID NOS: 349-394. In some embodiments, the Secretion Signal Peptide is a peptide having an amino acid sequence of SEQ ID NO: 66, the Immune Checkpoint Inhibitor Peptide is a peptide having an amino acid sequence of SEQ ID NO: 1, the Cleavable Peptide is a peptide having an amino acid sequence of SEQ ID NO: 123, the Glycoprotein Signal Peptide is a peptide having the amino acid sequence of SEQ ID NO. 396, the Glycoprotein Transmembrane Domain is a peptide having the amino acid sequence of SEQ ID NO. 398, and the Viral Matrix Protein, when present, is a peptide having the amino acid sequence of SEQ ID NO: 400, and the antigenic peptide is a peptide derived from an infectious agent, for example a virus, bacteria, parasite, fungus, or toxoid, or alternatively, a tumor associated antigen, or the antigenic peptide is selected from SEQ ID NOS: 349-394, and wherein x=1-10. In some embodiments, the Secretion Signal Peptide is a peptide having an amino acid sequence of SEQ ID NO: 66, the Immune Checkpoint Inhibitor Peptide is a peptide having an amino acid sequence of SEQ ID NO: 1, and the Cleavable Peptide is a peptide having an amino acid sequence of SEQ ID NO: 123, the Glycoprotein Signal Peptide is a peptide having the amino acid sequence of SEQ ID NO. 396, the Glycoprotein Transmembrane Domain is a peptide having the amino acid sequence of SEQ ID NO. 398, and the Viral Matrix Protein, when present, is a peptide having the amino acid sequence of SEQ ID NO: 400, and the antigenic peptide is a peptide derived from an infectious agent, for example a virus, bacteria, parasite, fungus, or toxoid, or alternatively, a tumor associated antigen, or the antigenic peptide is selected from SEQ ID NOS: 349-394, wherein x≥4. In some embodiments, the Secretion Signal Peptide is a peptide having an amino acid sequence of SEQ ID NO: 66, the Immune Checkpoint Inhibitor Peptide is a peptide having an amino acid sequence of SEQ ID NO: 1, the Cleavable Peptide is a peptide having an amino acid sequence of SEQ ID NO: 123, the Glycoprotein Signal Peptide is a peptide having the amino acid sequence of SEQ ID NO. 396, the Glycoprotein Transmembrane Domain is a peptide having the amino acid sequence of SEQ ID NO. 398, and the Viral Matrix Protein, when present, is a peptide having the amino acid sequence of SEQ ID NO: 400, and the antigenic peptide is a peptide derived from an infectious agent, for example a virus, bacteria, parasite, fungus, or toxoid, or alternatively, a tumor associated antigen, or the antigenic peptide is selected from SEQ ID NOS: 349-394, and wherein x=4, 5, or 6. In some embodiments, the Secretion Signal Peptide is a peptide having an amino acid sequence of SEQ ID NO: 66, the Immune Checkpoint Inhibitor Peptide is a peptide having an amino acid sequence of SEQ ID NO: 5, the Cleavable Peptide is a peptide having an amino acid sequence of SEQ ID NO: 123, the Glycoprotein Signal Peptide is a peptide having the amino acid sequence of SEQ ID NO. 396, the Glycoprotein Transmembrane Domain is a peptide having the amino acid sequence of SEQ ID NO. 398, and the Viral Matrix Protein, when present, is a peptide having the amino acid sequence of SEQ ID NO: 400, and the antigenic peptide is a peptide derived from an infectious agent, for example a virus, bacteria, parasite, fungus, or toxoid, or alternatively, a tumor associated antigen, or the antigenic peptide is selected from SEQ ID NOS: 349-394, wherein x=1-10. In some embodiments, the Secretion Signal Peptide is a peptide having an amino acid sequence of SEQ ID NO: 66, the Immune Checkpoint Inhibitor Peptide is a peptide having an amino acid sequence of SEQ ID NO: 5, the Cleavable Peptide is a peptide having an amino acid sequence of SEQ ID NO: 123, the Glycoprotein Signal Peptide is a peptide having the amino acid sequence of SEQ ID NO. 396, the Glycoprotein Transmembrane Domain is a peptide having the amino acid sequence of SEQ ID NO. 398, and the Viral Matrix Protein, when present, is a peptide having the amino acid sequence of SEQ ID NO: 400, and the antigenic peptide is a peptide derived from an infectious agent, for example a virus, bacteria, parasite, fungus, or toxoid, or alternatively, a tumor associated antigen, the antigenic peptide is selected from SEQ ID NOS: 349-394, wherein x≥4. In some embodiments, the Secretion Signal Peptide is a peptide having an amino acid sequence of SEQ ID NO: 66, the Immune Checkpoint Inhibitor Peptide is a peptide having an amino acid sequence of SEQ ID NO: 5, the Cleavable Peptide is a peptide having an amino acid sequence of SEQ ID NO: 123, the Glycoprotein Signal Peptide is a peptide having the amino acid sequence of SEQ ID NO. 396, the Glycoprotein Transmembrane Domain is a peptide having the amino acid sequence of SEQ ID NO. 398, and the Viral Matrix Protein, when present, is a peptide having the amino acid sequence of SEQ ID NO: 400, and the antigenic peptide is a peptide derived from an infectious agent, for example a virus, bacteria, parasite, fungus, or toxoid, or alternatively, a tumor associated antigen, or the antigenic peptide is selected from SEQ ID NOS: 349-394, wherein x=4, 5, or 6. In some embodiments, the encoded polypeptide comprises SEQ ID NOS. 325 or 333, the Glycoprotein Signal Peptide is a peptide having the amino acid sequence of SEQ ID NO. 396, the Glycoprotein Transmembrane Domain is a peptide having the amino acid sequence of SEQ ID NO. 398, and the Viral Matrix Protein, when present, is a peptide having the amino acid sequence of SEQ ID NO: 400, and the antigenic peptide is a peptide derived from an infectious agent, for example a virus, bacteria, parasite, fungus, or toxoid, or alternatively, a tumor associated antigen, or the antigenic peptide is selected from SEQ ID NOS: 349-394. In some embodiments, the encoded polypeptide comprises SEQ ID NO. 329 or 337, the Glycoprotein Signal Peptide is a peptide having the amino acid sequence of SEQ ID NO. 396, the Glycoprotein Transmembrane Domain is a peptide having the amino acid sequence of SEQ ID NO. 398, and the Viral Matrix Protein, when present, is a peptide having the amino acid sequence of SEQ ID NO: 400, and the antigenic peptide is a peptide derived from an infectious agent, for example a virus, bacteria, parasite, fungus, or toxoid, or alternatively, a tumor associated antigen, or the antigenic peptide is selected from SEQ ID NOS: 349-394.

In alternative embodiments, the rMVA viral vectors of the present invention, in addition to the ability to express multiple immune checkpoint inhibitor peptides, may further be constructed to encode and express one or more antigen peptides encoded on one or more separate nucleic acid inserts. In some embodiments, the nucleic acid sequence encoding multiple immune checkpoint inhibitor peptides as described herein is inserted into one gene locus of the rMVA, and one or more heterologous nucleic acid sequences encoding an antigenic peptide is inserted into a separate gene locus of the rMVA. The one or more antigen peptides can be derived from any of the targets described in the section Antigenic Targets, incorporated into this section in its entirety for all purposes. In some embodiments, the antigen peptides are derived from any of the amino acid sequences selected from SEQ ID NOS: 349-396, 398, or 400, or a fragment derived therefrom, or an amino acid sequence at least 85%, 90%, 95%, 96%, 97%, 98%, or 99% identical thereto. If inserted as a separate nucleic acid insert, a start codon encoding the amino acid residue methionine (M) can be included as the first residue of the antigen peptides are derived from any of the amino acid sequences selected from SEQ ID NOS: 349-396, 398, or 400, or a fragment derived therefrom, or an amino acid sequence at least 85%, 90%, 95%, 96%, 97%, 98%, or 99% identical thereto.

In certain embodiments, the rMVA, in addition to the polycistronic nucleic acid encoding the immune checkpoint inhibitor polypeptides described herein, further encodes an antigenic peptide comprising a chimeric peptide comprising an extracellular domain of an antigen and a transmembrane domain of a viral glycoprotein, and further encodes a viral matrix protein, wherein the chimeric peptide and viral matrix protein, when expressed, are capable of forming a virus-like particle (VLP) in vivo. In some embodiments, the transmembrane domain of the viral glycoprotein is derived from the amino acid of SEQ ID NO: 398, or a fragment thereof, or an amino acid sequence at least 85%, 90%, 95%, 96%, 97%, 98%, or 99% identical thereto. In some embodiments, the viral matrix protein is derived from Marburg virus VP40 protein, for example, as provided in SEQ ID NO: 404, or a fragment thereof, or an amino acid sequence at least 85%, 90%, 95%, 96%, 97%, 98%, or 99% identical thereto. In some embodiments, the rMVA encodes for the amino acid sequence of SEQ ID NO:329, or a fragment thereof, or an amino acid sequence at least 85%, 90%, 95%, 96%, 97%, 98%, or 99% identical thereto, the amino acid sequence of SEQ ID NO: 402, or a fragment thereof, or an amino acid sequence at least 85%, 90%, 95%, 96%, 97%, 98%, or 99% identical thereto, and the amino acid sequence of SEQ ID NO:404, or a fragment thereof, or an amino acid sequence at least 85%, 90%, 95%, 96%, 97%, 98%, or 99% identical thereto.

TABLE 11
MUC-Insert Sequences

| | |
|---|---|
| SEQ ID NO: | ATGACACCTGGAACACAATCTCCATTCTTCCTACTACTACTATTGACAGTACTAACA |
| 402-MUC-1- | GTAGTAACAGGATCTGGACATGCGTCTAGTACACCAGGTGGAGAGAAGGAAACAT |
| MARV GPTM- | CTGCGACTCAAAGATCTTCTGTACCATCTTCTACAGAGAAGAATGCGGTATCTATG |
| optimized | ACATCTAGTGTACTATCTTCTCATTCTCCTGGATCTGGATCTTCTACTACACAAGGA |
| nucleic acid | CAAGATGTAACACTAGCGCCAGCTACAGAACCAGCTTCTGGATCTGCTGCTACTTG |
| sequence | GGGTCAAGATGTTACTTCTGTTCCAGTAACAAGACCAGCGCTAGGATCTACAACAC |
| | CACCAGCGCATGATGTAACAAGTGCGCCAGATAATAAGCCAGCGCCTGGTTCTACT |
| | GCTCCACCAGCTCATGGTGTTACTTCAGCGCCTGATACAAGACCCGCACCCGGATC |
| | TACCGCTCCGCCTGCACACGGCGTCACATCTGCTCCCGACACTCGTCCAGCTCCTGG |
| | TAGCACAGCACCTCCAGCGCATGGAGTAACCAGTGCACCAGATACCCGACCTGCGC |
| | CGGGCAGTACTGCCCCACCGGCCCACGGGGTGACGAGCGCCCCGGACACGCGCCC |
| | AGCTCCAGGGTCAACGGCGCCCCCTGCTCATGGTGTTACAAGTGCACCTGATAATA |
| | GACCTGCGTTGGGATCTACTGCGCCTCCAGTTCATAATGTAACATCAGCGTCTGGA |
| | AGTGCGTCTGGTTCTGCGTCTACATTGGTTCATAATGGTACATCTGCGAGAGCGAC |
| | AACAACTCCAGCGTCTAAGTCTACACCATTCTCTATTCCATCTCATCATTCTGATAC |
| | ACCAACAACATTGGCGAGTCATTCTACAAAGACAGATGCGAGTTCTACACATCATT |
| | CTACTGTACCACCACTAACATCTTCTAATCATAGTACATCTCCACAACTATCTACTG |
| | GTGTATCTTTCTTCTTCCTATCCTTTCATATTTCTAATCTACAGTTCAATTCTAGTTT |
| | GGAAGATCCATCTACAGATTATTATCAAGAACTACAAAGAGATATTTCTGAAATGT |
| | TTCTACAAATATATAAACAAGGAGGATTTCTAGGACTATCTAATATTAAGTTTAGA |
| | CCAGGATCTGTAGTAGTTCAACTAACTCTAGCGTTTAGAGAAGGTACTATTAATGT |
| | ACATGATGTTGAAACACAGTTTAATCAATATAAGACAGAAGCGGCGTCTAGATATA |
| | ATCTAACAATTTCTGATGTATCTGTATCTGATGTTCCATTTCCATTCTCTGCGCAATC |
| | TGGTGCTGGTGTATGGTGGACATCTGATTGGGGAGTACTAACTAATCTAGGAATTC |
| | TACTATTGCTATCTATTGCGGTACTAATTGCGCTATCTTGTATATGTAGAAGAAAGA |
| | ATTATGGACAACTAGATATTTTCCCAGCGAGAGATACTTATCATCCAATGTCTGAA |
| | TATCCAACATATCATACACATGGAAGATATGTACCACCTTCTTCAACAGATAGATC |
| | TCCATATGAGAAGGTATCTGCGGGAAATGGTGGTTCTTCTCTATCTTATACAAATCC |
| | AGCGGTAGCGGCGACTTCTGCGAATCTATAA |
| SEQ ID NO: | MTPGTQSPFFLLLLLTVLTVVTGSGHASSTPGGEKETSATQRSSVPSSTEKNAVSMTSSV |
| 403-MUC-1- | LSSHSPGSGSSTTQGQDVTLAPATEPASGSAATWGQDVTSVPVTRPALGSTTPPAHDVT |
| MARV GPTM- | SAPDNKPAPGSTAPPAHGVTSAPDTRPAPGSTAPPAHGVTSAPDTRPAPGSTAPPAHGV |
| amino acid | TSAPDTRPAPGSTAPPAHGVTSAPDTRPAPGSTAPPAHGVTSAPDNRPALGSTAPPVHN |
| sequence | VTSASGSASGSASTLVHNGTSARATTTPASKSTPFSIPSHHSDTPTTLASHSTKTDASSTH |
| | HSTVPPLTSSNHSTSPQLSTGVSFFFLSFHISNLQFNSSLEDPSTDYYQELQRDISEMFLQ |
| | IYKQGGFLGLSNIKFRPGSVVVQLTLAFREGTINVHDVETQFNQYKTEAASRYNLTISDV |
| | SVSDVPFPFSAQSGAGVWWTSDWGVLTNLGILLLLSIAVLIALSCICRRKNYGOLDIFPA |
| | RDTYHPMSEYPTYHTHGRYVPPSSTDRSPYEKVSAGNGGSSLSYTNPAVAATSANL |
| SEQ ID NO: | ATGGCGTCTAGTTCTAATTATAATACTTATATGCAATATCTAAATCCACCACCATAT |
| 404-Marburg | GCGGATCATGGTGCTAATCAACTAATTCCAGCGGATCAACTATCTAATCAACATGG |
| virus VP40 | AATTACACCAAATTATGTTGGAGATCTAAATCTAGATGATCAGTTTAAAGGAAATG |
| nucleic acid | TTTGTCATGCGTTTACACTAGAAGCGATTATTGATATTTCTGCGTATAATGAAAGAA |
| sequence | CAGTAAAAGGTGTACCAGCTTGGCTACCACTAGGAATTATGTCTAATTTTGAATAT |
| | CCACTAGCGCATACAGTAGCGGCGCTATTGACAGGATCTTATACAATTACACAGTT |
| | TACACATAATGGACAAAAGTTTGTTAGAGTAAATAGACTAGGAACTGGAATACCA |
| | GCGCATCCACTAAGAATGCTAAGAGAAGGAAATCAAGCTTTTATTCAAAATATGGT |
| | TATTCCAAGAAATTTCTCTACAAATCAGTTTACTTATAATCTAACTAATCTAGTACT |
| | ATCTGTACAAAAGCTACCAGATGATGCTTGGAGACCATCTAAAGATAAACTAATTG |
| | GAAATACAATGCATCCAGCGATTTCTATTCATCCAAATCTACCACCAATAGTACTA |
| | CCAACTGTAAAGAAACAAGCGTATAGACAACATAAGAATCCAAATAATGGACCAC |
| | TATTGGCGATTTCTGGAATTCTACATCAACTAAGAGTAGAAAAGGTACCAGAAAAG |
| | ACATCTTTGTTTAGAATTTCTCTACCAGCGGATATGTTTTCTGTAAAAGAAGGAATG |
| | ATGAAGAAAAGAGGAGAATCTTCTCCAGTAGTATATTTTCAAGCGCCAGAAAATTT |
| | TCCATTGAATGGTTTTAATAATAGACAAGTAGTACTAGCGTATGCGAATCCAACAC |
| | TATCTGCGATATAA |
| SEQ ID NO: | MASSSNYNTYMQYLNPPPYADHGANQLIPADQLSNQHGITPNYVGDLNLDDQFKGNV |
| 405-Marburg | CHAFTLEAIIDISAYNERTVKGVPAWLPLGIMSNFEYPLAHTVAALLTGSYTITQFTHNG |
| virus VP40 | QKFVRVNRLGTGIPAHPLRMLREGNQAFIQNMVIPRNFSTNQFTYNLTNLVLSVQKLPD |
| amino acid | DAWRPSKDKLIGNTMHPAISIHPNLPPIVLPTVKKQAYRQHKNPNNGPLLAISGILHQLR |
| sequence | VEKVPEKTSLFRISLPADMFSVKEGMMKKRGESSPVVYFQAPENFPLNGFNNRQVVLA |
| | YANPTLSAI |

### Sequence Optimization

One or more nucleic acid sequences comprising the polycistronic nucleic acid insert of the rMVA provided herein may be optimized for use in an MVA vector. Optimization includes codon optimization, which employs silent mutations to change selected codons from the native sequences into synonymous codons that are optimally expressed by the host-vector system. Other types of optimization include the use of silent mutations to interrupt homopolymer stretches or transcription terminator motifs. Each of these optimization strategies can improve the stability of the gene, improve the stability of the transcript, or improve the level of protein expression from the sequence. In exemplary embodiments, the number of homopolymer stretches in the heterologous DNA insert sequence will be reduced to stabilize the construct. A silent mutation may be provided for anything similar to a vaccinia termination signal.

In exemplary embodiments, the sequences are codon optimized for expression in MVA; sequences with runs of >5 deoxyguanosines, >5 deoxycytidines, >5 deoxyadenosines, and >5 deoxythymidines are interrupted by silent mutation to minimize loss of expression due to frame shift mutations.

In particular, the nucleic acid for insertion can be optimized by codon optimizing the original DNA sequence. For example, the “Invitrogen GeneArt Gene Software” can be used to codon optimize the DNA sequence. To fully optimize the gene sequence, homopolymer sequences (G/C or T/A rich areas) are interrupted via silent mutation(s). To the extent present in the nucleic acid insert sequence, the MVA transcription terminator (T5NT (UUUUUNU)) is interrupted via silent mutation(s). Further optimizations can include, for example, adding a Kozak sequence (GCCACC/ATG), adding a second stop codon, and adding a vaccinia virus transcription terminator, specifically “TTTTTAT”, or variations and/or combinations thereof.

### Pharmaceutical Compositions

The recombinant MVA viral vectors of the present invention are readily formulated as pharmaceutical compositions for veterinary or human use, either alone or in combination. The pharmaceutical composition may comprise a pharmaceutically acceptable diluent, excipient, carrier, or adjuvant, or, in an alternative embodiment, one or more antigenic agents, for example a antigen derived from an infectious disease or, in an alternative embodiment, a tumor associated antigen.

In one embodiment, the rMVA is used as an adjuvant effective in enhancing immunogenicity to an infectious agent to protect against and/or treat an infection, the rMVA comprising a polycistronic nucleic acid insert that encodes at least two or more immune checkpoint inhibitor peptides as described herein. In alternative embodiments, the rMVA is used as a vaccine effective in enhancing immunogenicity to an infectious agent to protect against and/or treat an infection, the rMVA comprising a polycistronic nucleic acid insert that encodes at least two or more immune checkpoint inhibitor peptides and one or more antigenic peptides as described herein.

As used herein, the phrase “pharmaceutically acceptable carrier” encompasses any of the standard pharmaceutical carriers, such as those suitable for parenteral administration, such as, for example, by intramuscular, intraarticular (in the joints), intravenous, intradermal, intraperitoneal, and subcutaneous routes. Examples of such formulations include aqueous and non-aqueous, isotonic sterile injection solutions, which contain antioxidants, buffers, bacteriostats, and solutes that render the formulation isotonic with the blood of the intended recipient, and aqueous and nonaqueous sterile suspensions that can include suspending agents, solubilizers, thickening agents, stabilizers, and preservatives. One exemplary pharmaceutically acceptable carrier is physiological saline. Carriers include excipients and diluents and must be of sufficiently high purity and sufficiently low toxicity to render them suitable for administration to the patient being treated. The carrier can be inert or it can possess pharmaceutical benefits of its own. The amount of carrier employed in conjunction with the compound is sufficient to provide a practical quantity of material for administration per unit dose of the compound.

Other physiologically acceptable diluents, excipients, carriers, or additional adjuvants and their formulations are known to those skilled in the art.

In some embodiments, additional adjuvants are used as further immune response enhancers. In various embodiments, the additional immune response enhancer is selected from the group consisting of alum-based adjuvants, oil based adjuvants, Specol, RIBI, TiterMax, Montanide ISA50 or Montanide ISA 720, GM-CSF, nonionic block copolymer-based adjuvants, dimethyl dioctadecyl ammoniumbromide (DDA) based adjuvants AS-1, AS-2, Ribi Adjuvant system based adjuvants, QS21, Quil A, SAF (Syntex adjuvant in its microfluidized form (SAF-m), dimethyl-dioctadecyl ammonium bromide (DDA), human complement based adjuvants m. vaccae, ISCOMS, MF-59, SBAS-2, SBAS-4, Enhanzyn®, RC-529, AGPs, MPL-SE, QS7, Escin; Digitonin; Gypsophila; and Chenopodium quinoa saponins.

The compositions utilized in the methods described herein can be administered by a route selected from, e.g., parenteral, intramuscular, intraarterial, intravascular, intravenous, intraperitoneal, subcutaneous, dermal, transdermal, ocular, inhalation, buccal, sublingual, perilingual, nasal, topical administration, and oral administration. The preferred method of administration can vary depending on various factors (e.g., the components of the composition being administered and the severity of the condition being treated). Formulations suitable for oral administration may consist of liquid solutions, such as an effective amount of the composition dissolved in a diluent (e.g., water, saline, or PEG-400), capsules, sachets or tablets, each containing a predetermined amount of the vaccine. The pharmaceutical composition may also be an aerosol formulation for inhalation, e.g., to the bronchial passageways. Aerosol formulations may be mixed with pressurized, pharmaceutically acceptable propellants (e.g., dichlorodifluoromethane, propane, or nitrogen).

For the purposes of this invention, pharmaceutical compositions suitable for delivering a therapeutic or biologically active agent can include, e.g., tablets, gelcaps, capsules, pills, powders, granulates, suspensions, emulsions, solutions, gels, hydrogels, oral gels, pastes, eye drops, ointments, creams, plasters, drenches, delivery devices, suppositories, enemas, injectables, implants, sprays, or aerosols. Any of these formulations can be prepared by well-known and accepted methods of art. See, for example, Remington: The Science and Practice of Pharmacy (21 st ed.), ed. A. R. Gennaro, Lippincott Williams & Wilkins, 2005, and Encyclopedia of Pharmaceutical Technology, ed. J. Swarbrick, Informa Healthcare, 2006, each of which is hereby incorporated by reference.

Formulations suitable for oral administration can consist of (a) liquid solutions, such as an effective amount of the vaccine dissolved in diluents, such as water, saline or PEG 400; (b) capsules, sachets or tablets, each containing a predetermined amount of the vaccine, as liquids, solids, granules or gelatin; (c) suspensions in an appropriate liquid; (d) suitable emulsions; and (e) polysaccharide polymers such as chitins. The vaccine, alone or in combination with other suitable components, may also be made into aerosol formulations to be administered via inhalation, e.g., to the bronchial passageways. Aerosol formulations can be placed into pressurized acceptable propellants, such as dichlorodifluoromethane, propane, nitrogen, and the like.

Suitable formulations for rectal administration include, for example, suppositories, which consist of the vaccine with a suppository base. Suitable suppository bases include natural or synthetic triglycerides or paraffin hydrocarbons. In addition, it is also possible to use gelatin rectal capsules which consist of a combination of the vaccine with a base, including, for example, liquid triglycerides, polyethylene glycols, and paraffin hydrocarbons. The vaccines of the present invention may also be co-administered with cytokines to further enhance immunogenicity. The cytokines may be administered by methods known to those skilled in the art, e.g., as a nucleic acid molecule in plasmid form or as a protein or fusion protein.

In addition to the active compounds, the pharmaceutical formulations can contain other additives, such as pH-adjusting additives. In particular, useful pH-adjusting agents include acids, such as hydrochloric acid, bases or buffers, such as sodium lactate, sodium acetate, sodium phosphate, sodium citrate, sodium borate, or sodium gluconate. Further, the formulations can contain antimicrobial preservatives. Useful antimicrobial preservatives include methylparaben, propylparaben, and benzyl alcohol. An antimicrobial preservative is typically employed when the formulations is placed in a vial designed for multi-dose use. The pharmaceutical formulations described herein can be lyophilized using techniques well known in the art.

When aqueous suspensions and/or elixirs are desired for oral administration, the compositions of the presently disclosed matter can be combined with various sweetening agents, flavoring agents, coloring agents, emulsifying agents and/or suspending agents, as well as such diluents as water, ethanol, propylene glycol, glycerin and various like combinations thereof.

In yet another embodiment, the pharmaceutical composition is provided as an injectable, stable, sterile formulation comprising a rMVA as described herein, in a unit dosage form in a sealed container. The rMVA can be provided in the form of a lyophilizate, which is capable of being reconstituted with a suitable pharmaceutically acceptable carrier to form liquid formulation suitable for injection thereof into a host.

Classes of carriers include, but are not limited to binders, buffering agents, coloring agents, diluents, disintegrants, emulsifiers, flavorants, glidents, lubricants, preservatives, stabilizers, surfactants, tableting agents, and wetting agents. Some carriers may be listed in more than one class, for example vegetable oil may be used as a lubricant in some formulations and a diluent in others. Pharmaceutically acceptable carriers are carriers that do not cause any severe adverse reactions in the human body when dosed in the amount that would be used in the corresponding pharmaceutical composition. Exemplary pharmaceutically acceptable carriers include sugars, starches, celluloses, powdered tragacanth, malt, gelatin; talc, and vegetable oils. Optional active agents may be included in a pharmaceutical composition, which do not substantially interfere with the activity of the morphic form or pharmaceutical composition of the present invention.

Formulations suitable for administration to the lungs can be delivered by a wide range of passive breath driven and active power driven single/-multiple dose dry powder inhalers (DPI). The devices most commonly used for respiratory delivery include nebulizers, metered-dose inhalers, and dry powder inhalers. Several types of nebulizers are available, including jet nebulizers, ultrasonic nebulizers, and vibrating mesh nebulizers. Selection of a suitable lung delivery device depends on parameters, such as nature of the drug and its formulation, the site of action, and pathophysiology of the lung.

In certain embodiments, a pharmaceutical composition comprising a rMVA described herein is administered as a pharmaceutical composition comprising one or more excipients from the Handbook of Pharmaceutical Excipients 9^{th} Edition (or earlier).

Additional-non-limiting examples of pharmaceutically acceptable excipients include vegetable oil, an animal oil, a fish oil or a mineral oil. For example an oil selected from the group consisting of medium chain fatty acid triglyceride, amaranth oil, apricot oil, apple oil, argan oil, artichokes oil, avocado oil, almond oil, acai berry extract, arachis oil, buffalo pumpkin oil, borage seed oil, borage oil, babassu oil, coconut oil, corn oil, cottonseed oil (cotton seed oil), cashew oil, carob oil, Coriander oil, camellia oil (Camellia oil), Cauliflower oil, cape chestnut oil, cassis oil, deer oil, evening primrose oil, grape syrup Oila oil (hibiscus oil), grape seed oil, gourd oil, hazelnut oil, hemp oil, kapok oil, krill oil, linseed oil, macadamia nut oil, Mongolia oil, moringa oil, malula oil, meadowfoam oil, mustard oil, niger seed oil, olive oil, okrao oil Hibiscus oil), palm oil, palm kernel oil, peanut oil, pecan oil, pine oil, pistachio oil, pumpkin oil, papaya oil, perilla oil (perilla oil), poppy seed oil, prune oil, saw palm oil, quinoa oil, rapeseed oil, rice germ oil, rice bran oil, rice oil, rarelman cheer oil, Safflower oil (safflower oil), soybean oil, sesame oil, sunflower oil, thistle oil, tomato oil, wheat germ oil, walnut oil, watermelon oil, docosahexaenoic acid (DHA), eicosapentaenoic acid (EPA), vitamin A oil, vitamin D oil, vitamin E oil, vitamin K oil, and derivatives thereof, and glycerophospholipids such as lecithin, and any combination thereof.

In certain embodiments, the excipient in the present invention may be a liquid (such as a fat oil) or a solid (a fat or the like) at room temperature.

### Methods of Use

The compositions of the invention can be used as adjuvants to enhance, or vaccines for inducing, an immune response.

In exemplary embodiments, the present invention provides an adjuvant for use in a method of preventing an infection in a subject in need thereof (e.g., an unexposed subject), said method comprising administering the composition of the present invention to the subject in combination with an effective amount of an antigenic agent. Alternatively, the present invention provides a vaccine for use in a method of preventing an infection in a subject in need thereof (e.g., an unexposed subject), said method comprising administering the composition of the present invention to the subject. The result of the method is that the subject is partially or completely immunized against the infection.

In other exemplary embodiments, the present invention provides an adjuvant for use in a method of treating a condition such as a cancer in a subject in need thereof, said method comprising administering the composition of the present invention to the subject in combination with an effective amount of an tumor associated antigenic agent. Alternatively, the present invention provides a vaccine for use in a method of treating a condition such as a cancer in a subject in need thereof, said method comprising administering the composition of the present invention to the subject.

In exemplary embodiments, the present invention provides an adjuvant for use in a method of a treating an infectious agent (e.g., an exposed subject, such as a subject who has been recently exposed but is not yet symptomatic, or a subject who has been recently exposed and is only mildly symptomatic), said method comprising administering the composition of the present invention to the subject in combination with a therapeutically effective amount of an antigenic agent targeting the infectious agent. In exemplary embodiments, the present invention provides a vaccine for use in a method of a treating an infectious agent (e.g., an exposed subject, such as a subject who has been recently exposed but is not yet symptomatic, or a subject who has been recently exposed and is only mildly symptomatic), said method comprising administering the composition of the present invention to the subject. The result of treatment is a subject that has an improved therapeutic profile. The result is an improved therapeutic profile. In some instances, as compared with an equivalent untreated control, treatment may ameliorate a disorder or a symptom thereof by, e.g., 5%, 10%, 20%, 30%, 40%, 50%, 60%, 70%, 80%, 90%, 95%, or 100% as measured by any standard technique. In some instances, treating can result in the inhibition of infectious agent replication, a decrease in infectious agent titers or load, eradication or clearing of the infectious agent. In other embodiments, treatment may result in amelioration of one or more symptoms of the infection, including any symptom identified above. According to this embodiment, confirmation of treatment can be assessed by detecting an improvement in or the absence of symptoms.

A subject to be treated according to the methods described may be one who has been diagnosed by a medical practitioner as having such a condition. Diagnosis may be performed by any suitable means. A subject in whom the development of an infection is being prevented may or may not have received such a diagnosis. One skilled in the art will understand that a subject to be treated according to the present invention may have been identified using standard tests or may have been identified, without examination, as one at high risk due to the presence of one or more risk factors (e.g., exposure to 2019-nCoV, etc.).

In other embodiments, treatment may result in reduction or elimination of the ability of the subject to transmit the infection to another, uninfected subject. Confirmation of treatment according to this embodiment is generally assessed using the same methods used to determine amelioration of the disorder, but the reduction in viral titer or viral load necessary to prevent transmission may differ from the reduction in viral titer or viral load necessary to ameliorate the disorder.

In one embodiment, the present invention is a method of inducing an immune response in a subject (e.g., a human) by administering to the subject a recombinant MVA viral vector described herein encoding two or more immune checkpoint inhibitor peptides in combination with an antigenic agent. The immune response may be a cellular immune response or a humoral immune response, or a combination thereof.

The composition may be administered, e.g., by injection (e.g., intramuscular, intraarterial, intravascular, intravenous, intraperitoneal, or subcutaneous).

It will be appreciated that more than one route of administering the vaccines of the present invention may be employed either simultaneously or sequentially (e.g., boosting). In addition, the adjuvants or vaccines of the present invention may be employed in combination with traditional immunization approaches such as employing protein antigens, vaccinia virus and inactivated virus, as vaccines. Thus, in one embodiment, the vaccines of the present invention are administered to a subject (the subject is “primed” with a vaccine of the present invention) and then a traditional vaccine is administered (the subject is “boosted” with a traditional vaccine). In another embodiment, a traditional vaccine is first administered to the subject followed by administration of the adjuvant or vaccine of the present invention. In yet another embodiment, a traditional vaccine and an adjuvant or vaccine of the present invention are co-administered.

While not to be bound by any specific mechanism, it is believed that upon inoculation with a pharmaceutical composition as described herein, the immune system of the host responds to the adjuvant in combination with an antigenic agent, or vaccine by producing antibodies, both secretory and serum, specific for the infectious agent or tumor associated antigen; and by producing a cell-mediated immune response specific for the targeted agent. As a result of the vaccination, the host becomes at least partially or completely immune to the targeted infection, or resistant to developing moderate or severe disease caused by the targeted infection.

In some embodiments, administration is one time. In some embodiments, administration is repeated at least twice, at least 3 times, at least 4 times, at least 5 times, at least 6 times, at least 7 times, at least 8 times, or more than 8 times.

In one embodiment, administration is repeated twice.

In one embodiment, about 2-8, about 4-8, or about 6-8 administrations are provided.

In one embodiment, about 1-4-week, 2-4 week, 3-4 week, 1 week, 2 week, 3 week, 4 week or more than 4 week intervals are provided between administrations.

In one specific embodiment, a 4-week interval is used between 2 administrations.

### Dosage

The adjuvants in combination with an antigenic agent or vaccines are administered in a manner compatible with the dosage formulation, and in such amount as will be therapeutically effective, immunogenic and protective. The quantity to be administered depends on the subject to be treated, including, for example, the capacity of the immune system of the individual to synthesize antibodies, and, if needed, to produce a cell-mediated immune response. Precise amounts of active ingredient required to be administered depend on the judgment of the practitioner and may be monitored on a patient-by-patient basis. However, suitable dosage ranges are readily determinable by one skilled in the art and generally range from about 5.0 χ 10⁶ TCID₅₀ to about 5.0 χ 10⁹ TCID₅₀. The dosage may also depend, without limitation, on the route of administration, the patient's state of health and weight, and the nature of the formulation.

The pharmaceutical compositions of the invention are administered in such an amount as will be therapeutically effective to enhance the immunogenicity of a targeted antigen. The dosage administered depends on the subject to be treated (e.g., the manner of administration and the age, body weight, capacity of the immune system, and general health of the subject being treated). The composition is administered in an amount to provide a sufficient level of expression that enhances or elicits an immune response without undue adverse physiological effects. Preferably, the composition of the invention is administered at a dosage of, e.g., between 1.0 χ 104 and 9.9 χ 10¹² TCID₅₀ of the viral vector, preferably between 1.0 χ 10⁵ TCID₅₀ and 1.0 χ 10¹¹ TCID₅₀ pfu, more preferably between 1.0 χ 10⁶ and 1.0 χ 1010 TCID₅₀ pfu, or most preferably between 5.0 χ 10⁶ and 5.0 χ 10⁹ TCID₅₀. The composition may include, e.g., at least 5.0 χ 10⁶ TCID₅₀ of the viral vector (e.g., 1.0 χ 10⁸ TCID₅₀ of the viral vector). A physician or researcher can decide the appropriate amount and dosage regimen.

The composition of the method may include, e.g., between 1.0 χ 10⁴ and 9.9 χ 10¹² TCID₅₀ of the viral vector, preferably between 1.0 χ 10⁵ TCID₅₀ and 1.0 χ 10¹¹ TCID₅₀ pfu, more preferably between 1.0 χ 10⁶ and 1.0 χ 1010 TCID₅₀ pfu, or most preferably between 5.0 χ 10⁶ and 5.0 χ 109 TCID₅₀. The composition may include, e.g., at least 5.0 χ 10⁶ TCID₅₀ of the viral vector (e.g., 1.0 χ 108 TCID₅₀ of the viral vector). The method may include, e.g., administering the composition to the subject two or more times.

The term “effective amount” is meant the amount of a composition administered to improve, inhibit, or ameliorate a condition of a subject, or a symptom of a disorder, in a clinically relevant manner (e.g., improve, inhibit, or ameliorate infection by arenavirus or provide an effective immune response to infection). Any improvement in the subject is considered sufficient to achieve treatment. Preferably, an amount sufficient to treat is an amount that prevents the occurrence or one or more symptoms of, or is an amount that reduces the severity of, or the length of time during which a subject suffers from, one or more symptoms of a targeted infection or cancer (e.g., by at least 10%, 20%, or 30%, more preferably by at least 50%, 60%, or 70%, and most preferably by at least 80%, 90%, 95%, 99%, or more, relative to a control subject that is not treated with a composition of the invention).

In some instances, it may be desirable to combine the rMVA of the present invention with immunogenic compositions which induce protective responses to more than one infectious agents, particularly other viruses. For example, the adjuvant compositions of the present invention can be administered simultaneously, separately or sequentially with other genetic immunization vaccines such as those for influenza (Ulmer, J. B. et al., Science 259: 1745-1749 (1993); Raz, E. et al., PNAS (USA) 91:9519-9523 (1994)), malaria (Doolan, D. L. et al., J. Exp. Med. 183:1739-1746 (1996); Sedegah, M. et al., PNAS (USA) 91:9866-9870 (1994)), and tuberculosis (Tascon, R. C. et al., Nat. Med. 2:888-892 (1996)).

### Administration

As used herein, the term “administering” refers to a method of giving a dosage of a pharmaceutical composition of the invention to a subject. The compositions utilized in the methods described herein can be administered by a route selected from, e.g., parenteral, dermal, transdermal, ocular, inhalation, buccal, sublingual, perilingual, nasal, rectal, topical administration, and oral administration. Parenteral administration includes intravenous, intraperitoneal, subcutaneous, intraarterial, intravascular, and intramuscular administration. The preferred method of administration can vary depending on various factors (e.g., the components of the composition being administered, and the severity of the condition being treated).

Administration of the pharmaceutical compositions (e.g., adjuvant or vaccines) of the present invention can be by any of the routes known to one of skill in the art. Administration may be by, e.g., intramuscular injection. The compositions utilized in the methods described herein can also be administered by a route selected from, e.g., parenteral, dermal, transdermal, ocular, inhalation, buccal, sublingual, perilingual, nasal, rectal, topical administration, and oral administration. Parenteral administration includes intravenous, intraperitoneal, subcutaneous, and intramuscular administration. The preferred method of administration can vary depending on various factors, e.g., the components of the composition being administered, and the severity of the condition being treated.

In addition, single or multiple administrations of the compositions of the present invention may be given to a subject. For example, subjects who are particularly susceptible to the targeted antigenic agent may require multiple treatments to establish and/or maintain protection against the virus. Levels of induced immunity provided by the pharmaceutical compositions described herein can be monitored by, e.g., measuring amounts of neutralizing secretory and serum antibodies. The dosages may then be adjusted or repeated as necessary to maintain desired levels of protection against viral infection.

### EMBODIMENTS

Provided herein are at least the following embodiments:
1. A recombinant modified vaccinia Ankara (rMVA) viral vector comprising a heterologous, polycistronic nucleic acid, wherein the polycistronic nucleic acid encodes (M)(Secretion Signal Peptide-Immune Checkpoint Inhibitor Peptide-Cleavable Peptide)ₓ, wherein x=2-10, and M is methionine.
2. An rMVA viral vector comprising a heterologous, polycistronic nucleic acid, wherein the polycistronic nucleic acid encodes ((M)(Secretion Signal Peptide-Immune Checkpoint Inhibitor Peptide-Cleavable Peptide)ₓ(Secretion Signal Peptide-Immune Checkpoint Inhibitor Peptide)), wherein x=1-10, and M is methionine.
3. The rMVA of embodiments 1 or 2, wherein the immune checkpoint inhibitor peptide comprises an amino acid sequence selected from SEQ ID NOS. 1-56, or an amino acid sequence at least 95% identical thereto.
4. The rMVA of embodiments 1-3, wherein the immune checkpoint inhibitor peptide comprises an amino acid sequence selected from SEQ ID NOS: 1-15, or an amino acid sequence at least 95% identical thereto.
5. The rMVA of embodiments 1-4, wherein the immune checkpoint inhibitor peptide comprises an amino acid sequence selected from SEQ ID NOS: 1 or 5, or an amino acid sequence at least 95% identical thereto.
6. The rMVA of embodiments 1-5, wherein the immune checkpoint inhibitor peptide comprises the amino acid sequence of SEQ ID NO: 1, or an amino acid sequence at least 95% identical thereto.
7. The rMVA of embodiments 1-5, wherein the immune checkpoint inhibitor peptide comprises the amino acid sequence of SEQ ID NO: 5, or an amino acid sequence at least 95% identical thereto.
8. The rMVA of embodiments 1-7, wherein the secretion signal peptide comprises an amino acid sequence selected from SEQ ID NOS: 57-90, or an amino acid sequence at least 95% identical thereto.
9. The rMVA of embodiments 1-8, wherein the secretion signal peptide comprises an amino acid sequence selected from SEQ ID NO: 65, or an amino acid sequence at least 95% identical thereto.
10. The rMVA of embodiments 1-8, wherein the secretion signal peptide comprises an amino acid sequence selected from SEQ ID NO: 66, or an amino acid sequence at least 95% identical thereto.
11. The rMVA of embodiments 1-10, wherein the cleavable peptide comprises an amino acid sequence selected from SEQ ID NOS: 91-127, or an amino acid sequence at least 95% identical thereto.
12. The rMVA of embodiments 1-11, wherein the cleavable peptide comprises an amino acid sequence selected from SEQ ID NOS: 93, 120, and 123, or an amino acid sequence at least 95% identical thereto.
13. The rMVA of embodiments 1-11, wherein the cleavable peptide comprises an amino acid sequence RX(R/K)R, wherein X=any amino acid (SEQ ID NO: 91).
14. The rMVA of embodiments 1-11, wherein the cleavable peptide comprises an amino acid sequence RX(R/K)R, wherein X=R, K, or H (SEQ ID NO: 92).
15. The rMVA of embodiments 1-12, wherein the cleavable peptide is RAKR (SEQ ID NO: 93).
16. The rMVA of embodiments 1-11, wherein the cleavable peptide is RRRR (SEQ ID NO: 94).
17. The rMVA of embodiments 1-11, wherein the cleavable peptide is RKRR (SEQ ID NO: 95).
18. The rMVA of embodiments 1-11, wherein the cleavable peptide is RRKR (SEQ ID NO: 96).
19. The rMVA of embodiments 1-11, wherein the cleavable peptide is RKKR (SEQ ID NO: 97).
20. The rMVA of embodiments 1-11, wherein the cleavable peptide is an amino acid sequence of SEQ ID NOS: 123-127, or an amino acid sequence at least 95% identical thereto.
21. The rMVA of embodiments 1-12, wherein the cleavable peptide is the amino acid of SEQ ID NOS: 123, or an amino acid sequence at least 95% identical thereto.
22. The rMVA of embodiments 1-2, wherein the polycistronic nucleic acid encodes an amino acid sequence selected from SEQ ID NOS: 309-324, or an amino acid sequence at least 95% identical thereto.
23. The rMVA of embodiments 1-22, wherein x≥4.
24. The rMVA of embodiments 1-22, wherein x=3, 4, or 5.
25. The rMVA of embodiments 1-2, wherein the polycistronic nucleic acid encodes an amino acid sequence selected from SEQ ID NOS: 325-340, or an amino acid sequence at least 95% identical thereto.
26. The rMVA of embodiments 1-2, wherein the polycistronic nucleic acid encodes an amino acid sequence selected from SEQ ID NOS: 341-344, or an amino acid sequence at least 95% identical thereto.
27. The rMVA of embodiments 1-2, wherein the polycistronic nucleic acid encodes an amino acid sequence selected from SEQ ID NOS: 345-348, or an amino acid sequence at least 95% identical thereto.
28. The rMVA of embodiments 1-2, wherein the polycistronic nucleic acid encodes the amino acid sequence of SEQ ID NO: 325, or an amino acid sequence at least 95% identical thereto.
29. The rMVA of embodiments 1-2, wherein the polycistronic nucleic acid encodes the amino acid sequence of SEQ ID NO: 329, or an amino acid sequence at least 95% identical thereto.
30. The rMVA of embodiments 1-2, wherein the polycistronic nucleic acid encodes the amino acid sequence of SEQ ID NO: 333, or an amino acid sequence at least 95% identical thereto.
31. The rMVA of embodiments 1-2, wherein the polycistronic nucleic acid encodes the amino acid sequence of SEQ ID NO: 337, or an amino acid sequence at least 95% identical thereto.
32. The rMVA of embodiments 1-31, wherein the polycistronic nucleic acid further encodes an antigenic peptide.
33. The rMVA of embodiment 32, wherein the antigenic peptide is derived from the group consisting of an infectious agent and tumor associated antigen.
34. The rMVA of embodiment 33, wherein the infectious agent is a virus, bacterium, fungi, parasite, or amoeba.
35. The rMVA of embodiment 34, wherein the virus is selected from the group consisting of Adenovirus; Herpesvirus; a Poxvirus; a single stranded DNA; a Parvovirus; a double stranded RNA virus; Reovirus; a positive-single stranded RNA virus; Coronavirus; Picornavirus; Togavirus; a negative-single stranded RNA virus; a Orthomyxovirus; a Rhabdovirus; a single-stranded RNA-Retrovirus; a double-stranded DNA-Retrovirus; a Flaviviridae virus; Alphavirus virus, Filoviridae virus; a Paramyxoviridae virus; Rhabdoviridae virus; a Nyamiviridae virus; an Arenaviridae virus; a Bunyaviridae virus; or Ophioviridae virus; and Orthomyxoviridae virus.
36. The rMVA of embodiment 32, wherein the antigenic peptide is derived from the Ebola virus, the envelope glycoprotein of Ebola virus, the matrix protein VP40 of Ebola virus; the Lassa virus, Lassa virus protein Z; the Zika virus, Zika virus non-structural protein 1 (NSP-1); the Marburg virus; the Marburg virus glycoprotein; the Marburg VP40 matrix protein; the Plasmodium sp. parasite; Plasmodium falciparum; Plasmodium sp. circumsporozoite protein (CSP); Plasmodium sp. male gametocyte surface protein P230p (Pfs230 antigen); Plasmodium sp. sporozoite micronemal protein essential for cell traversal (SPECT2); Plasmodium sp. GTP-binding protein; putative antigen; the human immunodeficiency virus; HIV Env protein; HIV gp41; HIV gp120; HIV gp160; HIV Gag protein; HIV MA; HIV CA; HIV SP1; HIV NC; HIV SP2; HIV P6; HIV Pol protein; HIV RT; HIV RNase H; HIV IN; and HIV PR; or fragment thereof.
37. The rMVA of embodiment 32, wherein the antigenic peptide is derived from the group consisting of the SARS-CoV2; the SARS-CoV2 full-length S protein Wuhan Strain, the SARS-CoV2 S protein with K417T, E484K, and N501Y substitutions; the SARS-CoV2 full-length S protein Delta variant; the SARS-CoV2 full-length S protein Delta variant plus; the SARS-CoV2 full-length S protein stabilized by 2 proline substitutions; the SARS-CoV2 full-length stabilized S protein; the SARS-CoV2 full-length stabilized S protein with K417T, E484K, and N501Y substitutions; the SARS-CoV2 full-length stabilized S protein Delta variant; the SARS-CoV2 full-length stabilized S protein Delta variant plus; the SARS-CoV2 E protein; the SARS-CoV2 M protein; the SARS-CoV2 PP1ab polyprotein amino acid sequence; the SARS-CoV2 PP1a polyprotein amino acid sequence (Wuhan Hu1); the SARS-CoV2 NSP1-3 amino acid sequence (Wuhan Hu1); the SARS-CoV2 NSP4-11 amino acid sequence (Wuhan Hu1); the SARS-CoV2 ORF1b polyprotein NSP12-16 amino acid sequence (Wuhan Hu1); the SARS-CoV2 NSP12 amino acid sequence (Wuhan Hu1); the SARS-CoV2 NSP13-14 amino acid sequence (Wuhan Hu1); the SARS-CoV2 NSP15-16 amino acid sequence (Wuhan Hu1); the MUC-1 MARV GPTM amino acid sequence; the Marburg virus VP40 amino acid sequence; and the MUC-1-ECD-MARVTM-ICD sequence; or fragment thereof.
38. The rMVA of embodiment 33, wherein the tumor associated antigen is derived from an oncofetal tumor associate antigen, an oncoviral tumor associate antigen, overexpressed/accumulated tumor associate antigen, cancer-testis tumor associate antigen, lineage-restricted tumor associate antigen, mutated tumor associate antigen, or idiotypic tumor associate antigen, or fragment thereof.
39. The rMVA of embodiment 33, wherein the tumor associated antigen is derived from the b melanoma antigen (BAGE) family, cancer-associated gene (CAGE) family, G antigen (GAGE) family, melanoma antigen (MAGE) family, sarcoma antigen (SAGE) family and X antigen (XAGE) family, CT9, CT10, NY-ESO-1, L antigen (LAGE) 1, Melanoma antigen preferentially expressed in tumors (PRAME), and synovial sarcoma X (SSX) 2, melanoma antigen recognized by T cells-1/2 (Melan-A/MART-1/2), Gp100/pmel17, tyrosine-related protein (TRP) 1 and 2, P. polypeptide, melanocortin 1 receptor (MC1R), and prostate-specific antigen, β-catenin, breast cancer antigen (BRCA) 1/2, cyclin-dependent kinase (CDK) 4, chronic myelogenous leukemia antigen (CML) 66, fibronectin, p53, Ras, or TGF-βRII, or fragment thereof.
40. The rMVA of embodiment 32, wherein the antigenic peptide is derived from mucin 1, or fragment thereof.
41. The rMVA of embodiment 40, wherein the mucin 1 is encoded by the nucleic acid sequence of SEQ ID NO: 402, or a nucleic acid sequence at least 95% identical thereto.
42. The method of embodiment 40, wherein the mucin 1 comprises the amino acid sequence of SEQ ID NO: 349, or an amino acid sequence at least 95% identical thereto.
43. The rMVA of embodiment 40, wherein the mucin 1 comprises the amino acid sequence of SEQ ID NO: 403, or an amino acid sequence at least 95% identical thereto.
44. The rMVA of embodiment 40, wherein the mucin 1 comprises an extracellular domain fragment of human mucin 1.
45. The rMVA of embodiment 44, wherein the extracellular domain fragment of human mucin 1 is selected from SEQ ID NO: 358-361, or an amino acid sequence at least 95% identical thereto.
46. The rMVA of embodiment 40, wherein the mucin 1 comprises an intracellular domain fragment of human mucin 1.
47. The rMVA of embodiment 46, wherein the intracellular domain fragment of human mucin 1 comprises the amino acid sequence of SEQ ID NO: 362, or an amino acid sequence at least 95% identical thereto.
48. The method of embodiment 40, wherein the mucin 1 is selected from SEQ ID NO: 363-364, or an amino acid sequence at least 95% identical thereto.
49. The method of embodiment 48, wherein the mucin 1 comprises the amino acid sequence of SEQ ID NO: 363, or an amino acid sequence at least 95% identical thereto.
50. The method of embodiment 48, wherein the mucin 1 comprises the amino acid sequence of SEQ ID NO: 364, or an amino acid sequence at least 95% identical thereto.
51. The rMVA of embodiment 32, wherein the antigenic peptide is derived from an amino acid sequence selected from SEQ ID NOS: 349-357, or an amino acid sequence at least 95% identical thereto.
52. The rMVA of embodiment 32, wherein the antigenic peptide is derived from an amino acid sequence selected from SEQ ID NOS: 358-394, or an amino acid sequence at least 95% identical thereto.
53. The rMVA of embodiments 51-52, wherein the antigenic peptide is derived from an amino acid sequence selected from SEQ ID NOS: 350, 354, 356, 365, 366, 367, 368, 369, 377, 379, or an amino acid sequence at least 95% identical thereto.
54. The rMVA of embodiments 32-53, wherein the antigenic peptide includes a secretion signal.
55. The rMVA of embodiment 54, wherein the secretion signal is fused to the N-terminus of the antigenic peptide.
56. The rMVA of embodiment 55, wherein the secretion signal is selected from an amino acid sequence of SEQ ID NOS: 57-90, or an amino acid sequence at least 95% identical thereto.
57. The rMVA of embodiment 56, wherein the secretion signal comprises the amino acid sequence of SEQ ID NO. 65, or an amino acid sequence at least 95% identical thereto.
58. The rMVA of embodiment 56, wherein the secretion signal comprises the amino acid sequence of SEQ ID NO. 66, or an amino acid sequence at least 95% identical thereto.
59. The rMVA of embodiments 1-58, wherein the polycistronic nucleic acid is inserted between two essential and highly conserved MVA genes.
60. The rMVA of embodiments 1-58, wherein the polycistronic nucleic acid is inserted into a natural deletion site.
61. The rMVA of embodiments 1-58, wherein the polycistronic nucleic acid is inserted into the MVA at a site selected from between MVA genes I8R and G1L, between MVA genes A50R and B1R in a restructured and modified deletion site III, or between MVA genes A5 and A6L.
62. The rMVA of embodiments 1-58, wherein the polycistronic nucleic acid is inserted into the rMVA at a site selected from between MVA genes I8R and G1L.
63. The rMVA of embodiments 1-58, wherein the polycistronic nucleic acid is inserted into the rMVA at a site selected from between MVA genes A50R and B1R in a restructured and modified deletion site III.
64. The rMVA of embodiments 1-58, wherein the polycistronic nucleic acid is inserted into the rMVA at a site selected from between MVA genes A5 and A6L.
65. The rMVA of embodiments 32-64, wherein the nucleic acid encoding the antigenic peptide amino acid sequence is in an open reading frame downstream of a Methionine (M) start codon.
66. A method of increasing an immune response to a target antigen in a patient comprising administering to the patient an effective amount of an rMVA viral vector of embodiments 1-65, wherein the patient has been or is being administered an effective amount of the target antigen.
67. The method of embodiment 66, wherein the rMVA viral vector is administered concomitantly with or subsequent to the administration of the target antigen.
68. The method of embodiments 66-67, wherein the target antigen is selected from the group consisting of an infectious agent and tumor associated antigen.
69. The method of embodiment 68, wherein the infectious agent is a virus, bacterium, fungi, parasite, or amoeba.
70. The method of embodiment 69, wherein the virus is selected from the group consisting of Adenovirus; Herpesvirus; a Poxvirus; a single stranded DNA; a Parvovirus; a double stranded RNA virus; Reovirus; a positive-single stranded RNA virus; Coronavirus; Picornavirus; Togavirus; a negative-single stranded RNA virus; a Orthomyxovirus; a Rhabdovirus; a single-stranded RNA-Retrovirus; a double-stranded DNA-Retrovirus; a Flaviviridae virus; Alphavirus virus, Filoviridae virus; a Paramyxoviridae virus; Rhabdoviridae virus; a Nyamiviridae virus; an Arenaviridae virus; a Bunyaviridae virus; or Ophioviridae virus; and Orthomyxoviridae virus.
71. The method of embodiments 66-67, wherein the target antigen is derived from the Ebola virus, the envelope glycoprotein of Ebola virus, the matrix protein VP40 of Ebola virus; the Lassa virus, Lassa virus protein Z; the Zika virus, Zika virus non-structural protein 1 (NSP-1); the Marburg virus; the Marburg virus glycoprotein; the Marburg VP40 matrix protein; the Plasmodium sp. parasite; Plasmodium falciparum; Plasmodium sp. circumsporozoite protein (CSP); Plasmodium sp. male gametocyte surface protein P230p (Pfs230 antigen); Plasmodium sp. sporozoite micronemal protein essential for cell traversal (SPECT2); Plasmodium sp. GTP-binding protein; putative antigen; the human immunodeficiency virus; HIV Env protein; HIV gp41; HIV gp120; HIV gp160; HIV Gag protein; HIV MA; HIV CA; HIV SP1; HIV NC; HIV SP2; HIV P6; HIV Pol protein; HIV RT; HIV RNase H; HIV IN; and HIV PR, or fragment thereof.
72. The method of embodiments 66-67, wherein the target antigen is derived from the group consisting of the SARS-CoV2; the SARS-CoV2 full-length S protein Wuhan Strain, the SARS-CoV2 S protein with K417T, E484K, and N501Y substitutions; the SARS-CoV2 full-length S protein Delta variant; the SARS-CoV2 full-length S protein Delta variant plus; the SARS-CoV2 full-length S protein stabilized by 2 proline substitutions; the SARS-CoV2 full-length stabilized S protein; the SARS-CoV2 full-length stabilized S protein with K417T, E484K, and N501Y substitutions; the SARS-CoV2 full-length stabilized S protein Delta variant; the SARS-CoV2 full-length stabilized S protein Delta variant plus; the SARS-CoV2 E protein; the SARS-CoV2 M protein; the SARS-CoV2 PP1ab polyprotein amino acid sequence; the SARS-CoV2 PP1a polyprotein amino acid sequence (Wuhan Hu1); the SARS-CoV2 NSP1-3 amino acid sequence (Wuhan Hu1); the SARS-CoV2 NSP4-11 amino acid sequence (Wuhan Hu1); the SARS-CoV2 ORF1b polyprotein NSP12-16 amino acid sequence (Wuhan Hu1); the SARS-CoV2 NSP12 amino acid sequence (Wuhan Hu1); the SARS-CoV2 NSP13-14 amino acid sequence (Wuhan Hu1); and the SARS-CoV2 NSP15-16 amino acid sequence (Wuhan Hu1); or fragment thereof.
73. The method of embodiment 68, wherein the tumor associated antigen is derived from an oncofetal tumor associate antigen, an oncoviral tumor associate antigen, overexpressed/accumulated tumor associate antigen, cancer-testis tumor associate antigen, lineage-restricted tumor associate antigen, mutated tumor associate antigen, or idiotypic tumor associate antigen, or fragment thereof.
74. The method of embodiment 68, wherein the tumor associated antigen is derived from the b melanoma antigen (BAGE) family, cancer-associated gene (CAGE) family, G antigen (GAGE) family, melanoma antigen (MAGE) family, sarcoma antigen (SAGE) family and X antigen (XAGE) family, CT9, CT10, NY-ESO-1, L antigen (LAGE) 1, Melanoma antigen preferentially expressed in tumors (PRAME), and synovial sarcoma X (SSX) 2, melanoma antigen recognized by T cells-1/2 (Melan-A/MART-1/2), Gp100/pmel17, tyrosine-related protein (TRP) 1 and 2, P. polypeptide, melanocortin 1 receptor (MC1R), and prostate-specific antigen, β-catenin, breast cancer antigen (BRCA) 1/2, cyclin-dependent kinase (CDK) 4, chronic myelogenous leukemia antigen (CML) 66, fibronectin, p53, Ras, or TGF-βRII, or fragment thereof.
75. The method of embodiments 66-67, wherein the target antigen is derived from mucin 1, or fragment thereof.
76. The method of embodiment 75, wherein the mucin 1 is encoded by the nucleic acid sequence of SEQ ID NO: 402, or a nucleic acid sequence at least 95% identical thereto.
77. The method of embodiment 75, wherein the mucin 1 comprises the amino acid sequence of SEQ ID NO: 349, or an amino acid sequence at least 95% identical thereto.
78. The method of embodiment 75, wherein the mucin 1 comprises the amino acid sequence of SEQ ID NO: 403, or an amino acid sequence at least 95% identical thereto.
79. The method of embodiment 75, wherein the mucin 1 comprises an extracellular domain fragment of human mucin 1.
80. The method of embodiment 79, wherein the extracellular domain fragment of human mucin 1 is selected from SEQ ID NO: 358-361, or an amino acid sequence at least 95% identical thereto.
81. The method of embodiment 75, wherein the mucin 1 comprises an intracellular domain fragment of human mucin 1.
82. The method of embodiment 81, wherein the intracellular domain fragment of human mucin 1 comprises the amino acid sequence of SEQ ID NO: 362, or an amino acid sequence at least 95% identical thereto.
83. The method of embodiment 75, wherein the mucin 1 is selected from SEQ ID NO: 363-364, or an amino acid sequence at least 95% identical thereto.
84. The method of embodiment 83, wherein the mucin 1 comprises the amino acid sequence of SEQ ID NO: 363, or an amino acid sequence at least 95% identical thereto.
85. The method of embodiment 83, wherein the mucin 1 comprises the amino acid sequence of SEQ ID NO: 364, or an amino acid sequence at least 95% identical thereto.
86. The method of embodiments 66-67, wherein the target antigen is derived from an amino acid sequence selected from SEQ ID NOS: 349-357, or an amino acid sequence at least 95% identical thereto.
87. The method of embodiments 66-67, wherein the target antigen is derived from an amino acid sequence selected from SEQ ID NOS: 358-394, or an amino acid sequence at least 95% identical thereto.
88. The method of embodiments 66-67, wherein the target antigen is derived from an amino acid sequence selected from SEQ ID NOS: 350, 354, 356, 365, 366, 367, 368, 369, 377, 379, or an amino acid sequence at least 95% identical thereto.
89. An rMVA viral vector comprising a heterologous, polycistronic nucleic acid, wherein the polycistronic nucleic acid encodes (M)(Secretion Signal Peptide-Immune Checkpoint Inhibitor Peptide-Cleavable Peptide)ₓ(Secretion Signal Peptide-Antigenic Peptide), wherein x=1-10, and M is methionine.
90. An rMVA viral vector comprising a heterologous, polycistronic nucleic acid, wherein the polycistronic nucleic acid encodes (M)(Secretion Signal Peptide-Immune Checkpoint Inhibitor Peptide-Cleavable Peptide)ₓ(Glycoprotein Signal Peptide-Antigenic Peptide-Glycoprotein Transmembrane Peptide), wherein x=1-10, and M is methionine.
91. An rMVA viral vector comprising a heterologous, polycistronic nucleic acid, wherein the polycistronic nucleic acid encodes (M)(Secretion Signal Peptide-Immune Checkpoint Inhibitor Peptide-Cleavable Peptide)ₓ(Glycoprotein Signal Peptide-Antigenic Peptide-Glycoprotein Transmembrane Peptide-Cleavable Peptide)(Viral Matrix Protein), wherein x=1-10, and M is methionine.
92. A recombinant modified vaccinia Ankara (rMVA) viral vector comprising a heterologous polycistronic nucleic acid insert encoding a polypeptide wherein the polypeptide comprises ((M)(Secretion Signal Peptide-Immune Checkpoint Inhibitor Peptide-Cleavable Peptide)ₓ(Antigenic Peptide)), wherein x=1-10, and M is methionine.
93. The rMVA of embodiments 89-92, wherein the immune checkpoint inhibitor peptide comprises an amino acid sequence selected from SEQ ID NOS. 1-56, or an amino acid sequence at least 95% identical thereto.
94. The rMVA of embodiments 89-93, wherein the immune checkpoint inhibitor peptide comprises an amino acid sequence selected from SEQ ID NOS. 1-15, or an amino acid sequence at least 95% identical thereto.
95. The rMVA of embodiments 89-94, wherein the immune checkpoint inhibitor peptide comprises an amino acid sequence selected from SEQ ID NOS. 1 or 5, or an amino acid sequence at least 95% identical thereto.
96. The rMVA of embodiments 89-95, wherein the immune checkpoint inhibitor peptide comprises the amino acid sequence of SEQ ID NO. 1, or an amino acid sequence at least 95% identical thereto.
97. The rMVA of embodiments 89-95, wherein the immune checkpoint inhibitor peptide comprises the amino acid sequence of SEQ ID NO. 5, or an amino acid sequence at least 95% identical thereto.
98. The rMVA of embodiments 89-97, wherein the secretion signal peptide comprises an amino acid sequence selected from SEQ ID NOS. 57-90, or an amino acid sequence at least 95% identical thereto.
99. The rMVA of embodiments 89-98, wherein the secretion signal peptide comprises the amino acid sequence of SEQ ID NO. 65, or an amino acid sequence at least 95% identical thereto.
100. The rMVA of embodiments 89-98, wherein the secretion signal peptide comprises the amino acid sequence of SEQ ID NO. 66, or an amino acid sequence at least 95% identical thereto.
101. The rMVA of embodiments 89-100 wherein the cleavable peptide comprises an amino acid sequence selected from SEQ ID NOS. 91-126, or an amino acid sequence at least 95% identical thereto.
102. The rMVA of embodiments 89-101, wherein the cleavable peptide comprises an amino acid sequence selected from SEQ ID NOS. 93, 120, and 123.
103. The rMVA of embodiments 89-101, wherein the cleavable peptide comprises an amino acid sequence RX(R/K)R, wherein X=any amino acid (SEQ ID NO: 91).
104. The rMVA of embodiments 89-101, wherein the cleavable peptide comprises an amino acid sequence RX(R/K)R, wherein X=R, K, or H (SEQ ID NO: 92).
105. The rMVA of embodiments 89-102, wherein the cleavable peptide is RAKR (SEQ ID NO: 93).
106. The rMVA of embodiments 89-101, wherein the cleavable peptide is RRRR (SEQ ID NO: 94).
107. The rMVA of embodiments 89-101, wherein the cleavable peptide is RKRR (SEQ ID NO: 95).
108. The rMVA of embodiments 89-101, wherein the cleavable peptide is RRKR (SEQ ID NO: 96).
109. The rMVA of embodiments 89-101, wherein the cleavable peptide is RKKR (SEQ ID NO: 97).
110. The rMVA of embodiments 89-101, wherein the cleavable peptide comprises an amino acid sequence selected from SEQ ID NOS. 123-127, or an amino acid sequence at least 95% identical thereto.
111. The rMVA of embodiments 89-102, wherein the cleavable peptide comprises the amino acid sequence of SEQ ID NO. 123, or an amino acid sequence at least 95% identical thereto.
112. The rMVA of embodiments 89-111, wherein the antigenic peptide is derived from the group consisting of an infectious agent and tumor associated antigen.
113. The rMVA of embodiment 112, wherein the infectious agent is a virus, bacterium, fungi, parasite, or amoeba.
114. The rMVA of embodiment 113, wherein the virus is selected from the group consisting of Adenovirus; Herpesvirus; a Poxvirus; a single stranded DNA; a Parvovirus; a double stranded RNA virus; Reovirus; a positive-single stranded RNA virus; Coronavirus; Picornavirus; Togavirus; a negative-single stranded RNA virus; a Orthomyxovirus; a Rhabdovirus; a single-stranded RNA-Retrovirus; a double-stranded DNA-Retrovirus; a Flaviviridae virus; Alphavirus virus, Filoviridae virus; a Paramyxoviridae virus; Rhabdoviridae virus; a Nyamiviridae virus; an Arenaviridae virus; a Bunyaviridae virus; or Ophioviridae virus; and Orthomyxoviridae virus.
115. The rMVA of embodiments 89-111, wherein the antigenic peptide is derived from the Ebola virus, the envelope glycoprotein of Ebola virus, the matrix protein VP40 of Ebola virus; the Lassa virus, Lassa virus protein Z; the Zika virus, Zika virus non-structural protein 1 (NSP-1); the Marburg virus; the Marburg virus glycoprotein; the Marburg VP40 matrix protein; the Plasmodium sp. parasite; Plasmodium falciparum; Plasmodium sp. circumsporozoite protein (CSP); Plasmodium sp. male gametocyte surface protein P230p (Pfs230 antigen); Plasmodium sp. sporozoite micronemal protein essential for cell traversal (SPECT2); Plasmodium sp. GTP-binding protein; putative antigen; the human immunodeficiency virus; HIV Env protein; HIV gp41; HIV gp120; HIV gp160; HIV Gag protein; HIV MA; HIV CA; HIV SP1; HIV NC; HIV SP2; HIV P6; HIV Pol protein; HIV RT; HIV RNase H; HIV IN; and HIV PR; or fragment thereof.
116. The rMVA of embodiments 89-111, wherein the antigenic peptide is derived from the group consisting of the SARS-CoV2; the SARS-CoV2 full-length S protein Wuhan Strain, the SARS-CoV2 S protein with K417T, E484K, and N501Y substitutions; the SARS-CoV2 full-length S protein Delta variant; the SARS-CoV2 full-length S protein Delta variant plus; the SARS-CoV2 full-length S protein stabilized by 2 proline substitutions; the SARS-CoV2 full-length stabilized S protein; the SARS-CoV2 full-length stabilized S protein with K417T, E484K, and N501Y substitutions; the SARS-CoV2 full-length stabilized S protein Delta variant; the SARS-CoV2 full-length stabilized S protein Delta variant plus; the SARS-CoV2 E protein; the SARS-CoV2 M protein; the SARS-CoV2 PP1ab polyprotein amino acid sequence; the SARS-CoV2 PP1a polyprotein amino acid sequence (Wuhan Hu1); the SARS-CoV2 NSP1-3 amino acid sequence (Wuhan Hu1); the SARS-CoV2 NSP4-11 amino acid sequence (Wuhan Hu1); the SARS-CoV2 ORF1b polyprotein NSP12-16 amino acid sequence (Wuhan Hu1); the SARS-CoV2 NSP12 amino acid sequence (Wuhan Hu1); the SARS-CoV2 NSP13-14 amino acid sequence (Wuhan Hu1); and the SARS-CoV2 NSP15-16 amino acid sequence (Wuhan Hu1); or fragment thereof.
117. The rMVA of embodiment 112, wherein the tumor associated antigen is derived from an oncofetal tumor associate antigen, an oncoviral tumor associate antigen, overexpressed/accumulated tumor associate antigen, cancer-testis tumor associate antigen, lineage-restricted tumor associate antigen, mutated tumor associate antigen, or idiotypic tumor associate antigen, or fragment thereof.
118. The rMVA of embodiment 112, wherein the tumor associated antigen is derived from the b melanoma antigen (BAGE) family, cancer-associated gene (CAGE) family, G antigen (GAGE) family, melanoma antigen (MAGE) family, sarcoma antigen (SAGE) family and X antigen (XAGE) family, CT9, CT10, NY-ESO-1, L antigen (LAGE) 1, Melanoma antigen preferentially expressed in tumors (PRAME), and synovial sarcoma X (SSX) 2, melanoma antigen recognized by T cells-1/2 (Melan-A/MART-1/2), Gp100/pmel17, tyrosine-related protein (TRP) 1 and 2, P. polypeptide, melanocortin 1 receptor (MC1R), and prostate-specific antigen, β-catenin, breast cancer antigen (BRCA) 1/2, cyclin-dependent kinase (CDK) 4, chronic myelogenous leukemia antigen (CML) 66, fibronectin, p53, Ras, or TGF-βRII, or fragment thereof.
119. The rMVA of embodiments 89-111, wherein the antigenic peptide is derived from mucin 1, or fragment thereof.
120. The rMVA of embodiment 119, wherein the mucin 1 is encoded by the nucleic acid sequence of SEQ ID NO: 402, or a nucleic acid sequence at least 95% identical thereto.
121. The method of embodiment 119, wherein the mucin 1 comprises the amino acid sequence of SEQ ID NO: 349, or an amino acid sequence at least 95% identical thereto.
122. The rMVA of embodiment 119, wherein the mucin 1 comprises the amino acid sequence of SEQ ID NO: 403, or an amino acid sequence at least 95% identical thereto.
123. The rMVA of embodiment 119, wherein the mucin 1 comprises an extracellular domain fragment of human mucin 1.
124. The rMVA of embodiment 123, wherein the extracellular domain fragment of human mucin 1 is selected from SEQ ID NO: 358-361, or an amino acid sequence at least 95% identical thereto.
125. The rMVA of embodiment 119, wherein the mucin 1 comprises an intracellular domain fragment of human mucin 1.
126. The rMVA of embodiment 125, wherein the intracellular domain fragment of human mucin 1 comprises the amino acid sequence of SEQ ID NO: 362, or an amino acid sequence at least 95% identical thereto.
127. The method of embodiment 119, wherein the mucin 1 is selected from SEQ ID NO: 363-364, or an amino acid sequence at least 95% identical thereto.
128. The method of embodiment 127, wherein the mucin 1 comprises the amino acid sequence of SEQ ID NO: 363, or an amino acid sequence at least 95% identical thereto.
129. The method of embodiment 127, wherein the mucin 1 comprises the amino acid sequence of SEQ ID NO: 364, or an amino acid sequence at least 95% identical thereto.
130. The rMVA of embodiments 89-111, wherein the antigenic peptide is derived from an amino acid sequence selected from SEQ ID NOS: 349-357, or an amino acid sequence at least 95% identical thereto.
131. The rMVA of embodiments 89-111, wherein the antigenic peptide is derived from an amino acid sequence selected from SEQ ID NOS: 358-394, or an amino acid sequence at least 95% identical thereto.
132. The rMVA of embodiments 89-111, wherein the antigenic peptide is derived from an amino acid sequence selected from SEQ ID NOS: 403, or an amino acid sequence at least 95% identical thereto.
133. The rMVA of embodiments 89-132, wherein the glycoprotein signal peptide is derived from a Filoviridae.
134. The rMVA of embodiments 89-133, wherein the glycoprotein signal peptide comprises the amino acid sequence of SEQ ID NO. 396, or an amino acid sequence at least 95% identical thereto.
135. The rMVA of embodiments 89-133, wherein the glycoprotein transmembrane peptide comprises the amino acid sequence of SEQ ID NO. 398, or an amino acid sequence at least 95% identical thereto.
136. The rMVA of embodiments 89-135, wherein the viral matrix protein comprises the amino acid sequence of SEQ ID NO. 400, or an amino acid sequence at least 95% identical thereto.
137. The rMVA of embodiments 89-136, wherein x≥4.
138. The rMVA of embodiments 89-136, wherein x is 3, 4, or 5.
139. The rMVA of embodiments 89-138, wherein the polycistronic nucleic acid is inserted between two essential and highly conserved MVA genes.
140. The rMVA of embodiments 89-138, wherein the polycistronic nucleic acid is inserted into a natural deletion site.
141. The rMVA of embodiments 89-138, wherein the polycistronic nucleic acid is inserted into the MVA at sites selected from between MVA genes I8R and G1L, between MVA genes A50R and B1R in a restructured and modified deletion site III, or between MVA genes A5 and A6L.
142. The rMVA of embodiments 1-58, wherein the polycistronic nucleic acid is inserted into the rMVA at a site selected from between MVA genes I8R and G1L.
143. The rMVA of embodiments 1-58, wherein the polycistronic nucleic acid is inserted into the rMVA at a site selected from between MVA genes A50R and B1R in a restructured and modified deletion site III.
144. The rMVA of embodiments 1-58, wherein the polycistronic nucleic acid is inserted into the rMVA at a site selected from between MVA genes A5 and A6L.
145. The rMVA of embodiments 89-144, wherein the nucleic acid encoding the antigenic peptide amino acid sequence is in an open reading frame downstream of a Methionine (M) start codon.
146. A recombinant modified vaccinia Ankara (rMVA) viral vector comprising:
   a) a first nucleic acid sequence encoding an amino acid sequence comprising (M)(Secretion Signal Peptide-Immune Checkpoint Inhibitor Peptide-Cleavage Peptide)x (Secretion Signal Peptide-Immune Checkpoint Inhibitor Peptide), wherein x=1-10, and M is methionine; and
   b) a second nucleic acid sequence encoding an antigenic peptide;
   wherein the Immune Checkpoint Inhibitor Peptide is selected from an amino acid having the sequence of SEQ ID NO:1-57; and,
   wherein the first nucleic acid sequence and the second nucleic acid sequence are under the control of one or more vaccinia virus promoters.
147. A recombinant modified vaccinia Ankara (rMVA) viral vector comprising:
   a) a first nucleic acid sequence encoding an amino acid sequence comprising (M)(Secretion Signal Peptide-Immune Checkpoint Inhibitor Peptide-Cleavage Peptide)x)(Secretion Signal Peptide-Immune Checkpoint Inhibitor Peptide), wherein x=1-10, and M is methionine; and
   b) a second nucleic acid sequence encoding an antigenic peptide;
   wherein the Immune Checkpoint Inhibitor Peptide is SEQ ID NO:1, and the first nucleic acid sequence and the second nucleic acid sequence are under the control of one or more vaccinia virus promoters.
148. A recombinant modified vaccinia Ankara (rMVA) viral vector comprising:
   a) a first nucleic acid sequence encoding an amino acid sequence comprising (M)(Secretion Signal Peptide-Immune Checkpoint Inhibitor Peptide-Cleavage Peptide)x (Secretion Signal Peptide-Immune Checkpoint Inhibitor Peptide), wherein x=1-10, and M is methionine; and
   b) a second nucleic acid sequence encoding an antigenic peptide;
   wherein the Immune Checkpoint Inhibitor Peptide is SEQ ID NO:5, and the first nucleic acid sequence and the second nucleic acid sequence are under the control of one or more vaccinia virus promoters.
149. The rMVA of embodiments 146-148, wherein the secretion signal peptide comprises an amino acid sequence selected from SEQ ID NOS. 57-90, or an amino acid sequence at least 95% identical thereto.
150. The rMVA of embodiments 146-149, wherein the secretion signal peptide comprises the amino acid sequence of SEQ ID NO. 65, or an amino acid sequence at least 95% identical thereto.
151. The rMVA of embodiments 146-149, wherein the secretion signal peptide comprises the amino acid sequence of SEQ ID NO. 66, or an amino acid sequence at least 95% identical thereto.
152. The rMVA of embodiments 146-151, wherein the vaccinia virus promoter is selected from the nucleic acid sequence of SEQ ID NO:128-308.
153. The rMVA of embodiment 152, wherein the antigenic peptide is derived from the group consisting of an infectious agent and tumor associated antigen.
154. The rMVA of embodiment 153, wherein the infectious agent is a virus, bacterium, fungi, parasite, or amoeba.
155. The rMVA of embodiment 154, wherein the virus is selected from the group consisting of Adenovirus; Herpesvirus; a Poxvirus; a single stranded DNA; a Parvovirus; a double stranded RNA virus; Reovirus; a positive-single stranded RNA virus; Coronavirus; Picornavirus; Togavirus; a negative-single stranded RNA virus; a Orthomyxovirus; a Rhabdovirus; a single-stranded RNA-Retrovirus; a double-stranded DNA-Retrovirus; a Flaviviridae virus; Alphavirus virus, Filoviridae virus; a Paramyxoviridae virus; Rhabdoviridae virus; a Nyamiviridae virus; an Arenaviridae virus; a Bunyaviridae virus; or Ophioviridae virus; and Orthomyxoviridae virus.
156. The rMVA of embodiment 152, wherein the antigenic peptide is derived from the Ebola virus, the envelope glycoprotein of Ebola virus, the matrix protein VP40 of Ebola virus; the Lassa virus, Lassa virus protein Z; the Zika virus, Zika virus non-structural protein 1 (NSP-1); the Marburg virus; the Marburg virus glycoprotein; the Marburg VP40 matrix protein; the Plasmodium sp. parasite; Plasmodium falciparum; Plasmodium sp. circumsporozoite protein (CSP); Plasmodium sp. male gametocyte surface protein P230p (Pfs230 antigen); Plasmodium sp. sporozoite micronemal protein essential for cell traversal (SPECT2); Plasmodium sp. GTP-binding protein; putative antigen; the human immunodeficiency virus; HIV Env protein; HIV gp41; HIV gp120; HIV gp160; HIV Gag protein; HIV MA; HIV CA; HIV SP1; HIV NC; HIV SP2; HIV P6; HIV Pol protein; HIV RT; HIV RNase H; HIV IN; and HIV PR; or fragment thereof.
157. The rMVA of embodiment 152, wherein the antigenic peptide is derived from the group consisting of the SARS-CoV2; the SARS-CoV2 full-length S protein Wuhan Strain, the SARS-CoV2 S protein with K417T, E484K, and N501Y substitutions; the SARS-CoV2 full-length S protein Delta variant; the SARS-CoV2 full-length S protein Delta variant plus; the SARS-CoV2 full-length S protein stabilized by 2 proline substitutions; the SARS-CoV2 full-length stabilized S protein; the SARS-CoV2 full-length stabilized S protein with K417T, E484K, and N501Y substitutions; the SARS-CoV2 full-length stabilized S protein Delta variant; the SARS-CoV2 full-length stabilized S protein Delta variant plus; the SARS-CoV2 E protein; the SARS-CoV2 M protein; the SARS-CoV2 PP1ab polyprotein amino acid sequence; the SARS-CoV2 PP1a polyprotein amino acid sequence (Wuhan Hu1); the SARS-CoV2 NSP1-3 amino acid sequence (Wuhan Hu1); the SARS-CoV2 NSP4-11 amino acid sequence (Wuhan Hu1); the SARS-CoV2 ORF1b polyprotein NSP12-16 amino acid sequence (Wuhan Hu1); the SARS-CoV2 NSP12 amino acid sequence (Wuhan Hu1); the SARS-CoV2 NSP13-14 amino acid sequence (Wuhan Hu1); and the SARS-CoV2 NSP15-16 amino acid sequence (Wuhan Hu1); or fragment thereof.
158. The rMVA of embodiment 152, wherein the antigenic peptide is derived from an amino acid sequence selected from SEQ ID NOS: 358-394, or an amino acid sequence at least 95% identical thereto.
159. The rMVA of embodiments 146-158, wherein the first nucleic acid sequence and the second nucleic acid sequence are inserted into the MVA between essential MVA genes.
160. The rMVA of embodiments 146-158, wherein the first nucleic acid sequence is inserted into the MVA between essential MVA genes.
161. The rMVA of embodiments 146-160, wherein the second nucleic acid sequence is inserted into the MVA between essential MVA genes.
162. The rMVA of embodiments 146-158, wherein the first nucleic acid sequence and the second nucleic acid sequence are inserted into the MVA at sites selected from between MVA genes I8R and G1L, between MVA genes A50R and B1R in a restructured and modified deletion site III, or between MVA genes A5 and A6L.
163. The rMVA of embodiments 146-158, wherein the first nucleic acid sequence is inserted into the MVA at sites selected from between MVA genes I8R and G1L, between MVA genes A50R and B1R in a restructured and modified deletion site III, or between MVA genes A5 and A6L.
164. The rMVA of embodiments 146-158, wherein the second nucleic acid sequence is inserted into the MVA at sites selected from between MVA genes I8R and G1L, between MVA genes A50R and B1R in a restructured and modified deletion site III, or between MVA genes A5 and A6L.
165. The rMVA of embodiments 146-164, wherein the vaccinia virus promoter is a nucleic acid sequence of SEQ ID NOS:128-130, or a nucleic acid sequence at least 95% identical thereto.
166. The rMVA of embodiments 146-165, wherein the vaccinia virus promoter is SEQ ID NO:130, or a nucleic acid sequence at least 95% identical thereto.
167. The rMVA of embodiments 146-166, wherein the nucleic acid encoding the antigenic peptide amino acid sequence is in an open reading frame downstream of a Methionine (M) start codon.
168. The rMVA of embodiments 146-167, wherein x≥4.
169. The rMVA of embodiments 146-167, wherein x is 3, 4, or 5.
170. A recombinant modified vaccinia ankara (rMVA) viral vector comprising:
   i) a first nucleic acid sequence encoding an amino acid sequence comprising (Mucin 1 Extracellular Fragment Peptide-Glycoprotein Transmembrane Peptide-Mucin 1 Intracellular Fragment Peptide); and
   ii) a second nucleic acid sequence encoding an amino acid sequence comprising a Marburg virus (MARV) VP40 Protein; and
   iii) a third nucleic acid sequence encoding an amino acid sequence comprising (M)(Secretion Signal Peptide-Immune Checkpoint Inhibitor Peptide-Cleavage Peptide)ₓ(Secretion Signal Peptide-Immune Checkpoint Inhibitor Peptide), wherein x=1-10, and M is methionine;
   wherein the first nucleic acid sequence, the second nucleic acid sequence, and the third nucleic acid sequence are under the control of a vaccinia virus promoter; and wherein upon expression, the chimeric amino acid sequence and VP40 matrix protein are capable of assembling together to form virus-like particles (VLPs).
171. A recombinant modified vaccinia ankara (rMVA) viral vector comprising:
   i) a first nucleic acid sequence comprising the nucleic acid sequence of SEQ ID NO: 402 encoding a chimeric amino acid sequence;
   ii) a second nucleic acid sequence comprising the nucleic acid sequence of SEQ ID NO: 404;
   iii) a third nucleic acid sequence encoding an amino acid sequence comprising (M)(Secretion Signal Peptide-Immune Checkpoint Inhibitor Peptide-Cleavage Peptide)ₓ(Secretion Signal Peptide-Immune Checkpoint Inhibitor Peptide), wherein x=1-10, and M is methionine;
   wherein the first nucleic acid sequence, the second nucleic acid sequence, and the third nucleic acid sequence are under the control of a vaccinia virus promoter; and wherein upon expression, the chimeric amino acid sequence and VP40 matrix protein are capable of assembling together to form virus-like particles (VLPs).
172. A recombinant modified vaccinia ankara (rMVA) viral vector comprising:
   i) a first nucleic acid sequence encoding a chimeric amino acid sequence comprising the amino acid sequence of SEQ ID NO: 403; and
   ii) a second nucleic acid sequence encoding a MARV VP40 matrix protein comprising the amino acid sequence of SEQ ID NO: 405; and
   iii) a third nucleic acid sequence encoding an amino acid sequence comprising (M)(Secretion Signal Peptide-Immune Checkpoint Inhibitor Peptide-Cleavage Peptide)ₓ(Secretion Signal Peptide-Immune Checkpoint Inhibitor Peptide), wherein x=1-10, and M is methionine;
   wherein the first nucleic acid sequence, the second nucleic acid sequence, and the third nucleic acid sequence are under the control of a vaccinia virus promoter; and wherein upon expression, the chimeric amino acid sequence and VP40 matrix protein are capable of assembling together to form virus-like particles (VLPs).
173. The rMVA of embodiments 170-172, wherein the third nucleic acid sequence comprises the nucleic sequence of SEQ ID NO: 408, or a nucleic acid sequence at least 95% identical thereto.
174. The rMVA of embodiments 170-172, wherein the third nucleic acid sequence comprises the nucleic sequence of SEQ ID NO: 409, or a nucleic acid sequence at least 95% identical thereto.
175. The rMVA of embodiments 170-172, wherein the third nucleic acid sequence is an amino acid sequence selected from SEQ ID NOS: 1, 5, or 309-348, or an amino acid at least 95% identical thereto.
176. The rMVA of embodiment 175, wherein the third nucleic acid sequence encodes an immune checkpoint inhibitor peptide comprising the amino acid sequence of SEQ ID NOS: 325, or an amino acid sequence at least 95% identical thereto.
177. The rMVA of embodiment 175, wherein the third nucleic acid sequence encodes an immune checkpoint inhibitor peptide comprising the amino acid sequence of SEQ ID NOS: 329, or an amino acid sequence at least 95% identical thereto.
178. The rMVA of embodiment 175, wherein the third nucleic acid sequence encodes an immune checkpoint inhibitor peptide comprising the amino acid sequence of SEQ ID NOS: 333, or an amino acid sequence at least 95% identical thereto.
179. The rMVA of embodiment 175, wherein the third nucleic acid sequence encodes an immune checkpoint inhibitor peptide comprising the amino acid sequence of SEQ ID NOS: 337, or an amino acid sequence at least 95% identical thereto.
180. The rMVA of embodiments 170-179, wherein the first nucleic acid sequence, the second nucleic acid sequence, and the third nucleic acid sequence are inserted between two essential and highly conserved MVA genes.
181. The rMVA of embodiments 170-179, wherein the first nucleic acid sequence, the second nucleic acid sequence, and the third nucleic acid sequence are inserted into the rMVA at a site selected from between MVA genes I8R and GIL, between MVA genes A50R and B1R in a restructured and modified deletion site III, or between MVA genes A5 and A6L.
182. The rMVA of embodiments 170-179, wherein the first nucleic acid sequence is inserted between MVA genes I8R and GIL.
183. The rMVA of embodiments 170-179, wherein the second nucleic acid sequence is inserted between MVA genes A50R and B1R in the restructured and modified deletion site III.
184. The rMVA of embodiments 170-179, wherein the third nucleic acid sequence is inserted between the two essential MVA genes A5R and A6L.
185. The rMVA of embodiments 170-179, wherein the first nucleic acid sequence is inserted between MVA genes I8R and GIL, the second nucleic acid sequence is inserted between MVA genes A50R and B1R in the restructured and modified deletion site III, and the third nucleic acid sequence is inserted between the two essential MVA genes A5R and A6L.
186. The rMVA of embodiments 170-185, wherein the vaccinia virus promoter is a nucleic acid sequence selected from SEQ ID NOS: 128-308.
187. The rMVA of embodiment 170-186, wherein the vaccinia virus promoter is SEQ ID NO:130, or a nucleic acid sequence at least 95% identical thereto.
188. A pharmaceutical composition comprising at least one rMVA of embodiments 89-187 and a pharmaceutically acceptable carrier.
189. A method of preventing, treating, or inducing an immune response against, a target antigen in a patient in need thereof, said method comprising administering an effective amount of the pharmaceutical composition of embodiment 188, wherein the pharmaceutical composition enhances immunity directed against the target antigen.
190. The method of embodiment 189, wherein the target antigen is selected from the group consisting of a tumor associated antigen and an infectious agent.
191. The method of embodiment 190, wherein the tumor associated antigen is derived from an oncofetal tumor associate antigen, an oncoviral tumor associate antigen, overexpressed/accumulated tumor associate antigen, cancer-testis tumor associate antigen, lineage-restricted tumor associate antigen, mutated tumor associate antigen, or idiotypic tumor associate antigen, or fragment thereof.
192. The method of embodiment 190, wherein the tumor associated antigen is derived from the b melanoma antigen (BAGE) family, cancer-associated gene (CAGE) family, G antigen (GAGE) family, melanoma antigen (MAGE) family, sarcoma antigen (SAGE) family and X antigen (XAGE) family, CT9, CT10, NY-ESO-1, L antigen (LAGE) 1, Melanoma antigen preferentially expressed in tumors (PRAME), and synovial sarcoma X (SSX) 2, melanoma antigen recognized by T cells-1/2 (Melan-A/MART-1/2), Gp100/pmel17, tyrosine-related protein (TRP) 1 and 2, P. polypeptide, melanocortin 1 receptor (MC1R), and prostate-specific antigen, β-catenin, breast cancer antigen (BRCA) 1/2, cyclin-dependent kinase (CDK) 4, chronic myelogenous leukemia antigen (CML) 66, fibronectin, p53, Ras, or TGF-βRII, or fragment thereof.
193. The method of embodiments 189-192, wherein the patient is a human having a cancer.
194. The method of embodiment 193, wherein the cancer is selected from bowel cancer, ovarian cancer, breast cancer, malignant melanoma, hepatoma, testicular cancer, prostate cancer, multiple myeloma, lymphoma, colorectal cancer, bile duct cancer, pancreatic cancer, lung cancer, melanoma, soft tissue sarcoma, or colon cancer.
195. The method of embodiment 190, wherein the infectious agent is a virus, bacterium, fungi, parasite, or amoeba.
196. The method of embodiment 195, wherein the virus is selected from the group consisting of Adenovirus; Herpesvirus; a Poxvirus; a single stranded DNA; a Parvovirus; a double stranded RNA virus; Reovirus; a positive-single stranded RNA virus; Coronavirus; Picornavirus; Togavirus; a negative-single stranded RNA virus; a Orthomyxovirus; a Rhabdovirus; a single-stranded RNA-Retrovirus; a double-stranded DNA-Retrovirus; a Flaviviridae virus; Alphavirus virus, Filoviridae virus; a Paramyxoviridae virus; Rhabdoviridae virus; a Nyamiviridae virus; an Arenaviridae virus; a Bunyaviridae virus; or Ophioviridae virus; and Orthomyxoviridae virus.
197. The method of embodiment 190, wherein the infectious agent is derived from the Ebola virus, the envelope glycoprotein of Ebola virus, the matrix protein VP40 of Ebola virus; the Lassa virus, Lassa virus protein Z; the Zika virus, Zika virus non-structural protein 1 (NSP-1); the Marburg virus; the Marburg virus glycoprotein; the Marburg VP40 matrix protein; the Plasmodium sp. parasite; Plasmodium falciparum; Plasmodium sp. circumsporozoite protein (CSP); Plasmodium sp. male gametocyte surface protein P230p (Pfs230 antigen); Plasmodium sp. sporozoite micronemal protein essential for cell traversal (SPECT2); Plasmodium sp. GTP-binding protein; putative antigen; the human immunodeficiency virus; HIV Env protein; HIV gp41; HIV gp120; HIV gp160; HIV Gag protein; HIV MA; HIV CA; HIV SP1; HIV NC; HIV SP2; HIV P6; HIV Pol protein; HIV RT; HIV RNase H; HIV IN; and HIV PR; SARS-CoV2; the SARS-CoV2 full-length S protein Wuhan Strain, the SARS-CoV2 S protein with K417T, E484K, and N501Y substitutions; the SARS-CoV2 full-length S protein Delta variant; the SARS-CoV2 full-length S protein Delta variant plus; the SARS-CoV2 full-length S protein stabilized by 2 proline substitutions; the SARS-CoV2 full-length stabilized S protein; the SARS-CoV2 full-length stabilized S protein with K417T, E484K, and N501Y substitutions; the SARS-CoV2 full-length stabilized S protein Delta variant; the SARS-CoV2 full-length stabilized S protein Delta variant plus; the SARS-CoV2 E protein; the SARS-CoV2 M protein; the SARS-CoV2 PP1ab polyprotein amino acid sequence; the SARS-CoV2 PP1a polyprotein amino acid sequence (Wuhan Hu1); the SARS-CoV2 NSP1-3 amino acid sequence (Wuhan Hu1); the SARS-CoV2 NSP4-11 amino acid sequence (Wuhan Hu1); the SARS-CoV2 ORF1b polyprotein NSP12-16 amino acid sequence (Wuhan Hu1); the SARS-CoV2 NSP12 amino acid sequence (Wuhan Hu1); the SARS-CoV2 NSP13-14 amino acid sequence (Wuhan Hu1); and the SARS-CoV2 NSP15-16 amino acid sequence (Wuhan Hu1); or fragment thereof.
198. The method of embodiments 195-197, wherein the patient is a human exposed to the infectious agent.
199. The method of embodiment 198, wherein the exposed human is symptomatic.
200. The method of embodiment 198, wherein the exposed human is asymptomatic.
201. The method of embodiments 195-197, wherein the patient is a human unexposed to the infectious agent.
202. The method of embodiments 188-201, wherein the rMVA administration is selected from intramuscular, intraarterial, intravascular, intravenous, intraperitoneal, or subcutaneous injection.
203. The method of embodiments 188-202, wherein the rMVA comprises an adjuvant for enhancing an immune response.
204. The method of embodiments 188-202, wherein the rMVA comprises a vaccine for inducing an immune response.
205. The method of embodiments 192-204, wherein the patient is administered the pharmaceutical composition at least 2 or more times.
206. The method of embodiment 205, wherein the administrations are separated by at least a 4-week interval.
207. A method of enhancing an immune response in a patient comprising administering to the patient an effective amount of an rMVA of embodiments 89-187.
208. A method of inducing an immune response to a MUC1 antigen in a patient comprising administering to the patient an effective amount of an rMVA of embodiments 119-145 or 170-187.
209. The method of embodiments 207-208, wherein the patient is human.

### EXAMPLES

The claimed invention is further described by way of the following non-limiting examples. Further aspects and embodiments of the present invention will be apparent to those of ordinary skill in the art, in view of the above disclosure and following experimental exemplification, included by way of illustration and not limitation, and with reference to the attached figures.

### Example 1. Mice

All animal experiments were carried out in strict accordance with the Policy on Humane Care and Use of Laboratory Animals of the United States Public Health Service. The protocol was approved by the Institutional Animal Care and Use Committee (IACUC) at The Rockefeller University. Mice were euthanized using CO₂, and every effort was made to minimize suffering. Human fetal liver samples were obtained via a non-profit partner (Advanced Bioscience Resources, Alameda, CA). As no information was obtained that would identify the subjects from whom the samples were derived, Institutional Review Board approval for their use was not required. (See Huang J. et al., “An AAV vector-mediated gene delivery approach facilitates reconstitution of functional human CD8⁺ T cells in mice”, PLoS One, 2014 Feb. 6, 9(2), e88205. doi: 10.1371/journal.pone.0088205. eCollection 2014.PMID:24516613)

Six to eight-week-old female BALB/c mice were purchased from The Jackson Laboratory (Bar Harbor, ME). NOD.Cgtm1Unc Prkdcscid Il2rgtm1Wjl/SzJ (NSG) mice exhibiting features of both severe combined immunodeficiency mutations and interleukin (IL)-2 receptor gamma-chain deficiency were also purchased from Jackson Laboratories and maintained under specific pathogen-free conditions in the animal facilities at The Rockefeller University Comparative Bioscience Center. All mice were maintained under standard conditions in the Laboratory Animal Research Center of The Rockefeller University and the protocol was approved by the Institutional Animal Care and Use Committee at The Rockefeller University (Assurance no. A3081-01).

### Example 2. Generation of HIS-CD8 Mice

Preparation of the recombinant AAV9 (rAAV9) vectors encoding human IL-3, IL-15, GM-CSF, and HLA-A*0201 were constructed. (See Huang J., et al., “An AAV vector-mediated gene delivery approach facilitates reconstitution of functional human CD8⁺ T cells in mice”, PLoS One, 2014 Feb. 6, 9(2), e88205. doi: 10.1371/journal.pone.0088205. eCollection 2014.PMID:24516613)

Four-week-old NSG mice were transduced with rAAV9 encoding HLA-A*0201 by perithoracic injection and with rAAV9 encoding HLA-A*0201 and AAV9 encoding human IL-3, IL-15, and GM-CSF, by IV injection. (See Huang J., et al., “An AAV vector-mediated gene delivery approach facilitates reconstitution of functional human CD8⁺ T cells in mice”, PLoS One, 2014 Feb. 6, 9(2), e88205. doi: 10.1371/journal.pone.0088205. eCollection 2014.PMID:24516613)

Two weeks later, mice were subjected to 150-Gy total body sub-lethal irradiation for myeloablation, and several hours later, each transduced, irradiated mouse was engrafted intravenously with 1×10⁵ HLA-A*0201+ matched, CD34⁺ human hematopoietic stem cells (HSCs). CD34+ HSCs among lymphocytes derived from HLA-A*0201+ fetal liver samples were isolated using a Human CD34 Positive Selection kit (Stem Cell Technologies Inc. Vancouver, BC, Canada; See Lepus C M, et al., “Comparison of human fetal liver, umbilical cord blood, and adult blood hematopoietic stem cell engraftment in NOD-scid/gammac−/−, Balb/c-Rag1−/−gammac−/−, and C.B-17-scid/bg immunodeficient mice”, Hum Immunol., 2009 October, 70(10), 790-802. doi: 10.1016/j.humimm.2009.06.005. Epub 2009 Jun. 12. PMID: 19524633). At 14 weeks after HSC engraftment, the reconstitution status of human CD45+ cells in the blood of HIS-CD8 mice was determined by flow cytometric analysis. (See Huang J, et al., “An AAV vector-mediated gene delivery approach facilitates reconstitution of functional human CD8⁺ T cells in mice”, PLoS One, 2014 Feb. 6, 9(2), e88205. doi: 10.1371/journal.pone.0088205. eCollection 2014.PMID:24516613)

### Example 3. AdPyCS and AdPfCS Vaccines

Preparation of the recombinant serotype 5 adenovirus that expressed P. yoelii circumsporozoite protein (PyCS), AdPyCS, was constructed. (See Rodrigues E G, et al., “Single immunizing dose of recombinant adenovirus efficiently induces CD8⁺ T cell-mediated protective immunity against malaria”, J Immunol., 1997 Feb. 1, 158(3), 1268-74. PMID: 9013969).

### Example 4. ELISpot Assay and Flow Cytometry to Measure Antigen-Specific CD8⁺ T Cells

The relative numbers of splenic PyCS-specific, IFN-γ-secreting CD8⁺ T cells of AdPyCS-immunized mice were determined by an ELISpot assay, using a mouse IFN-γ ELISpot kit (Abcam, Cambridge, MA) and a synthetic 9-mer peptide, SYVPSAEQI (SEQ ID NO: 406) (Peptide 2.0 Inc., Chantilly, VA) corresponding to the immunodominant CD8⁺ T cell epitope within PyCS. (See Li X, et al., “Human CD8⁺ T cells mediate protective immunity induced by a human malaria vaccine in human immune system mice”, Vaccine, 2016 Aug. 31, 34(38), 4501-4506. doi: 10.1016/j.vaccine.2016.08.006. Epub 2016 Aug. 5.; PMID: 27502569). After the collection of splenocytes from mice 12 days after AdPyCS immunization, 5×10⁵ splenocytes were placed on each well of the 96-well ELISpot plates were pre-coated with IFN-γ antibody and incubated with the SYVPSAEQI (SEQ ID NO: 406) peptide at 5 μg/mL for 24 h at 37° C., in a CO₂ incubator. After the ELISpot plates were washed, they were incubated with biotinylated anti-mouse IFN-γ antibody for 2-3 h at RT, followed by incubation with avidin-conjugated with horseradish peroxidase for 45 min at RT in the dark. Finally, the spots were developed after the addition of the ELISpot substrate (Abcam). To identify the number of IFN-γ-secreting CD8⁺ T cells in each well, the mean number of spots (for duplicates) counted in the wells incubated with splenocytes in the presence of the peptide was subtracted by the mean number of spots (for duplicates) counted in the wells that were incubated with splenocytes only. The percentage of IFN-γ⁺ T cells among splenocytes of immunized mice were determined by a flow cytometry. After isolating splenocytes the cells were washed twice and blocked for 5 min on ice using inactivated normal mouse serum supplemented with anti-CD16/CD32 (clone 93—BioLegend, San Diego, CA, USA).

### Example 5. Staining with HLA-Ar0201 Tetramer Loaded with YLNKIQNSL Peptide

The Allophyocyanin (APC)-labeled human HLA-A*0201 tetramer loaded with the peptide YLNKIQNSL (SEQ ID NO: 407), corresponding to the PfCSP CD8⁺ T-cell epitope, was provided by the NIH Tetramer Core Facility (See Blum-Tirouvanziam U, et al., “Localization of HLA-A2.1-restricted T cell epitopes in the circumsporozoite protein of Plasmodium falciparum”, J Immunol., 1995 Apr. 15, 154(8), 3922-31; PMID: 7535817; 43; Bonelo A, et al., “Generation and characterization of malaria-specific human CD8⁺ lymphocyte clones: effect of natural polymorphism on T cell recognition and endogenous cognate antigen presentation by liver cells”, Eur J Immunol., 2000 November, 30(11), 3079-88; doi: 10.1002/1521-4141(200011)30:11<3079::AID-IMMU3079>3.0.CO;2-7. PMID: 11093122) (Table 12).

TABLE 12
Synthetic 9-mer Peptide Sequences

| SEQ ID NO: | Peptide Sequence: |
|---|---|
| 406 | SYVPSAEQI |
| 407 | YLNKIQNSL |

Twelve days after immunization of HIS-CD8 mice with AdPfCS, the spleens were harvested from the mice, and splenocytes were stained with APC-labeled human HLA-A*0201 tetramer loaded with YLNKIQNSL (SEQ ID NO: 407) and PE-labeled anti-human CD8 antibody (BioLegend, San Diego, CA). The percentage of HLA-A*0201-restricted, PfCSP-specific CD8⁺ T cells among the total human CD8⁺ T-cell population was determined using a BD LSR II flow cytometer (Franklin Lakes, NJ). (See Li X, et al., “Human CD8⁺ T cells mediate protective immunity induced by a human malaria vaccine in human immune system mice”, Vaccine, 2016 Aug. 31, 34(38), 4501-4506; doi: 10.1016/j.vaccine.2016.08.006. Epub 2016 Aug. 5. PMID: 27502569)

### Example 6. MVA Construction, Seed Stock Preparation, VLP Formation, and Protein Expression

Two recombinant MVAs, MVA-5x.LD01 and MVA-5x.LD10, were constructed that encode an optimized nucleic acid sequence of five repeats of LD01 (SEQ ID NO: 408) or LD10 (SEQ ID NO: 409) in polycistronic format (Table 13). A signal sequence (SEQ ID NO: 66) was added prior to LD01 or LD10 to route the peptides for secretion from the cell and a dual cleavage site (SEQ ID NO: 123) was added following the sequences to facilitate production of monomer peptides from the polycistronic design. The resultant LD01 insert encoded for the amino acid sequence of SEQ ID NO:332. The resultant LD10 insert encoded for the amino acid sequence of SEQ ID NO: 337. The starting material for recombinant virus production was parental MVA that had been harvested in 1974, before the appearance of Bovine Spongiform Encephalopathy/Transmissible Spongiform Encephalopathy (BSE/TSE) and plaque purified 3 times using certified reagents from sources free of BSE. A shuttle vector was used to insert the LD01 or LD10 sequences between two essential genes I8R/G1L of MVA by means of homologous recombination. The chosen insertion site has been identified as supporting high expression and insert stability. All inserted sequences were codon optimized for MVA as below:

TABLE 13
Sequence Optimization

| SEQ ID | | |
|---|---|---|
| NO: | Identifier | Nucleic Acid Sequence |
| 408 | 5xLD01 | ATGGACGCCATGAAGAGAGGACTTTGTTGCGTCCTACTACTATGCGGAG |
| | | CGGTATTCGTATCTCCGTCGCAAGAAATTCACGCGAGATTCAGAAGAGG |
| | | TGCCAGATGCAGAAGAACATCTACCGGACAGATCTCCACCTTGAGAGT |
| | | AAATATCACAGCGCCGCTATCTCAGAGAGCCAAGAGAGGATCGGGAGC |
| | | GACAAACTTCTCGCTATTGAAACAAGCGGGAGATGTCGAAGAGAACCC |
| | | AGGACCAGATGCTATGAAGAGAGGACTTTGCTGCGTATTGCTATTGTGC |
| | | GGAGCCGTCTTCGTCTCACCATCTCAAGAAATCCATGCCAGATTCAGAA |
| | | GAGGTGCTAGATGTAGAAGAACCTCCACGGGACAAATCAGTACCCTAA |
| | | GAGTTAACATCACCGCGCCGTTGAGTCAAAGAGCTAAGAGAGGTTCCG |
| | | GAGCCACCAACTTCAGTTTGCTAAAGCAGGCGGGAGATGTGGAAGAGA |
| | | ATCCTGGTCCTGACGCAATGAAGAGAGGACTTTGCTGCGTTCTATTGCT |
| | | ATGCGGTGCCGTCTTTGTTTCTCCGAGTCAAGAGATACACGCTAGATTC |
| | | AGAAGAGGTGCAAGATGTAGAAGAACCTCGACCGGTCAAATCTCGACG |
| | | CTTAGAGTCAATATTACCGCGCCATTGTCGCAGAGAGCGAAGAGAGGA |
| | | TCGGGAGCCACTAATTTCAGTCTACTTAAGCAAGCGGGAGATGTAGAG |
| | | GAGAATCCTGGACCGGATGCCATGAAGAGAGGACTTTGTTGCGTTCTGT |
| | | TGCTTTGCGGAGCTGTGTTCGTCAGTCCTTCTCAAGAGATTCATGCAAG |
| | | ATTCAGAAGAGGTGCAAGATGCAGAAGAACCAGTACGGGACAGATTTC |
| | | GACATTAAGAGTGAACATTACTGCGCCTTTGTCTCAAAGAGCGAAGAG |
| | | AGGTTCCGGAGCGACGAATTTCTCGTTGCTCAAGCAAGCGGGAGATGT |
| | | AGAAGAGAACCCAGGACCTGATGCAATGAAGAGAGGACTTTGTTGCGT |
| | | ATTACTTCTTTGCGGTGCAGTGTTTGTCTCGCCGTCACAAGAGATCCAC |
| | | GCAAGATTCAGAAGAGGTGCCAGATGTAGAAGAACTAGTACAGGACAA |
| | | ATCTCCACGCTAAGAGTAAACATAACGGCACCACTATCTCAATAA |
| 409 | 5xLD10 | ATGGACGCCATGAAGAGAGGACTTTGTTGCGTCCTACTACTATGCGGAG |
| | | CGGTATTCGTATCTCCGTCGCAAGAAATTCACGCGAGATTCAGAAGAGG |
| | | TGCCAGATCTACAGGACAGATCTCTACCCTAAGAGTCAATATCACAGCG |
| | | CCGCTATCTCAGAGAGCGAAGAGAGGATCGGGAGCGACAAACTTCTCG |
| | | CTATTGAAACAAGCGGGAGATGTCGAGGAGAACCCAGGACCAGATGCT |
| | | ATGAAGAGAGGACTTTGCTGCGTATTGCTATTGTGCGGAGCCGTGTTCG |
| | | TCTCGCCATCTCAAGAAATCCATGCCAGATTCAGAAGAGGTGCTAGAA |
| | | GTACCGGACAAATCTCCACGTTGAGAGTAAACATTACCGCGCCGTTGTC |
| | | GCAAAGAGCTAAGAGAGGTTCCGGAGCCACTAACTTCAGTTTGCTAAA |
| | | GCAGGCGGGAGATGTGGAAGAGAATCCTGGTCCTGACGCAATGAAGAG |
| | | AGGACTTTGCTGCGTTCTATTGCTATGCGGTGCCGTCTTTGTTTCTCCGA |
| | | GTCAAGAGATACACGCTAGATTCAGAAGAGGTGCTAGATCCACGGGAC |
| | | AAATCAGTACCCTTAGAGTGAACATCACGGCGCCACTTTCTCAAAGAGC |
| | | CAAGAGAGGTTCCGGAGCGACCAATTTCTCGTTGCTAAAGCAAGCGGG |
| | | AGATGTAGAAGAGAATCCCGGACCGGATGCCATGAAGAGAGGACTTTG |
| | | TTGCGTGCTGTTGCTTTGCGGAGCTGTGTTCGTCAGTCCTTCTCAAGAGA |
| | | TTCATGCAAGATTCAGAAGAGGTGCAAGATCGACCGGTCAAATTTCGA |
| | | CGCTAAGAGTTAACATAACGGCGCCCTTGAGTCAGAGAGCCAAGAGAG |
| | | GATCGGGAGCCACTAACTTCTCGTTGTTGAAGCAGGCGGGAGATGTAG |
| | | AAGAGAATCCGGGTCCAGATGCAATGAAGAGAGGACTTTGTTGCGTAT |
| | | TACTTCTTTGCGGTGCAGTGTTTGTCTCGCCGTCACAAGAGATCCACGC |
| | | AAGATTCAGAAGAGGTGCCAGAAGTACGGGTCAAATTAGTACCTTGAG |
| | | AGTCAATATTACGGCGCCTTTGTCACAGTAATGA |

Silent mutations were introduced to interrupt homo-polymer sequences (>4G/C and >4A/T), which reduce RNA polymerase errors that possibly lead to frameshift mutations. All vaccine inserts were placed under control of the modified H5 early/late vaccinia promoter (SEQ ID NO: 130). Vectors, Research Seed Virus (RSV), and Research Stocks (RS) were prepared in a dedicated room with full traceability and complete documentation of all steps using BSE/TSE-free raw materials, and therefore can be directly used for production of cGMP Master Seed Virus (MSV). For production of RSV for animal studies, a chicken embryo fibroblast cell line, DF-1 cells (ATCC, CRL-12203), were seeded into sterile tissue culture flasks and infected with MVA-5x.LD01 or MVA-5x.LD10 at an MOI of 0.01. Cells were recovered 3 days post-infection, disrupted by sonication, and bulk harvest material clarified by low-speed centrifugation. The clarified viral harvest was purified using sucrose cushion ultracentrifugation twice. The purified viruses were titrated by limiting dilution in DF1 cells, diluted to 1×10⁸ TCID50/mL in sterile PBS+7% sucrose, dispensed into sterile vials, and stored at −80° C.

### Example 7. Production of Anti-LD01/LD10 mAb

KLH conjugated LD01 peptide formulated in Sigma adjuvant system (Cat No. S6322) was used to immunize SJL/J mice intramuscularly. Following two similar intramuscular boosts at 2-week intervals, the mice were culled and spleens and lymph nodes were collected. Splenocytes and lymphocytes were isolated and fused to HL-1 mouse myeloma cells and cultured for 13 days. On day 13, colonies were picked manually and transferred to selection media. Culture supernatants were screened for specificity by ELISA using plate coated BSA conjugated peptides. Supernatants were screened against BSA-conjugated LD01 peptide as well as LD10. Two clones (3F11 and 7G10) were selected based on their high level of binding to both peptides as well as the high concentration of supernatant antibody. Monoclonal cultures of these two clones were expanded and the supernatants were used to purify the antibodies. Cell suspensions, containing at least 8.0×10⁷ cells in 2×T-75 flasks, were aseptically transferred to 2×50 mL centrifuge tubes and centrifuged at 1000 rpm for 5 minutes. The resulting cell pellet was re-suspended in 25 mL of HyClone HYQSFMMAB media+5% FBS and slowly added to 250 mL bag containing 225 mL of HyClone HYQSFMMAB media+5% FBS. The bag was placed in an incubator set at 5% CO₂, 37° C. for 10-14 days. After 10-14 days of growth, the contents of the 250 mL bag were transferred to a 250 mL centrifuge bottle, 10 mL of Neutralization Buffer (1M TRIS, 1.5M NaCl, pH 8.5) was added to it, and centrifuged at 8600 rpm for 10 min using a Sorvall GSA rotor. The supernatant was filtered using a 0.45 m bottle top filter. A 5 mL protein A column connected to a FPLC Purification System was washed with 25 mL of ultra-pure water followed by 25 mL of 50 mM TRIS, 250 mM NaCl, pH 8.0. The filtered supernatant was loaded onto the column at a flow rate of 7 mL/minute. The column was further washed with 15 mL of 50 mM TRIS, 250 mM NaCl, pH 8.0. Elution fractions were collected in 15 mL tubes containing 800 μL of Neutralization Buffer (1M Tris Base, 1.5M NaCl, pH 7.4). The antibody was eluted with 20 mL of 50 mM Glycine, pH 3.0 and dialyzed against 1-2L of 1×PBS pH 7.4 (depending on volume of purified Ab) on a stirrer at 4° C. overnight. The dialyzed antibody was sterile filtered and aliquoted for storage.

### Example 8. Dot Blot Assay

DF-1 cells were infected at a multiplicity of infection of 0.5 with parental MVA, MVA-5X.LD01 or MVA-5X.LD10 and 48 hours later the supernatant was collected. In order to concentrate secreted peptide, supernatant was passed through Pierce C-18 tips (Thermofisher, Cat. No. 87782). Twenty microliters from each sample and 125 ng of synthetic LD01 peptide were spotted onto a PVDF membrane, allowed to dry at room temperature, then blocked with Intercept blocking buffer (Li-Cor, Cat. No. 927-70001) for 30 mins at room temperature. The membrane was incubated overnight at 4° C. in primary antibody (Leidos, clone: 7G10) diluted in blocking buffer at 1:1000. Three washes with PBST (PBS with 0.05% Tween-20) were performed, and the membrane was probed for 1 h with anti-mouse-680RD (Invitrogen, Cat. No. A-21058) (1:10,000). The membrane was then washed again and imaged using Odyssey imager.

### Example 9. Immunocytochemistry Assay

DF-1 cells were infected at a multiplicity of infection of 0.5 with parental MVA, MVA-5X.LD01 or MVA-5X.LD10 for 48 hours, subsequently cells were fixed in 1:1 methanol:acetone and washed with water. Cells were then probed with a mouse anti-LD01/LD10 antibody (Leidos, clone: 3F11) at room temperature for 1 hour. Three washes with water were performed and the cells were stained for 1 hour with anti-mouse-HRP at 1:1000 dilution (VWR, Cat. No. 10150-400). The cells were then washed again and developed with AEP substrate kit (Abcam Cat. No. ab64252). Images of stained cells were captured at 20× magnification using light microscopy.

### Example 10. Data Analysis

Statistical analyses were performed using GraphPad Prism (GraphPad Software, Inc., La Jolla, CA). The two-tailed Unpaired t-test was used to determine between two groups. Data are expressed as the mean±SEM and P<0.05 was considered statistically significant.

### Example 11. MVA Vector Construction

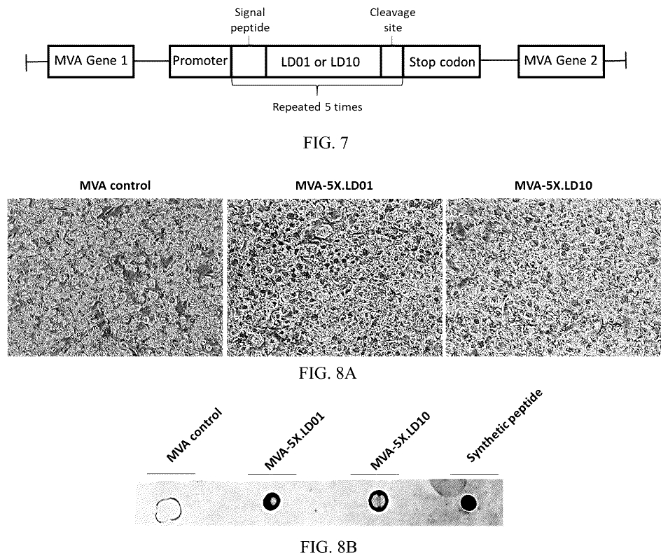

To establish whether LD10 could be expressed by a viral vector, a recombinant MVA virus that encodes five repeats of the LD10 sequence in polycistronic format (MVA-5x.LD10) (FIG. 7) and a similar recombinant MVA virus expressing five repeats of the LD01 sequence was constructed (MVA-5x.LD01) (FIG. 7) according to Example 6. To facilitate peptide secretion, a signal sequence was added prior to LD01 or LD10, and a dual cleavage site was added following the sequences in order to facilitate production of the monomer LD01 or LD10 from the polycistronic design.

Immunohistochemistry on infected cells was performed using a mAb cross reactive to LD01 and LD10; to initially determine whether the recombinant MVA vectors express LD01 or LD10. Cells were fixed and permeabilized with 50:50 methanol/acetone.

### Example 12. LD01 and LD10 are Produced by MVA-Infected Cells

A dot blot was performed on infected cell supernatants to establish that LD01 or LD10 is being secreted by the recombinant MVA vector. The parental MVA vector showed negligible signal as shown in FIG. 8B. Liquid chromatography tandem mass spectrometry of the cell supernatants identified LD01 and LD10 fragments corroborated the dot blot results.

Cells infected with the parental MVA vector showed no specific staining, however, cells infected with either MVA-5X.LD01 or MVA-5X.LD10 vectors showed positive staining as shown in FIG. 8A; indicating the intracellular expression of the peptides. Both MVA-5X.LD01 and MVA-5X.LD10 vector samples demonstrated positive staining, arguing for secretion of LD01 and LD10. LD01 and LD10 are expressed and secreted by the recombinant MVA vectors. The above technique was used to generate the image in FIG. 8.

### Example 13. Delivery of LD01 or LD10 Via a Viral Vector Enhances Expansion of Vaccine-Induced, Antigen-Specific CD8⁺ T Cells

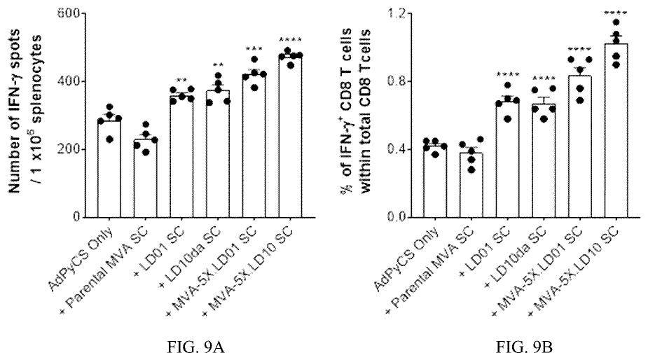

Having confirmed that LD01 and LD10 are expressed in and secreted from cells infected with peptide-encoding MVA constructs (FIG. 8A and FIG. 8B), AdPyCS-specific CD8+ T cell expansion following treatment with MVA-encoding LD01 or LD10 was assessed. A parental MVA vector was included as a negative control, while synthetic LD01 and LD10da served as positive controls. As shown in FIG. 9, treatment with 100 μg of LD01 or LD10da directly following vaccination significantly increased antigen-specific CD8+ T cell numbers relative to AdPyCS alone. Similarly, injection of 10⁸ TCID₅₀ of MVA-5X.LD01 or MVA-5X.LD10 enhanced antigen-specific CD8+ T cell expansion, which contrasted the treatment with the parental MVA vector (FIG. 9). Taken together, these in vivo results indicate that the delivery of LD01 or LD10 via the MVA vector results in increased activation of immune effector cells and immunomodulatory activity that is likely due to their expression in vivo. As such, these results corroborate that peptide-based immunomodulators can be successfully delivered by viral vector and induce significantly enhanced immune response.

### Example 14. MVA-VLP-MUC-1-LD10 Construction and Validation of Insert Integrity

Starting with parental MVA virus, shuttle vectors were used to insert the optimized MUC-1 and Marburg virus (MARV) transmembrane glycoprotein (GP) transmembrane domain (TM) chimeric nucleic acid sequence (SEQ ID NO: 402) encoding a MUC-1-MARV GPTM amino acid sequence (SEQ ID NO: 403) between MVA genes I8R and GIL, the MARV VP40 nucleic acid sequence (SEQ ID NO: 404) encoding a MARV VP40 amino acid sequence (SEQ ID NO: 405) between MVA genes A50R and B1R in the restructured and modified deletion site III, and the 5×LD10 (SEQ ID NO: 409) nucleic acid sequence encoding a 5×LD10 amino acid sequence (SEQ ID NO: 337) between the two essential MVA genes A5R and A6L by means of homologous recombination. These insertion sites were previously demonstrated to support high expression and stability of transgenes. Silent mutations were introduced to interrupt homo-polymer sequences (>4G/C and >4A/T), which reduce RNA polymerase errors that possibly lead to frameshift mutations. The inserted sequences were codon optimized for expression under control of the modified H5 early/late vaccinia promoter (SEQ ID NO: 130) by the MVA virus.

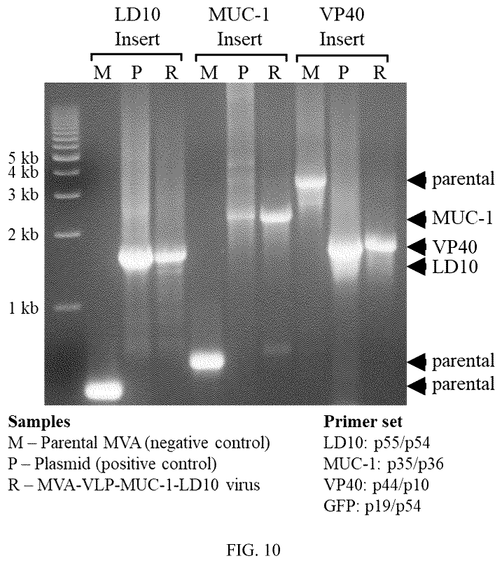

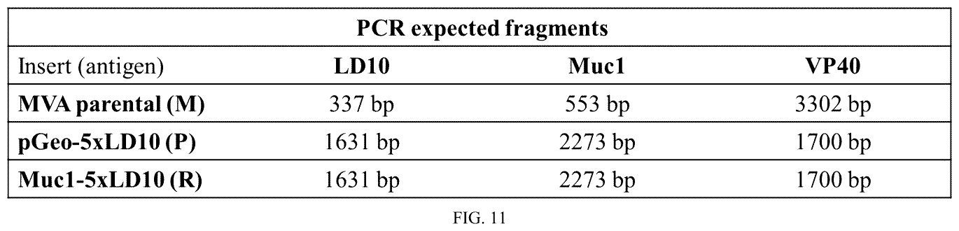

Viral vectors, Research Seed Virus (RSV), and Research Stocks (RS) were prepared in a dedicated room with full traceability and complete documentation of all steps using BSE/TSE-free raw materials capable of production of cGMP Master Seed Virus (MSV), as described previously (Example 6). The chicken embryo fibroblast cell line, DF-1 cells (ATCC, CRL-12203), was seeded in sterile tissue culture flasks and infected with either MVA parental or MVA-VLP-MUC-1-LD10 recombinant virus at a multiplicity of infection of 0.01. Viral DNA samples harvested from these cells were analyzed by PCR to examine transgene insert integrity (FIG. 10), using specific primers upstream and downstream of each insert (Table 14). MVA parental viral DNA use used as a negative control and the DNA from three different plasmids, containing the Muc1, VP40 or LD10 genes, was used as a positive control. The bands identified matched the expected sizes (FIG. 11).

TABLE 14
Primer Sequences

| SEQ | Sequence | |
|---|---|---|
| ID NO: | Description | Nucleic Acid Sequence |
| 410 | p55 LD10 F | AGATCGGAGATGACTGCGATG |
| 411 | p54 LD10R/GFP R | CGATGGATGGTCAGATTGTCC |
| 412 | p35 MUC-1 F | GAGAGGACGGGAGAATTAACTA |
| 413 | p36 MUC-1 R | TGGTAGGAATACCAGATACGAC |
| 414 | p44 VP40 F | GGAGCAGAGTTTACATCTTCCAA |
| 415 | p10 VP40 R | CTCCGTGAGAATATCCTTGCTC |

### Example 15. Validation of Recombinant Protein Production by MVA-VLP-MUC-1-LD10 Infected DF-1 Cells

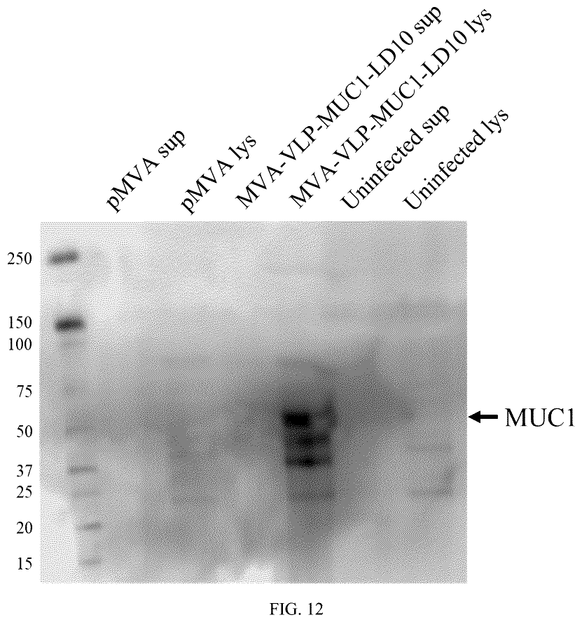

To establish the expression of MUC-1 and VP40 protein from the recombinant MVA-VLP-MUC-1-LD10 viral vector, DF1 cells were cultured in 6-well plates and infected with either parental modified vaccinia Ankara (pMVA) or recombinant MVA virus encoding VLP-MUC-1-LD10. Cellular supernatant and lysate were harvested and analyzed by SDS-PAGE on a Mini-Protean TGX gel and transferred to a PVDF membrane. The membranes were then probed with MUC1 antibody (mouse monoclonal VU4H5, Santa Cruz #sc-7313, 1:200). The expected size of MUC-1 protein is 63 kDa. Robust expression of MUC-1 protein was observed only in MVA-VLP-MUC-1-LD10 lysate and not in the supernatant fraction of cells infected with the recombinant MVA virus encoding VLP-MUC-1-LD10 (FIG. 12). Negligible signal was observed in all other negative control samples.

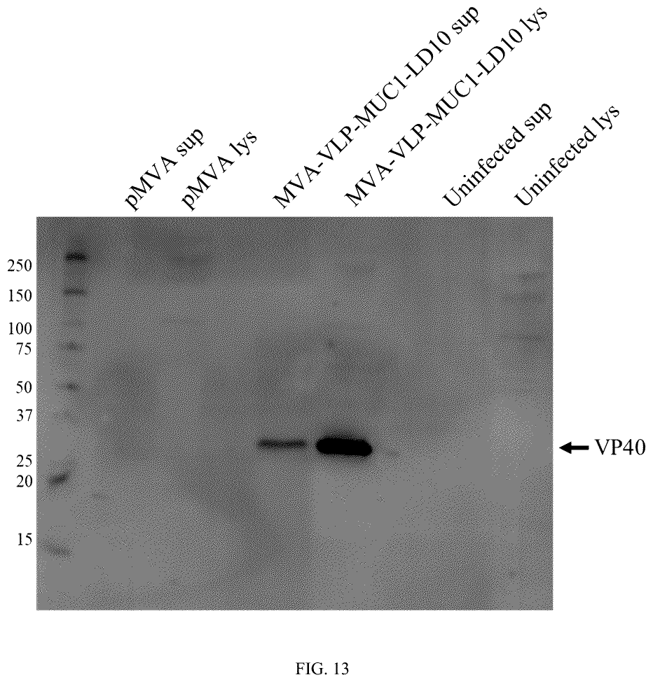

Transferred membranes were similarly probed with VP40 antibody (rabbit polyclonal, IBT Bioservices #0303-001, 1:1000). The expected size of recombinant VP40 protein is 32 kDa. Robust expression of VP40 protein was observed in MVA-VLP-MUC-1-LD10 cellular supernatant and lysate, suggesting that VP40 is expressed and also secreted in cells infected with the recombinant MVA virus encoding VLP-MUC-1-LD10 (FIG. 13).

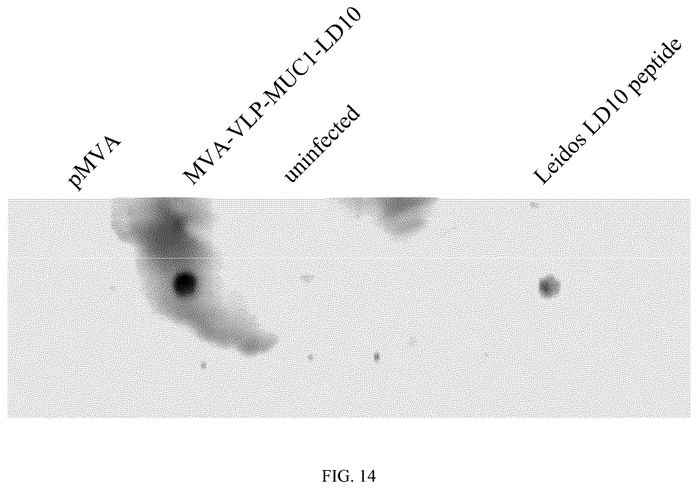

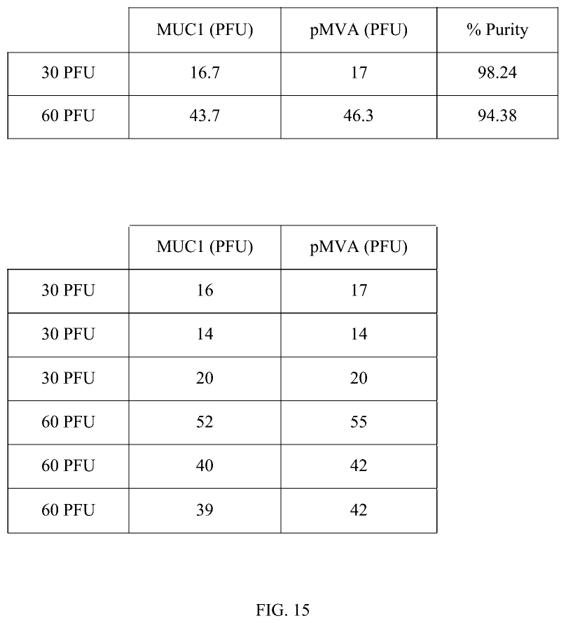

To confirm expression of LD10 peptide, a dot blot was performed on infected cell lysates. As a positive control, 20 ng of a Leidos LD10 peptide was included. The membrane was probed with LD10 antibody (mouse, Leidos 014, 7G10). Labeling of peptide and the MVA-VLP-MUC_1-LD10 sample confirmed LD10 expression in MVA-VLP-MUC-1-LD10-infected cells (FIG. 14).

### Example 16. Establishing MVA Vaccine Purity of DF-1 Cells Infected with MVA-VLP-MUC-1-LD10

DF1 cells were infected in technical triplicate with 30 plaque forming units (PFU) of virus, and separately, in technical triplicate with 60 PFU of virus in a 6-well plate. All wells were probed with MUC-1 antibody (mouse monoclonal VU4H5, Santa Cruz #sc-7313, 1:200) and the number of plaques were counted (FIG. 15). The wells were washed before being probed again with MVA antibody and MVA positive plaques were counted. The percentage of MUC1 plaques versus the number of MVA plaques was calculated to observe purity of the vaccine. Approximately 95% or greater MVA-positive plaques were also positive for MUC-1 expression at both infection quantities.

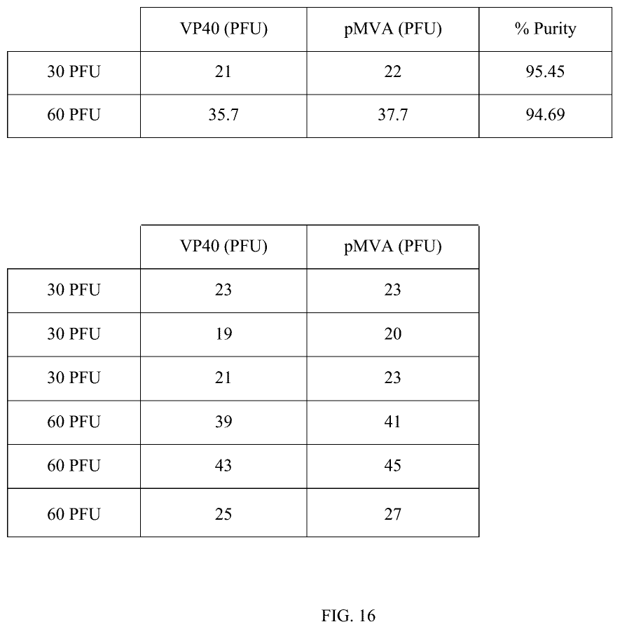

DF1 cells were infected in technical triplicate with 30 PFU of virus, and separately, in technical triplicate with 60 PFU of virus in a 6-well plate. All wells were probed with VP40 antibody (rabbit polyclonal, IBT Bioservices #0303-001, 1:1000) and the number of plaques were counted (FIG. 16). The wells were washed before being probed again with MVA antibody and MVA positive plaques were counted. The percentage of VP40 plaques vs the number of MVA plaques was calculated to observe purity of the vaccine. Approximately 95% or greater MVA-positive plaques were also positive for VP40 expression at both infection quantities.

What is claimed is:

1. An rMVA viral vector comprising an immune checkpoint inhibitor peptide comprising an amino acid sequence selected from the group consisting of SEQ ID NOs: 1-15, or an amino acid sequence at least 95% identical thereto.

2. The rMVA of claim 1, wherein the immune checkpoint inhibitor peptide comprises the amino acid sequence of SEQ ID NO: 1, or an amino acid sequence at least 95% identical thereto.

3. The rMVA of claim 2, wherein the immune checkpoint inhibitor peptide comprises the amino acid sequence of SEQ ID NO: 1,

4. The rMVA of claim 1, wherein the immune checkpoint inhibitor peptide comprises the amino acid sequence of SEQ ID NO: 5, or an amino acid sequence at least 95% identical thereto.

5. The rMVA of claim 4, wherein the immune checkpoint inhibitor peptide comprises the amino acid sequence of SEQ ID NO: 5.

6. An rMVA viral vector comprising a heterologous, polycistronic nucleic acid, wherein the polycistronic nucleic acid encodes ((M)(Secretion Signal Peptide-Immune Checkpoint Inhibitor Peptide-Cleavable Peptide)x(Secretion Signal Peptide-Immune Checkpoint Inhibitor Peptide)), wherein x=1-10, wherein M is methionine, and wherein the Immune Checkpoint Inhibitor comprises an amino acid sequence selected from the group consisting of SEQ ID NOS: 1-15, or an amino acid sequence at least 95% identical thereto.

7. The rMVA of claim 6, wherein the immune checkpoint inhibitor peptide comprises the amino acid sequence of SEQ ID NO: 1, or an amino acid sequence at least 95% identical thereto.

8. The rMVA of claim 7, wherein the immune checkpoint inhibitor peptide comprises the amino acid sequence of SEQ ID NO: 1.

9. The rMVA of claim 6, wherein the immune checkpoint inhibitor peptide comprises the amino acid sequence of SEQ ID NO: 5, or an amino acid sequence at least 95% identical thereto.

10. The rMVA of claim 9, wherein the immune checkpoint inhibitor peptide comprises the amino acid sequence of SEQ ID NO: 5.

11. The rMVA of claim 6, wherein the secretion signal peptide comprises an amino acid sequence selected from the group consisting of SEQ ID NOs: 57-90, or an amino acid sequence at least 95% identical thereto.

12. The rMVA of claim 11, wherein the secretion signal peptide comprises an amino acid sequence selected from SEQ ID NO: 65, or an amino acid sequence at least 95% identical thereto.

13. The rMVA of claim 11, wherein the secretion signal peptide comprises an amino acid sequence selected from SEQ ID NO: 66, or an amino acid sequence at least 95% identical thereto.

14. The rMVA of claim 6, wherein the cleavable peptide comprises an amino acid sequence selected from the group consisting of SEQ ID NOs: 91-127, or an amino acid sequence at least 95% identical thereto.

15. The rMVA of claim 14, wherein the cleavable peptide comprises an amino acid sequence selected from the group consisting of SEQ ID NOs: 93, 120, and 123, or an amino acid sequence at least 95% identical thereto.

16. The rMVA of claim 14, wherein the cleavable peptide comprises an amino acid sequence of SEQ ID NO: 93.

17. The rMVA of claim 14, wherein the cleavable peptide comprises an amino acid sequence SEQ ID NO: 120.

18. The rMVA of claim 14, wherein the cleavable peptide comprises an amino acid sequence SEQ ID NO: 123.

19. The rMVA of claim 6, wherein the polycistronic nucleic acid encodes an amino acid sequence selected from the group consisting of SEQ ID NOs: 309-324, or an amino acid sequence at least 95% identical thereto.

20. The rMVA of claim 6, wherein x=3, 4, or 5.

21. The rMVA of claim 6, wherein the polycistronic nucleic acid encodes an amino acid sequence selected from the group consisting of SEQ ID NOs: 325-340, or an amino acid sequence at least 95% identical thereto.

22. The rMVA of claim 6, wherein the polycistronic nucleic acid encodes an amino acid sequence selected from the group consisting of SEQ ID NOs: 341-344, or an amino acid sequence at least 95% identical thereto.

23. The rMVA of claim 6, wherein the polycistronic nucleic acid encodes an amino acid sequence selected from SEQ ID NOs: 345-348, or an amino acid sequence at least 95% identical thereto.

24. The rMVA of claim 21, wherein the polycistronic nucleic acid encodes the amino acid sequence of SEQ ID NO: 325, or an amino acid sequence at least 95% identical thereto.

25. The rMVA of claim 21, wherein the polycistronic nucleic acid encodes the amino acid sequence of SEQ ID NO: 329, or an amino acid sequence at least 95% identical thereto.

26. The rMVA of claim 21, wherein the polycistronic nucleic acid encodes the amino acid sequence of SEQ ID NO: 333, or an amino acid sequence at least 95% identical thereto.

27. The rMVA of claim 21, wherein the polycistronic nucleic acid encodes the amino acid sequence of SEQ ID NO: 337, or an amino acid sequence at least 95% identical thereto.

28. The rMVA of claim 6, wherein the polycistronic nucleic acid further encodes an antigenic peptide.

29. The rMVA of claim 28, wherein the antigenic peptide is derived from an amino acid sequence selected from the group consisting of SEQ ID NOs: 349-357, or an amino acid sequence at least 95% identical thereto.

30. The rMVA of claim 28, wherein the antigenic peptide is derived from an amino acid sequence selected from the group consisting of SEQ ID NOs: 358-394, or an amino acid sequence at least 95% identical thereto.

31. An rMVA viral vector comprising an amino acid sequence of SEQ ID NO: 332, or an amino acid sequence at least 95% identical thereto.

32. The rMVA viral vector of claim 31, comprising an amino acid sequence of SEQ ID NO: 332.

33. An rMVA viral vector comprising an amino acid sequence of SEQ ID NO: 337, or an amino acid sequence at least 95% identical thereto.

34. The rMVA viral vector of claim 33, comprising an amino acid sequence of SEQ ID NO: 337.
